SEQUENCE LISTING (1 sequence)

SEQ ID NO: 1 - protein, 566 aa, Influenza A virus (A/Netherlands/602/2009(H1N1))
  Hemagglutinin
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

(19) United States
(12) Patent Application Publication
(10) Pub. No.: US 2023/0295097 A1
(43) Pub. Date: Sep. 21, 2023

(54) LIPIDIC COMPOUNDS COMPRISING AT LEAST ONE TERMINAL RADICAL OF FORMULA -NH-CX-A OR -NH-CX-NH-A, COMPOSITIONS CONTAINING THEM AND USES THEREOF

(71) Applicant: SANOFI PASTEUR, Lyon (FR)

(72) Inventors: Jean HAENSLER, Paris (FR); Luc EVEN, Paris (FR); Benoît FRISCH, Paris (FR); Manon RIPOLL, Paris (FR)

(21) Appl. No.: 18/016,625

(22) PCT Filed: Jul. 16, 2021
(86) PCT No.: PCT/EP21/70026

§ 371 Date: Jan. 17, 2023

(30) Foreign Application Priority Data
Jul. 17, 2020 (EP) 20305825.0

(51) Int. Cl. C07D 233/66; C07D 213/72; A61K 48/00; A61K 9/127; A61P 31/16

(52) U.S. Cl. CPC C07D 233/66; C07D 213/72; A61K 48/0008; A61K 9/1271; A61P 31/16; A61K 2039/51

(57) ABSTRACT

The disclosure relates to novel lipidic compounds, lipid nanoparticles (LNPs) containing thereof, and the use of the lipidic compounds or the LNPs for the delivery of nucleic acid. The lipidic compounds as disclosed herein comprise at least one terminal radical of formula (I): *—NH—CX—(NH)n-A (I) wherein: —*- represents a single bond linking said radical of formula (I), directly or not, to to one C₁₀ to C₅₅ lipophilic or hydrophobic tail-group; —n is 0 or 1; —X is an oxygen or sulfur atom, and —A represents an optionally substituted 5- or 6-membered unsaturated heterocyclic radical or 5- or 6-membered heteroaromatic ring radical, both containing at least one nitrogen atom; or one of the pharmaceutically acceptable salts of said radical of formula (I); and with said compound that is in all the possible racemic, enantiomeric and diastereoisomeric isomer forms.

### TECHNICAL FIELD

The present disclosure is in the field of novel lipidic compounds which can be used to form lipid nanoparticles for delivery of therapeutic agents, such as nucleic acid, for example in combination with other lipid components, such as neutral lipids, steroid alcohols or esters thereof and polymer conjugated lipids. For example, the formulations prepared with the lipidic compounds as disclosed herein enable to induce an immune response after administration of antigen-coding polynucleotides.

### TECHNICAL BACKGROUND

The polynucleotide therapeutics field has seen remarkable progress over the recent years. Polynucleotides include various nucleic acids-based compounds such as messenger RNA (mRNA), antisense oligonucleotides, ribozymes, DNAzymes, plasmids, or immune stimulating nucleic acids. Some nucleic acids, such as mRNA, plasmids and ssDNA can be used to induce the expression of specific cellular products useful in the treatment of, for example, diseases related to a deficiency of a protein or enzyme, or for the expression of a vaccine antigen to induce specific immune responses. The therapeutic applications of translatable nucleotide delivery are extremely broad as constructs can be synthesized to produce any chosen protein sequence, whether or not indigenous to the system. The expression products of the nucleic acid can augment existing levels of protein, replace missing or non-functional versions of a protein, or introduce new protein and associated functionality in a cell or organism or expose to a foreign protein in order to induce a specific immune response.

However, there are many challenges associated with the delivery of polynucleotides to affect a desired response in a biological system and the effective delivery of polynucleotides to their intracellular sites of action remains a major issue. To be efficiently delivered to their site of action, the polynucleotides must be (i) protected from enzymatic and non-enzymatic degradation, (ii) appropriately distributed in the biologic compartment of interest, (iii) effectively and efficiently internalized by the targeted cells, and then (iv) delivered to the intracellular compartment where the relevant translation machinery resides.

Lipid nanoparticles formed from cationic lipids formulated with other lipid components, such as neutral lipids, cholesterol, and PEGylated lipids have been used to protect the polynucleotide from degradation and facilitate its cellular uptake.

While lipid nanoparticles-based vehicles that comprise a cationic lipid component have shown promising results with regards to encapsulation, stability and site localization, there remains a great need for improvement of lipid nanoparticles-based delivery systems.

There remains a need for improved cationic and ionizable lipids that demonstrate improved pharmacokinetic properties, and which are capable of delivering various types of polynucleotides to a wide variety cell types and tissues with enhanced efficiency. Importantly, there also remains a need for novel cationic ionizable lipids having reduced toxicity and are capable of efficiently delivering encapsulated polynucleotides to targeted cells, tissues and organs. Improved cationic lipids and lipid nanoparticles for the delivery of polynucleotides would also provide optimal polynucleotide/lipid ratios, protect the polynucleotides from degradation and clearance in serum, be suitable for systemic or local delivery, and provide intracellular delivery of the polynucleotide. In addition, the lipid-polynucleotide particles should be well-tolerated and provide an adequate therapeutic index, such that patient treatment at an effective dose of the polynucleotide is not associated with unacceptable toxicity and/or risk to the patient. In addition, the lipid-nucleic acid particles should be stable as liquid formulations when stored at 4-8° C. for long periods of time in a pharmaceutically acceptable buffer.

The present disclosure provides these and related advantages.

### SUMMARY OF THE DISCLOSURE

Accordingly, one of the objects of the present disclosure relates to a cationic and/or ionizable lipidic compound comprising at least one terminal radical of formula (I):

*—NH—CX—(NH)ₙ-A (I)
wherein:
*- represents a single bond linking said radical of formula (I), directly or not, to one C₁₀ to C₅₅ lipophilic or hydrophobic tail-group;
n is 0 or 1;
X is an oxygen or a sulfur atom;
A represents an optionally substituted 5- or 6-membered unsaturated heterocyclic radical or 5- or 6-membered heteroaromatic ring radical both containing at least one nitrogen atom;
   or one of the pharmaceutically acceptable salts of said radical of formula (I); and with said lipidic compound that is in all the possible racemic, enantiomeric and diastereoisomeric isomer forms.

For example, the lipidic compounds of the instant disclosure are cationic lipids. Another object of the disclosure relates to one compound of formula (II):

R1-Z—NH—CX—(NH)ₙ-A (II)
wherein:
X, n and A are as defined in formula (I);
R1 is a C₁₀ to C₅₅ lipophilic or hydrophobic tail-group;
   —Z is a spacer arm having from 2 to 24, for example from 2 to 18, for example from 4 to 12 carbon atoms in a branched or unbranched linear, saturated or unsaturated, hydrocarbon chain, said chain that is interrupted by one or several atoms of oxygen and/or moieties selected among —S—S—; (C═O)—O; —O(O═C)—; —S—; —NH—, —NH—(O═C)—; —(O═C)—NH— and —NH—(C═O)—O— and/or ended by an oxygen atom or a moiety selected among —NH—(O═C)—O—(O═C)— and —(O═C)— to be linked to the hydrophobic tail-group.
p is O or 1; and
or one of the pharmaceutically acceptable salts of said compound of formula (II); and any of its racemic, enantiomeric and diastereoisomeric isomer forms.

According to one embodiment, the compounds of formula (II) are selected in the group consisting of the following compounds. In remark, in the following developed formula, a secondary amino moiety may be indifferently written —NH— or —N—.

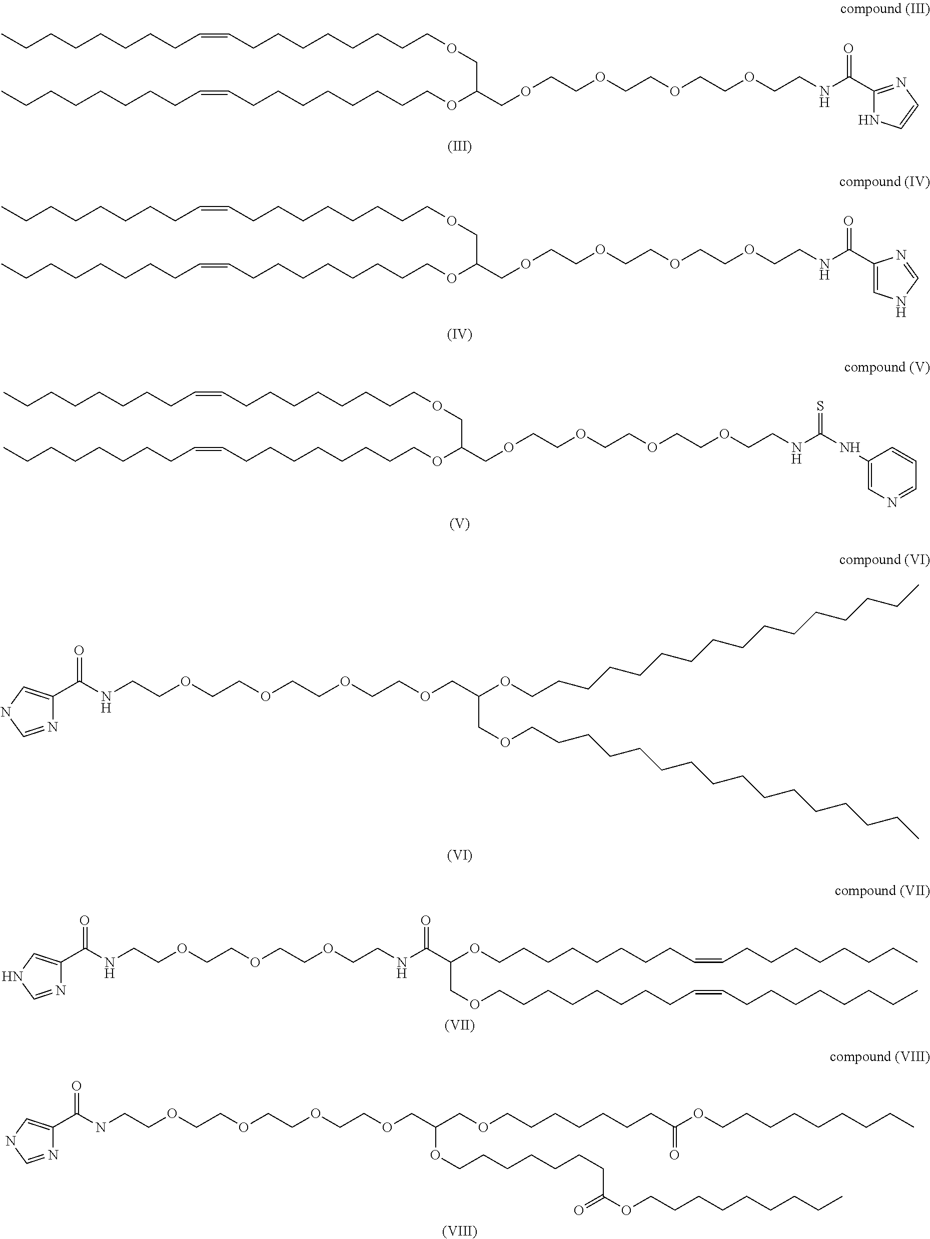

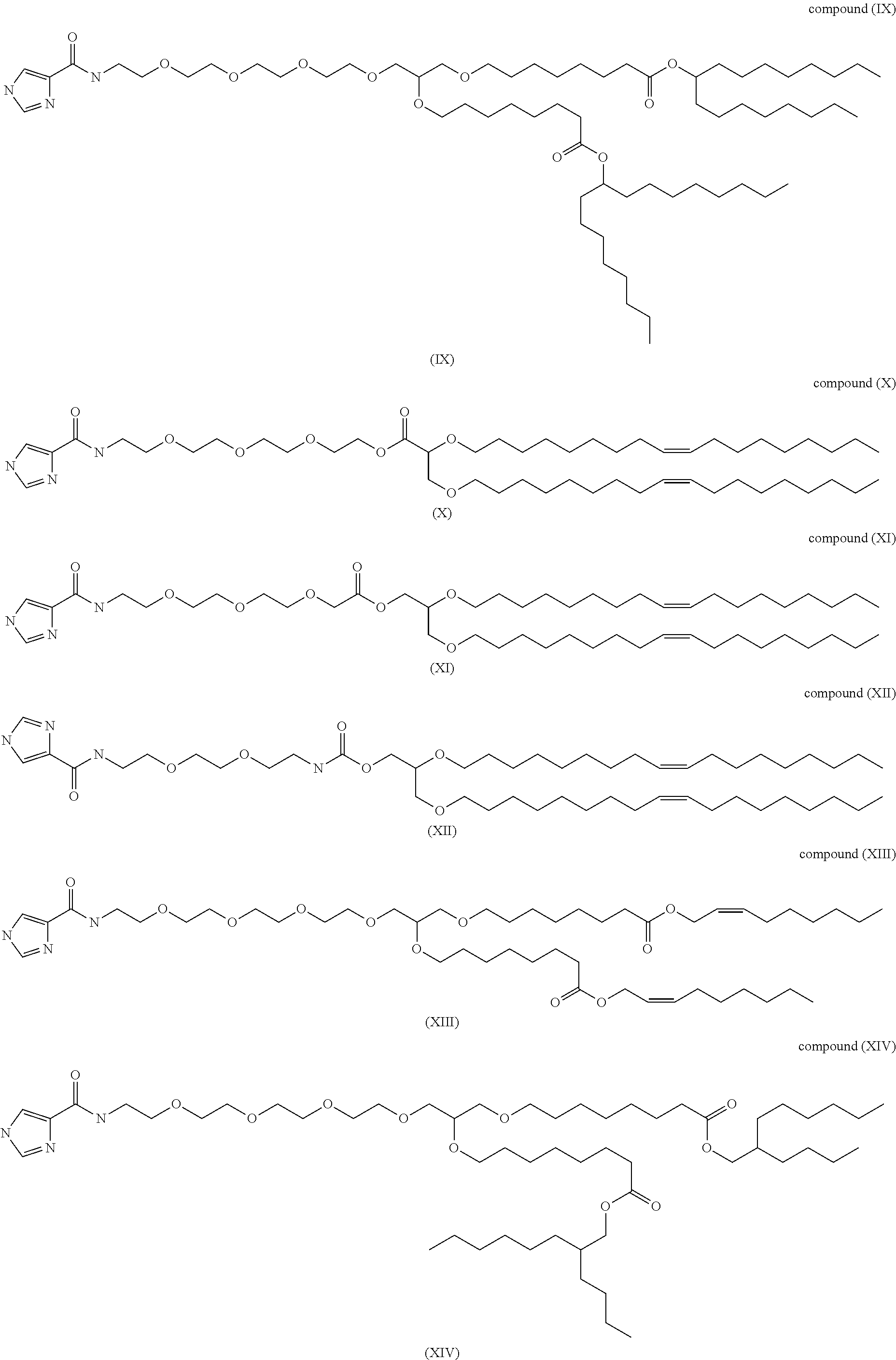

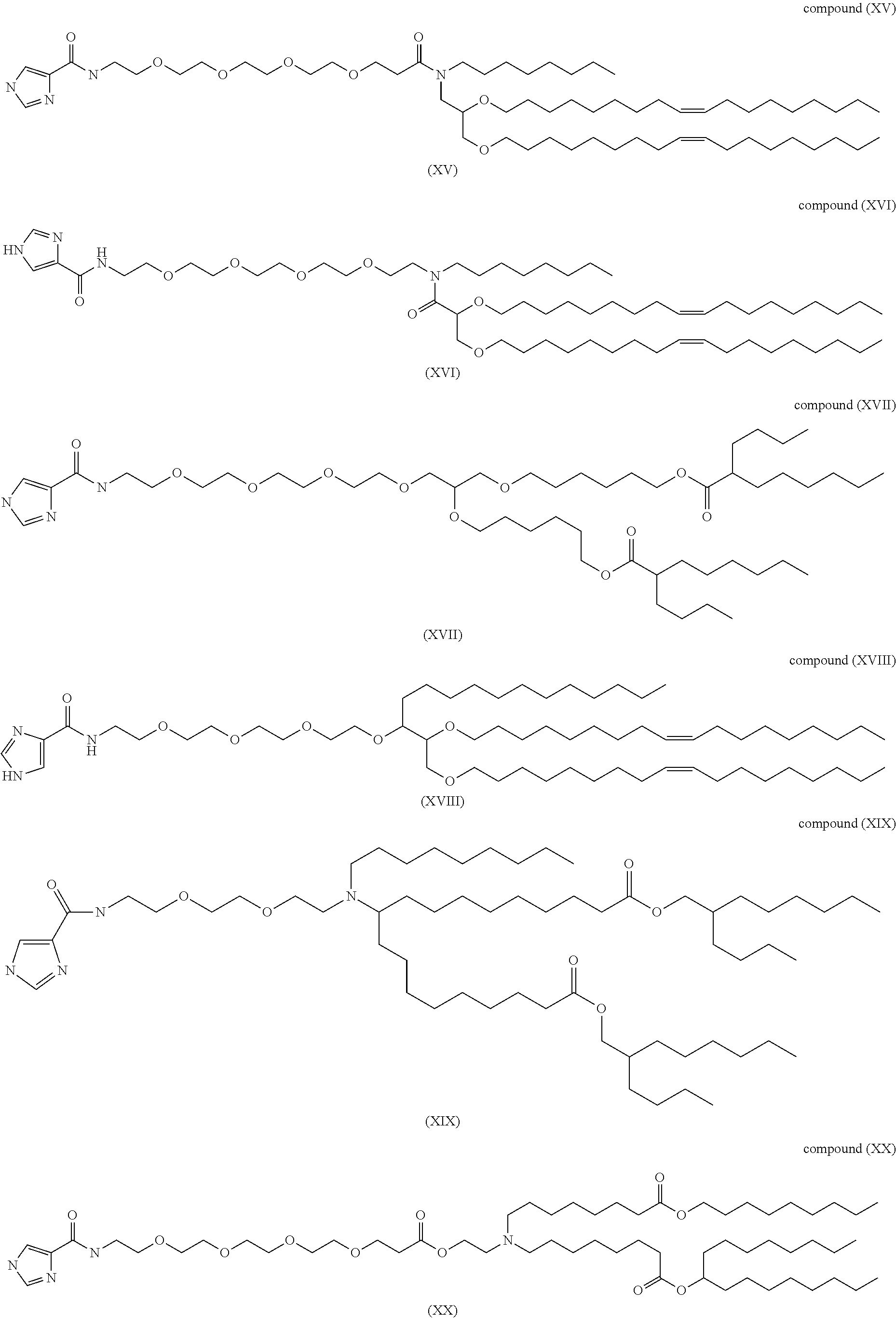

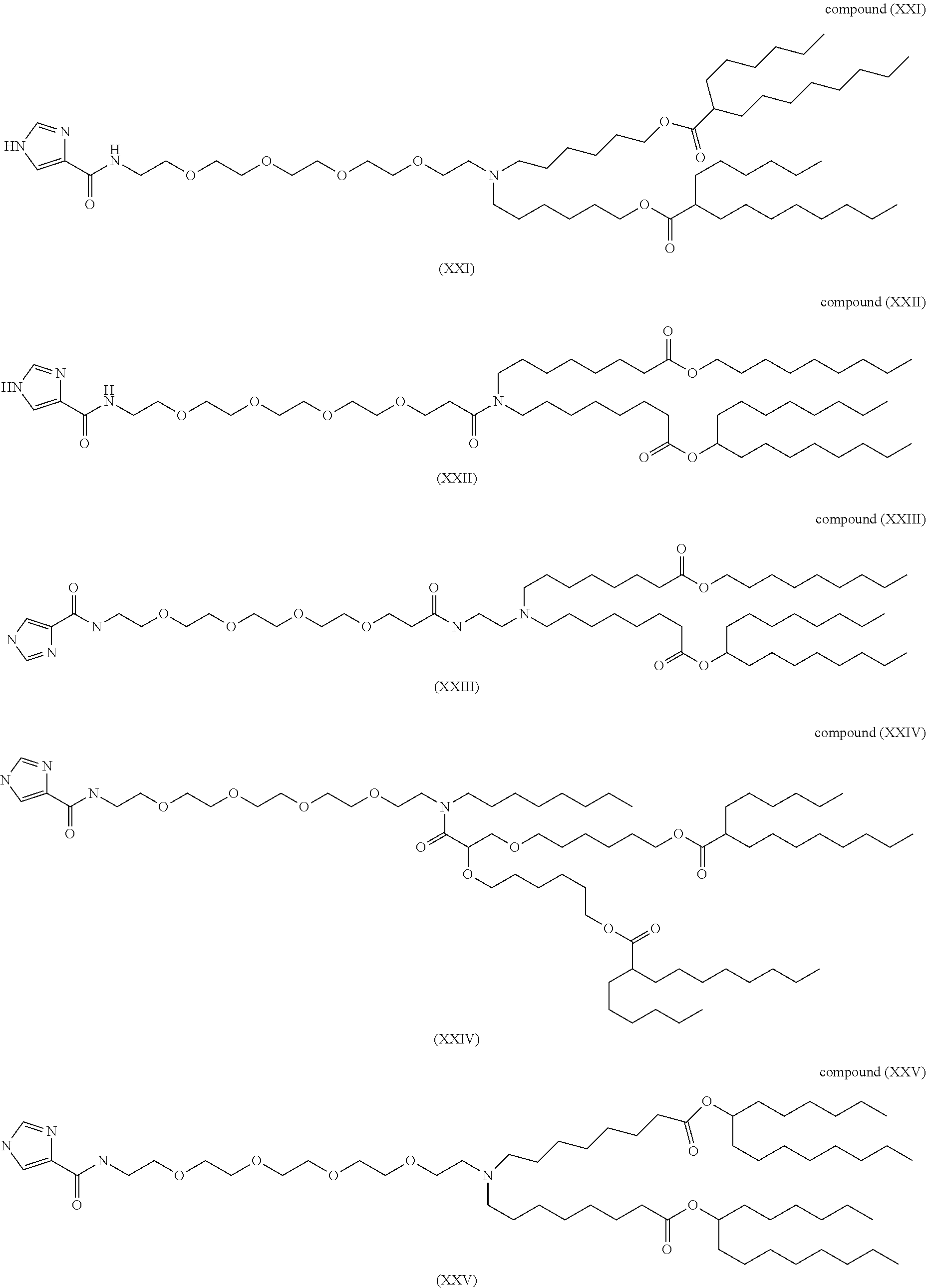

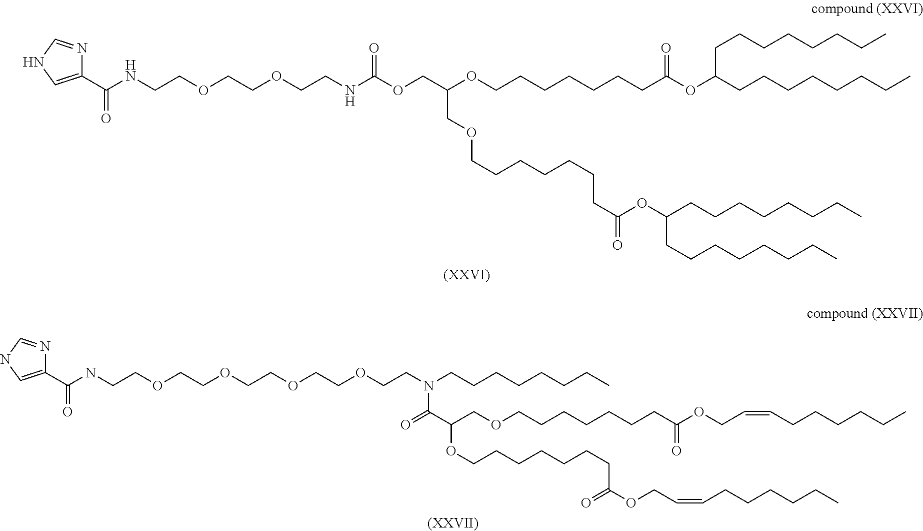

and their pharmaceutically acceptable salts, and their racemic, enantiomeric and diastereoisomeric isomer forms.

For example, the compound of formula (II) may be any one of the compounds (III), (IV), (V), (VIII), (IX), (XII), (XVI), (XIX), or (XXII), or any one of the compounds (IV), (VIII), (IX), (XII), (XVI), (XIX), or (XXII), or any one of the compounds (IV), (IX), (XII), or (XVI), or any one of the compound (IV) or (XII), or for example of formula (III), (IV) or (V), or the compound (IV) (also named DOG-IM4), or one of their salts or racemic, enantiomeric and diastereoisomeric isomer forms.

Surprisingly, and as detailed in the Examples section, the inventors have observed that the novel lipidic compounds as disclosed herein enable the formulation of improved compositions, such as lipid nanoparticles, for the in vitro and in vivo delivery of mRNA and/or other oligonucleotides or oligonucleotides. Furthermore, the so-formed compositions may also be stored under a stabilized liquid form at a temperature ranging from 4 to 8° C.

As shown in the Examples sections, the lipid nanoparticles of the invention have proven to be very stable at 5° C., 25° C. and even 37° C. in terms of pH, osmolality, particle size, mRNA encapsulation, and/or mRNA integrity. This strong stability allows versatile applications of the lipid nanoparticles of the invention. For example, they may allow storage of pharmaceutical compositions, such as vaccines, at room temperature instead of cold temperatures.

The improved lipid nanoparticles are useful for expression of protein encoded by mRNA. The lipid nanoparticles as disclosed herein may be used for regulation, up-regulation or down-regulation, of protein expression by delivering either miRNA or miRNA inhibitors for modulating expression of endogenous protein, or mRNA or plasmids for expression of transgenes. Also, the lipid nanoparticles as disclosed herein may be used for inducing a pharmacological effect resulting from expression of a protein or a protection against infection through delivery of mRNA encoding for a suitable antigen or antibody.

Also, the lipid nanoparticles as disclosed herein may be used for inducing a pharmacological effect resulting from expression of a protein, such as erythropoietin, useful for the treatment of metabolic diseases or diseases resulting from protein deficiency.

Another object of the disclosure relates to a composition comprising at least one lipidic compound as disclosed herein and at least one lipid selected from the group consisting of neutral lipids, steroid alcohols or esters thereof, and PEGylated lipids.

Another object of the disclosure relates to a lipid nanoparticle comprising at least one lipidic compound as disclosed herein and at least one nucleic acid.

Another object of the disclosure relates to a pharmaceutical composition comprising (i) at least one nucleic acid and at least one lipidic compound as disclosed herein, or (ii) at least one nucleic acid at least one composition as disclosed herein, or (iii) at least one lipid nanoparticle as disclosed herein.

A pharmaceutical composition as disclosed herein may be an immunogenic composition. Therefore, another object of the disclosure relates to an immunogenic composition comprising (i) at least one nucleic encoding for an antigen and at least one lipidic compound as disclosed herein, or (ii) at least one nucleic encoding for an antigen and at least one composition as disclosed herein, or (iii) at least one lipid nanoparticle as disclosed herein wherein the nucleic acid encodes for at least one antigen.

Another object of the disclosure relates to a composition comprising (i) at least one nucleic acid and at least one lipidic compound as disclosed herein, or (ii) at least one nucleic acid and at least one composition as disclosed herein, or (iii) at least one lipid nanoparticle as disclosed herein, for use as a medicament.

Another object of the disclosure relates to a composition comprising (i) at least one nucleic acid and at least one lipidic compound as disclosed herein, or (ii) at least one nucleic acid and at least one composition as disclosed herein, or (iii) at least one lipid nanoparticle as disclosed herein, for use in a therapeutic method for preventing and/or treating a disease selected in a group consisting of infectious diseases, allergies, autoimmune diseases, rare blood disorders, rare metabolic diseases, rare neurologic diseases, and tumor or cancer diseases.

The term “rare diseases” is used herein according to its meaning acknowledge in the art to mean diseases with an average prevalence threshold between 40 and 50 cases/100,000 people (Richter et al., Value Health. 2015 September; 18(6):906-14).

Another object of the disclosure relates to a composition comprising (i) at least one nucleic acid encoding for an antigen and at least one lipidic compound as disclosed herein, or (ii) at least one nucleic acid encoding for an antigen and at least one composition as disclosed herein, or (iii) at least one lipid nanoparticle as disclosed herein, for use as an immunogenic composition.

In some embodiments, the disclosure also relates to a use of a composition comprising (i) at least one nucleic acid encoding for an antigen and at least one lipidic compound as disclosed herein, or (ii) at least one nucleic acid encoding for an antigen and at least one composition as disclosed herein, or (iii) at least one lipid nanoparticle as disclosed herein for the manufacture of a medicament for preventing and/or treating infectious diseases, allergies, autoimmune diseases, rare blood disorders, rare metabolic diseases, rare neurologic diseases, and tumour or cancer diseases.

Another object of the disclosure relates to a method of preventing and/or treating a disease in an individual in need thereof, wherein the method comprises administering an effective amount of (i) at least one nucleic acid and at least one lipidic compound as disclosed herein, or (ii) at least one nucleic acid and at least one composition as disclosed herein, or (iii) at least one lipid nanoparticle as disclosed herein, to said individual. A method as disclosed herein may be for preventing and/or treating infectious diseases, allergies, autoimmune diseases, rare blood disorders, rare metabolic diseases, rare neurologic diseases, and tumour or cancer diseases.

Another object of the disclosure relates to a method of transfecting at least one isolated target cell with a nucleic acid, wherein said method comprises contacting the at least one target cell with an effective amount of (i) at least one nucleic acid and at least one lipidic compound as disclosed herein, or (ii) at least one nucleic acid and at least one composition as disclosed herein, or (iii) at least one lipid nanoparticle as disclosed herein, such that the at least one target cell are transfected with said nucleic acid.

Another object of the disclosure relates to a method of producing a polypeptide in at least one target cell, wherein said method comprises contacting the at least one target cell with an effective amount of (i) at least one nucleic acid encoding said polypeptide and at least one compound as disclosed herein, or (ii) at least one nucleic acid encoding said polypeptide and at least one composition as disclosed herein, or (iii) at least one lipid nanoparticle as disclosed herein wherein the nucleic acid encodes said polypeptide, such that the at least one target cell are transfected with a nucleic acid operably encoding said polypeptide.

Another object of the disclosure relates to a method for manufacturing nucleic acid charged lipid nanoparticles, wherein the method comprises at least the steps of:
a) solubilizing, in a water miscible organic solvent, at least one lipidic compound as disclosed herein,
b) mixing the organic solvent obtained at step a) with at an aqueous solvent comprising at least one nucleic acid to be charged, and
c) obtaining said lipid nanoparticles in the aqueous solvent.

In the description of the various embodiments of the present disclosure, various embodiments or individual features are disclosed. As will be apparent to the ordinarily skilled practitioner, all combinations of such embodiments and features are possible and can result in executions of the present disclosure. While various embodiments and individual features of the present disclosure have been illustrated and described, various other changes and modifications can be made without departing from the spirit and scope of the disclosure. As will also be apparent, all combinations of the embodiments and features taught in the foregoing disclosure are possible and can result in executions of the disclosure.

### DESCRIPTION OF THE FIGURES

FIG. 1: Hemagglutination inhibiting antibody mean titers (HI titers) measured in serum from mice post-1 immunization (at D20) with LNPs L319, LNPs Lip. (III), LNPs Lip. (IV) or LNPs Lip. (V) (manufactured with lipidic compound of formula (III), (IV) or (V)) each loaded with mRNA encoding full-length hemagglutinin (HA) of influenza virus strain A/Netherlands/602/2009 (H1N1). Total injected mRNA is 0.5, 1, 2.5 or 5.0 μg/dose for LNPs L319 and 1 or 5 μg/dose for LNPs Lip. (III), LNPs Lip. (IV) and LNPs Lip. (V). As negative control group, mice were immunized with PBS buffer and as positive control group, mice received 10 μg of monovalent Flu vaccine A/California/07/2009 (H1N1) strain derived from Vaxigrip™. Geometric mean titers and individual HI titers are indicated for each group.

FIG. 2: Hemagglutination inhibiting antibody mean titers (HI titers) measured in serum from mice post-2 immunization (D42) with LNPs L319, LNPs Lip. (III), LNPs Lip. (IV) or LNPs Lip. (V) (manufactured with lipidic compound of formula (III), (IV) or (V)) each loaded with mRNA encoding full-length hemagglutinin (HA) of influenza virus strain A/Netherlands/602/2009 (H1N1). Total injected mRNA is 0.5, 1, 2.5 or 5.0 μg/dose for LNPs L319 and 1 or 5 μg/dose for LNPs (III), LNPs (IV) and LNPs (V). As negative control group, mice were immunized with PBS buffer and as positive control group, mice received 10 μg of monovalent Flu vaccine A/California/07/2009 (H1N1) strain derived from Vaxigrip™. Geometric mean titers and individual HI titers are indicated for each group.

FIG. 3: Hemagglutination inhibiting antibody mean titers (HI titers) measured in sera collected at D42 in mice immunized at DO and D21 with different LNPs L319 and LNPs Lip. (IV) containing DOPE as neutral lipid and each loaded with mRNA encoding full-length hemagglutinin (HA) of influenza virus strain A/Netherlands/602/2009 (H1N1). mRNAs loaded in LNPs Lip. (IV) were containing either natural (Nat) or modified uridine base (Mod). mRNAs loaded in LNPs L319 was containing natural uridine base. Geometric mean titers and individual HI titers are indicated for each group.

FIG. 4: Hemagglutination inhibiting antibody mean titers (HI titers) measured in sera collected at D42 in mice immunized at DO and D21 with different LNPs L319 and LNPs Lip. (IV). LNPs Lip. (IV) were containing either DSPC or DOPE as neutral lipid. LNP L319 was containing DSPC as neutral lipid. LNPs were loaded with mRNA encoding full-length hemagglutinin (HA) of influenza virus strain A/Netherlands/602/2009 (H1N1) containing natural uridine base. Geometric mean titers and individual HI titers are indicated for each group.

FIG. 5: Hemagglutination inhibiting antibody mean titers (HI titers) measured in sera collected at D42 in mice immunized at D0 and D21 with LNPs Lip. (IV) containing DSPC as neutral lipid and loaded with mRNA encoding full-length hemagglutinin (HA) of influenza virus strain A/Netherlands/602/2009 (H1N1) containing natural uridine base. LNPs were stored for different period of time before use: 0, 6 and 12 months. Three independent experiments carried out over time covering a period of one year. Geometric mean titers and individual HI titers are indicated for each group.

FIG. 6: Bioluminescence signal acquisition monitoring protein expression in injected site (quadriceps) following intramuscular administration of LNPs 319 or LNPs Lip. (IV) loaded with 5 μg of mRNA encoding Luciferase (mRNA-Luc) in female BALB/c ByJ mice. The luminescence level was evaluated by an ROI applied to the injection site zone at 6 h, 24, 48 h and 72 h and the results are expressed as total flux (ph/s) in function of time (hours) post the injection of LNPs/mRNA-Luc. A buffer PBS was used as control.

FIG. 7: shows the scheme of synthesis of compound (XIII).

FIG. 8: shows the scheme of synthesis of compound (XIV).

FIG. 9: shows the scheme of synthesis of compound (XVII).

FIG. 10: shows the scheme of synthesis of compound (XXI).

FIG. 11: shows the scheme of synthesis of compound (XXII).

FIG. 12: chromatogram of LNPs Lip. (IV)/DSPC containing hEPO mRNA recorded as a function of time.

FIG. 13: shows the stability of pH of the LNPs Lip. (IV)/DSPC over time at different storage temperatures.

FIG. 14: shows the stability of osmolality of the LNPs Lip. (IV)/DSPC over time at different storage temperatures.

FIG. 15: shows the stability of particle sizes of the LNPs Lip. (IV)/DSPC over time at different storage temperatures.

FIG. 16: shows the stability of mRNA encapsulation rate of the LNPs Lip. (IV)/DSPC over time at different storage temperatures.

FIG. 17: shows the mRNA integrity in the LNPs Lip. (IV)/DSPC over time at different storage temperatures.

FIG. 18: shows the stability of lipid chromatogram for LNPs Lip. (IV)/DSPC over time at different storage temperatures. FIG. 18A: upper panel: shows the LNPs Lip.(IV)/DSPC after 18 weeks at 4° C.; lower panel shows the same LNPs at TO. FIG. 18B: upper panel: shows the LNPs Lip.(IV)/DSPC after 18 weeks at 25° C.; lower panel shows the same LNPs at T0. FIG. 18C: upper panel: LNPs Lip.(IV)/DSPC after 18 weeks at 37° C.; lower panel shows the same LNPs at T0.

FIG. 19: shows the stability of hEPO expression from LNPs Lip. (IV)/DSPC over time at different storage temperatures.

FIG. 20: shows the immunogenicity of LNPs comprising influenza HA mRNA on cynomolgus macaques immunized twice four weeks apart (D0, D28) with 50 μg of mRNA in LNPs injected IM into the biceps under a volume of 500 μl.

FIG. 21: Hemagglutination inhibiting antibody mean titers (HI titers) measured in sera collected at D21 in mice immunized at D0 and D21 with LNPs L319, LNPs Lip. (IV) [DOG-IM4], LNPs Lip. (IX), LNPs Lip. (XII) and LNPs Lip. (XVI) containing DSPC as neutral lipid and loaded with mRNA encoding full-length hemagglutinin (HA) of influenza virus strain A/Netherlands/602/2009 (H1N1).

### DETAILED DESCRIPTION

### Definitions

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them. The following definitions are provided for the present specification, including the claims.

The term “terminal radical” means that said radical is a head-group or a tail-group.

The term “pharmaceutically acceptable salts” includes addition salts of compounds as disclosed herein derived from the combination of such compounds with for example non-toxic acid addition salts.

The term “acid addition salts” include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

The pharmaceutically acceptable salts of compounds as disclosed herein can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present disclosure.

In the context of the present disclosure the chemical terms below have the following meanings:
a halogen atom: a fluorine, a chlorine, a bromine or an iodine;
Ct-Cz: a carbon chain that can have from t to z carbon atoms, where t and z may have the values from 1 to 7; for example, C₁-C₄ is a carbon chain that may have from 1 to 4 carbon atoms;
C₁-C₄ alkyl as used herein respectively refers to C₁-C₄ normal, secondary or tertiary saturated hydrocarbon. Non limiting examples are methyl, ethyl, propyl, isopropyl, butyl, isobutyl or tertbutyl;
C₁-C₄ alkoxy is intended to mean an —O—(C₁-C₄)alkyl radical where the C₁-C₄ alkyl group is as defined above. Non limiting examples are methoxy, ethoxy, propoxy, isopropoxy, butoxy, sec-butoxy or tert-butoxy;
a heteroatom is understood to mean nitrogen, oxygen or sulphur;
an heteroaromatic ring denotes a 5- or 6-membered aromatic ring comprising 1 or 2 heteroatoms;
an aromatic ring refers to a mono or polycyclic, for example a monocyclic, aromatic hydrocarbon radical of 6-20 atoms, for example 6 atoms, derived by the removal of one hydrogen from a carbon atom of a parent aromatic ring system. An aromatic ring as disclosed herein is for example a phenyl group;
when n is 0 in formula (I) of the present disclosure, it means that the moiety —NH is not present.

As used in this specification and the appended claims, the singular forms “a”, “an” and “the” include plural referents unless the content clearly dictates otherwise.

The term “about” or “approximately” as used herein refer to the usual error range for the respective value readily known to the skilled person in this technical field. Reference to “about” a value or parameter herein includes (and describes) embodiments that are directed to that value or parameter per se. In some embodiments, the term “about” refers to ±10% of a given value. However, whenever the value in question refers to an indivisible object, such as a nucleotide or other object that would lose its identity once subdivided, then “about” refers to ±1 of the indivisible object.

The term “antigen” comprises any molecule, for example a peptide or protein, which comprises at least one epitope that will elicit an immune response and/or against which an immune response is directed. For example, an antigen is a molecule which, optionally after processing, induces an immune response, which is for example specific for the antigen or cells expressing the antigen. After processing, an antigen may be presented by MHC molecules and reacts specifically with T lymphocytes (T cells). Thus, an antigen or fragments thereof should be recognizable by a T cell receptor and should be able to induce in the presence of appropriate co-stimulatory signals, clonal expansion of the T cell carrying the T cell receptor specifically recognizing the antigen or fragment, which results in an immune response against the antigen or cells expressing the antigen.

According to the present disclosure, any suitable antigen may be envisioned which is a candidate for an immune response. An antigen may correspond to or may be derived from a naturally occurring antigen. Such naturally occurring antigens may include or may be derived from allergens, viruses, bacteria, fungi, parasites and other infectious agents and pathogens or an antigen may also be a tumor antigen.

As used herein, the term “aqueous solution” or “aqueous solvent” refers to a composition comprising water.

Within the disclosure the terms “cationic group” or “cationic ammonium group” refers to an ion or group of ions having a positive charge and comprising at least one ionizable nitrogen atom. The cationic group as disclosed herein is comprised of the radical of formula (I): —NH—CX—(NH)n-A as defined herein.

It is understood that aspects and embodiments of the present disclosure described herein include “having,” “comprising,” “consisting of,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the disclosure. Depending on the context, the term “comprise” may also specify strictly the stated features, integers, steps or components, and therefore in such case it may be replaced with “consist”.

The term “charged lipid” refers to any of a number of lipid species that exist in either a positively charged or negatively charged form within a useful physiological range e.g. pH ˜3 to pH ˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g. DOTAP, DOTMA), dialkyldimethylaminopropanes, ethyl phosphocholines, dimethylaminoethane carbamoyl sterols (e.g. DC-Choi).

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

The term “neutral lipid” refers to any of a number of lipid species that is either not ionizable or is a neutral zwitterionic compound at a selected pH, for example at physiological pH. Such lipids include, but are not limited to, phosphatidylcholines, phophatidylethanolamines sphingomyelins (SM), or ceramides. Neutral lipids may be synthetic or naturally derived.

As used herein, the term “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

The term “lipid” refers to a group of organic compounds that include, but are not limited to, esters of fatty acids and are generally characterized by being poorly soluble in water, but soluble in many organic solvents.

Lipid is a generic term encompassing fats, fatty oils, essential oils, waxes, phospholipids, glycolipids, sulfolipids, aminolipids, chromolipids (lipochromes), and fatty acids. Within the disclosure, “lipid” encompasses neutral lipids, steroid alcohol or ester thereof, and PEGylated lipids.

The term “lipid nanoparticle” (LNP) refers to particles having at least one dimension on the order of nanometers (e.g., 1-1 000 nm) which may be formulated with at least one of the lipidic compounds as disclosed herein. In some embodiments, lipid nanoparticles are included in a formulation that can be used to deliver an active agent or therapeutic agent, such as a nucleic acid to a target site of interest (e.g., cell, tissue, organ, tumor, and the like). Such lipid nanoparticles typically comprise a lipidic compound as disclosed herein and at least one ingredient selected from neutral lipids, steroid alcohols or esters thereof, and polymer conjugated lipids.

As used herein, “lipid encapsulated” refers to a lipid nanoparticle that provides an active agent or therapeutic agent, such as a nucleic acid with full encapsulation, partial encapsulation, or both. In an embodiment, the polynucleotide is fully encapsulated in the lipid nanoparticle.

It should be noted that the terms “head-group” and “tail-group” as used in the instant specification, describe parts of the compounds of the present disclosure, such as functional groups of such compounds. They are used to describe the orientation of one or more functional groups relative to other functional groups in said compounds. They are both “end group”.

As used herein, the terms “lipophilic or hydrophobic tail-group” indicate in qualitative terms that the tail the tail has an affinity for lipids (and typically is lipid-soluble) and is water-avoiding (and typically is not water soluble).

The term “PEGylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

Within the disclosure the term “nucleic acids”, “polynucleotide” and “oligonucleotides” are used interchangeably. They refer to a polymeric form of at least two nucleotides, either deoxyribonucleotides or ribonucleotides, or analogs thereof. Nucleic acids may have any three-dimensional structure, and may perform any function, known or unknown. They may be linear or cyclic. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, closed-ended DNA (ceDNA), self-amplifying RNA, stranded DNA (ssDNA), small interfering RNA (siRNA) and micro RNA (miRNA), recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “complement of a polynucleotide” denotes a polynucleotide molecule having a complementary base sequence and reverse orientation as compared to a reference sequence, such that it could hybridize with a reference sequence with complete fidelity. “Recombinant” as applied to a polynucleotide means that the polynucleotide is the product of various combinations of in vitro cloning, restriction and/or ligation steps, and other procedures that result in a construct that can potentially be expressed in a host cell.

The term “steroid alcohol” or “sterol” refers to a group of lipids comprised of a sterane core bearing a hydroxyl moiety. As example of steroid alcohol, one may cite cholesterol, campesterol, sitosterol, stigmasterol and ergosterol. Esters of steroid alcohol or of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments the alkyl group may be a C₁-C₂₀ alkyl group. In other embodiments, the carboxylic acid may be a fatty acid.

As used herein, the terms “prevent”, “preventing” or “delay progression of” (and grammatical variants thereof) with respect to a disease or disorder relate to prophylactic treatment of a disease, e.g., in an individual suspected to have the disease, or at risk for developing the disease. Prevention may include, but is not limited to, preventing or delaying onset or progression of the disease and/or maintaining at least one symptom of the disease at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein.

Within the disclosure, the term “significantly” used with respect to change intends to mean that the observe change is noticeable and/or it has a statistic meaning.

Within the disclosure, the term “substantially” used in conjunction with a feature of the disclosure intends to define a set of embodiments related to this feature which are largely but not wholly similar to this feature.

As used herein, “target cells” or “targeted cells” refer to cells of interest. The cells may be found in vitro, in vivo, in situ or in the tissue or organ of an organism. The organism may be an animal, for example a mammal, for example a human, and for example a human patient. In some embodiments, a target cell is a cell isolated from an individual.

The terms “treat” or “treatment” or “therapy” in the present text refers to the administration or consumption of a composition as disclosed herein with the purpose to cure, heal, alleviate, relieve, alter, remedy, ameliorate, improve, or affect a disorder, the symptoms of the condition, or to prevent or delay the onset of the symptoms, complications, or otherwise arrest or inhibit further development of the disorder in a statistically significant manner.

As used herein, the terms “therapeutically effective amount” and “prophylactically effective amount” refer to an amount that provides a therapeutic benefit in the treatment, prevention, or management of pathological processes considered. The specific amount that is therapeutically effective can be readily determined by an ordinary medical practitioner and may vary depending on factors such as the type and stage of pathological processes considered, the patient's medical history and age, and the administration of other therapeutic agents.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” . . . list of items . . . “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

Detailed Definition of Radicals of Formula (I) and Lipidic Compounds

As above specified, the lipidic compounds as disclosed herein are ionizable, and for example are cationic lipidic compounds.

Lipidic compounds as disclosed herein are for example ionizable since they are amine-containing lipidic compounds. As such compounds can be readily protonated, their pKa change according to the value of pH. For example, the compounds as disclosed herein have for example a pKa lower than 7 and for example ranging from 4.5 to 6.7.

The lipidic compounds as disclosed herein may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present disclosure. In addition, the cationic lipids disclosed herein may exist as tautomers and both tautomeric forms are intended to be encompassed by the scope of the disclosure, even though only one tautomeric structure is depicted.

The pharmaceutically acceptable salts of compounds as disclosed herein have one or several counter ions which are generally physiologically acceptable. As possible counter ions may be for example cited halides, phosphate, trifluroroacetate, sulfite, nitrate, gluconate, glucuronate, galacturonic acid radical, alkylsulfonate, alkylcarboxylate, propionic sulfonate and methanesulfonic acid radical.

The compounds as disclosed herein and the pharmaceutically acceptable salts thereof can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present disclosure.

For example, the lipidic compounds as disclosed herein have one hydrophilic head group formed by one radical of formula (I) also named terminal radical to illustrate the fact that it is linked, directly or not, to an end of the hydrophobic or lipophilic tail.

The radical of formula (I) has the following definition:

*—NH—CX—(NH)ₙ-A (I)
wherein:
represents a single bond linking said radical of formula (I), directly or not, to one C₁₀ to C₅₅ lipophilic or hydrophobic tail-group;
n is 0 or 1;
X is an oxygen or sulfur atom, and
A represents an optionally substituted 5- or 6-membered unsaturated heterocyclic radical or 5- or 6-membered heteroaromatic ring radical, both containing at least one nitrogen atom.

The compound comprising at least one radical of formula (I) can also be one of its pharmaceutically acceptable salts; and one of its possible racemic, enantiomeric and diastereoisomeric isomer forms.

As the nitrogen atom of the amide function may be protonated the lipidic compounds as disclosed herein may be protonated. Thus, and as previously stated, the lipidic compounds as disclosed herein have an apparent pKa that may change according to the value of pH.

According to one embodiment, the compounds as disclosed herein have a pKa lower than 7.

According to another specific embodiment, the compounds as disclosed herein have a pKa ranging from 4.5 to 6.7. Such a pKa may be determined by any conventional methods.

According to one embodiment, X is a sulfur atom and A is a pyridinyl radical.

According to one embodiment, A is a 3-pyridinyl radical.

According to another embodiment, X is an oxygen atom and A is a 5 membered heteroaromatic ring containing at least one nitrogen atom. According to one embodiment A is an imidazolyl radical. For example, A may be the 4-imidazolyl radical.

One radical of formula (I) is directly or not, attached to a hydrophobic (lipophilic) tail group (eg, a covalent bond).

The hydrophobic or lipophilic tail is generally in C₁₀ to C₅₅.

For example, it is an optionally substituted branched or unbranched linear saturated or unsaturated C₁₀ to C₅₅ hydrocarbon radical, and which hydrocarbon skeleton that is optionally interrupted by one or several atoms of oxygen or nitrogen and/or one or several —O—CO— or —CO—O— groups and which one nitrogen atom if present in the skeleton can be, directly or not, linked to the radical of formula (I).

For example, the hydrophobic or lipophilic tail may comprise at least two, three or more hydrocarbon chains each one independently being selected from optionally substituted C₈-C₂₄, for example C₁₀-C₂₀, alkyl chain, optionally substituted variably saturated or unsaturated C₈-C₂₄, for example C₁₀-C₂₀, alkenyl chain and optionally substituted saturated, variably saturated or unsaturated C₈-C₂₄, for example C₁₀-C₂₀, acyl chain with said alkyl, alkenyl or acyl chains can be interrupted by one or several atoms of oxygen or nitrogen and/or one or several moieties like —O—CO— or —CO—O—. and preferably by at least one moiety like —O—CO— or —CO—O—.

Each hydrocarbon chain may be substituted by at least one radical selected from —OH, and CO₂H.

According to one embodiment, the hydrophobic or lipophilic tail is selected in the group consisting of:

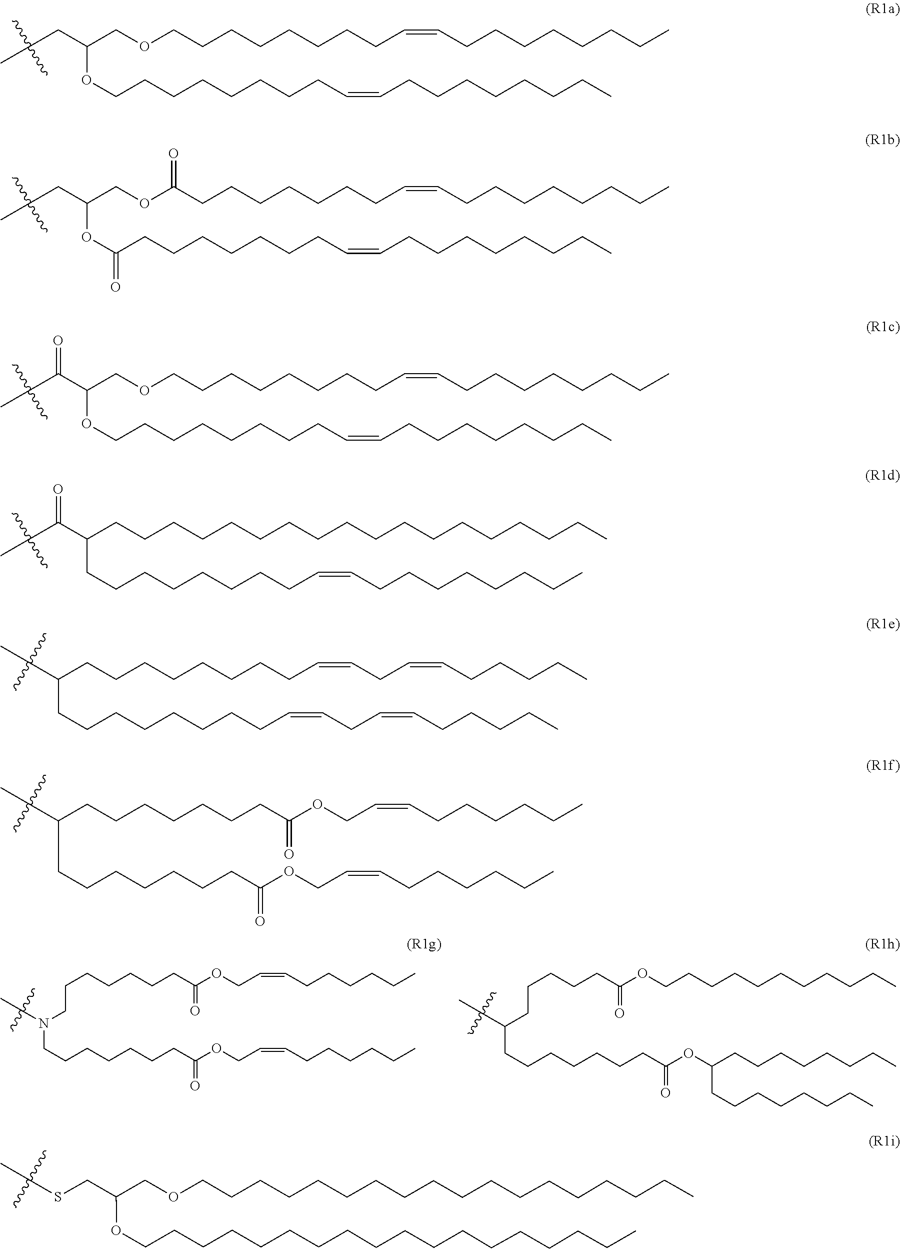

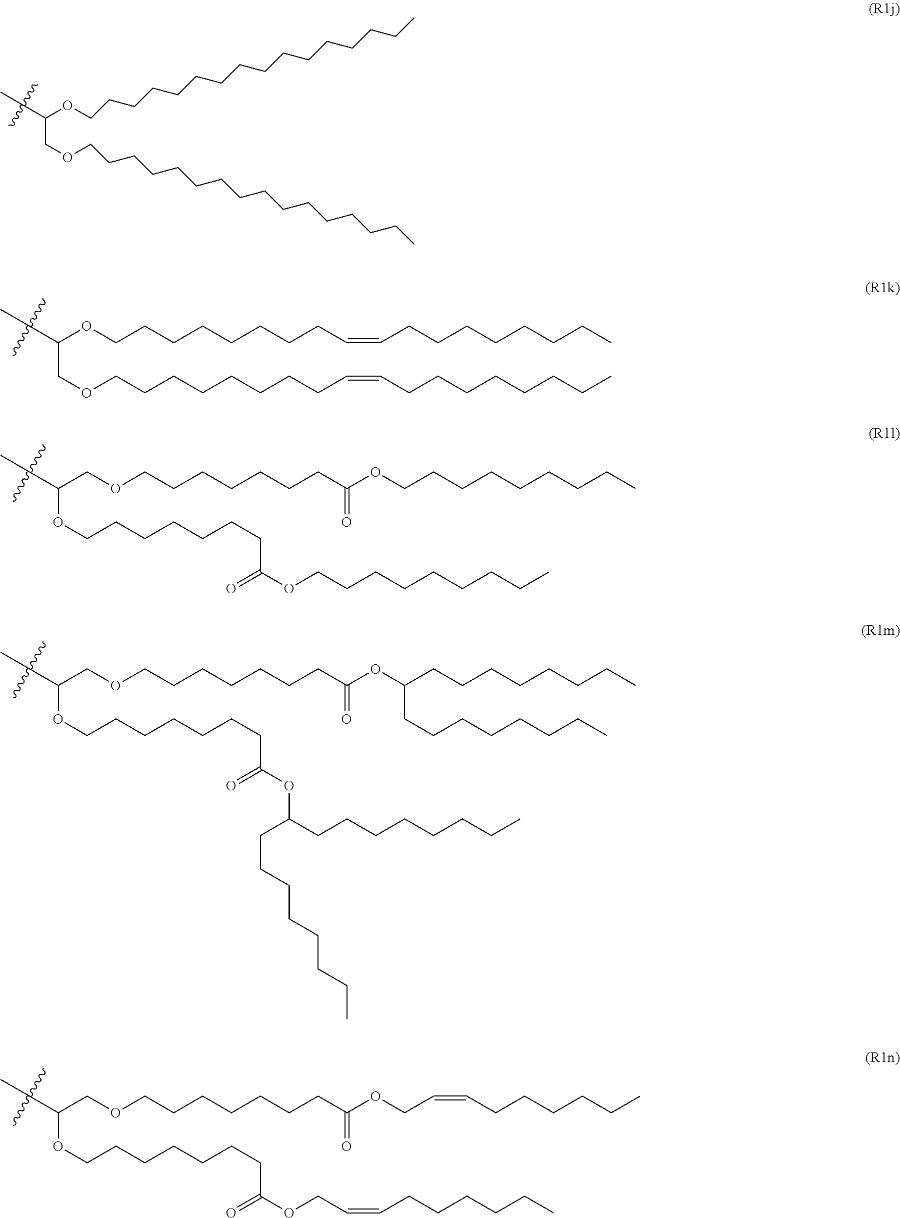 [SMILES: *C(COCCCCCCCCCCCCCCCC)OCCCCCCCCCCCCCCCC ; *C(COCCCCCCCC/C=C\CCCCCCCC)OCCCCCCCC/C=C\CCCCCCCC ; *C(COCCCCCCCC(=O)OCCCCCCCCC)OCCCCCCCC(=O)OCCCCCCCCC ; *C(COCCCCCCCC(=O)OC(CCCCCCCC)CCCCCCCC)OCCCCCCCC(=O)OC(CCCCCCCC)CCCCCCCC ; *C(COCCCCCCCC(=O)OC/C=C\CCCCCC)OCCCCCCCC(=O)OC/C=C\CCCCCC]

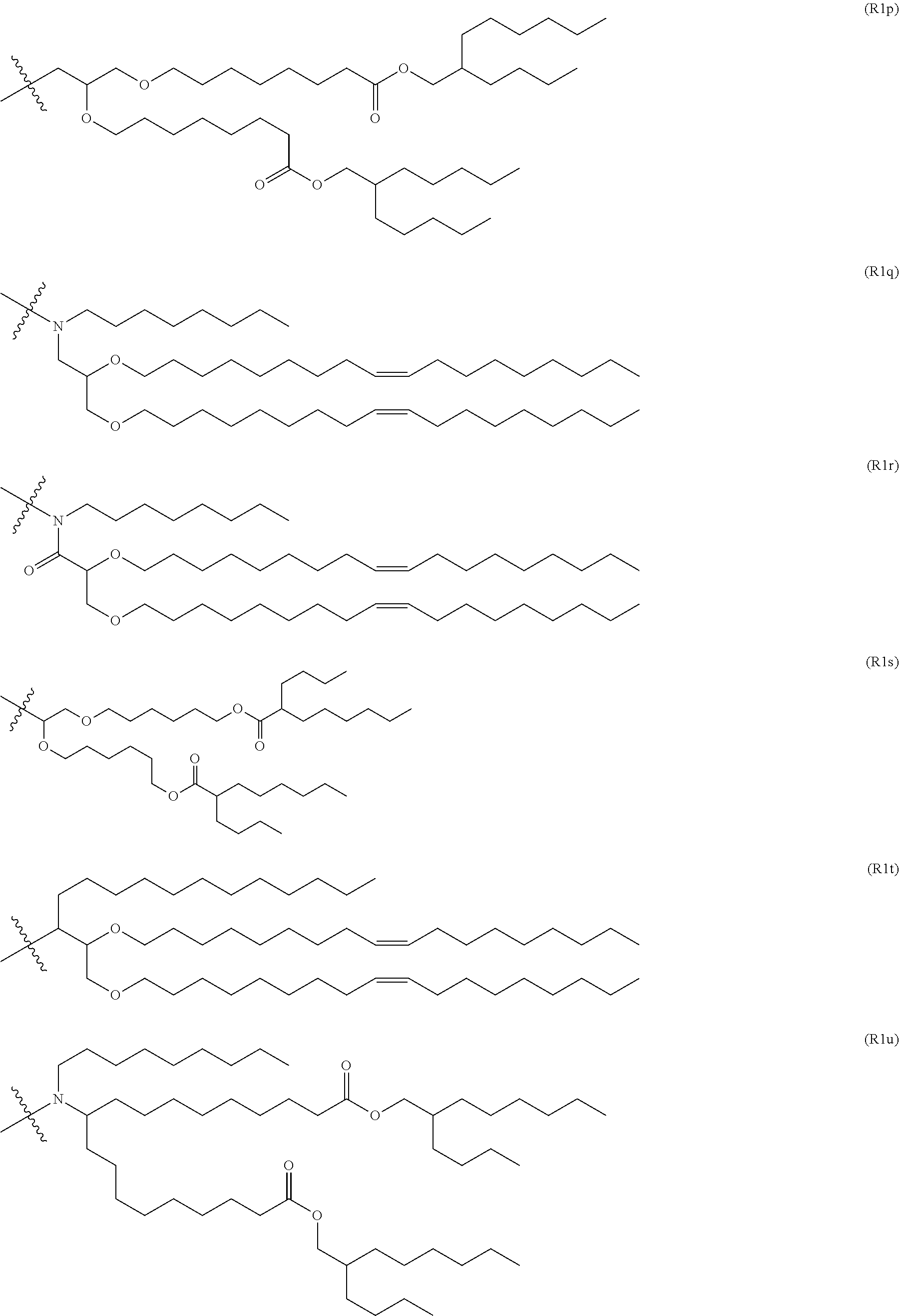 [SMILES: *CC(COCCCCCCCC(=O)OCC(CCCC)CCCCCC)OCCCCCCCC(=O)OCC(CCCCC)CCCCC ; *N(CCCCCCCC)CC(COCCCCCCCC/C=C\CCCCCCCC)OCCCCCCCC/C=C\CCCCCCCC ; *N(CCCCCCCC)C(=O)C(COCCCCCCCC/C=C\CCCCCCCC)OCCCCCCCC/C=C\CCCCCCCC ; *C(COCCCCCCOC(=O)C(CCCC)CCCCCC)OCCCCCCOC(=O)C(CCCC)CCCCCC ; *C(CCCCCCCCCCCC)C(COCCCCCCCC/C=C\CCCCCCCC)OCCCCCCCC/C=C\CCCCCCCC ; *N(CCCCCCCCC)C(CCCCCCCCC(=O)OCC(CCCC)CCCCCC)CCCCCCCCC(=O)OCC(CCCC)CCCCCC]

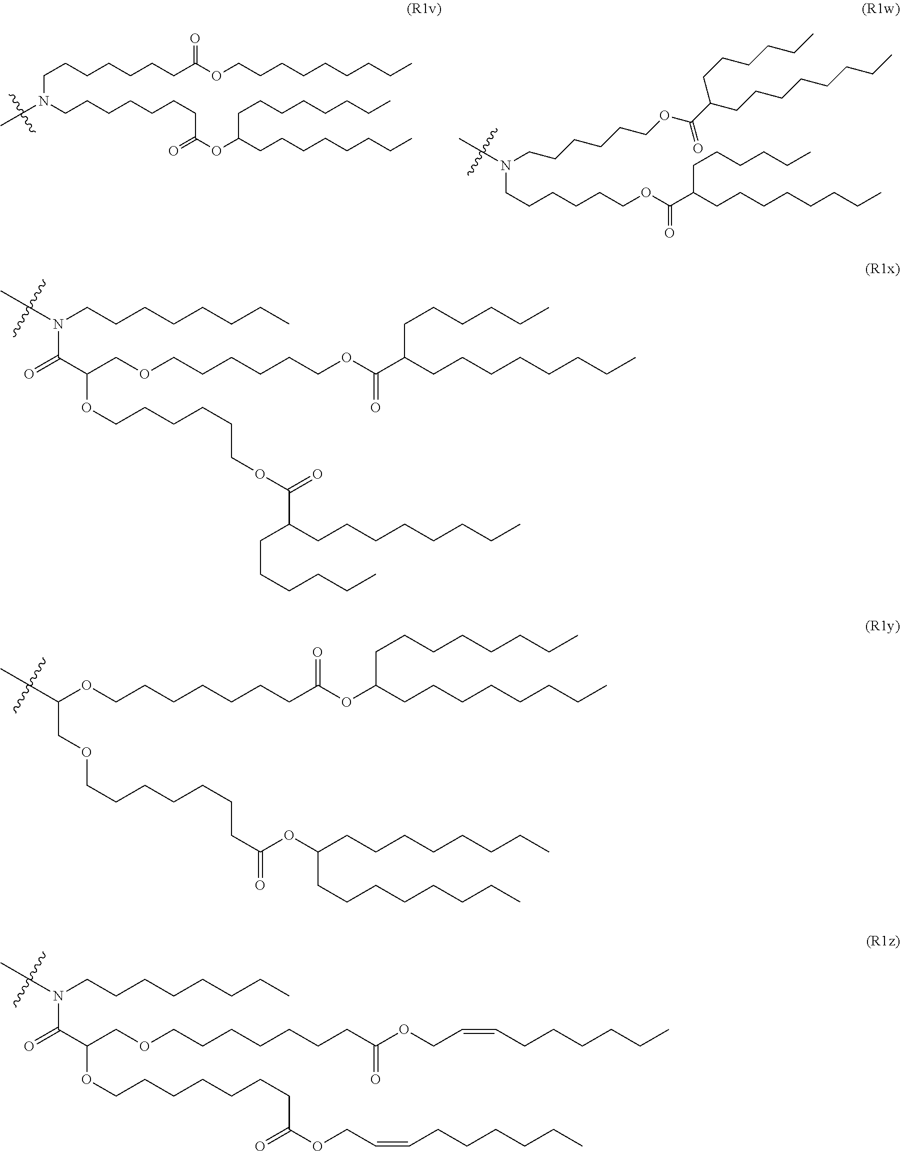 [SMILES: *N(CCCCCCCC(=O)OCCCCCCCCC)CCCCCCCC(=O)OC(CCCCCCCC)CCCCCCCC ; *N(CCCCCCOC(=O)C(CCCCCC)CCCCCCCC)CCCCCCOC(=O)C(CCCCCC)CCCCCCCC ; *N(CCCCCCCC)C(=O)C(COCCCCCCOC(=O)C(CCCCCC)CCCCCCCC)OCCCCCCOC(=O)C(CCCCCC)CCCCCCCC ; *C(COCCCCCCCC(=O)OC(CCCCCCCC)CCCCCCCC)OCCCCCCCC(=O)OC(CCCCCCCC)CCCCCCCC ; *N(CCCCCCCC)C(=O)C(COCCCCCCCC(=O)OC/C=C\CCCCCC)OCCCCCCCC(=O)OC/C=C\CCCCCC]

In one embodiment, the hydrophobic or lipophilic tail of compounds according to the invention contains at least one amino moiety involved in its linking to the spacer. In this specific embodiment, the hydrophobic or lipophilic tail is in particular selected among Rig, h, q, r, u, v, w and z.

In another embodiment, the hydrophobic or lipophilic tail of compounds according to the invention may also contain at least three or more hydrocarbon chains like for example in the hydrophobic or lipophilic tails R1m, p, q, r, s, u, v, w, x, y and z. Each hydrocarbon chain may be selected among substituted C₈-C₂₄, for example C₁₀-C₂₀, alkyl chain and substituted variably saturated or unsaturated C₈-C₂₄, for example C₁₀-C₂₀, alkenyl chain and with said alkyl or alkenyl chains optionally and preferably being interrupted by one or several moieties like —O—CO— or —CO— O—.

In a specific embodiment, the hydrophobic or lipophilic tail of compounds according to the invention is the tail (Ria) or (Rib) also respectively named DOG alkyl or DOG ether.

According to another embodiment, the cationic and/or ionizable lipidic compounds as disclosed herein are of formula (II)

R1-Z—NH—CX—(NH)ₙ-A (II)

wherein:
X, n and A are as previously defined
R1 is one C₁₀ to C₅₅ lipophilic or hydrophobic tail-group, for example as previously defined;
Z is a spacer arm having from 2 to 24, for example from 2 to 18, for example from 4 to 12 carbon atoms, or for example from 2 to 12 carbon atoms, in a branched or unbranched linear saturated or unsaturated hydrocarbon chain, said chain that is interrupted by one or several atoms of oxygen and/or moieties selected among —S—S—; —(O═C)—; —(C═O)—O—; —O—(O═C)—; —S—; —NH—, —NH—(O═C)—; —(O═C)—NH— and —NH—(C═O)—O— and preferably by —(C═O)—O—; —O—(O═C) and —NH—(C═O)—O— and optionally ended by an oxygen atom or a moiety selected among —NH—(O═C)—O—(O═C)—; —(C═O)—O—; and —(O═C)—to be linked to the hydrophobic tail-group
p is 0 or 1;
or one of the pharmaceutically acceptable salts of said compounds of formula (II); and any of its racemic, enantiomeric and diastereoisomeric isomer forms.

Regarding the spacer arm, it is like the ones conventionally considered in the field of the lipid cationic compounds. Accordingly, the choice of such spacer arm does not raise any difficulty for the man skilled in the art. It needs to be inert or not prejudicial to the efficiency of the lipid compound.

Generally, the spacer has 2 to 24 and for example from 4 to 12 carbon atoms, or for example from 2 to 12 carbon atoms, and comprises at least one or several ethylene oxide units and optionally one or several moieties as previously disclosed.

As examples of spacer arms convenient for the disclosure, it may be cited the following ones which the right end being the one linked to the lipophilic or hydrophobic tail-group:

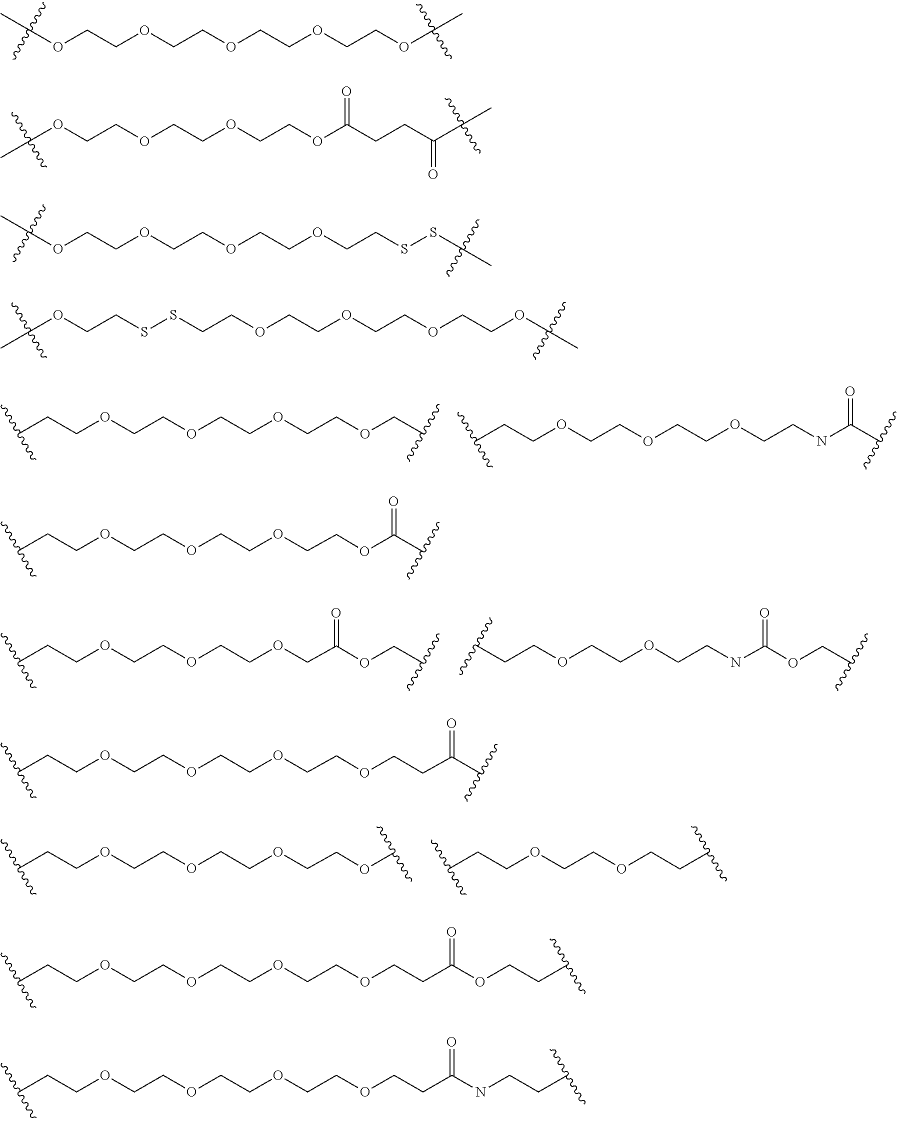

According to one embodiment, the spacer consists in ethylene oxide units and may comprise from 1 to 24, for example from 2 to 15, for example from 3 to 12, for example from 4 to 10, and for example from 6 to 8 ethylene oxide units.

According to another specific embodiment, the spacer may be of formula

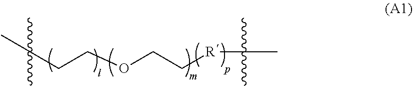

wherein:
the right end is the one linked to the lipophilic or hydrophobic tail-group,
l is 0 or 1;
m ranges from 1 to 24, for example from 2 to 15, for example from 3 to 12, for example is 2, 3, 4, 5, 6, 7, 8, or 9;
p is 0 or 1; and
R′ represents, when p is 1, one atom of oxygen or a moiety selected from —C═O—; —NH—; —O—CH₂—; —NH—C(═O)—; —NH—C(═O)—O—CH₂—; O—C(═O)—; C═O—NH—(CH₂)₂—; OCH₂, C(═O)—O—; —C(═O)—O—(CH₂)₂— and —S—S— and in particular —NH—C(═O)—; —NH—C(═O)—O—CH₂—; —O—C(═O)—; —C═O—NH—(CH₂)₂—; and; —C(═O)—O—(CH₂)₂.

According to another embodiment, the spacer may comprise from 1 to 24, for example from 2 to 15, for example from 3 to 12, for example from 4 to 10, and for example from 6 to 8 ethylene oxide units and preferably incorporates at least one moiety selected among —(C═O)—O—; —O—(O═C)—; —NH—(O═C)—; —(O═C)—NH— and; —NH— (C═O)—O— and more preferably at least one —NH—(C═O)—O—.

In one embodiment, in the lipidic compound of formula (II), X is a sulfur atom and A is a pyridinyl radical. For example, A may be a 3-pyridinyl radical.

According to this embodiment, the compound of formula (II) is for example the compound of formula (V)

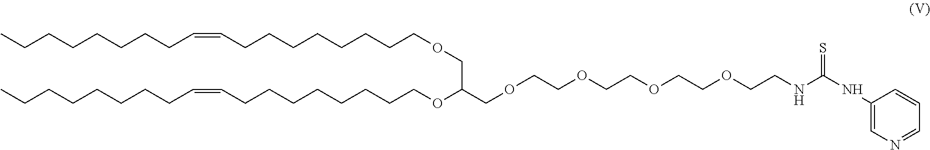 [SMILES: CCCCCCCC/C=C\CCCCCCCCOCC(COCCOCCOCCOCCNC(=S)Nc1cccnc1)OCCCCCCCC/C=C\CCCCCCCC]

or one of its salts, or one of its racemic, enantiomeric and diastereoisomeric isomer forms.

For example, this compound (V) or derivatives thereof is not salified. For example, it is under the form of its free base.

In another embodiment, in the lipidic compound of formula (II), X is an oxygen atom and A is a 5 membered heteroaromatic ring radical containing at least one nitrogen atom. Thus, according to one embodiment A is an imidazolyl radical, and for example A may be a 4-imidazolyl radical.

According to this embodiment, the compound of formula (II) is for example selected among the following compounds (III) to (XXVII) and theirs salts or racemic, enantiomeric and diastereoisomeric isomer forms. In remark, in the following developed formula, a secondary amino moiety may be indifferently written —NH— or —N—.

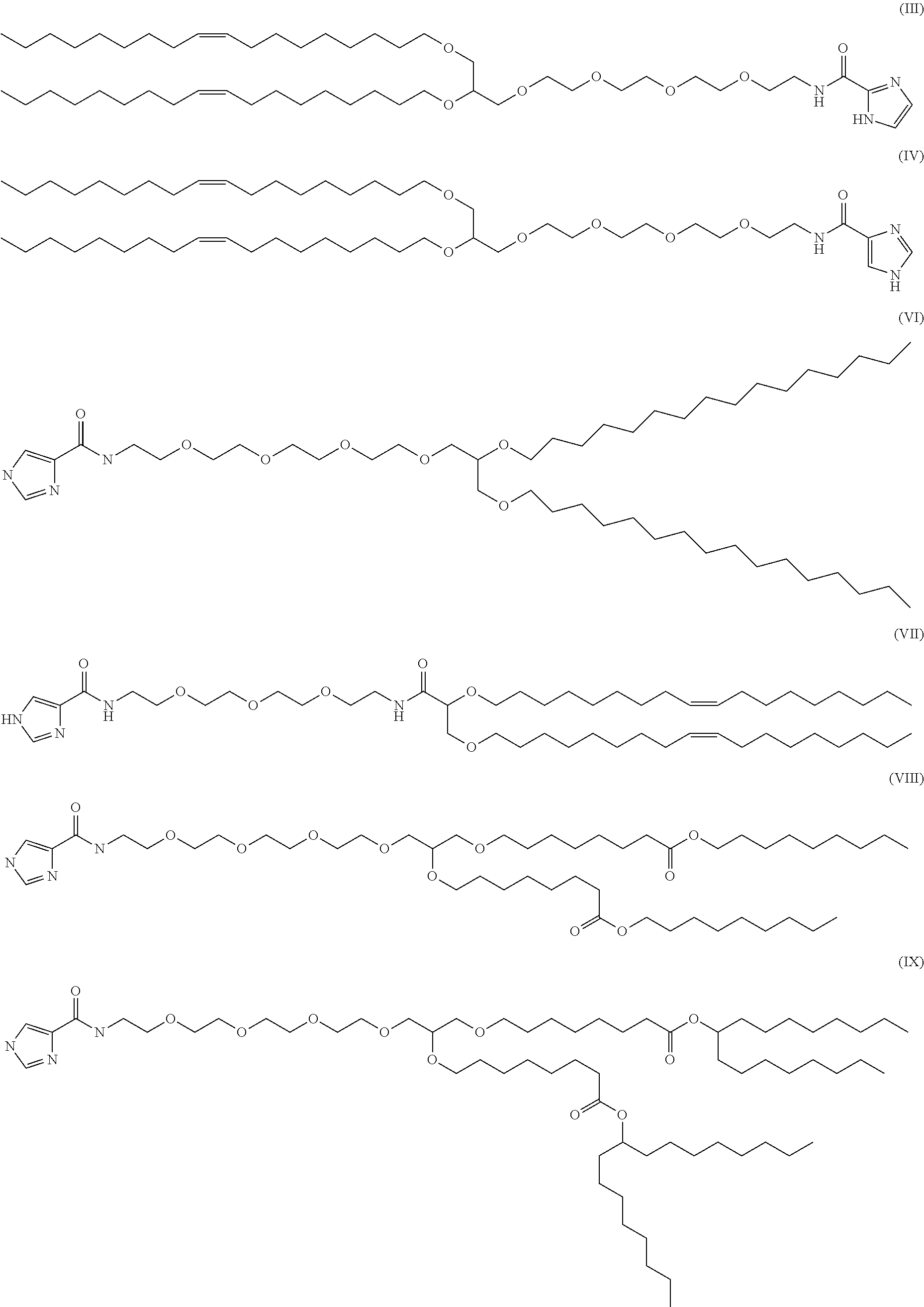 [SMILES: CCCCCCCC/C=C\CCCCCCCCOCC(COCCOCCOCCOCCNC(=O)c1ncc[nH]1)OCCCCCCCC/C=C\CCCCCCCC ; CCCCCCCC/C=C\CCCCCCCCOCC(COCCOCCOCCOCCNC(=O)c1c[nH]cn1)OCCCCCCCC/C=C\CCCCCCCC ; CCCCCCCCCCCCCCCCOCC(COCCOCCOCCOCCNC(=O)c1c[nH]cn1)OCCCCCCCCCCCCCCCC ; CCCCCCCC/C=C\CCCCCCCCOCC(OCCCCCCCC/C=C\CCCCCCCC)C(=O)NCCOCCOCCOCCNC(=O)c1c[nH]cn1 ; CCCCCCCCCOC(=O)CCCCCCCOCC(COCCOCCOCCOCCNC(=O)c1c[nH]cn1)OCCCCCCCC(=O)OCCCCCCCCC ; CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

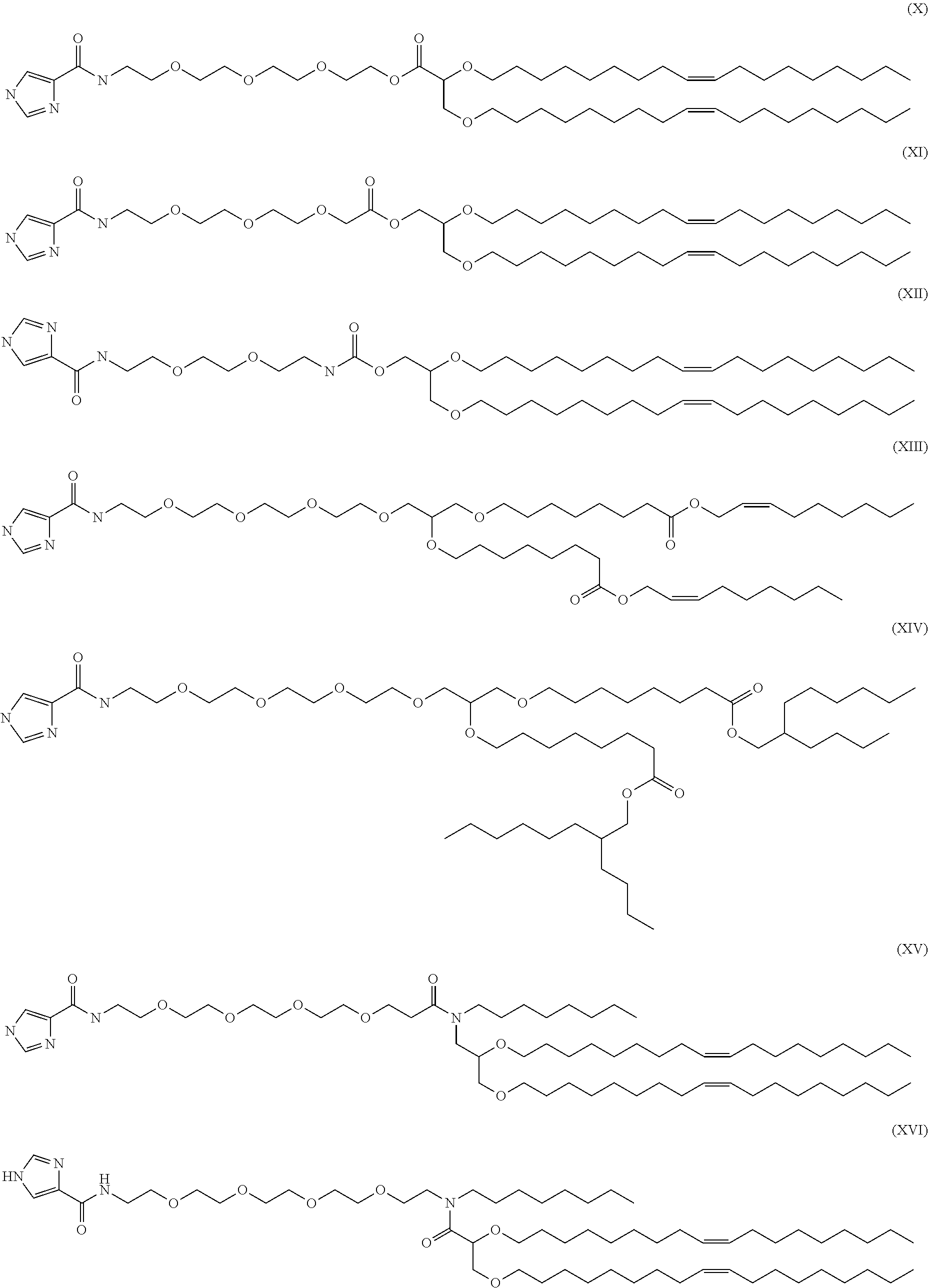

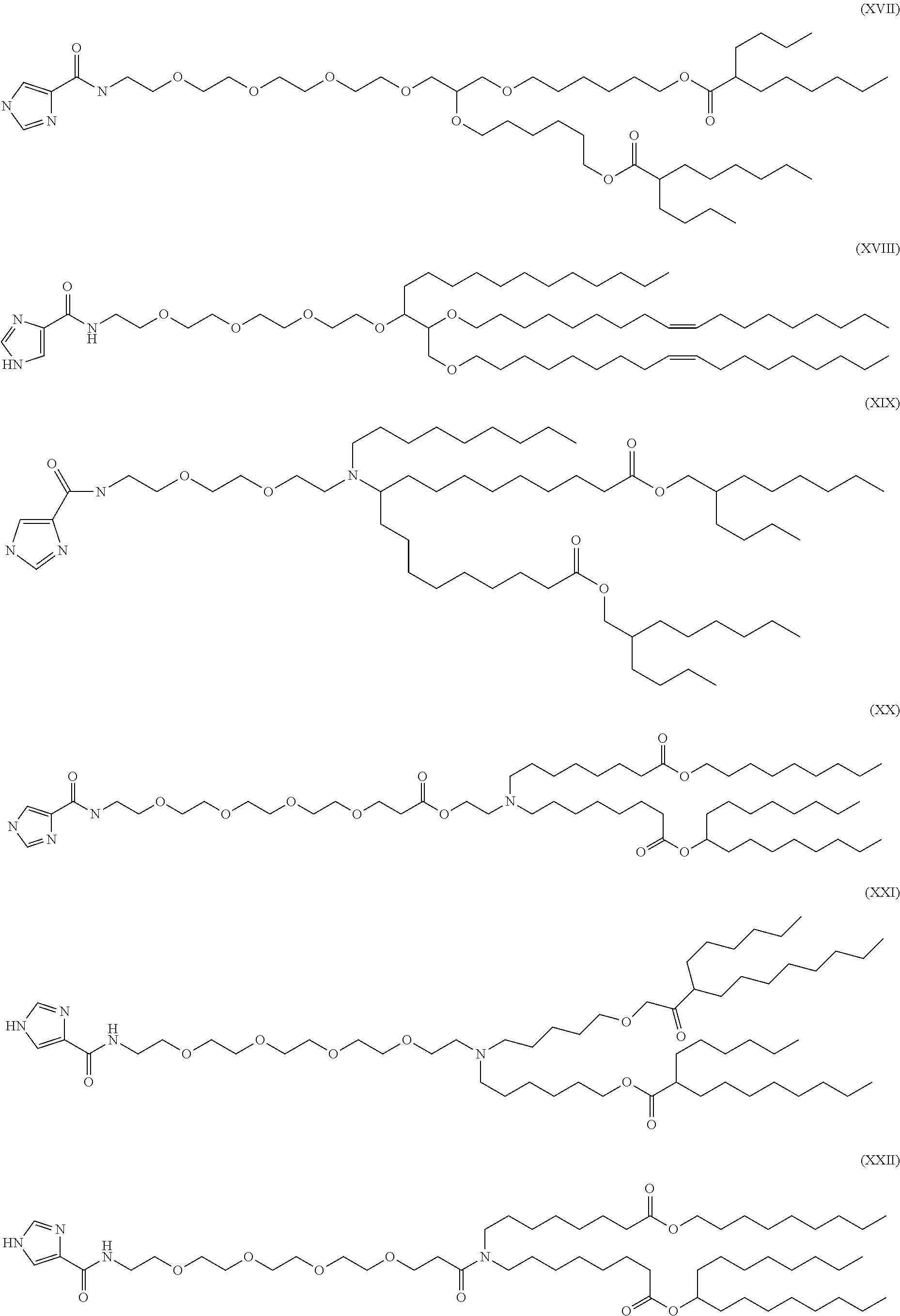

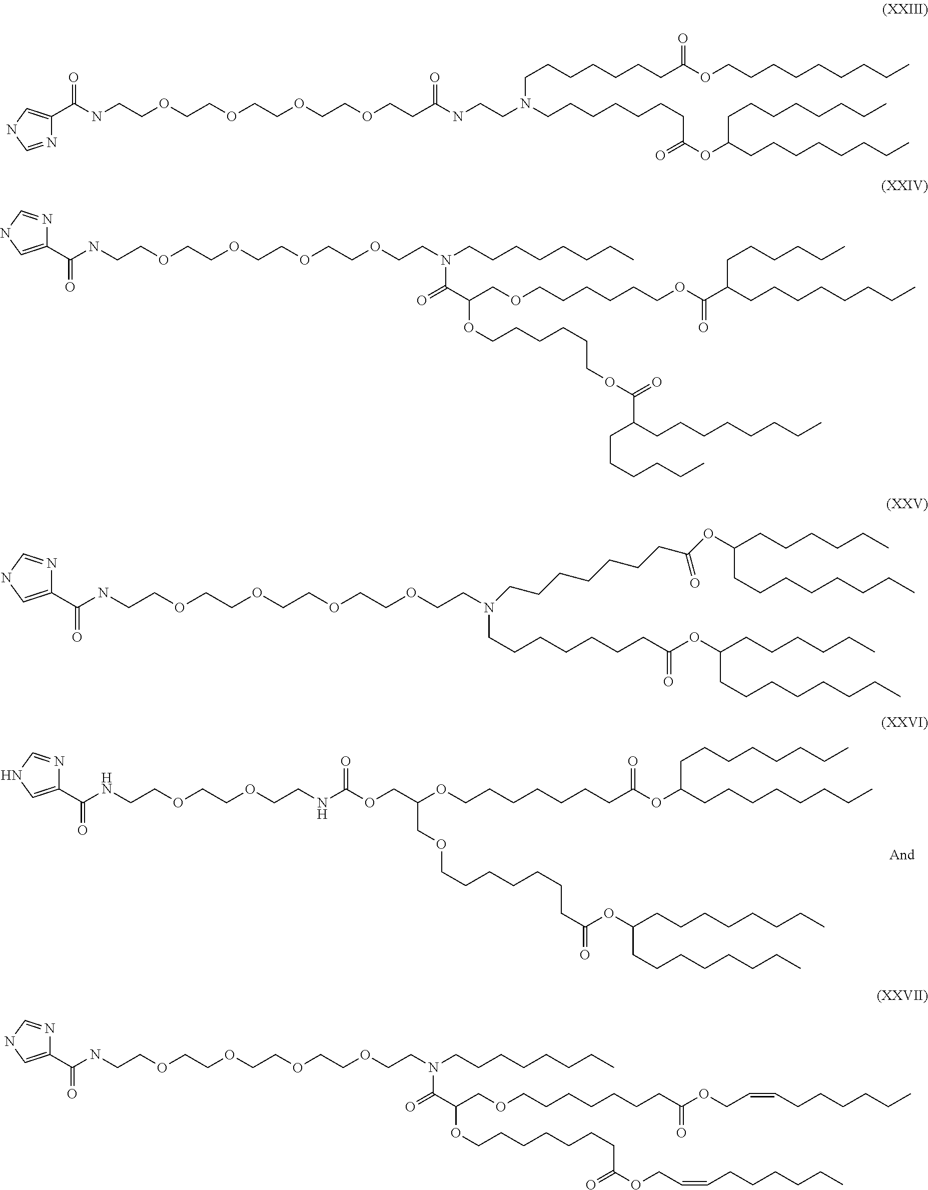

More particularly, the compounds (IV), (VIII), (IX), (XII), (XVI), (XIX) and (XXII), in particular the compound (IV), (IX), (XII), or (XVI), more particularly the compound (IV) or (XII), and all particularly the compound (IV), are interesting like theirs salts or racemic, enantiomeric and diastereoisomeric isomer forms. For example, they may be under their free base.

As shown in the Examples section, the compounds (IV), (VIII), (IX), (XII), (XVI), (XIX) and (XXII) are for example efficient for the formulation of stable LNPs (stable in a liquid form at 4-8° C.) capable of delivering a functional mRNA into target tissues after parenteral administrations and for the induction of the expression of a protein such as EPO or an immune response in the case the delivered mRNA codes for an antigen.

Preparation of Cationic Lipids

The compounds according to the disclosure can be prepared from readily commercially available or described in the literature starting materials using methods and procedures known from the skilled person.

For example, the lipidic compounds of formula (II) may be obtained by covalent coupling, between a precursor of the radical of formula (I) and a lipidic compound or derivative thereof having a terminal reactive group able to react with said precursor.

This terminal reactive group may be located directly on the end of the hydrophobic or lipophilic part of the lipidic compound to transform or on the end of a spacer already linked to the hydrophobic or lipophilic part of the lipidic compound.

The choice of the convenient precursor of the radical of formula (I) intended to react with the lipidic compound for forming the expected covalent linkage is clearly within the competence of the man skilled in the art. The precursor only needs to have a group able to chemically react with the one of the lipidic compound for forming the covalent linking.

Regarding these starting compounds i.e. precursor of the radical of formula (I) and the lipidic compound or derivative thereof to transform they may be easily produced by a man skilled in the art, for example according to the methods of preparation submitted in the following examples.

The covalent coupling may be also performed according to methods that that are known to those skilled in the art in regard of the chemical nature of the reactive group of the precursor of the radical of formula (I) and the one on the lipidic compound or derivative thereof to be transformed.

Generally, the covalent linking may be formed by an esterification, amidation or cabamation.

As representative of a convenient precursor for a radical of formula (I) having a pyridinyl group for A, it can be cited its corresponding pyridyl isothiocyanate like, for example, the 3-pyridyl isothicyanate.

As representative of a convenient precursor for a radical of formula (I) having an imidazolyl group for A, it can be cited the corresponding imidazolcarboxylic acids.

One specific approach for obtaining a compound of formula (I) as the compound of formula (IV) is depicted in Scheme 1 below.

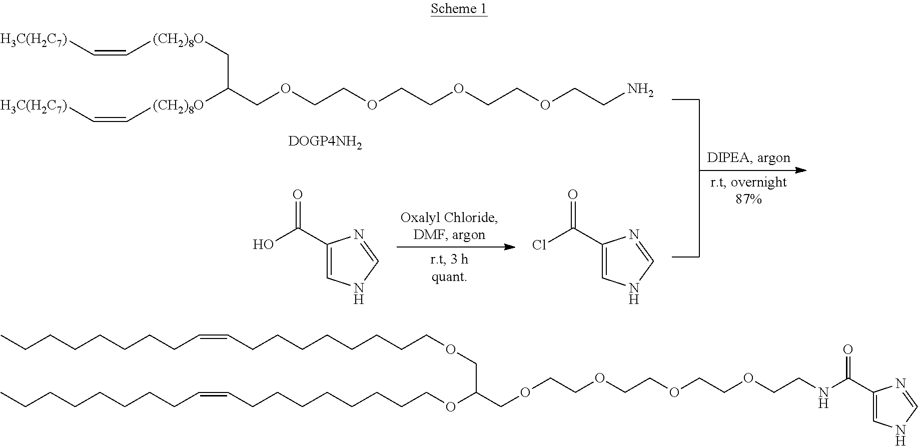

It will be appreciated that where typical or particular experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

The optional salification may be performed by conventional way to form the expected cationic form.

The coupling reaction may advantageously be followed by subsequent steps of purifying and/or isolating of the final product obtained. Convenient methods of purification are detailed in the following examples. For example, the purification of the obtained compounds may be performed by preparative high-performance liquid chromatography (HPLC).

The instant disclosure will be better understood from the examples that follow, all of which are intended for illustrative purposes only and are not meant to limit the scope of the instant disclosure in any way.

Compositions, Lipid Nanoparticles, and Manufacturing Processes

The present disclosure relates to compositions which comprises at least one lipidic compound as disclosed herein, as above described. A composition as disclosed herein may further comprise at least one lipid selected from the group consisting of neutral lipids, steroid alcohols or esters thereof, and PEGylated lipids.

A composition as disclosed herein may be formulated as lipid nanoparticles containing at least one nucleic acid.

The compositions or the lipid nanoparticles as disclosed herein may further comprise at least one therapeutic, anionic or polyanionic, agent, for example at least one nucleic acid.

Neutral Lipids

A composition or lipid nanoparticles as disclosed herein may include a neutral lipid. The presence of neutral lipids may improve structural stability of the lipid nanoparticles. The neutral lipid can be appropriately selected in view of the delivery efficiency of nucleic acid.

The neutral lipids are distinct from the lipidic compound as disclosed herein. Neutral lipids are either not ionizable or are neutral zwitterionic compounds at a selected pH.

Neutral lipids useful for the disclosure may be selected from the group consisting of phosphatidylcholines, phosphatidylethanolamines, sphingomyelins, and ceramides.

Phosphatidylcholines and phosphatidylethanolamines are zwitterionic lipids. Sphingomyelins and ceramides are not ionizable lipids.

As examples of phosphatidylcholines useful for the disclosure, one may mention DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine).

As examples of phosphatidylethanolamines useful for the disclosure one may mention DOPE (1,2-dioleyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), DSPE (1,2-distearoyl-s/i-glycero-3-phosphoethanolamine), DLPE (1,2-dilauroyl-SM-glycero-3-phosphoethanolamine), 16-O-monomethyl PE, 16-O-dimethyl PE, 18-1-trans PE, or 1-stearoyl-2-oleoyl-phosphatidyethanolamine (SOPE).

A neutral lipid may be selected from the group consisting of phosphatidylcholines, such as DSPC, DPPC, DMPC, POPC, DOPC; phosphatidylethanolamines, such as DOPE, DPPE, DMPE, DSPE, DLPE; sphingomyelins; and ceramides.

In one embodiment, a neutral lipid suitable for the disclosure may be DSPC, DOPC, and DOPE, and for example may be DSPC or DOPE.

Neutral lipids may be present at step a) of the method for formulating the lipid nanoparticles as disclosed herein in a molar amount ranging from about 0% to about 50%, for example from about 5% to about 45%, for example from about 8% to about 40%, and for example from about 10% to about 30% relative to the total molar amount of the lipid and lipidic compound as disclosed herein.

Neutral lipids may be present in a composition or lipid nanoparticles as disclosed herein in a molar amount ranging from about 0% to about 50%, for example from about 5% to about 45%, for example from about 8% to about 40%, and for example from about 10% to about 30% relative to the total molar amount of the lipid and lipidic compound as disclosed herein.

Neutral lipids may be present in a composition or lipid nanoparticles as disclosed herein in a molar ratio lipidic compound:neutral lipid which may range from about 70:1 to about 1:2, for example from about 30:1 to about 1:1, for example from about 15:1 to about 2:1, for example from about 10:1 to about 4:1, and more for example is about 5:1.

Steroid Alcohols or Esters Thereof

A composition or lipid nanoparticles as disclosed herein may include a steroid alcohol (or sterol) or an ester thereof. The presence of sterol or ester of sterol may also improve structural stability of the lipid nanoparticles.

Sterols or steroid alcohols useful for the disclosure may be selected from the group consisting of cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol).

Esters of steroid alcohol or of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments the alkyl group may be a C₁-C₂₀ saturated or unsaturated, linear or branched, alkyl group, for example a C₂-C₁₈, for example a C₄-C₁₆, for example C₈-C₁₂ saturated or unsaturated, linear or branched, alkyl group, In other embodiments, the carboxylic acid may be a fatty acid. For example, a fatty acid may be caprylic acid, capric acid, lauric acid, stearic acid, margaric acid, oleic acid, linoleic acid, or arachidic acid.

In one embodiment, an ester of sterol suitable for the disclosure may be a cholesteryl ester.

Esters of sterol or of steroid alcohol useful for the disclosure may be selected from the group consisting of cholesteryl margarate (cholest-5-en-38-yl heptadecanoate), cholesteryl oleate, and cholesteryl stearate.

Sterols or steroid alcohols or esters thereof useful for the disclosure may be selected from selected from the group consisting of cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (Δ5,7-cholesterol), dihydrolanosterol (24,25-dihydrolanosterol), zymosterol (5α-cholesta-8,24-dien-3β-ol), lathosterol (5α-cholest-7-en-3β-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), 24-methylene cholesterol (5,24(28)-cholestadien-24-methylen-3β-ol), cholesteryl margarate (cholest-5-en-38-yl heptadecanoate), cholesteryl oleate, and cholesteryl stearate.

Alternatively, a sterol useful for the disclosure may be a cholesterol derivative such as an oxidized cholesterol.

Oxidized cholesterols suitable for the disclosure may be 25-hydroxycholesterol, 27-hydroxycholesterol, 20α-hydroxycholesterol, 6-keto-5α-hydroxycholesterol, 7-keto-cholesterol, 7β,25-hydroxycholesterol and 7β-hydroxycholesterol. For example, oxidized cholesterols may be 25-hydroxycholesterol and 20α-hydroxycholesterol, and for example it may be 20α-hydroxycholesterol.

In one embodiment, a sterol or steroid alcohol, or ester thereof, suitable for the disclosure may be cholesterol, a cholesteryl ester, or a cholesterol derivative, for example an oxidized cholesterol. In one embodiment, a sterol or steroid alcohol suitable for the disclosure may be cholesterol or a cholesteryl ester, and for example may be cholesterol.

Sterols or steroid alcohols, or esters thereof, may be present in a composition or lipid nanoparticles as disclosed herein in molar amount ranging from about 0 to about 60%, for example from about 10% to about 50%, and for example from about 20% to about 50% relative to the total molar amount of the lipid and lipidic compound as disclosed herein which may be present in the composition or the lipid nanoparticles.

Sterols or steroid alcohols, or esters thereof, may be present in a composition or lipid nanoparticles as disclosed herein in a molar ratio lipidic compound:steroid alcohol, or ester thereof, which may range from about 4:1 to about 1:2, for example from about 3.5:1 to about 1:1.8, for example from about 2:1 to about 1:1.5, for example from about 1.5:1 to about 1:1.2, and for example is about 1.3:1 to about 1:1.3.

PEGylated Lipids

A composition or lipid nanoparticles as disclosed herein may include a PEGylated (or PEG-) lipid.

Contemplated PEG-modified lipids include, but are not limited to, a polyethylene glycol chain of up to 5 kDa in length covalently attached to a lipid with alkyl chain(s) of C₆-C₂₀ length. The addition of PEG-modified lipids to a composition of lipid nanoparticles as disclosed herein may prevent complex aggregation and may also provide a means for increasing circulation lifetime and increasing the delivery of the composition or lipid nanoparticles to the target cells.

A suitable PEGylated lipid may be, for example, a pegylated diacylglycerol (PEG-DAG), such as 1-(monomethoxy-polyethyleneglycol)-2,3-dimyristoylglycerol (DMG-PEG), a pegylated phosphatidylethanoloamine (PEG-PE), a PEG succinate diacylglycerol (PEG-S-DAG) such as 4-0-(2′,3′-di(tetradecanoyloxy)propyl-1-0-(co-methoxy(polyethoxy) ethyl)butanedioate (PEG-S-DMG), a pegylated ceramide (PEG-cer), or a PEG dialkoxypropylcarbamate, such as ω-methoxy(polyethoxy)ethyl-N-(2,3-di(tetradecanoxy)propyl)carbamate, 2,3-di(tetradecanoxy)propyl-N-(co-methoxy(polyethoxy)ethyl)carbamate, or mPEG-N,N-ditetradecylacetamide (also known as 2-[(polyethylene glycol)-2000]-N,N-ditetradecylacetamide or ALC-0159).

In one embodiment, a PEGylated lipid suitable for the disclosure may be selected from the group consisting of PEG-DAG, DMG-PEG, PEG-PE, PEG-S-DAG, PEG-S-DMG, PEG-cer, or mPEG-N,N-ditetradecylacetamide, or a PEG-dialkyoxypropylcarbamate.

For example, a PEGylated lipid suitable for the disclosure may be DMG-PEG, PEG-PE, or mPEG-N,N-ditetradecylacetamide.

In some embodiment, a PEGylated lipid suitable for the disclosure may be DMG-PEG or PEG-PE.

In some embodiment, a PEGylated lipid suitable for the disclosure may be mPEG-N,N-ditetradecylacetamide.

Compositions or lipid nanoparticles as disclosed herein may comprise PEGylated lipid in a molar amount ranging from about 1 to about 15%, for example from about 1% to about 10%, for example from about 1% to about 5%, and for example from about 1% to about 3.5% relative to the total molar amount of the lipid and lipidic compound.

PEGylated lipid and lipidic compound may be present in a molar ration lipidic compound to PEGylated lipid from about 70:1 to about 4:1, for example from about 40:1 to about 10:1, for example from about 35:1 to about 15:1, and for example is about 33:1 or about 14:1.

In one embodiment, compositions or lipid nanoparticles may comprise, further to the lipidic compound as above-described, at least one neutral lipid, at least one steroid alcohol, or an ester thereof, and at least one PEGylated lipid.

The neutral lipids, the steroid alcohol or an ester thereof and the PEGylated lipids may be as above indicated.

In one embodiment, the compositions or lipid nanoparticles described herein may comprise a lipidic compound as disclosed herein, a neutral lipid, a steroid alcohol or an ester thereof, and a PEGylated lipid in a molar amount of about 30% to about 70% of lipidic compound, of about 0% to about 50% of neutral lipid, of 20% to about 50% of steroid alcohol or an ester thereof, and of about 1% to about 15% of PEGylated, relative to the total amount of lipid and lipidic compound.

In one embodiment, the compositions or lipid nanoparticles described herein may comprise a lipidic compound as disclosed herein, a neutral lipid, a steroid alcohol or an ester thereof, and a PEGylated lipid in a molar amount of about 30% to about 60% of lipidic compound, of about 5% to about 30% of neutral lipid, of about 30% to about 48% of steroid alcohol or an ester thereof, and of about 1.5% to about 5% of PEGylated, relative to the total amount of lipid and lipidic compound.

In one embodiment, the compositions or lipid nanoparticles described herein may comprise a lipidic compound as disclosed herein, a neutral lipid, a steroid alcohol or an ester thereof, and a PEGylated lipid in a molar amount of about 35% to about 50% of lipidic compound, of about 10% to about 16% of neutral lipid, of about 38.5% to about 46.5% of steroid alcohol or an ester thereof, and of about 1.5% of PEGylated, relative to the total amount of lipid and lipidic compound.

As one embodiment, the compositions or lipid nanoparticles as disclosed herein may comprise about 35% of lipidic compound as disclosed herein, about 16% of neutral lipid, about 46.5% of steroid alcohol or an ester thereof, and of about 1.5% of PEGylated, relative to the total amount of lipid and lipidic compound.

As another embodiment, the compositions or lipid nanoparticles as disclosed herein may comprise about 50% of lipidic compound as disclosed herein, about 10% of neutral lipid, about 38.5% of steroid alcohol or an ester thereof, and of about 1.5% of PEGylated, relative to the total amount of lipid and lipidic compound.

In one embodiment, the molar ratio of the lipidic compound as disclosed herein and of the neutral lipid, the steroid alcohol or an ester thereof, and the PEGylated lipid may be of about 35/16/46.5/1.5, of about 50/10/38.5/1.5, of about 57.2/7.1/34.3/1.4, of about 40/15/40/5, of about 50/10/35/4.5/0.5, of about 50/10/35/5, of about 40/10/40/10; of about 35/15/40/10, of about 52/13/30/5.

In one embodiment, the molar ratio of the lipidic compound as disclosed herein and of the neutral lipid, the steroid alcohol or an ester thereof, and the PEGylated lipid may be of about 35/16/46.5/1.5 or about 50/10/38.5/1.5.

In another embodiment, the lipidic compound as disclosed herein may be any of the compounds (III) to (XXVII), or the compounds (III), (IV), (V), (VIII), (IX), (XII), (XVI), (XIX), or (XXII), or the compounds (IV), (VIII), (IX), (XII), (XVI), (XIX), or (XXII), or the compounds (IV), (IX), (XII), or (XVI), or the compound (IV) or (XII), and for example is compound (IV), the neutral lipid may be DSPC or DOPE, the steroid alcohol may be cholesterol, and the PEGylated lipid may be PEG-PE (PEG2000-PE) or PEG-DMG (PEG2000-DMG).

In another embodiment, the lipidic compound as disclosed herein may be the compounds (III), (IV), (V), (VIII), (IX), (XII), (XVI), (XIX), or (XXII), the neutral lipid may be DSPC, the steroid alcohol may be cholesterol, and the PEGylated lipid may be PEG-PE (PEG2000-PE).

In another embodiment, the lipidic compound as disclosed herein may be the compounds (IV), (VIII), (IX), (XII), (XVI), (XIX), or (XXII), the neutral lipid may be DSPC, the steroid alcohol may be cholesterol, and the PEGylated lipid may be PEG-PE (PEG2000-PE).

In another embodiment, the lipidic compound as disclosed herein may be the compounds (IV), (IX), (XII), or (XVI), the neutral lipid may be DSPC, the steroid alcohol may be cholesterol, and the PEGylated lipid may be PEG-PE (PEG2000-PE).

In another embodiment, the lipidic compound as disclosed herein may be the compounds (IV) or (XII), the neutral lipid may be DSPC, the steroid alcohol may be cholesterol, and the PEGylated lipid may be PEG-PE (PEG2000-PE).

In another embodiment, the lipidic compound as disclosed herein may be compound (IV), the neutral lipid may be DSPC, the steroid alcohol may be cholesterol, and the PEGylated lipid may be PEG-PE (PEG2000-PE).

Lipid Nanoparticles (LNPs)

The disclosure relates to lipid nanoparticles containing at least on lipidic compound as disclosed herein and at least one nucleic acid.

In one embodiment, the lipid nanoparticles as disclosed herein may contain a lipidic compound as disclosed herein of formula (III) to (XXVII), or the compounds (III), (IV), (V), (VIII), (IX), (XII), (XVI), (XIX), or (XXII), or the compounds (IV), (VIII), (IX), (XII), (XVI), (XIX), or (XXII), or the compounds (IV), (IX), (XII), or (XVI), or the compounds (IV) or (XII), or for example of formula (III), (IV) or (V), and for example of formula (IV).

Further, the lipid nanoparticles as disclosed herein may comprise at least one lipid selected from the group consisting of neutral phospholipids or sphingolipids, steroid alcohols or esters thereof, and PEGylated lipids.

According to one embodiment, a composition as disclosed herein, as above described, may be formulated as lipid nanoparticles.

The lipid nanoparticles may have a diameter making them suitable for systemic, for example parenteral, or for intramuscular, intradermic, or subcutaneous administration. Typically, the lipid nanoparticles have a Z-average size of less than 600 nanometers (nm), for example of less than 400 nm.

In one embodiment, the LNPs have a Z-average size of less than 200 nm. Such size is advantageously compatible with sterile filtration and most appropriate for migration through the lymphatic vessels after intramuscular or subcutaneous administration. This size is also appropriate for intravenous administration, since larger particle injection could induce capillary thrombosis.

In some embodiments, the lipid nanoparticles may have a Z-average size in the range of from about 20 nm to about 300 nm, for example from about 20 nm to about 250 nm, for example from about 30 nm to about 200 nm, from about 40 nm to about 180 nm, from about 60 nm to about 170 nm, from about 80 to about 160 nm, and from about 90 to about 150 nm. In one embodiment, the nanoparticles may have a diameter in the range of about 90 to about 150 nm.

The “a Z-average size” of the lipid nanoparticles may be determined by dynamic light scattering (DLS). The Z-Average size or Z-Average mean used in dynamic light scattering is a parameter also known as the cumulants mean. It is the primary and most stable parameter produced by the technique. The Z-Average mean is defined as the ‘harmonic intensity averaged particle diameter’. A Z-average size may be measured with a zeta sizer Nano ZS light scattering instrument (Malvern Instruments). For accurate particle sizing with the Nano ZS, the viscosity of the buffer and the refractive index of the material had to be provided to the equipment software (PBS: v=1.02 cP, RI=1.45).

As minor variations in size may arise during the manufacturing process, a variation up to 20-30% of the stated measurement is acceptable and considered to be within the stated size. Alternatively, size may be determined by filtration screening assays. For example, a particle preparation is less than a stated size, if at least 90%, for example at least 95%, and for example at least 97% of the particles pass through a “screen-type” filter of the stated size.

The “polydispersity index” is a measurement of the homogeneous or heterogeneous size distribution of the individual lipid nanoparticles in a lipid nanoparticles mixture and indicates the breadth of the particle distribution in a mixture. The PI can be determined, for example, as described herein.

In one embodiment, the polydispersity index of the nanoparticles described herein as measured by dynamic light scattering is 0.5 or less, for example 0.4 or less, for example 0.3 or less, or even for example 0.2 or less.

In one embodiment, the lipid nanoparticles are colloidally stable in the sense that no, or substantially no, aggregation, precipitation or increase of size and polydispersity index as measured by dynamic light scattering may be observed over a given period of time, e.g. over at least two hours to over several months, for example at least 1, 2, 3, 4, 5, 6 or 12 months.

The lipid nanoparticles as disclosed herein have a pKa ranging from 4.5 to 6.7.

This pKa may be determined using a fluorescent probe 2-(p-toluidino)-6-napthalene sulfonic acid (TNS) and preformed LNPs composed of cationic lipid/DOPE/cholesterol/PEG-lipid (35:16:35:2.5 mol %) in PBS at a concentration of ˜6 mM total lipid. In brief, TNS is prepared as a 100 μM stock solution in distilled water. LNPs are diluted to 100 μM of total lipids in 90 μL of buffered solutions (triplicates) containing 10 mM HEPES, 10 mM 4-morpholineethanesulfonic acid, 10 mM ammonium acetate, 130 mM NaCl, where the pH ranges from 2.71 to 11.5. Ten microliters of stock TNS is added to the LNP solutions and mixed well in a black 96-well plate. Fluorescence intensity is monitored in a Tecan Pro200 plate reader using excitation and emission wavelengths of 321 and 445 nm. With the resulting fluorescence values, a sigmoidal plot of fluorescence versus buffer pH is created. The log of the inflection point of this curve is the apparent pKa of the LNP formulation. Such a method is for example detailed in Semple, S. C. et al. Rational design of cationic lipids for siRNA delivery. Nat. Biotechnol. 28, 172-176 (2010).

The lipid nanoparticles may comprise or encapsulate at least one nucleic acid.

The nucleic acid may be encapsulated in and/or adsorbed on an exterior surface of the lipid nanoparticles. The lipidic compound may form a complex with and/or encapsulates the nucleic acid. Alternatively, the lipidic compound may be comprised in a vesicle encapsulating the nucleic acid.

The lipid nanoparticles have a global surface charge which is the sum of the negative and positive electric charges at the surface of the particles and which is represented by the zeta potential. The zeta potential is the potential difference between the dispersion medium and the stationary layer of fluid attached to the dispersed particle. Zeta potential is widely used for quantification of the magnitude of the electrical charge at the double layer.

Zeta potential can be calculated using theoretical models and experimentally determined using electrophoretic mobility or dynamic electrophoretic mobility measurements. Electrophoresis may be used for estimating zeta potential of particulates. In practice, the zeta potential of a dispersion can be measured by applying an electric field across the dispersion. Particles within the dispersion with a zeta potential will migrate toward the electrode of opposite charge with a velocity proportional to the magnitude of the zeta potential. This velocity may be measured using the technique of the Laser Doppler Anemometer. The frequency shift or phase shift of an incident laser beam caused by these moving particles may be measured as the particle mobility, and this mobility may be converted to the zeta potential by inputting the dispersant viscosity and dielectric permittivity, and the application of the Smoluchowski theories. Electrophoretic velocity is proportional to electrophoretic mobility, which is the measurable parameter. There are several theories that link electrophoretic mobility with zeta potential.

Suitable systems such as the Nicomp 380 ZLS system or the Malvern nanoZS can be used for determining the zeta potential. Such systems usually measure the electrophoretic mobility and stability of charged particles in liquid suspension. These values are a predictor of the repulsive forces being exerted by the particles in suspension and are directly related to the stability of the colloidal system.

At pH neutral, the zeta potential of the lipid nanoparticles as disclosed herein is close to neutral.

In one advantage, to have a zeta potential close to zero facilitates particle mobility in the body, reduces opsonization and augment access to target tissues.

In one embodiment, at pH from 6.0 to 7.5, the zeta potential of the nanoparticles may range from about −30 mV to about +5 mV, for example from about −20 mV to about 0 mV, and for example from about −10 mV to about 0 mV.

The lipid nanoparticles described herein can be formed by adjusting, for example at the time of the preparation, a positive to negative charge, depending on the charge ratio of the lipidic compound as disclosed herein (cationic charges from the quaternary ammonium: N of the lipidic compound) to the nucleic acid (anionic charges from the phosphate: P) and mixing the nucleic acid and the lipidic compound. The charges of the lipidic compound and of the nucleic acid are charges at a selected pH, such as a physiological pH, which is from about 6.5 to about 7.5.

The +/− (N/P) charge ratio of the lipidic as disclosed herein to the nucleic acids in the lipid nanoparticles as disclosed herein can be calculated by the following equation. (+/− charge ratio)=[(cationic lipid amount (mol))*(the total number of positive charges in the cationic lipid)][(nucleic acid amount (mol))*(the total number of negative charges in nucleic acid)].

The nucleic acid amount and the lipidic compound amount can be easily determined by one skilled in the art in view of a loading amount upon preparation of the nanoparticles.

According to an embodiment, the ratio of positive to negative charge in nanoparticles suitable for the disclosure is such that they may have a global negative charge or a global charge at or near the neutrality.

In one embodiment, the charge ratio of positive charges to negative charges in the nanoparticles is ranging from about 4:1 to about 15:1, for example from about 5:1 to about 12:1, for example from about 6:1 to about 9:1, and for example is from about 6:1 to about 8:1.

In one embodiment lipid nanoparticles as disclosed herein encapsulating a nucleic acid may have a Z-average size of about 80-200 nm and a charge ratio N/P of about 4-8:1.

Lipid Nanoparticles Manufacturing Process

The present disclosure relates to methods for manufacturing lipid nanoparticles using the lipidic compounds as disclosed herein, for example lipid nanoparticles comprising at least one nucleic acid.

In one embodiment, the nucleic acid containing lipid nanoparticles as disclosed herein may be obtainable by a method comprising at least the steps of:
a) solubilizing, in a water miscible organic solvent containing at least one lipidic compound as disclosed herein, and for example as above described,
b) mixing the organic solvent obtained at step a) with an aqueous solvent comprising at least one nucleic acid, and
c) obtaining said lipid nanoparticles containing said nucleic acid in the aqueous solvent.

In one embodiment, a method for manufacturing lipid nanoparticles as disclosed herein may comprise at least steps of:
a) solubilizing, in a water miscible organic solvent, at least one lipidic compound as disclosed herein and at least one lipid selected from the group consisting of neutral lipids, steroid alcohols or esters thereof, and PEGylated lipids,
b) mixing the organic solvent obtained at step a) with an aqueous solvent comprising at least one nucleic acid, and
c) obtaining said lipid nanoparticles containing said nucleic acid in the aqueous solvent.

The lipidic compound as disclosed herein may be present in an amount sufficient to structure the lipid nanoparticles and to encapsulate any loads to be encapsulated. The amount of ionizable lipidic compound to be used in the lipid nanoparticles may be determined by the skilled person according to any known techniques and is adapted according to the nature and amount of the load, and nature and amount of other lipids susceptible to be present.

In a one embodiment, the step a) further comprises solubilizing in the organic solvent at least one lipid selected from the group consisting of neutral lipids, steroid alcohols or esters thereof, and PEGylated lipids.

Neutral lipids, steroid alcohols or esters thereof, and PEGylated lipids suitable for the disclosure may be as described herein.

In a another embodiment, the step a) may further comprise solubilizing in the organic solvent at least one neutral lipid, at least one steroid alcohol or an ester thereof, and at least one PEGylated lipid, and wherein said lipidic compound, said neutral lipid, said steroid alcohol or ester thereof, and said PEGylated lipid are present in the organic solvent at a molar amount of about 30% to about 70% of lipidic compound, of about 0% to about 50% of neutral lipid, of 20% to about 50% of steroid alcohol or an ester thereof, and of about 1% to about 15% of PEGylated, relative to the total amount of lipid and lipidic compound.

Useful water-miscible organic solvents may be any water-miscible organic solvent capable to solubilize the lipidic compound as disclosed herein and any other added lipids. As example of suitable organic solvents, one may cite ethanol or methanol, 1-propanol, isopropanol, t-butanol, THF, DMSO, acetone, acetonitrile, diglyme, DMF, 1-4 dioxane, ethylene glycol, glycerine, hexamethylphosphoramide, hexamethylphosphorous triamide. In one embodiment, the organic solvent may be ethanol and isopropanol.

Aqueous solvents usable at step b) include aqueous buffered solutions.

As examples of suitable aqueous buffered solution, one may mention acidic buffer, such as include citrate buffer, sodium acetate buffer, succinate buffer, borate buffer or a phosphate buffer. For example, an aqueous buffered solvent may be a citrate buffered solution or an acetate buffered solution.

The pH of the aqueous solvent may range from about 4.5 to about 7.0, for example from about 5.0 to about 6.5, and for example from about 5.5 to about 6.0, and for example may be at about 6.5.

At step b), the organic and aqueous solvents may be mixed at a ratio organic solvent:aqueous solvent ranging from about 1:1 to about 1:6. In one embodiment, the ratio may range from about 1:2 to about 1:4, and for example may be a ratio of about 1:3.

According to one embodiment, the organic solvent and the aqueous solvent may be mixed at step b) at a flow rate ranging from about 0.01 ml/min to about 12 ml/min. In some embodiments, the flow rate may range from about 0.02 ml/min to about 10 ml/min, from about 0.5 ml/min to about 8 ml/min, from about 1 ml/min to about 6 ml/min, or at about 4 ml/min

The step of mixing may be carried by any known method in the art. For instance, both solvents may be mixed with a T-tube or a Y-connector. Alternatively, the mixing may be carried out by laminar flow mixing with a microfluidic micromixer as described by Belliveau et al. (2012).

As indicated, the aqueous solvent at step b) comprises a nucleic acid. In one embodiment, a nucleic acid may encode at least one antigen. A suitable nucleic acid may be for example as detailed below.

The method may further comprise, if necessary, a step of increasing the pH from acidic to neutral.

In a further embodiment, the method may comprise a step d) of increasing the pH of the aqueous solvent containing the lipid nanoparticles obtained at step c) at a pH ranging from about 5.5 to about 7.5, for example from about 6.0 to about 7.0, and for example from about 6.5 to about 7.0.

The step of increasing the pH may be carried by any known method in the art.

For example, the change in pH may carried by a dialyzing or diafiltration step.

According to one embodiment, step d) of the method as disclosed herein may further comprise at least one step of dialyzing or diafiltrating the lipid nanoparticles. The dialysis or diafiltration step may be made against an aqueous solvent with a pH ranging from about 5.5 to about 7.5, for example from about 6.0 to about 7.0, for example from about 6.5 to about 7.0, and for example at about 6.5.

An aqueous solvent usable at step d) may further contain a carbohydrate to improve stabilization of the lipid nanoparticles and osmolarity of the solution. Suitable carbohydrate may be sucrose, mannitol, glucose, dextrose or trehalose. The carbohydrate may be present in an amount, relative to the total amount of the aqueous solvent, of about 5% to about 10%, and for example at about 8%.

According to another embodiment, step d) of the method as disclosed herein may comprise at least two steps of dialyzing the lipid nanoparticles. A first dialyzing step may be made against a similar aqueous solvent (similar in terms of pH and content) and may remove the organic solvent. A second dialysis step may be made against a different aqueous solvent (different in term of pH and possibly in term of content). In such case a pH of the dialyzing solution may range from about 5.5 to about 7.5, for example from about 6.0 to about 7.0, for example from about 6.5 to about 7.0, and for example at about 6.5. The dialyzing solution of the second dialysis may be a buffer solution, for example a phosphate buffer, a TRIS buffer, a Hepes buffer, a histidine buffer, or a glycine buffer. Osmolarity of the buffer may be adjusted with a salt, such as NaCl, or with a carbohydrate, such as glycerol, sucrose, mannitol, glucose, dextrose or trehalose.

In one embodiment, osmolarity is adjusted to reach a final osmolality close to 290 mOsmol/kg as to inject isotonic solution into the body.

Further to step c) and/or d), a method may comprise any further step suitable to harvest, purify, concentrate and/or sterilize the lipid nanoparticles to further formulate them as a pharmaceutical composition, for example as an immunogenic composition.

According to one embodiment, the disclosure relates to lipid nanoparticles obtainable according to the manufacturing method as disclosed herein.

According to another embodiment, the disclosure relates to a method for manufacturing a pharmaceutical composition comprising at least the steps of:
i) admixing at least one nucleic acid and at least one lipidic compound as disclosed herein, or admixing at least one nucleic acid and at least one composition as disclosed herein, or manufacturing at least one lipid nanoparticle according to the method as disclosed herein, and
ii) combining mixed nucleic acid and lipidic compound as disclosed herein, or the mixed nucleic acid and composition as disclosed herein, or the lipid nanoparticles obtained at step i) with at least one pharmaceutically acceptable excipient or carrier.

According to another embodiment, the disclosure relates to a method for manufacturing an immunogenic composition comprising at least the steps of:
i) admixing at least one nucleic acid and at least one lipidic compound as disclosed herein, or admixing at least one nucleic acid and at least one composition as disclosed herein, or manufacturing at least one nucleic acid containing lipid nanoparticle according to the method as disclosed herein, wherein the nucleic acid encodes for at least one antigen, and
ii) combining the mixed nucleic acid and lipidic compound as disclosed herein, or the mixed nucleic acid and composition as disclosed herein, or the lipid nanoparticles obtained at step i) with at least one pharmaceutically acceptable excipient or carrier.

The pharmaceutical and immunogenic compositions suitable for the disclosure are more detailed thereafter.

In one embodiment, the compositions or lipid nanoparticles as disclosed herein may be manufactured with a lipidic compound as disclosed herein of formula (III) to (XXVII), or the compounds (III), (IV), (V), (VIII), (IX), (XII), (XVI), (XIX), or (XXII), or the compounds (IV), (VIII), (IX), (XII), (XVI), (XIX), or (XXII), or the compounds (IV), (IX), (XII), or (XVI), or the compounds (IV) or (XII), or for example of formula (III), (IV) or (V), and for example with compound (IV).

In another embodiment, the lipid nanoparticles as disclosed herein may be manufactured with a neutral lipid that is DSPC or DOPE, a steroid alcohol that is cholesterol, and a PEGylated lipid that is PEG-PE (PEG2000-PE) or DMG-PEG (DMG-PEG2000).

In another embodiment, the lipid nanoparticles as disclosed herein may be manufactured with a lipidic compound of formula (III) to (XXVII), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (III), (IV), (V), (VIII), (IX), (XII), (XVI), (XIX), or (XXII), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (IV), (VIII), (IX), (XII), (XVI), (XIX), or (XXII), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (IV), (IX), (XII), or (XVI), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (IV) or (XII), a neutral lipid that is DSPC, a steroid alcohol that is cholesterol, and a PEGylated lipid that is PEG-PE (PEG2000-PE).

In another embodiment, the lipid nanoparticles as disclosed herein may be manufactured with a lipidic compound of formula (IV), a neutral lipid that is DSPC, a steroid alcohol that is cholesterol, and a PEGylated lipid that is PEG-PE (PEG2000-PE).

Nucleic Acids

The compositions or the lipid nanoparticles as disclosed herein may comprise at least one, anionic or polyanionic, therapeutic agent. A therapeutic agent suitable for the disclosure may be a nucleic acid.

A nucleic acid as disclosed herein may be a deoxyribonucleic acid (DNA) or a ribonucleic acid (RNA), for example RNA, for example an in vitro transcribed RNA (IVT RNA) or synthetic RNA.

Nucleic acids according to the disclosure include genomic DNA, cDNA, mRNA, recombinantly produced and chemically synthesized molecules. A nucleic acid may be in the form of a molecule which is single stranded or double stranded and linear or closed covalently to form a circle. A nucleic can be employed for introduction into, i.e. transfection of, cells, for example, in the form of RNA which can be prepared by in vitro transcription from a DNA template. The RNA can moreover be modified before application by stabilizing sequences, capping, and polyadenylation.

A nucleic acid may be of eukaryotic or prokaryotic origin, and for example of human, animal, plant, bacterial, yeast or viral origin and the like. It may be obtained by any technique known to persons skilled in the art. and for example by screening libraries, by chemical synthesis or alternatively by mixed methods including chemical or enzymatic modification of sequences obtained by screening libraries. They may be chemically modified.

Nucleic acids may be comprised in a vector. Vectors are known to the skilled person and may include plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or PT artificial chromosomes (PAC). The vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment, the nucleic acid may be selected from a group consisting of a double stranded RNA (dsRNA); a single stranded RNA (ssRNA); a double stranded DNA (dsDNA); a single stranded DNA (ssDNA); and combinations thereof.

In one embodiment, the nucleic acid may be selected from a group consisting of messenger RNA (mRNA); an antisense oligonucleotide (ASO); a short interference RNA (siRNA): a self-amplifying RNA (saRNA); a micro RNA (miRNA); a small nuclear RNA (snRNA); a small nucleolar RNA (snoRNA); self-amplifying RNA (saRNA); a plasmid DNA (pDNA); closed-ended DNA (ceDNA), and combinations thereof.

In another embodiment, the nucleic acid may be selected from a group consisting of messenger RNA (mRNA); an antisense oligonucleotide (ASO); a short interference RNA (siRNA): a self-amplifying RNA (saRNA); a micro RNA (miRNA); a plasmid DNA (pDNA); and combinations thereof.

In another embodiment, the nucleic acid may be selected from a group consisting of messenger RNA (mRNA); a short interference RNA (siRNA): a self-amplifying RNA (saRNA); a micro RNA (miRNA); and combinations thereof.

In another embodiment, the nucleic acid may be a messenger RNA (mRNA).

In one embodiment, the nucleic acid is an mRNA. In certain embodiments, the nucleic acid may be an RNA encoding a protein or enzyme. Such polynucleotides may be used as a therapeutic that is capable of being expressed by target cells to facilitate the production of a functional enzyme or protein. For example, in certain embodiments, upon the expression of at least one polynucleotide by target cells the production of a functional enzyme or protein in which a cell or an individual is deficient.

The target cells are cells to which a composition or lipid nanoparticles as disclosed herein are to be directed or targeted. The target cells may comprise a particular tissue or organ. In some embodiments, the target cells may be hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, antigen presenting cells such as dendritic cells, reticulocytes, leukocytes, granulocytes and tumor cells.

mRNA

The term “RNA” relates to a molecule which comprises ribonucleotide residues and for example that is entirely or substantially composed of ribonucleotide residues. “Ribonucleotide” relates to a nucleotide with a hydroxyl group at the 2′-position of a β-D-ribofuranosyl group.

The term includes double stranded RNA, single stranded RNA, isolated RNA such as partially purified RNA, essentially pure RNA, synthetic RNA, or recombinantly produced RNA.

These may be sequences of natural or artificial origin, and for example mRNA (messenger RNA), tRNA (transfer RNA), rRNA (ribosomal RNA), siRNA (silencing RNA), miRNA (micro RNA), mtRNA (mitochondrial RNA), shRNA (short hairpin RNA), tmRNA (transfer-messenger RNA), vRNA (viral RNA), single-stranded, double-stranded and/or base-paired RNA (ssRNA; dsRNA and bpRNA respectively), blunt-ended RNA or not, mature and immature mRNAs, coding and non-coding RNAs, hybrid sequences or synthetic or semisynthetic sequences of oligonucleotides, modified or otherwise, and mixtures thereof.

Accordingly, these may be messenger RNAs (mRNA), which includes mature and immature mRNAs, such as precursor mRNAs (pre-mRNA) or heterogeneous nuclear mRNAs (hnRNA) and mature mRNAs. Thus, RNA molecules as disclosed herein also encompass monocistronic and polycistronic messenger RNAs.

For the sake of clarity, a mRNA encompasses any coding RNA molecule, which may be translated by a eukaryotic host into a protein. A coding RNA molecule generally refers to a RNA molecule comprising a sequence coding for a protein of interest and which may be translated by the eukaryotic host, said sequence starting with a start codon (ATG) and for example terminated by a stop codon (i.e. TAA, TAG. TGA).

An RNA may be a naturally occurring RNA or a modified RNA that differs from naturally occurring RNA by the addition, deletion, substitution and/or alteration of at least one nucleotide. Such alterations can include addition of non-nucleotide material, such as to the end(s) of a RNA or internally, for example at least one nucleotide of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally occurring RNA.

In one embodiment, the RNA is an mRNA (messenger RNA). An mRNA may be a transcript which may be produced using DNA as template and encodes a peptide or protein.

mRNA typically comprises 5′Cap, a 5′ non translated region (5-UTR), a protein or peptide coding region and a 3′ non translated region (3′-UTR), and 3′ polyA tail. mRNA has a limited halftime in cells and in vitro. For example, mRNA is produced by in vitro transcription using a DNA template. Alternatively, the RNA may be obtained by chemical synthesis. The in vitro transcription methodology is known to the skilled person. For example, there is a variety of in vitro transcription kits commercially available.

The RNA may be in vitro synthesized in a cell-free system, using appropriate cell extracts and an appropriate DNA template. For example, cloning vectors are applied for the generation of transcripts. The promoter for controlling transcription can be any promoter for any RNA polymerase. Some examples of RNA polymerases are the T7, T3, and SP6 RNA polymerases. A DNA template for in vitro transcription may be obtained by cloning of a nucleic acid, for example cDNA, and introducing it into an appropriate vector for in vitro transcription. The cDNA may be obtained by reverse transcription of RNA. For example, cloning vectors are used for producing transcripts which generally are designated transcription vectors.

In one embodiment, the RNA may encode for a protein or a peptide. That is, if present in the appropriate environment, for example within a cell, such as an antigen-presenting cell, for example a dendritic cell, the RNA can be expressed to produce a protein or peptide it encodes.

The stability and translation efficiency of an RNA may be modified as required. A modification of an RNA within the present disclosure refers to any modification of RNA which is not naturally present in said RNA.

According to a general embodiment, a mRNA as disclosed herein may comprise or consist of the following general formula:

[5′Cap]w-[5′UTR]x-[Gene of Interest]-[3′UTR]y-[PolyA]z
wherein [5′UTR] and [3′UTR] are untranslated regions (UTR),
wherein [5′UTR] contains a Kozak sequence,
wherein [Gene of Interest] is any gene coding for a protein of interest,
wherein [5′Cap] contains a methyl guanine nucleotide linked to mRNA via a 5′ to 5′ linkage,
wherein [PolyA] is a poly(A) tail, and
wherein w, x, y, and z, are identical or different, and equal to 0 or 1.

According to one embodiment, a mRNA as disclosed herein may consist of the following general formula:

[5′Cap]-[5′UTR]-[Gene of Interest]-[3′UTR]-[PolyA]
wherein [5′UTR] and [3′UTR] are untranslated regions,
wherein [5′UTR] contains a Kozak sequence,
wherein [Gene of Interest] is any nucleic acid coding for a protein of interest,
wherein [5′Cap] contains a methyl guanine nucleotide linked to mRNA via a 5′ to 5′ linkage, and
wherein [PolyA] is a poly(A) tail.

It is reminded that a Kozak sequence refers to a sequence, which is generally a consensus sequence, occurring on eukaryotic mRNAs and which plays a major role in the initiation of the translation process. Kozak sequences and Kozak consensus sequences are well known in the art.

It is also reminded that a poly(A) tail consists of multiple adenosine monophosphates that is well known in the art. A poly(A) tail is generally produced during a step called polyadenylation that is one of the post-translation modifications which generally occur during the production of mature messenger RNAs; such poly(A) tail contribute to the stability and the half-life of said mRNAs, and can be of variable length. For example, a poly(A) tail may be equal or longer than 10 A nucleotides, which includes equal or longer than 20 A nucleotides, which includes equal or longer than 100 A nucleotides, and for example about 120 A nucleotides.

The [3′UTR] does not express any proteins. The purpose of the [3′UTR] is to increase the stability of the mRNA. According to one embodiment, the a-globin UTR is chosen because it is known to be devoid of instability.

Advantageously, the sequence corresponding to the gene of interest may be codon-optimized in order to obtain a satisfactory protein production within the host which is considered.

RNA molecules as disclosed herein may be of variable length. Thus, they may be short RNA molecules, for instance RNA molecules shorter than about 100 nucleotides, or long RNA molecules, for instance longer than about 100 nucleotides, or even longer than about 300 nucleotides.

RNA, such as mRNAs, may encompass synthetic or artificial RNA molecules, but also naturally occurring RNA molecules.

According to the disclosure, a RNA molecule, such as a mRNA, may encompasse the following species:
(i) capped unmodified RNA molecule;
(ii) capped modified RNA molecule;
(iii) uncapped unmodified RNA molecule;
(iv) uncapped modified RNA molecule.

Capped and Uncapped RNA Molecules

According to a most general embodiment, a “capped RNA molecule” refers to a RNA molecule of which the 5′end is linked to a guanosine or a modified guanosine, for example a 7-methylguanosine (m⁷G), connected to a 5′ to 5′ triphosphate linkage or analog. This definition is commensurate with the most widely-accepted definition of a 5′cap, for example of a naturally-occurring and/or physiological cap.

In the sense of the disclosure, “cap analogs” include caps which are biologically equivalent to a 7-methylguanosine (m⁷G), connected to a 5′ to 5′ triphosphate linkage, and which can thus be also substituted without impairing the protein expression of the corresponding messenger RNA in the eukaryotic host.

As example of caps, one may mention m⁷GpppN, m⁷GpppG, m⁷GppₛpG, m⁷GppₛpₛpG, m⁷GppₛpₛpG, m⁷Gppppm⁷G, m₂^{7′,3′-O} GpppG, m₂^{7,2′-O}GpppG, m₂^{7′,2′-O}GppspsG, or m₂^{7′,2′-O}GpppₛpₛG.

Examples of synthetic caps and/or cap analogs can be selected in a list consisting of: glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranos 1 nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Other examples of synthetic caps or cap analogs include ARCA cap analogs, N1-methyl-guanosine, 2′-fluoro-guanosine, 7-deaza-guanosine, 8-oxo-guanosine, 2-amino-guanosine, LNA-guanosine, and 2-azido-guanosine.

Of note, among synthetic caps, some of the above-mentioned caps are suitable as analogs, but not others which may on the contrary hinder protein expression. Such distinction is understood by the man skilled in the art.

For reference, and in a non-limitative manner, the structure of an Anti Reverse Cap Analog (ARCA) 3′-O-Me-m⁷G(5′)ppp(5′)G Cap analog is presented herebelow:

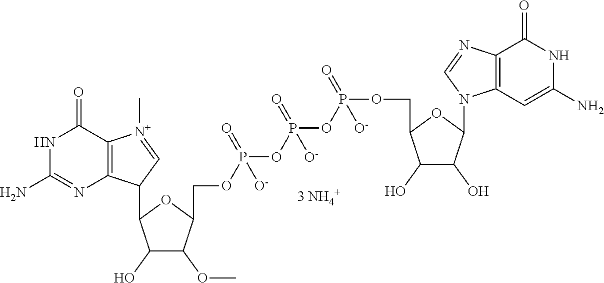 [SMILES: COC1C(COP(=O)([O-])OP(=O)([O-])OP(=O)([O-])OCC2OC(n3cnc4c(=O)[nH]c(N)cc43)C(O)C2O)OC(C2C=[N+](C)c3c2nc(N)[nH]c3=O)C1O]

The ARCA cap analog is, for instance, an example of cap analog used during in vitro transcription: it is a modified cap in which the 3′OH group (closer to m⁷G) is replaced with —OCH₃. However, 100% of the transcripts synthesized with ARCA at the 5′ end are translatable leading to a strong stimulatory effect on translation.

Providing an RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

An “uncapped RNA molecule” refers to any RNA molecule that does not belong to the definition of a “capped RNA molecule”.

Thus, according to a general embodiment, an “uncapped mRNA” may refer to a mRNA of which the 5′end is not linked to a 7-methylguano sine, through a 5′ to 5′ triphosphate linkage, or an analog as previously defined.

An uncapped RNA molecule, such as a messenger RNA, may be an uncapped RNA molecule having a (5′)ρρρ(5′), a (5′)ρρ(5′), a (5′)ρ(5′) or even a (5′)OH extremity. Such RNA molecules may be respectively abbreviated as 5′ρρρRNA; 5′ ρρRNA; 5′ ρRNA; 5′_{OH}RNA. For example, an uncapped RNA molecule as disclosed herein is a messenger 5′ρρρRNA.

Thus, when the RNA molecule is a single-stranded RNA molecule, it may be respectively abbreviated as _{5′ppp}ssRNA; _{5′pp}ssRNA; _{5′p}ssRNA; _{5′OH}ssRNA.

Thus, when the RNA molecule is a double-stranded RNA molecule, it may be respectively abbreviated as _{5′ppp}dsRNA; _{5′pp}dsRNA; _{5′p}dsRNA; _{5′OH}dsRNA.

In one embodiment, an uncapped mRNA as disclosed herein is an uncapped single-stranded mRNA.

According to one embodiment, an uncapped single-stranded mRNA may be an uncapped messenger _{5′ppp}ssRNA.

In a non-limitative manner, the first base of said uncapped RNA molecule may be either an adenosine, a guanosine, a cytosine, or an uridine.

Thus, an uncapped RNA molecule may be an uncapped RNA molecule having a (5′)ppp(5′), a (5′)pp(5′), a (5′)p(5′) or even a blunt-ended 5′ guanosine extremity.

In one embodiment of the disclosure, the RNA may not have uncapped 5′-triphosphates. Removal of such uncapped 5′-triphosphates can be achieved by treating RNA with a phosphatase.

Modified and Unmodified RNA Molecules

The RNA may comprise further modifications. For example, a further modification of the RNA used in the present disclosure may be an extension or truncation of the naturally occurring poly(A) tail or an alteration of the 5′- or 3-untranslated regions (UTR) such as introduction of a UTR which is not related to the coding region of said RNA, for example, the exchange of the existing 3′-UTR with or the insertion of at least one, for example two copies of a 3′-UTR derived from a globin gene, such as alpha 2-globin, alpha 1-globin, beta-globin, for example beta-globin, and for example human beta-globin.

Within the disclosure, a “modified RNA molecule” refers to a RNA molecule which contains at least one modified nucleotide, nucleoside or base, such as a modified purine or a modified pyrimidine. A modified nucleoside or base can be any nucleoside or base that is not A, U, C or G (respectively Adenosine, Uridine, Cytidine or Guanosine for nucleosides; and Adenine, Uracil, Cytosine or Guanine when referring solely to the sugar moiety).

Accordingly, an “unmodified RNA molecule” refers to any RNA molecule that is not commensurate with the definition of a modified RNA molecule.

In the sense of the disclosure, the terms “modified and unmodified” are considered distinctly from the terms “capped and uncapped”, as the latter specifically relates to the base at the 5′-end of an RNA molecule in the sense of the disclosure.

In one embodiment, a nucleic acid, for example an RNA, may comprise at least one modified nucleotide, for example a modified ribonucleotide. The presence of modified nucleotide may increase the stability and/or decrease cytotoxicity of the nucleic acid.

The term stability of RNA relates to the half-life of RNA, that is the period of time which is needed to eliminate half of the activity, amount, or number of molecules. In the context of the present disclosure, the half-life of an RNA is indicative for the stability of said RNA. The half-life of RNA may influence the duration of expression of the RNA. It can be expected that RNA having a long half-life will be expressed for an extended time period.

According to one embodiment, a “modified RNA molecule” refers to a RNA molecule, such as a mRNA, which contains at least one base or sugar modification as described above, and for example at least one base modification as described herein.

For example, in one embodiment, in an RNA suitable for the disclosure 5-methylcytidine may be substituted partially or completely, for example completely, for cytidine. Alternatively, or additionally, in one embodiment, it may be substituted partially or completely, for example completely, for uridine.

In a non-limitative manner, examples of modified nucleotides, nucleosides and bases are disclosed in WO 2015/024667A1.

Thus, a modified RNA molecule may contain modified nucleotides, nucleosides or bases, including backbone modifications, sugar modifications or base modifications.

A backbone modification in connection with the present disclosure includes modifications, in which phosphates of the backbone of the nucleotides contained in a RNA molecule as defined herein are chemically modified

A sugar modification in connection with the present disclosure includes chemical modifications of the sugar of the nucleotides of the RNA molecule as defined herein.

A base modification in connection with the present disclosure includes chemical modifications of the base moiety of the nucleotides of the RNA. In this context nucleotide analogues or modifications are for example selected from nucleotide analogues which are suitable for transcription and/or translation of the RNA molecule in a eukaryotic cell.

Sugar modifications may consist in replacement or modification of the 2′ hydroxy (OH) group, which can be modified or replaced with a number of different “oxy” or “deoxy” substituents.

Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises at least one of the atoms C, N, and O

The sugar group can also contain at least one carbon that possess the opposite stereochemical configuration than that of the corresponding carbon in ribose. Thus, a modified RNA can include nucleotides containing, for instance, arabinose as the sugar.

The phosphate backbone may further be modified and incorporated into the modified RNA molecule, as described herein. The phosphate groups of the backbone can be modified by replacing at least one of the oxygen atoms with a different substituent. Further, the modified nucleosides and nucleotides can include the full replacement of an unmodified phosphate moiety with a modified phosphate as described herein.

Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

The modified nucleosides and nucleotides, which may be incorporated into the modified RNA molecule, as described herein, can further be modified in the nucleobase moiety. For example, the nucleosides and nucleotides described herein can be chemically modified on the major groove face. In some embodiments, the major groove chemical modifications can include an amino group, a thiol group, an alkyl group, or a halo group.

For examples, the nucleotide analogues/modifications are selected from base modifications selected in a list consisting of: 2-amino-6-chloropurineriboside-5′-triphosphate, 2-aminopurine-riboside-5′-triphosphate; 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, and xanthosine-5′-triphosphate.

In some embodiments, modified nucleosides may be selected from a list consisting of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridinei 4-methoxy-pseudouridine, and 4-methoxy-2-thio-pseudouridine.

In some embodiments, modified nucleosides and nucleotides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group.

Modified bases and/or modified RNA molecules are known in the art and are, for instance, further taught in Warren et al. (“Highly Efficient Reprogramming to Pluripotency and Directed Differentiation of Human Cells with Synthetic Modified mRNA”; Cell Stem Cell; 2010).

In view of the above, a modified base may be a modified purine base or a modified pyrimidine base.

In a non-limitative manner, examples of modified purine bases include modified adenosine and/or modified guanosine, such as hypoxanthine; xanthine; 7-methylguanine; inosine; xanthosine and 7-methylguanosine.

According to some embodiments, a modified RNA molecule or mRNA corresponds to an RNA for which each nucleoside corresponding to either Uridine, Cytidine, Adenosine and/or Ribothymidine is modified.

In a non-limitative manner, examples of modified pyrimidine bases include modified cytidine and/or modified uridine, such as 5,6-dihydrouracil; pseudouridine; 5-methylcytidine; 5-hydroxymethylcytidine; dihydrouridine and 5-methylcytidine.

In a non-limitative manner, a modified base as disclosed herein may be a modified uridine or cytidine, such a pseudouridine and 5-methylcytidine.

According to some embodiments, a modified RNA corresponds to a RNA for which at least one base corresponding to either U (for Uracile), C (for Cytosine), A (for Adenine) and/or T (for Thymine) is modified.

As example of modified bases, one may mention methyl-5 uridine (m5U), 2-thio-uridine (s2U), 2′-O-methyl-5 uridine (Ome5U), pseudouridine (Ψ), methyl-1 pseudouridine (m1Ψ), methyl-5 cytosine (m5C), 2′O-methyl-5 cytosine (Om5C), N6-methyl-adenosine (m6A), and N1-methyl-adenosine (m6A).

According to some embodiments, a modified mRNA may comprise as modified bases 2′-O-methyl-5 uridine (Ome5U) or methyl-1 pseudouridine (m1Ψ).

Capped and uncapped mRNAs, whether modified or unmodified, may also be obtained commercially.

RNA having an unmasked poly-A sequence is translated more efficiently than RNA having a masked poly-A sequence.

The term “poly(A) tail” or “poly-A sequence” relates to a sequence of adenyl (A) residues which typically is located on the 3′-end of a RNA molecule and “unmasked poly-A sequence” means that the poly-A sequence at the 3′ end of an RNA molecule ends with an A of the poly—A sequence and is not followed by nucleotides other than A located at the 3′ end, i.e. downstream, of the poly-A sequence. Furthermore, a long poly-A sequence of about 120 base pairs results in an optimal transcript stability and translation efficiency of RNA.

Therefore, in order to increase stability and/or expression of the RNA used according to the present disclosure, it may be modified so as to be present in conjunction with a poly-A sequence, for example having a length of 10 to 500, for example 30 to 300, even for example 65 to 200 and for example 100 to 150 adenosine residues. In one embodiment the poly-A sequence has a length of approximately 120 adenosine residues. To further increase stability and/or expression of the RNA used according to the disclosure, the poly-A sequence can be unmasked.

In addition, incorporation of a 3′-non translated region (UTR) into the 3′-non translated region of an RNA molecule can result in an enhancement in translation efficiency. A synergistic effect may be achieved by incorporating two or more of such 3′-non translated regions. The 3′-non translated regions may be autologous or heterologous to the RNA into which they are introduced. In one embodiment the 3′-non translated region is derived from the human β-globin gene.

A combination of the above described modifications, i.e. incorporation of a poly-A sequence, unmasking of a poly-A sequence and incorporation of at least one 3′-non translated regions, has a synergistic influence on the stability of RNA and increase in translation efficiency.

In order to increase expression of the RNA used according to the present disclosure, it may be modified within the coding region, i.e. the sequence encoding the expressed peptide or protein, for example without altering the sequence of the expressed peptide or protein, so as to increase the GC-content to increase mRNA stability and to perform a codon optimization and, thus, enhance translation in cells.

It is understood that an uncapped RNA molecule may be either a modified RNA molecule or an unmodified RNA molecule.

Accordingly, a capped RNA molecule may be either a modified RNA molecule or an unmodified RNA molecule.

In one embodiment, an RNA molecule as disclosed herein is a messenger RNA (mRNA).

An RNA molecule as disclosed herein is for example an uncapped messenger RNA, either in a modified or in an unmodified form.

An RNA molecule as disclosed herein is for example a capped messenger RNA, either in a modified or in an unmodified form.

In a non-limitative manner, an uncapped RNA molecule, such as a messenger RNA may also be an uncapped RNA molecule having only naturally occurring bases.

According to the disclosure, a “naturally occurring base” relates to a base that can be naturally incorporated in vivo into an RNA molecule, such as a messenger RNA, by the host. Thus, a “naturally occurring base” is distinct from a synthetic base for which there would be not natural equivalent within said host. However, a “naturally-occurring base” may or may not be a modified base, as both terms shall not be confused in the sense of the disclosure.

An uncapped messenger RNA may also be an uncapped and modified messenger RNA, and thus contain at least one modified base.

Thus, an uncapped messenger RNA may also be an uncapped and modified messenger RNA having a (5′)ppp(5′) guanosine extremity and containing at least one modified base.

An uncapped messenger RNA may also be an uncapped and modified messenger RNA having a (5′)ppp(5′) guanosine extremity and containing at least one pseudo-uridine and at least one 5-methylcytosine.

A capped messenger RNA may be a messenger RNA of which the 5′end is linked to a 7-methylguanosine, or analogue, connected to a 5′ to 5′ triphosphate linkage and containing naturally occurring bases or modified bases such as pseudo-urine or 5-methyl cytosine.

It is also understood that, when both modified and unmodified RNA molecules are used within one embodiment of the disclosure, they may be used either as mixtures and/or in purified forms.

Antigens

According to one embodiment, compositions as disclosed herein, such as lipid nanoparticles, may be nucleic acid immunogenic composition or nucleic acid vaccines comprising at least one polynucleotide, e.g. polynucleotide constructs, which encode at least one wild type or engineered antigen.

Antigen-containing compositions as disclosed herein may vary in their valency. Valency refers to the number of antigenic components in the composition or in the polynucleotide (e.g., RNA polynucleotide) or polypeptide. In some embodiments, the immunogenic compositions are monovalent. They may also be compositions comprising more than one valence such as divalent, trivalent or multivalent compositions. Multivalent immunogenic compositions or vaccines may comprise 2, 3, 4, 5, 6, 7, 8, 9, 10, 11, 12, 13, 14, 15, 16, 17, 18, 19, 20, or more antigens or antigenic moieties (e.g., antigenic peptides, etc.). The antigenic components may be on a single polynucleotide or on separate polynucleotides.

Compositions as disclosed herein may be used to protect, treat or cure infection arising from contact with an infectious agent, such as bacteria, viruses, fungi, protozoa and parasites.

Compositions as disclosed herein may be used to protect, treat or cure cancer diseases.

According to one embodiment, a nucleic acid may encode for at least one antigen selected in the group consisting of bacterial antigens, protozoan antigens, viral antigens, fungal antigens, parasite antigens or tumour antigens.

Bacterial Antigens

The bacterium described herein can be a Gram-positive bacterium or a Gram-negative bacterium. Bacterial antigens may be obtained from Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli 0157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Proteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

Viral Antigens

Viral antigens may be obtained from adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Hantavirus, Middle East Respiratory Coronavirus; SARS-Cov-2 virus; Chikungunya virus; Zika virus; parainfluenza virus; Human Enterovirus; Hanta virus; Japanese encephalitis virus; Vesicular exanthernavirus; Eastern equine encephalitisor; or Banna virus.

In one embodiment, the antigen is from a strain of Influenza A or Influenza B virus or combinations thereof. The strain of Influenza A or Influenza B may be associated with birds, pigs, horses, dogs, humans or non-human primates.

The nucleic acid may encode a hemagglutinin protein or fragment thereof. The hemagglutinin protein may be H1, H2, H3, H4, H5, H6, H7, H8, H9, H10, H11, H12, H13, H14, H15, H16, H17, H18, or a fragment thereof. The hemagglutinin protein may or may not comprise a head domain (HA1). Alternatively, the hemagglutinin protein may or may not comprise a cytoplasmic domain.

For example, embodiments, the hemagglutinin protein is a truncated hemagglutinin protein. The truncated hemagglutinin protein may comprise a portion of the transmembrane domain.

In some embodiments, the virus may be selected from the group consisting of HINi, H3N2, H7N9, H5N1 and H10N8 virus or a B strain virus.

In another embodiment, the antigen is from a coronavirus such as SARS-Cov-1 virus, SARS-Cov-2 virus, or MERS-Cov virus.

Fungal Antigens

Fungal antigens may be obtained from Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

Protozoan Antigens

Protozoan antigens may be obtained from Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

Parasitic Antigens

Parasitic antigens may be obtained from Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, Wuchereria bancrofti.

Tumour Antigens

In one embodiment, an antigen may be a tumor antigen, i.e., a constituent of cancer cells such as a protein or peptide expressed in a cancer cell. The term “tumor antigen” or for example relates to proteins that are under normal conditions specifically expressed in a limited number of tissues and/or organs or in specific developmental stages and are expressed or aberrantly expressed in at least one tumor or cancer tissue. Tumor antigens include, for example, differentiation antigens, for example cell type specific differentiation antigens, i.e., proteins that are under normal conditions specifically expressed in a certain cell type at a certain differentiation stage and germ line specific antigens. For example, a tumor antigen is presented by a cancer cell in which it is expressed.

For example, tumor antigens include the carcinoembryonal antigen, a 1-fetoprotein, isoferritin, and fetal sulphoglycoprotein, cc2-H-ferroprotein and γ-fetoprotein.

Other examples for tumor antigens that may be useful in the present disclosure are p53, ART-4, BAGE, beta-catenin/m, Bcr-abL CAMEL, CAP-1, CASP-8, CDC27/m, CD 4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gapl OO, HAGE, HER-2/neu, HPV-E7, HPV-E6, HAST-2, hTERT (or hTRT), LAGE, LDLR/FUT, MAGE-A, for example MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MCi R, Myosin/m, MUC1, MUM-1, -2, -3, NA88-A, NF1, NY-ESO-1, NY-BR-1, pl 90 minor BCR-abL, Pm 1/RARa, PRAME, proteinase 3, PSA, PSM, RAGE, RU1 or RU2, SAGE, SART-1 or SART-3, SCGB3A2, SCP 1, SCP2, SCP3, SSX, SURVrVIN, TEL/AML1, TPI/m, TRP-1, TRP-2, TRP-2/1NT2, TPTE and WT, for example WT-1.

Adjuvants

Nucleic acid containing compositions or lipid nanoparticles as disclosed herein may further comprise, or may be co-administered with, an adjuvant or immune potentiator.

Adjuvants useful in the present disclosure may include, but are not limited to, natural or synthetic adjuvants. They may be organic or inorganic.

Adjuvants may be selected from any of the classes (1) mineral salts, e.g., aluminium hydroxide and aluminium or calcium phosphate gels; (2) emulsions including: oil emulsions and surfactant based formulations, e.g., microfluidised detergent stabilised oil-in-water emulsion, purified saponin, oil-in-water emulsion, stabilised water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensioactive compounds; (9) carbohydrates; or combinations thereof.

Selection of appropriate adjuvants and appropriate amount of adjuvant will be evident to one of ordinary skill in the art.

Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derb/ed P40 Vaccine Adjuvant, MPL™ Adjuvant, AS04, AS02, ASO1, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hlFN-gamma/Interferon-g, Interleukin-ip, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, PAA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(LC), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant El 12K of Cholera Toxin mCT-El 12K, and/or Matrix-S.

Protein Expression

The compositions as disclosed herein or the lipid nanoparticles as disclosed herein encapsulating at least one nucleic acid may also be used for treating individuals deficient in a protein. Therefore, the lipid nanoparticles may be used in a method for treating individuals deficient in a protein comprising administering lipid nanoparticles comprising at least one nucleic acid, for example an mRNA, wherein the nucleic acid encodes a functional protein corresponding to the protein which is deficient in the individual. In embodiments, following expression of the nucleic acid by a target cell a functional protein is produced.

The disclosure also relates to methods of intracellular delivery of nucleic acids that are capable of correcting existing genetic defects and/or providing beneficial functions to at least one target cell. Following successful delivery to target tissues and cells, the compositions and nucleic acids of the present disclosure transfect that target cell and the nucleic acids (e.g., mRNA) can be translated into the gene product of interest (e.g., a functional protein or enzyme) or can otherwise modulate or regulate the presence or expression of the gene product of interest.

The compositions and methods provided herein are useful in the management and treatment of a large number of diseases, for example diseases which result from protein and/or enzyme deficiencies. Individuals suffering from such diseases may have underlying genetic defects that lead to the compromised expression of a protein or enzyme, including, for example, the non-synthesis of the protein, the reduced synthesis of the protein, or synthesis of a protein lacking or having diminished biological activity.

Alternatively, the nucleic acids may encode full length antibodies or smaller antibodies (e.g., both heavy and light chains) to confer immunity to a subject. In an alternative embodiment the compositions of the present disclosure encode antibodies that may be used to transiently or chronically effect a functional response in subjects. For example, the mRNA nucleic acids of the present disclosure may encode a functional monoclonal or polyclonal antibody, which upon translation (and as applicable, systemic excretion from the target cells) may be useful for targeting and/or inactivating a biological target (e.g., a stimulatory cytokine such as tumor necrosis factor). Similarly, the mRNA nucleic acids of the present disclosure may encode, for example, functional anti-nephritic factor antibodies useful for the treatment of membranoproliferative glomerulonephritis type II or acute hemolytic uremic syndrome, or alternatively may encode anti-vascular endothelial growth factor (VEGF) antibodies useful for the treatment of VEGF-mediated diseases, such as cancer.

Pharmaceutical Compositions

According to some embodiments, the disclosure relates to pharmaceutical compositions.

For the purposes of administration, the lipidic compounds of the present disclosure, for example formulated as lipid nanoparticles with a therapeutic agent, such as a nucleic acid, may be administered as pharmaceutical compositions. Pharmaceutical compositions of the present disclosure comprise a lipidic compound as disclosed herein and possibly at least one pharmaceutically acceptable carrier, diluent or excipient.

According to some embodiments, pharmaceutical compositions suitable for the disclosure may comprise (i) at least one nucleic acid and at least one lipidic compound as disclosed herein, or (ii) at least one nucleic acid and at least one composition as described herein, or (iii) at least one nucleic acid containing lipid nanoparticle as described herein, and at least one pharmaceutically acceptable excipient.

In some embodiments, a pharmaceutical composition may be an immunogenic composition. An immunogenic composition suitable for the disclosure may comprise (i) at least one nucleic acid and at least one lipidic compound as disclosed herein, or (ii) at least one nucleic acid and at least one composition as described herein, or (iii) at least one nucleic acid containing lipid nanoparticle as described herein, wherein the nucleic acid encodes for at least one antigen, and at least one pharmaceutically acceptable excipient. Further an immunogenic composition may comprise an adjuvant as described herein.

According to some embodiments, the disclosure relates to a composition comprising (i) at least one nucleic acid and at least one lipidic compound according to the disclosure, or (ii) at least one nucleic acid and at least one composition as described herein, or (iii) at least one nucleic acid containing lipid nanoparticle as described herein, for use as a medicament. Such a medicament may be used for the prevention and/or treatment of a disease as indicated herein.

According to some embodiments, the disclosure relates to a composition comprising (i) at least one nucleic acid and at least one lipidic compound according to the disclosure, or (ii) at least one nucleic acid and at least one composition as described herein, or (iii) at least one nucleic acid containing lipid nanoparticle as described herein, for use in a therapeutic method for preventing and/or treating a disease selected in a group consisting of infectious diseases, allergies, autoimmune diseases, rare blood disorders, rare metabolic diseases, rare neurologic diseases, and tumour or cancer diseases, and for example as herein described.

According to some embodiments, a composition comprising (i) at least one nucleic acid and at least one lipidic compound according to the disclosure, or (ii) at least one nucleic acid and at least one composition as described herein, or (iii) at least one nucleic acid containing lipid nanoparticle as described herein, wherein the nucleic acid encodes for at least one antigen, may be for use as an immunogenic composition.

Immunogenic compositions as disclosed herein may be used in the prevention and/or treatment of an infectious diseases as indicated herein. They may contain a nucleic acid encoding for an antigen as herein described.

In some embodiments, the lipidic compound of formula (I) may be present in a pharmaceutical or immunogenic composition in an amount which is effective to form lipid nanoparticles and deliver a therapeutic agent, for example a nucleic acid, for treating a particular disease or condition of interest.

Appropriate concentrations and dosages can be readily determined by one skilled in the art.

Administration of pharmaceutical and immunogenic compositions as disclosed herein may be carried out via any of the accepted modes of administration of compositions for serving similar utilities.

The compositions as disclosed herein may be formulated into preparations in solid, semi-solid, liquid forms, such as powders, solutions, suspensions or injections. Typical routes of administering such pharmaceutical compositions include, without limitation, oral, topical, transdermal, inhalation, parenteral, sublingual, buccal, intranasal. The term parenteral as used herein includes subcutaneous injections, intravenous, intramuscular, intradermal, intrasternal injection or infusion techniques.

In some embodiment, a composition as disclosed herein may be administered by transdermal, subcutaneous, intradermal or intramuscular route.

Compositions as disclosed herein are formulated so as to allow the active ingredients contained therein to be bioavailable upon administration of the composition to a patient.

Actual methods of preparing such dosage forms are known, or will be apparent, to those skilled in this art; for example, see Remington: The Science and Practice of Pharmacy, 20th Edition (Philadelphia College of Pharmacy and Science, 2000).

The compositions may contain at least one inert diluent or carrier.

In one embodiment, the composition may be in the form of a liquid, for example, a solution, an emulsion or a suspension. The liquid may be for delivery by injection. Compositions intended to be administered by injection may contain at least one of: a surfactant, preservative, wetting agent, dispersing agent, suspending agent, buffer, stabilizer and isotonic agent may be included. The liquid compositions as disclosed herein may include at least one of: sterile diluents such as water for injection, saline solution, for example physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose.

The parenteral preparation can be enclosed in ampoules, disposable syringes or multiple dose vials made of glass or plastic. An injectable pharmaceutical composition is for example sterile.

The pharmaceutical and immunogenic compositions as disclosed herein may be prepared by methodology well known in the pharmaceutical art. For example, a pharmaceutical composition intended to be administered by injection can be prepared by combining the lipid nanoparticles as disclosed herein with sterile, distilled water or other carrier so as to form a solution. A surfactant may be added to facilitate the formation of a homogeneous solution or suspension.

The compositions as disclosed herein are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Compositions as disclosed herein may also be administered simultaneously with, prior to, or after administration of at least one other therapeutic agent. Such combination therapy includes administration of a single pharmaceutical dosage formulation of a composition as disclosed herein and at least one additional active agent, as well as administration of the composition as disclosed herein and each active agent in its own separate pharmaceutical dosage formulation. Where separate dosage formulations are used, the compositions as disclosed herein and at least one additional active agent can be administered at essentially the same time, i.e., concurrently, or at separately staggered times, i.e., sequentially; combination therapy is understood to include all these regimens.

Methods of Treatment

In some embodiments, the disclosure also relates to a method of preventing and/or treating a disease in an individual in need thereof, wherein the method comprises administering an effective amount of (i) at least one nucleic acid and at least one lipidic compound as disclosed herein, or (ii) at least one composition as described herein containing a nucleic acid, or (iii) at least one lipid nanoparticle as described herein containing a nucleic acid, to said individual. For example, a composition containing the LNPs as disclosed herein may be for use in a therapeutic method for preventing and/or treating infectious diseases, allergies, autoimmune diseases, rare blood disorders, rare metabolic diseases, rare neurologic diseases, and tumour or cancer diseases

For example, diseases which may be concerned by the disclosure may infectious diseases such as viral infectious diseases, bacterial infectious diseases, fungal or parasitic infectious diseases. Diseases also concerned by the disclosure may be cancer or tumour diseases.

Viral infectious diseases may be acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, Covid-19, Respiratory Syncytial Virus (RSV) infection, and herpes zoster.

In one embodiment, the disease is influenza, a Respiratory Syncytial Virus (RSV) infection, or Covid-19, and for example is influenza.

Bacterial infectious diseases may be such as abscesses, actinomycosis, acute prostatitis, Aeromonas hydrophila, annual ryegrass toxicity, anthrax, bacillary peliosis, bacteremia, bacterial gastroenteritis, bacterial meningitis, bacterial pneumonia, bacterial vaginosis, bacterium-related cutaneous conditions, bartonellosis, BCG-oma, botryomycosis, botulism, Brazilian purpuric fever, Brodie abscess, brucellosis, Buruli ulcer, campylobacteriosis, caries, Carrion's disease, cat scratch disease, cellulitis, chlamydia infection, cholera, chronic bacterial prostatitis, chronic recurrent multifocal osteomyelitis, clostridial necrotizing enteritis, combined periodontic-endodontic lesions, contagious bovine pleuropneumonia, diphtheria, diphtheritic stomatitis, ehrlichiosis, erysipelas, piglottitis, erysipelas, Fitz-Hugh-Curtis syndrome, flea-borne spotted fever, foot rot (infectious pododermatitis), Garre's sclerosing osteomyelitis, Gonorrhea, Granuloma inguinale, human granulocytic anaplasmosis, human monocytotropic ehrlichiosis, hundred days' cough, impetigo, late congenital syphilitic oculopathy, legionellosis, Lemierre's syndrome, leprosy (Hansen's Disease), leptospirosis, listeriosis, Lyme disease, lymphadenitis, melioidosis, meningococcal disease, meningococcal septicaemia, methicillin-resistant Staphylococcus aureus (MRS A) infection, Mycobacterium avium-intracellulare (MAI), mycoplasma pneumonia, necrotizing fasciitis, nocardiosis, noma (cancrum oris or gangrenous stomatitis), omphalitis, orbital cellulitis, osteomyelitis, overwhelming post-splenectomy infection (OPSI), ovine brucellosis, pasteurellosis, periorbital cellulitis, pertussis (whooping cough), plague, pneumococcal pneumonia, Pott disease, proctitis, pseudomonas infection, psittacosis, pyaemia, pyomyositis, Q fever, relapsing fever (typhinia), rheumatic fever, Rocky Mountain spotted fever (RMSF), rickettsiosis, salmonellosis, scarlet fever, sepsis, serratia infection, shigellosis, southern tick-associated rash illness, staphylococcal scalded skin syndrome, streptococcal pharyngitis, swimming pool granuloma, swine brucellosis, syphilis, syphilitic aortitis, tetanus, toxic shock syndrome (TSS), trachoma, trench fever, tropical ulcer, tuberculosis, tularemia, typhoid fever, typhus, urogenital tuberculosis, urinary tract infections, vancomycin-resistant Staphylococcus aureus infection, Waterhouse-Friderichsen syndrome, pseudotuberculosis (Yersinia) disease, and yersiniosis.

Parasitic infectious diseases may be amoebiasis, giardiasis, trichomoniasis, African Sleeping Sickness, American Sleeping Sickness, leishmaniasis (Kala-Azar), balantidiasis, toxoplasmosis, malaria, Acanthamoeba keratitis, and babesiosis.

Fungal infectious diseases may be aspergilloses, blastomycosis, candidasis, coccidioidomycosis, cryptococcosis, histoplasmosis, mycetomas, paracoccidioidomycosis, and tinea pedis. Furthermore, persons with immuno-deficiencies are for example susceptible to disease by fungal genera such as Aspergillus, Candida, Cryptoccocus, Histoplasma, and Pneumocystis. Other fungi can attack eyes, nails, hair, and especially skin, the so-called dermatophytic fungi and keratinophilic fungi, and cause a variety of conditions, of which ringworms such as athlete's foot are common. Fungal spores are also a major cause of allergies, and a wide range of fungi from different taxonomic groups can evoke allergic reactions in some people.

Cancer or tumour diseases may be cancer or tumor diseases are for example selected from melanomas, malignant melanomas, colon carcinomas, lymphomas, sarcomas, blastomas, renal carcinomas, gastrointestinal tumors, gliomas, prostate tumors, bladder cancer, rectal tumors, stomach cancer, oesophageal cancer, pancreatic cancer, liver cancer, mammary carcinomas (=breast cancer), uterine cancer, cervical cancer, acute myeloid leukaemia (AML), acute lymphoid leukaemia (ALL), chronic myeloid leukaemia (CML), chronic lymphocytic leukaemia (CLL), hepatomas, various virus-induced tumors such as, for example, papilloma virus-induced carcinomas (e.g. cervical carcinoma=cervical cancer), adenocarcinomas, herpes virus-induced tumors (e.g. Burkitt's lymphoma, EBV-induced B-cell lymphoma), heptatitis B-induced tumors (hepatocell carcinomas), HTLV-1- and HTLV-2-induced lymphomas, acoustic neuroma, lung carcinomas (=lung cancer=bronchial carcinoma), small-cell lung carcinomas, pharyngeal cancer, anal carcinoma, glioblastoma, rectal carcinoma, astrocytoma, brain tumors, retinoblastoma, basalioma, brain metastases, medulloblastomas, vaginal cancer, pancreatic cancer, testicular cancer, Hodgkin's syndrome, meningiomas, Schneeberger disease, hypophysis tumor, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, oesophageal carcinoma (=oesophageal cancer), wart involvement, tumors of the small intestine, craniopharyngeomas, ovarian carcinoma, genital tumors, ovarian cancer (=ovarian carcinoma), pancreatic carcinoma (=pancreatic cancer), endometrial carcinoma, liver metastases, penile cancer, tongue cancer, gall bladder cancer, leukaemia, plasmocytoma, lid tumor, prostate cancer (=prostate tumors).

Diseases for which the present disclosure can be useful as a therapeutic intervention include diseases such as SMN1-related spinal muscular atrophy (SMA); amyotrophic lateral sclerosis (ALS); GALT-related galactosemia; Cystic Fibrosis (CF); SLC3A1-related disorders including cystinuria; COL4A5-related disorders including Alport syndrome; galactocerebrosidase deficiencies; X-linked adrenoleukodystrophy and adrenomyeloneuropathy; Friedreich's ataxia; Pelizaeus-Merzbacher disease; TSC1 and TSC2-related tuberous sclerosis; Sanfilippo B syndrome (MPS IIIB); CTNS-related cystinosis; the FMR1-related disorders which include Fragile X syndrome, Fragile X-Associated Tremor/Ataxia Syndrome and Fragile X Premature Ovarian Failure Syndrome; Prader-Willi syndrome; hereditary hemorrhagic telangiectasia (AT); Niemann-Pick disease Type C1; the neuronal ceroid lipofuscinoses-related diseases including Juvenile Neuronal Ceroid Lipofuscinosis (JNCL), Juvenile Batten disease, Santavuori-Haltia disease, Jansky-Bielschowsky disease, and PTT-1 and TPP1 deficiencies; EIF2B1, EIF2B2, EIF2B3, EIF2B4 and EIF2B5-related childhood ataxia with central nervous system hypomyelination/vanishing white matter; CACNA1A and CACNB4-related Episodic Ataxia Type 2; the MECP2-related disorders including Classic Rett Syndrome, MECP2-related Severe Neonatal Encephalopathy and PPM-X Syndrome; CDKL5-related Atypical Rett Syndrome; Kennedy's disease (SBMA); Notch-3 related cerebral autosomal dominant arteriopathy with subcortical infarcts and leukoencephalopathy (CADASIL); SCN1A and SCN1B-related seizure disorders; the Polymerase G-related disorders which include Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions; X-Linked adrenal hypoplasia; X-linked agammaglobulinemia; Fabry disease; and Wilson's disease.

In one embodiment, the nucleic acids, and for example mRNA, of the present disclosure may encode functional proteins or enzymes. For example, the compositions of the present disclosure may include mRNA encoding erythropoietin (EPO), al-antitrypsin, carboxypeptidase N, alpha galactosidase (GLA), ornithine carbamoyltransferase (OTC), or human growth hormone (hGH).

In other embodiments, the disclosure relates to methods of transfecting at least one isolated target cell with a nucleic acid, wherein said method comprises contacting the at least one target cell with an effective amount of at least one nucleic acid polynucleotide and (i) at least one nucleic acid and at least one lipidic compound as disclosed herein, or (ii) at least one composition as described herein containing a nucleic acid, or (iii) at least one lipid nanoparticle containing a nucleic acid as described herein, such that the at least one target cell are transfected with said nucleic acid.

Target cells include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, antigen presenting cells such as dendritic cells, reticulocytes, leukocytes, granulocytes and tumor cells.

In one embodiment, the cells targeted may be spleen, liver, lung, heart and kidney cells. In another embodiment, the cells targeted may be spleen and kidney cells, and for example may be spleen cells.

In some embodiments, lipid nanoparticles or compositions as disclosed herein which allow avoiding hepatic clearance may be of particular interest.

Following transfection of at least one target cell by, for example, the nucleic acid encapsulated in the lipid nanoparticles, the production of a polypeptide or a protein encoded by such nucleic acid may be for example stimulated and the capability of such target cells to express the nucleic acid and produce, for example, a polypeptide or protein of interest is enhanced. For example, transfection of a target cell by a composition encapsulating mRNA will enhance (i.e., increase) the production of the protein or enzyme encoded by such mRNA.

In other embodiments, the disclosure relates to methods of producing a polypeptide in at least one target cell, wherein said method comprises contacting the at least one target cell with an effective amount of (i) at least one nucleic acid and at least one lipidic compound as disclosed herein, or (ii) at least one composition as herein described containing a nucleic acid, or (iii) at least one lipid nanoparticle containing a nucleic acid as described herein, such that the at least one target cell are transfected with the nucleic acid operably encoding said polypeptide.

It is to be understood that the disclosure encompasses all variations, combinations, and permutations in which at least one limitation, element, clauss, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The following examples are provided for purpose of illustration and not limitation.

### EXAMPLES

Materials and Methods

Nuclear Magnetic Resonance Spectroscopy (H, C NMR)
H and C NMR spectra were recorded at room temperature on the following spectrometer: Brucker Advance 400 (NMR H: 400 MHz and NMR C: 75 MHz).

Recorded shifts were reported in parts per million (6) and calibrated using residual undeuterated 3: H 7.26 ppm; C 77.16 ppm, MeOH H 3.31 ppm; C: 49.0 ppm). Data were represented as follows, chemical shift, multiplicity (s=singlet, d=doublet, t=triplet, q=quartet and m=multiplet), coupling constant (J in Hz), integration and attribution.

NMR spectra were obtained using the commercial software NMRnotebook.
High-resolution mass spectra (HRMS) were obtained using an Agilent Q-TOF (time of flight) 6520 and Low-resolution mass spectra (LCMS) using an Agilent MSD 1200 SL (ESI/APCI) with an Agilent HPLC 1200 SL.

### Example 1: Synthesis of N-((Z)-14-(((E)-octadec-9-en-1-yl)oxy)-3,6,9,12,16-pentaoxatetratriacont-25-en-1-yl)-1H-imidazole-4-carboxamide (compound IV) (Also Named DOG-IM4)

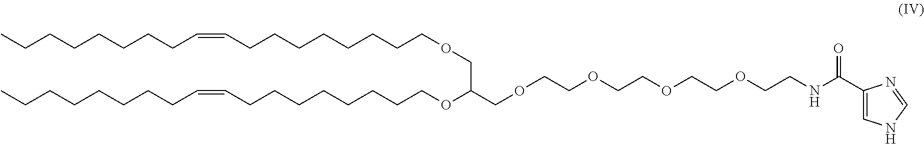 [SMILES: CCCCCCCC/C=C\CCCCCCCCOCC(COCCOCCOCCOCCNC(=O)c1c[nH]cn1)OCCCCCCCC/C=C\CCCCCCCC]

The compound IV is prepared according to the following schema of synthesis

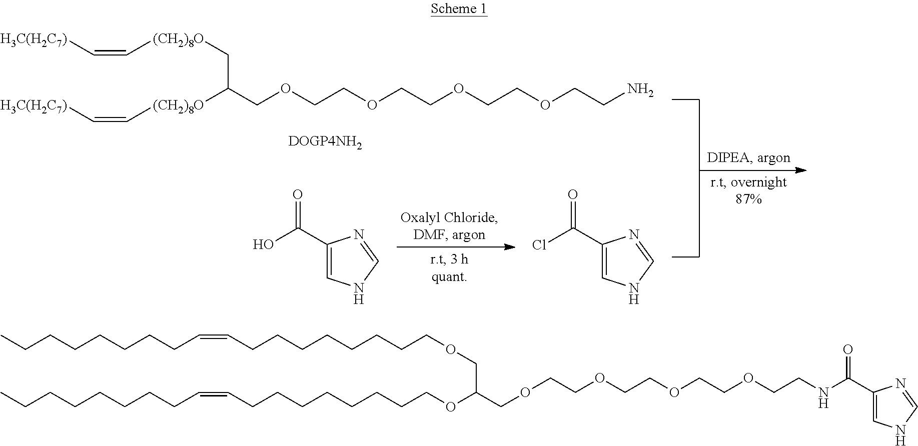

### 1.1 Synthesis of DOG-PEG₄-NH₂ Also Named DOGP4NH₂

Its schemes of synthesis are as follow:

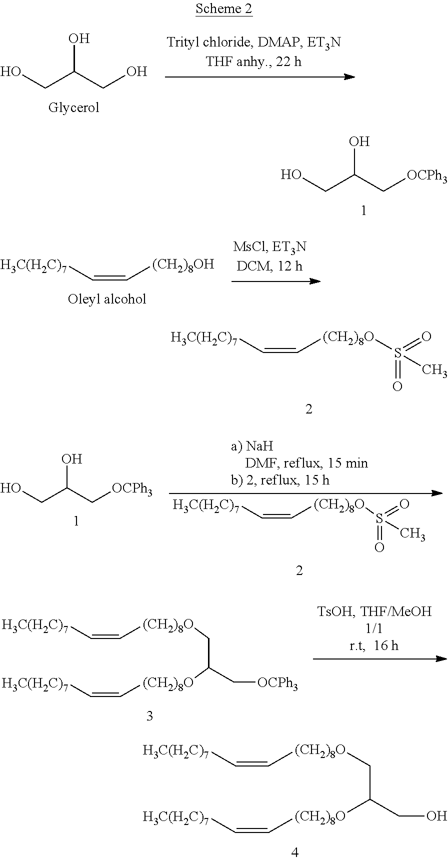

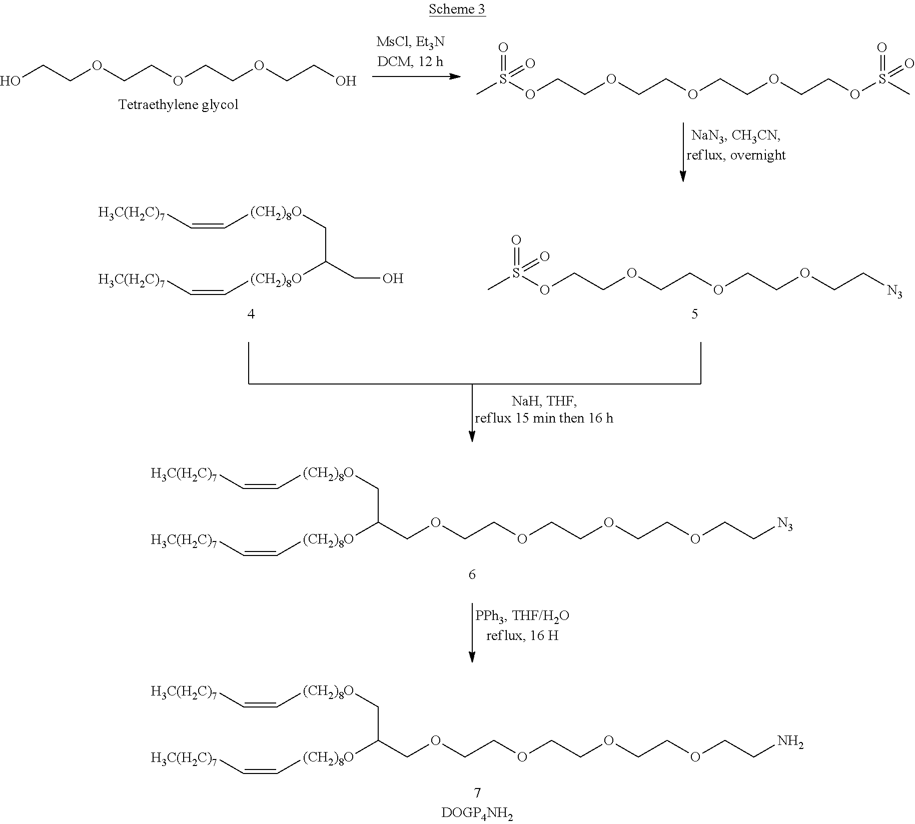

### 1.1.1 Synthesis of triphenylmethane-glycerol (1)

Glycerol (30.0 g; 325.8 mmoles), trityle cloride (22.5 g; 80.7 mmoles) and DMAP (225 mg; 1.84 mmole) were dissolved in 60 mL of anhydrous THF. After addition of triethylamine (13.5 mL; 96.9 mmoles), the mixture was stirred vigorously for 22 h at room temperature. 100 mL of ethyl acetate and 70 mL H₂O were then added to the solution. The aqueous phase was extracted with 2×70 mL ethyl acetate. The organic phases were combined, washed successively with 70 mL of 10% (w/v) NaHCO₃ and 70 mL of brine, dried over MgSO₄ and filtered. The obtained product was further purified by silica gel column chromatography (elution gradient CH₂Cl₂/MeOH) to yield compound 1 as a white solid (15.7 g; yield 58%).

RMN ¹H (300 MHz; CDCl3): δ: 7.49-7.29 (m; 15Hf-j), 3.93-3.90 (m; 1Hb), 3.76-3.62 (m; 2Ha), 3.35-3.24 (m; 2Hc).

ES-SM (N2) m/z: 357.1589 ([M+Na]+); exact mass: 334.1689 g·mol⁻¹

### 1.1.2 Synthesis of 1-methanesulfonyl-oleyl alcool (2)

Oleic alcohol (45.0 g; 167.6 mmoles) and triethylamine (38 mL; 272.0 mmoles) were dissolved in 600 mL of dichloromethane (CH₂Cl₂) and the mixture was stirred at 4° C. Methanesulfonyle chloride (17 mL; 217.0 mmoles) was added dropwise and the reaction mixture was placed under vigorous stirring under argon at room temperature. After 12 h, 250 mL H₂O were added and the aqueous phase was extracted with 2×250 ml de CH₂Cl₂. The organic layer was washed successively with 250 mL of 1N HCl, 250 mL of 10% (w/v) NaHCO₃, and 250 mL of brine and dried over MgSO₄. The solvent was then evaporated under vacuum. The obtained product was further purified by silica gel column chromatography (elution gradient cyclohexane/AcOEt from 10/0 to 10/1). Compound 2 was obtained as a yellowish oil (44 g; yield 76%).

RMN ¹H (300 MHz; CDCl3): δ: 5.39-5.31 (m; 2H9-10), 4.21 (t; J=6.4 Hz; 2H1), 2.99 (s; 3Ha), 2.14-1.88 (m; 4H8, 11), 1.80-1.67 (tt; J=6.8 Hz; 2H2), 1.52-1.14 (m; 22H3-7.12-17), 0.88 (t; J=6.8 Hz; 3H18).

ES-SM (N2) m/z: 385.3969 ([M+K]+); exact mass: 346.2989 g·mol⁻¹

### 1.1.3 Synthesis of triphenylmethane-dioleylglycerol (3)

To a suspension of NaH (6.0 g (60% in oil); 149.5 mmoles) in 35 mL of anhydrous DMF was added compound 1 (10.0 g; 29.2 mmoles) in solution in 145 mL of anhydrous DMF. The mixture was heated under reflux for 15 minutes and cooled to room temperature. Product 2 (25.9 g; 74.8 mmoles) in 90 ml of anhydrous DMF was added dropwise to the mixture which was then heated under reflux for 15 h. After cooling to RT, 120 mL of H₂O were added to eliminate remaining NaH. The aqueous layer was extracted with ethyl acetate (2×100 mL). Combined organic layers were washed with 2×240 mL of 1N HCl, 2×240 mL of 5% (w/v) NaHCO₃ and 240 mL of brine, dried over MgSO₄ and filtered. The solvent was evaporated under reduced pressure. Compound 3 obtained as a crude yellowish oil was used without further purification (17 g; yield 70%).

ES-SM (N2) m/z: 857.5507 ([M+Na]+); exact mass: 834.5607 g·mol⁻¹ (product was detected by MS)

### 1.1.4 Synthesis of dioleylglycerol (4)

Compound 3 (16.0 g; 19.5 mmoles) and para-toluenesulfonique acid (pTs-OH·H₂O) (1.2 g; 6.1 mmoles) were dissolved in 270 mL of THF/MeOH 1/1 and stirred for 16 h at room temperature. Triethylamine (860 μl; 6.1 mmoles) was then added to the mixture to eliminate excess of pTsOH·H₂O, and the solvent was evaporated under reduced pressure. The residual oil was purified by silica gel chromatography (cyclohexane/AcOEt) affording compound 4 as a colorless oil (6.5 g; yield 57%).

RMN ¹H: (300 MHz; CDCl3): δ: 5.38-5.32 (m; 4H9-10), 3.76-3.41 (m; 9Hb-a-c-1), 2.13-1.89 (m; 8H8, 11), 1.69-1.48 (m; 4H2), 1.47-1.12 (m; 44H3-7.12-17), 0.89 (t; J=6.6 Hz; 6H18).

ES-SM (N2) m/z: 615.5213 ([M+Na]+); exact mass: 592.5313 g·mol⁻¹

### 1.1.5 Synthesis of methanesulfonyloxy-ethoxy-ethoxy-ethoxy-ethyl-azoture (5)

Di-mesylate tetraethyleneglycol (25.0 g; 71.4 mmoles) was heated under reflux in 150 mL of CH₃CN in the presence of NaN₃ (5.8 g; 89.5 mmoles). After 19 h the mixture was cooled to RT and the precipitate was recovered by filtration and purified by silical gel chromatography (cyclohexane/AcOEt (7/3 to 3/7)) affording compound 5 as a yellow oil (8.7 g; Yield 41%).

RMN ¹H (200 MHz; CDCl3): δ: 4.26-4.22 (m; 2Hd), 3.66-3.61 (m; 2He), 3.60-3.52 (10Hf-j), 3.26 (t; J=5.4 Hz; 2Hk), 2.95 (s; 3H1).

ES-SM (N2) m/z: 320.0539 ([M+Na]+); exact mass: 297.0639 g·mol⁻¹

### 1.1.6 Synthesis of dioleylglycero-ethoxy-ethoxy-ethoxy-ethyl-azoture (6)

To a suspension of NaH (810 mg (60% in oil); 20.2 mmoles) in 13 mL of anhydrous THF was added compound 4 (4 g; 6.8 mmoles) in solution in 50 mL of anhydrous THF containing 13 mL of HMPA. The mixture was heated under reflux for 15 min and cooled to RT. Compound 5 (4 g; 13.5 mmoles) in solution in 25 mL of anhydrous THF was added dropwise. The resulting mixture was heated under reflux for 15 h, cooled to RT and excess NaH was eliminated by addition of 400 mL H₂O. The organic phase was collected and the aqueous phase was extracted with 3×400 mL AcOEt. The organic phases were combined and washed successively with 2×400 ml of 1N HCl, 2×400 ml of 5% (w/v) NaHCO₃ and 400 ml of brine and dried on MgSO₄. The solvent was evaporated under reduced pressure and the resulting oil was purified on a silica gel column eluted with cyclohexane/AcOEt to yield a yellowish oil (4 g; Yield 74%).

RMN ¹H (300 MHz; CDCl3): δ: 5.39-5.33 (m; 4H9-10), 3.70-3.50 (m; 23Ha-j, 1), 3.45-3.39 (t; J=5.3 Hz; 2Hk), 2.05-1.95 (m; 8H8, 11), 1.58-1.53 (m; 4H2), 1.43-1.21 (m; 44H3-7.12-17), 0.88 (t; J=6.8 Hz; 6H18)

ES-SM (N2) m/z: 816.6715 ([M+Na]+); Exact mass: 793.6815 g·mol⁻¹

### 1.1.7 Synthesis of 2-[2-[2-[2-[2,3-bis[(˜-Z})-octadec-9-enoxylpropoxylethoxyl ethoxylethoxylethanamine (DOG-PEG₄-NH₂) (7)

Compound 6 (1.8 g; 2.3 mmoles) was dissolved in 180 mL of THF in the presence of triphenylphosphine (1.8 g; 6.8 mmoles) and 400 mL H₂O. The mixture was heated under reflux for 15 h and the solvent was then evaporated under reduced pressure. The remaining oil was purified on a silica gel column (elution gradient CH₂Cl₂/MeOH/NH₄0H 9/0.9/0.1) to yield compound 7 as colorless oil (1.5 g; Yield 86%).

RMN ¹H (300 MHz; CDCl3/MeOD 1/1): δ: 5.35-5.29 (m; 4H9-10), 3.64-3.43 (m; 23Ha-j-1), 2.78-2.90 (m; 2Hk), 2.06-1.90 (m; 8H8, 11), 1.60-1.52 (m; 4H2), 1.40-1.19 (m; 44H3-7.12-17), 0.86 (t; J=7.1 Hz; 6H18).

ES-SM (N2) m/z: 768.6636 ([M]+); exact mass: 768.6636 g·mol⁻¹

### 1.2 Synthesis of N-((Z)-14-(((E)-octadec-9-en-1-yl)oxy)-3,6,9,12,16-pentaoxatetratriacont-25-en-1-yl)-1H-imidazole-4-carboxamide (compound IV; also named DOG-IM4)

4-Imidazolcarboxylic acid (50 mg, 446 μmol) was dissolved in 1 mL oxalyl chloride and a drop of DMF was added to catalyze the reaction. The reaction was stirred at room temperature under nitrogen atmosphere. After 3 hours, the organic phase was evaporated and the remaining yellow solid was dried overnight under vacuum pump to obtain without purification the corresponding acid chloride (58 mg, quant.).

DOG-PEG₄-NH₂ (30 mg, 39 μmol) was dissolved in 5 mL anhydrous DCM and the acid chloride (5.6 mg, 43 μmol) in 1.5 mL anhydrous DMF and DIPEA (25 μL) were added. The mixture was stirred at room temperature under nitrogen atmosphere overnight. The solvent was evaporated and the product was purified by flash chromatography (4 g column, DCM/MeOH/NH4OH 9/0.9/0.1) affording the desired compound (30 mg, 87%).

¹H-NMR (CDCl₃, 400 MHz): δ 7.69-7.61 (m, 3H, NH, N═CH—NH, NHCH═C), 5.40-5.29 (m, 4H, 2×CH═CH), 3.68-3.39 (m, 25H, 12×OCH₂, 1×OCH, CH₂NHC(O)), 2.06-1.90 (m, 8H, 2×CH₂CH═CHCH₂), 1.59-1.49 (m, 4H, 2×OCH₂CH₂), 1.39-1.20 (m, 44H, 22×oleyl-CH₂), 0.87 (t, J=6.8, 6H, 2×CH₃) ppm.

¹³C-NMR (CDCl₃, 75 MHz): δ 163.04 (NHC═O), 135.52 (N═CH—NH), 130.53, 130.43, 130.07, 129.97 (2×CH═CH, NHCH═C, CH═C), 78.06 (OCH), 71.88-70.15 (12×OCH₂), 39.09 (CH₂NHC(O)), 32.76-26.23 (oleyl), 22.83 (2×CH₃CH₂), 14.25 (2×CH₃) ppm.

HR-MS (direct injection, positive ionization): m/z=884.7039 [M+Na]⁺ (calculated: 884.71)

### Example 2: Synthesis of N-((Z)-14-(((E)-octadec-9-en-1-yl)oxy)-3,6,9,12,16-pentaoxatetratriacont-25-en-1-yl)-1H-imidazole-2-carboxamide (compound III)

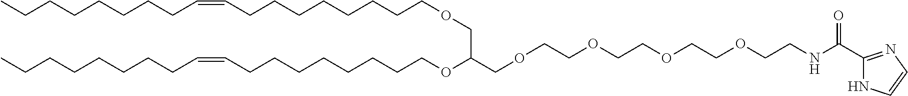 [SMILES: CCCCCCCC/C=C\CCCCCCCCOCC(COCCOCCOCCOCCNC(=O)c1ncc[nH]1)OCCCCCCCC/C=C\CCCCCCCC]

It is prepared according to the scheme 1 and the molar amounts considered in example 1 by using 2-Imidazolcarboxylic acid instead of 4-Imidazolcarboxylic acid. The product was purified by flash chromatography (4 g column, DCM/MeOH/NH4OH 9/0.9/0.1) affording the desired compound (45 mg, 65%).

¹H-NMR (CDCl₃, 400 MHz): δ 7.15 (m, 1H, N—CH═CH), 7.16 (m, 1H, CH═CH—NH), 5.40-5.29 (m, 4H, 2×CH═CH), 3.68-3.37 (m, 25H, 12×OCH₂, 1×OCH, CH₂NHC(O)), 2.2 (br s, 1H, NH signal), 2.1-1.90 (m, 8H, 2×CH₂CH═CHCH₂), 1.59-1.49 (m, 4H, 2×OCH₂CH₂), 1.37-1.20 (m, 44H, 22×oleyl-CH₂), 0.87 (t, J=6.8, 6H, 2×CH₃) ppm.

¹³C-NMR (CDCl₃, 75 MHz): δ 158.95 (NHC═O), 141.20 (N═CH—NH), 130.52, 130.44, 130.06, 129, 98, 129.87 (2×CH═CH, CH═CH—NH), 119.09 (N—CH═C), 78.06 (OCH), 71.83-69.80 (12×OCH₂), 39.30 (CH₂NHC(O)), 32.76-26.23 (oleyl), 22.83 (2×CH₃CH₂), 14.25 (2×CH₃) ppm.

HR-MS (direct injection, positive ionization): m/z=884.7057 [M+Na]⁺ (calculated: 884.71)

### Example 3: Synthesis of N-((Z)-14-(((E)-octadec-9-en-1-yl)oxy)-3,6,9,12,16-pentaoxatetratriacont-25-en-1-yl)-1H-pyridinyl-3-carboxamide (compound V)

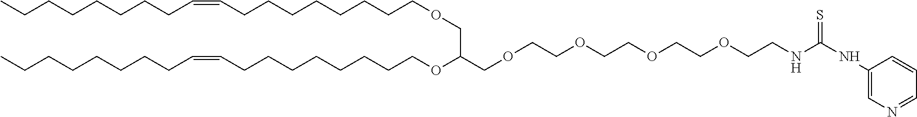 [SMILES: CCCCCCCC/C=C\CCCCCCCCOCC(COCCOCCOCCOCCNC(=S)Nc1cccnc1)OCCCCCCCC/C=C\CCCCCCCC]

It is prepared according to the scheme 1, the molar amounts considered in example 1 and by using 3-Pyridyl isothicyanate instead of 4-Imidazolcarboxylic acid.

¹H-NMR (CDCl₃, 400 MHz): δ 8.79-8.01 (m, 3H, pyridin), 7.26 (m, 1H, pyridin), 5.40-5.29 (m, 4H, 2×CH═CH), 3.94-3.29 (m, 25H, 12×OCH₂, 1×OCH, CH₂NHC(S)), 2.1-1.90 (m, 8H, 2×CH₂CH═CHCH₂), 1.61-1.45 (m, 4H, 2×OCH₂CH₂), 1.42-1.18 (m, 44H, 22×oleyl-CH₂), 0.87 (t, J=6.8, 6H, 2×CH₃) ppm.

¹H-NMR (MeOD, 400 MHz): δ 8.62, 8.29, 8.09, 7.39 (m, 4H, pyridin), 5.42-5.31 (m, 4H, 2×CH═CH), 3.88-3.39 (m, 25H, 12×OCH₂, 1×OCH, CH₂NHC(S)), 2.08-1.94 (m, 8H, 2×CH₂CH═CHCH₂), 1.61-1.49 (m, 4H, 2×OCH₂CH₂), 1.40-1.24 (m, 44H, 22×oleyl-CH₂), 0.90 (t, J=6.8, 6H, 2×CH₃) ppm.

¹³C-NMR (CDCl₃, 75 MHz): δ 181.82 (CH₂NHC(S)), 145.86, 145.01, 136.18, (3C, pyridin) 131.20-129.84 (1C pyridine, 2×CH═CH), 123.22 (1C, pyridin), 78.05 (OCH), 77.94, 72.72-70.22 (12×OCH₂), 44.86 (CH₂NHC(S)), 32.73-26.24 (oleyl), 22.80 (2×CH₃CH₂), 14.23 (2×CH₃) ppm.

HR-MS (direct injection, positive ionization): m/z=904.7166 [M+H]⁺ (calculated: 904.72)

### Example 4: Synthesis of N-[2-[2-[2-[2-[2,3-bis[(Z)-octadec-9-enoxy]propoxy]ethoxy]ethoxy]ethoxy]ethyl]-1H-imidazole-4-carboxamide (compound IV)

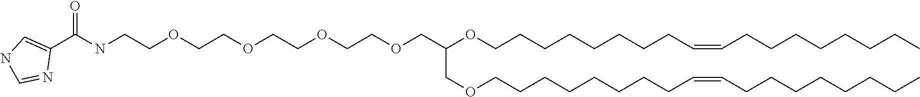 [SMILES: CCCCCCCC/C=C\CCCCCCCCOCC(COCCOCCOCCOCCNC(=O)c1c[nH]cn1)OCCCCCCCC/C=C\CCCCCCCC]

A mixture of 2-[2-[2-[2-[2,3-bis[(Z)-octadec-9-enoxy]propoxy]ethoxy]ethoxy]ethoxy]ethanamine (1.2 g, 1.56 mmol) in DCM (30 mL) was added a solution of 1H-imidazole-4-carbonyl chloride (0.612 g, 4.69 mmol) and DIEA (1.01 g, 7.81 mmol) in DMF (20 mL). The mixture was stirred for 16 h at ambient temperature. The mixture was concentrated and the residue was purified by column chromatography on silica gel eluting with 0%-10% MeOH in DCM to afford N-[2-[2-[2-[2-[2,3-bis[(Z)-octadec-9-enoxy]propoxy]ethoxy]ethoxy]ethoxy]ethyl]-1H-imidazole-4-carboxamide (0.504 g, 37.4%) as yellow oil.

¹H NMR (500 MHz, CDCl₃) δ 10.99 (s, 1H), 7.73-7.59 (m, 3H), 5.39-5.30 (m, 4H), 3.69-3.40 (m, 25H), 2.09-1.92 (m, 8H), 1.60-1.50 (m, 4H), 1.28 (t, J=14.5 Hz, 44H), 0.88 (t, J=6.9 Hz, 6H).

### Example 5: Synthesis of N-[2-[2-[2-[2-(2,3-dihexadecoxypropoxy)ethoxy]ethoxy]ethoxy]ethyl]-1H-imidazole-4-carboxamide (compound VI)

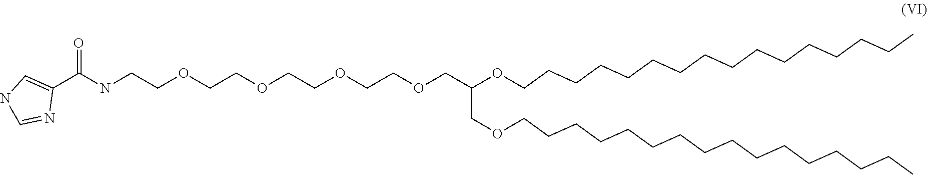 [SMILES: CCCCCCCCCCCCCCCCOCC(COCCOCCOCCOCCNC(=O)c1c[nH]cn1)OCCCCCCCCCCCCCCCC]

4-imidazolcarboxylic acid (2.5 g, 22.3 mmol) was dissolved in 60 mL oxalyl chloride and several drop of DMF was added to catalyze the reaction. The reaction was stirred at room temperature under nitrogen atmosphere overnight. The organic phase was evaporated, and the remaining yellow solid was dried overnight under vacuum pump to obtain without purification the corresponding acid chloride (2.5 g, quant.). 2-[2-[2-[2-(2,3-dihexadecoxypropoxy)ethoxy]ethoxy]ethoxy]ethanamine (400 mg, 0.5 mmol) was dissolved in 25 mL anhydrous DCM and the acid chloride (262 mg, 2 mmol) in 3 mL anhydrous DMF and DIPEA (0.325 g, 2.5 mmol) were added. The mixture was stirred at room temperature under nitrogen atmosphere overnight. The solvent was evaporated and the product purified by flash chromatography (12 g column, DCM/MeOH/NH4OH 9/0.9/0.1) affording N-[2-[2-[2-[2-(2,3-dihexadecoxypropoxy)ethoxy]ethoxy]ethoxy]ethyl]-1H-imidazole-4-carboxamide (250 mg, 0.293 mmol, 58.3% yield) as yellow solid.

¹H NMR (500 MHz, CDCl₃) δ 7.65 (s, 1H), 7.62 (s, 1H), 7.59 (s, 1H), 3.69-3.40 (m, 25H), 1.60-1.49 (m, 4H), 1.33-1.22 (m, 52H), 0.88 (t, J=6.9 Hz, 6H).

### Example 6: Synthesis of N-[2-[2-[2-[2-[2,3-bis[(Z)-octadec-9-enoxy]propanoylamino]ethoxy]ethoxy]ethoxy]ethyl]-1Himidazole-4-carboxamide (compound VII)

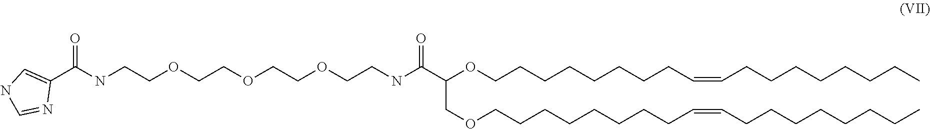 [SMILES: CCCCCCCC/C=C\CCCCCCCCOCC(OCCCCCCCC/C=C\CCCCCCCC)C(=O)NCCOCCOCCOCCNC(=O)c1c[nH]cn1]

The compound was synthesized based on the chemistry shown in Scheme (4).

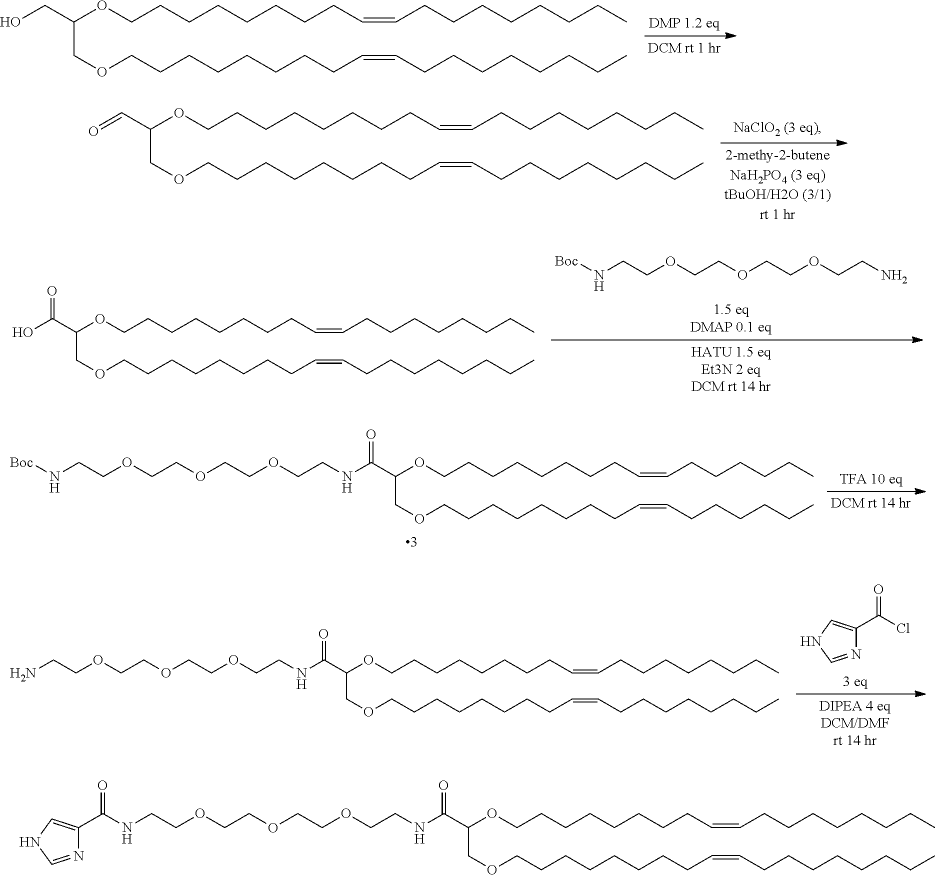

Synthesis

Step (1)

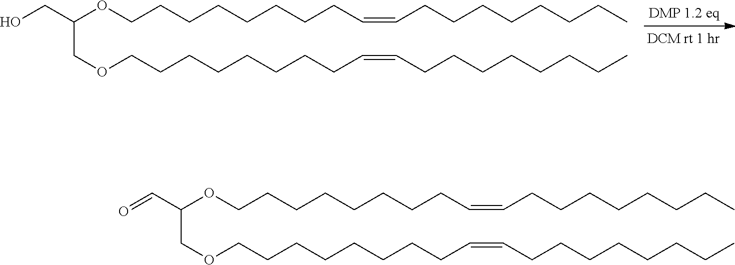 [SMILES: CCCCCCCC/C=C\CCCCCCCCOCC(CO)OCCCCCCCC/C=C\CCCCCCCC ; CCCCCCCC/C=C\CCCCCCCCOCC(C=O)OCCCCCCCC/C=C\CCCCCCCC]

To a solution of 2,3-bis[(Z)-octadec-9-enoxy]propan-1-ol (1 g, 1.69 mmol) in DCM (20 ml) at 0° C. was added Dess-Martin Periodinane (841 mg, 1.69 mmol) for 5 min. Then the mixture was stirred at 25° C. for 2 hrs under N₂. After reaction, the mixture was diluted DCM (30 ml), washed with NaHCO₃/Na₂S₂O₃(1/1) (50 ml×3) and brine (50 ml), dried over Na₂SO₄, filtered and concentrated to give 2,3-bis[(Z)-octadec-9-enoxy]propanal (1.2 g, crude) as a yellow oil which was taken into next step directly.

¹H NMR (400 MHz, CDCl₃) δ 9.72 (d, J=1.4 Hz, 1H), 5.35 (t, J=5.4 Hz, 4H), 3.84-3.79 (m, 1H), 3.74-3.56 (m, 5H), 3.44 (ddd, J=12.6, 9.4, 2.7 Hz, 3H), 2.03-1.96 (m, 8H), 1.63 (d, J=7.2 Hz, 2H), 1.54 (d, J=6.9 Hz, 2H), 1.26 (d, J=4.5 Hz, 44H), 0.90-0.87 (m, 6H).

Step (2)

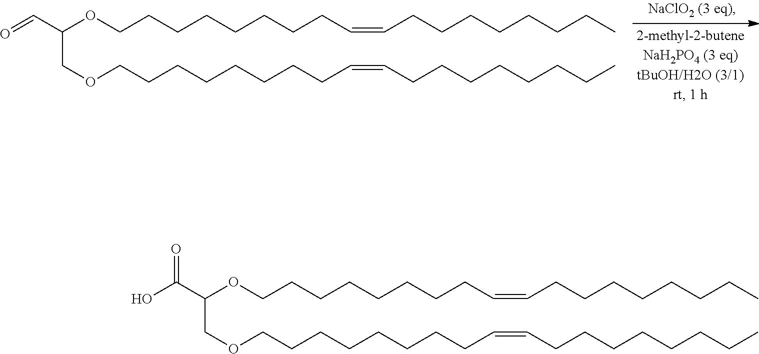 [SMILES: CCCCCCCC/C=C\CCCCCCCCOCC(C=O)OCCCCCCCC/C=C\CCCCCCCC ; CCCCCCCC/C=C\CCCCCCCCOCC(OCCCCCCCC/C=C\CCCCCCCC)C(=O)O]

2,3-bis[(Z)-octadec-9-enoxy]propanal (1.2 g, 1.62 mmol) was dissolved in t-BuOH: H₂O (3:1, 20 mL), containing NaH₂PO₄.2H₂0 (759 mg, 4.87 mmol), 2-methy-2-butene (3.4 mL) and sodium chlorite (411 mg, 4.87 mmol). The reaction was stirred for 1 hr at room temperature and was diluted with ethyl acetate. The aqueous layer was extracted with ethyl acetate. The combined organic layers were dried over sodium sulfate, filtered and concentrated to afford 2,3-bis[(Z)-octadec-9-enoxy]propanoic acid (778 mg, yield 78.9%) as colorless oil.

¹H NMR (400 MHz, CDCl₃) δ 5.40-5.31 (m, 4H), 4.04 (dd, J=5.0, 3.3 Hz, 1H), 3.80 (dd, J=10.5, 3.2 Hz, 1H), 3.70 (dd, J=10.5, 5.1 Hz, 1H), 3.62 (q, J=6.8 Hz, 2H), 3.51-3.44 (m, 2H), 2.01 (dd, J=14.7, 8.9 Hz, 8H), 1.65-1.54 (m, 4H), 1.27 (dd, J=6.7, 2.7 Hz, 44H), 0.88 (t, J=6.8 Hz, 6H).

Step (3)

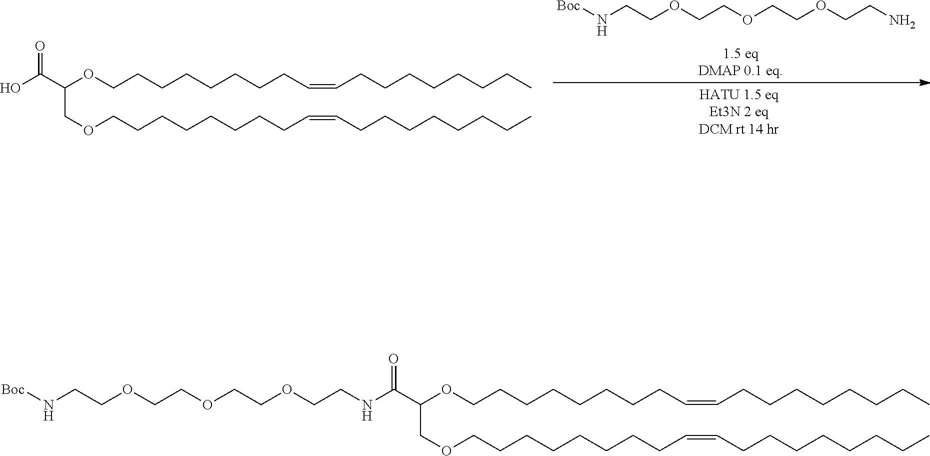 [SMILES: CC(C)(C)OC(=O)NCCOCCOCCOCCN ; CCCCCCCC/C=C\CCCCCCCCOCC(OCCCCCCCC/C=C\CCCCCCCC)C(=O)O ; CCCCCCCC/C=C\CCCCCCCCOCC(OCCCCCCCC/C=C\CCCCCCCC)C(=O)NCCOCCOCCOCCNC(=O)OC(C)(C)C]

To a solution of 2,3-bis[(Z)-octadec-9-enoxy]propanoic acid (100 mg, 0.165 mmol) in DCM (2 ml) was added tert-butyl N-[2-[2-[2-(2-aminoethoxy)ethoxy]ethoxy]ethyl]carbamate (48 mg, 0.165 mmol), 4-Dimethylaminopyridine (2 mg, 0.02 mmol), 0-(7-Azabenzotriazol-1-yl)-N,N,N′,N′-tetramethyluronium hexafluorophosphate (94 mg, 0.25 mmol) and triethylamine (33 mg, 0.33 mmol). The mixture was stirred at 25° C. for 18 hr. After reaction, the mixture was diluted with DCM (50 ml), washed with water (50 ml×2), brine (50 ml), dried over Na₂SO₄, filtered and concentrated. The residue was purified by flash chromatography eluted with 2% to 8% methanol in dichloromethane to give tert-butyl N-[2-[2-[2-[2-[2,3-bis[(Z)-octadec-9-enoxy]propanoylamino]ethoxy]ethoxy]ethoxy]ethyl]carbamate as light yellow oil.

Step (4)

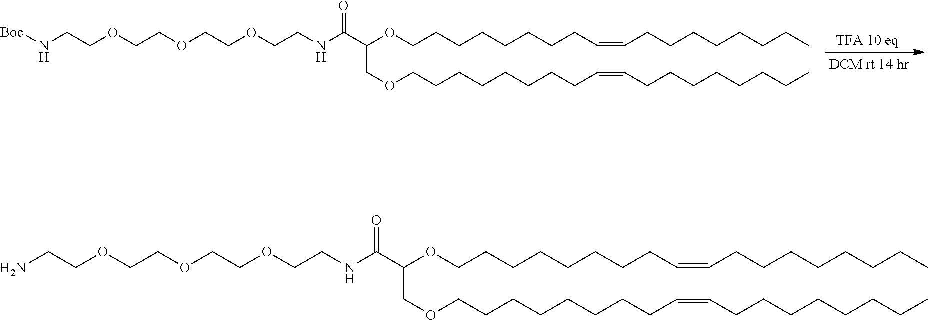 [SMILES: CCCCCCCC/C=C\CCCCCCCCOCC(OCCCCCCCC/C=C\CCCCCCCC)C(=O)NCCOCCOCCOCCNC(=O)OC(C)(C)C ; CCCCCCCC/C=C\CCCCCCCCOCC(OCCCCCCCC/C=C\CCCCCCCC)C(=O)NCCOCCOCCOCCN]

To a solution of tert-butyl N-[2-[2-[2-[2-[2,3-bis[(Z)-octadec-9-enoxy]propanoylamino]ethoxy]ethoxy]ethoxy]ethyl]carbamate (226 mg, 0.256 mmol) in DCM (2 ml) was added TFA (0.5 ml). The mixture was stirred at 25° C. for 3 hrs. After reaction, the mixture was concentrated to give tert-butyl N-[2-[2-[2-[2-[2,3-bis[(Z)-octadec-9-enoxy]propanoylamino]ethoxy]ethoxy]ethoxy]ethyl]carbamate (300 mg, crude).

¹H NMR (400 MHz, CDCl₃) δ 5.41-5.31 (m, 4H), 3.94 (s, 1H), 3.83-3.69 (m, 7H), 3.53 (dddd, J=26.7, 22.9, 11.7, 4.5 Hz, 15H), 2.06-1.91 (m, 8H), 1.54 (d, J=7.0 Hz, 4H), 1.26 (d, J=4.3 Hz, 44H), 0.88 (t, J=6.8 Hz, 6H).

Step (5)

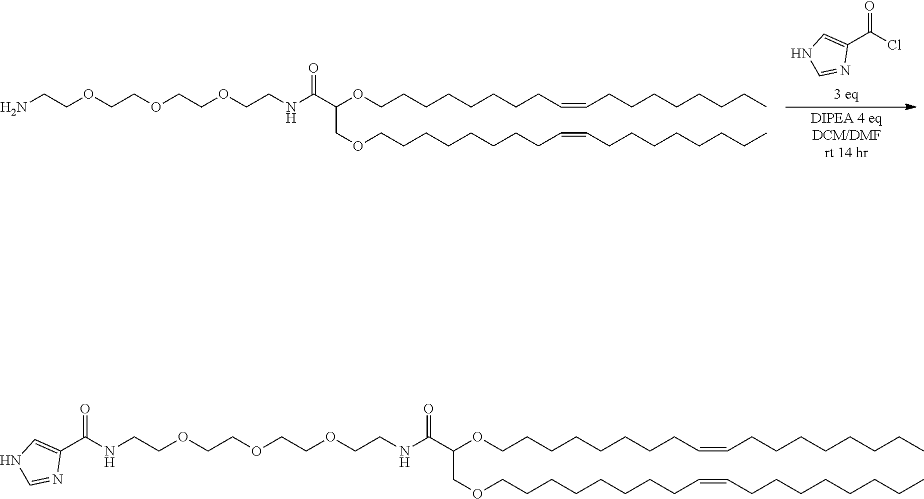 [SMILES: CCCCCCCC/C=C\CCCCCCCCOCC(OCCCCCCCC/C=C\CCCCCCCC)C(=O)NCCOCCOCCOCCN ; O=C(Cl)c1c[nH]cn1 ; CCCCCCCC/C=C\CCCCCCCCOCC(OCCCCCCCC/C=C\CCCCCCCC)C(=O)NCCOCCOCCOCCNC(=O)c1c[nH]cn1]

To a solution of N-[2-[2-[2-(2-aminoethoxy)ethoxy]ethoxy]ethyl]-2,3-bis[(Z)-octadec-9-enoxy]propanamide (400 mg, 0.512 mmol) in DCM (10 ml) was added N,N-Diisopropylethylamine (265 mg, 2.05 mmol), 1H-imidazole-4-carbonyl chloride (200 mg, 1.54 mmol) in DMF (2 ml). The mixture was stirred at 25° C. for 14 hr. After reaction, the mixture was diluted with EA (100 ml), washed with water (100 ml×2), brine (100 ml). The organic was concentrated and purified by flash (10% MeOH in DCM) to give N-[2-[2-[2-[2-[2,3-bis[(Z)-octadec-9-enoxy]propanoylamino]ethoxy]ethoxy]ethoxy]ethyl]-1H-imidazole-4-carboxamide (280 mg, yield 61.2%) as a colourless oil.

¹H NMR (400 MHz, CDCl₃) δ 10.35-10.08 (m, 1H), 7.64 (s, 1H), 7.59 (s, 1H), 7.48 (s, 1H), 7.05 (s, 1H), 5.44-5.31 (m, 4H), 3.89 (dd, J=5.6, 2.8 Hz, 1H), 3.77 (dd, J=10.6, 2.6 Hz, 1H), 3.69-3.39 (m, 21H), 2.10-1.93 (m, 7H), 1.63-1.52 (m, 5H), 1.26 (d, J=4.6 Hz, 44H), 0.88 (t, J=6.8 Hz, 6H).

### Example 7: Synthesis of nonyl 8-[3-[2-[2-[2-[2-(1H-imidazole-4-carbonylamino)ethoxy]ethoxy]ethoxy]ethoxy]-2-(8-nonoxy8-oxo-octoxy)propoxy]octanoate (compound VIII)

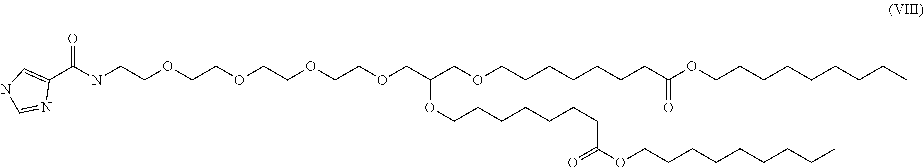 [SMILES: CCCCCCCCCOC(=O)CCCCCCCOCC(COCCOCCOCCOCCNC(=O)c1c[nH]cn1)OCCCCCCCC(=O)OCCCCCCCCC]

The compound (VIII) was synthesized based on the chemistry shown in Scheme (5).

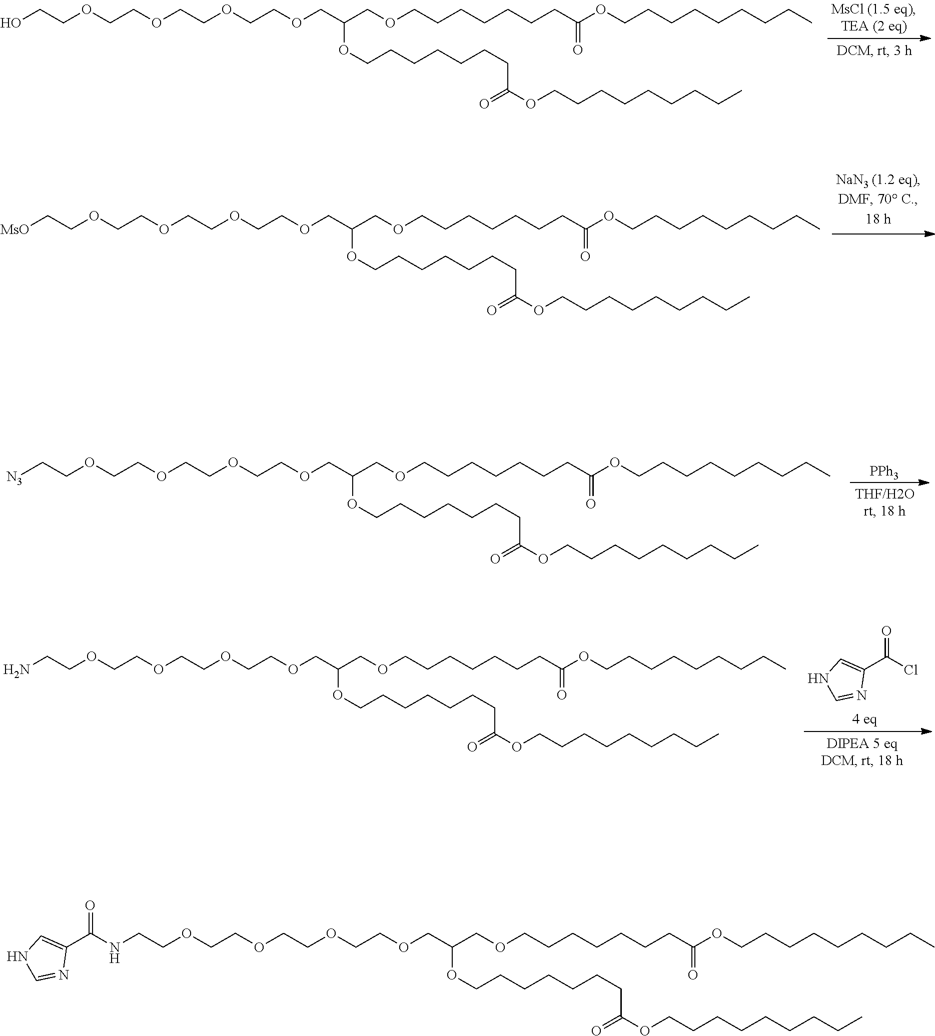

Synthesis

Step (1)

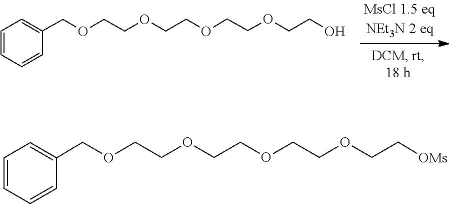 [SMILES: OCCOCCOCCOCCOCc1ccccc1 ; CS(=O)(=O)OCCOCCOCCOCCOCc1ccccc1]

2-[2-[2-(2-benzyloxyethoxy)ethoxy]ethoxy]ethanol (50 g, 176 mmol) and triethylamine (35.6 g, 352 mmol) in dry dichloromethane (500 mL) under nitrogen were cooled to −5° C. Methanesulfonyl chloride (30.2 g, 264 mmol) in dry DCM (20 mL) was added dropwise to this solution at 0° C. The mixture was allowed to warm to room temperature and stirred at room temperature for 18 h. Tri-ethylamine hydrochloride was filtered off, and the DCM solution was washed with 0.1 N HCl and dried over sodium sulfate. Removing the solvent afforded 2-[2-[2-(2-benzyloxyethoxy)ethoxy]ethoxy]ethyl methanesulfonate (69.1 g, 175 mmol, quant.) as light yellow oil which was used without further purification.

¹H NMR (400 MHz, CDCl₃) δ 7.37-7.27 (m, 5H), 4.56 (s, 2H), 4.39-4.33 (m, 2H), 3.78-3.73 (m, 2H), 3.69-3.60 (m, 12H), 3.06 (s, 3H).

Step (2)

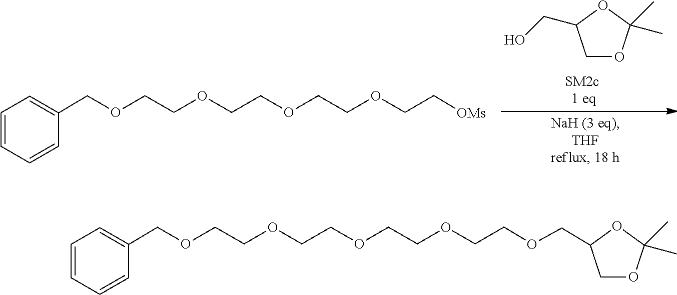

To the solution of (2,2-dimethyl-1,3-dioxolan-4-yl)methanol (24.4 g, 175 mmol) in THF (500 mL) was added NaH (14 g, 351 mmol) and the mixture was heated to reflux for 15 min. Then the reaction was cooled to room temperature and 2-[2-[2-(2-benzyloxyethoxy)ethoxy]ethoxy]ethyl methanesulfonate (69.1 g, 175 mmol) was added under nitrogen and the reaction was heated at 80° C. for 24 h. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy)ethoxy]ethoxy]ethoxymethyl]-2,2-dimethyl-1,3-dioxolane (54.4 g, 70% yield) as light yellow oil.

¹H NMR (400 MHz, CDCl₃) δ 7.38-7.27 (m, 5H), 4.57 (s, 2H), 4.28 (t, J=5.9 Hz, 1H), 4.05 (dd, J=8.3, 6.4 Hz, 1H), 3.72 (dd, J=8.3, 6.4 Hz, 1H), 3.70-3.61 (m, 16H), 3.57 (dd, J=10.0, 5.8 Hz, 1H), 3.49 (dd, J=10.0, 5.5 Hz, 1H), 1.42 (s, 3H), 1.35 (s, 3H).

Step (3)

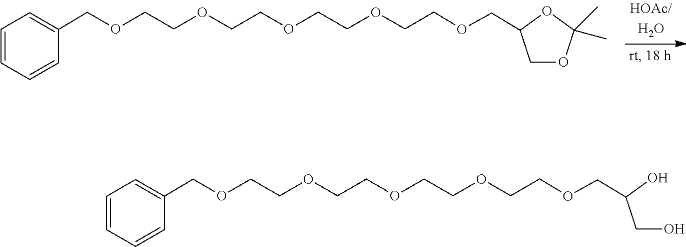 [SMILES: CC1(C)OCC(COCCOCCOCCOCCOCc2ccccc2)O1 ; OCC(O)COCCOCCOCCOCCOCc1ccccc1]

The mixture of 4-[2-[2-[2-(2-benzyloxyethoxy)ethoxy]ethoxy]ethoxymethyl]-2,2-dimethyl-1,3-dioxolane (54.4 g, 123 mmol) in AcOH (200 mL) and H₂O (200 mL) was stirred at room temperature for 18 h.

TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy)ethoxy]ethoxy]ethyl methanesulfonate (49 g, 123 mmol, quant.) as light yellow oil was obtained which was used without further purification.

¹H NMR (400 MHz, CDCl₃) δ 7.38-7.27 (m, 5H), 4.57 (s, 2H), 3.88-3.81 (m, 1H), 3.70-3.51 (m, 21H).

Step (4)

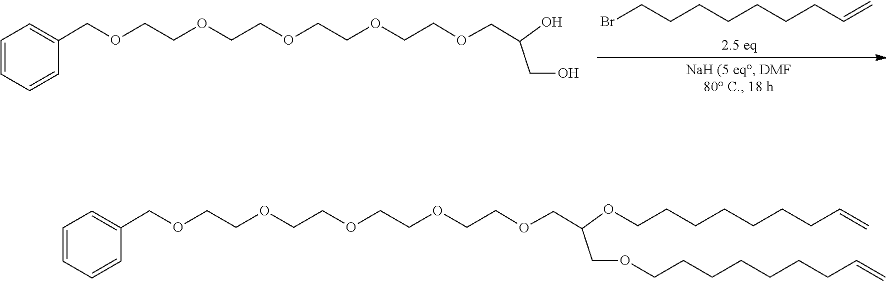

To a solution of 3-[2-[2-[2-(2-benzyloxyethoxy)ethoxy]ethoxy]ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h.

TLC (EA/PE=1/1, Rf. 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered and concentrated. The residue was purified by flash chromatography eluted with 20% to 50% ethyl acetate in petroleum ether to give 2-[2-[2-[2-[2,3-bis(non-8-enoxy)propoxy]ethoxy]ethoxy]ethoxy]ethoxymethylbenzene (9.3 g, 14.6 mmol, 24.2% yield) as light yellow oil.

¹H NMR (400 MHz, CDCl₃) δ 7.37-7.27 (m, 5H), 5.89-5.72 (m, 2H), 5.04-4.89 (m, 4H), 4.57 (s, 2H), 3.71-3.60 (m, 17H), 3.59-3.38 (m, 9H), 2.03 (q, J=6.7 Hz, 4H), 1.60-1.49 (m, 4H), 1.43-1.23 (m, 16H).

Step (5)

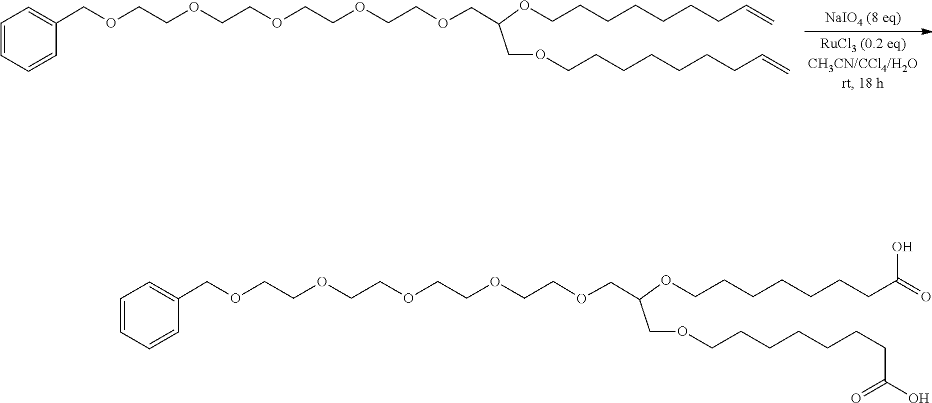 [SMILES: C=CCCCCCCCOCC(COCCOCCOCCOCCOCc1ccccc1)OCCCCCCCC=C ; O=C(O)CCCCCCCOCC(COCCOCCOCCOCCOCc1ccccc1)OCCCCCCCC(=O)O]

To a solution of 2-[2-[2-[2-[2,3-bis(non-8-enoxy)propoxy]ethoxy]ethoxy]ethoxy]ethoxymethylbenzene (9.3 g, 14.6 mmol) in MeCN (80 mL), CCl₄ (80 mL) and water (80 mL) was added NaIO₄ (24.9 g, 116 mmol) and RuCl₃ (656 mg, 2.91 mmol). The reaction mixture was stirred at room temperature for 24 h.

LCMS indicated that the title compound was the major product along with partial mono-aldehyde product. The reaction was filtered, and the filtrate was diluted with ethyl acetate (800 mL) and washed with 1N aq. HCl (400 mL). The organic layer was washed with Na₂S₂O₃ solution and then dried over sodium sulfate, filtered and concentrated to give 8-[3-[2-[2-[2-(2-benzyloxyethoxy)ethoxy]ethoxy]ethoxy]-2-(7-carboxyheptoxy)propoxy]octanoic acid (10 g, 12.4 mmol) as yellow oil which was used without further purification.

Step (6)

8-[3-[2-[2-[2-(2-benzyloxyethoxy)ethoxy]ethoxy]ethoxy]-2-(8-oxooctoxy)propoxy]octanoic acid (10 g, 8 mmol) was dissolved in t-BuOH:H2O (3:1, 160 mL), containing NaH₂PO₄·2H₂O (3.73 g, 24 mmol), 2-methy-2-butene (40 mL) and sodium chlorite (2.71 mg, 24 mmol). The reaction was stirred for 2 h at rt and LCMS indicated that the starting material was consumed. The reaction mixture was diluted with ethyl acetate. The aqueous layer was extracted with ethyl acetate. The combined organic layers were dried over sodium sulfate and concentrated to afford 8-[3-[2-[2-[2-(2-benzyloxyethoxy)ethoxy]ethoxy]ethoxy]-2-(7-carboxyheptoxy)propoxy]octanoic acid (10 g, 3.22 mmol, quant.) as light yellow oil.

¹H NMR (400 MHz, CDCl3) δ 7.38-7.27 (m, 5H), 4.57 (s, 2H), 3.71-3.61 (m, 17H), 3.59-3.37 (m, 9H), 2.33 (t, J=7.3 Hz, 4H), 1.69-1.51 (m, 8H), 1.39-1.28 (m, 14H).

Step (7)

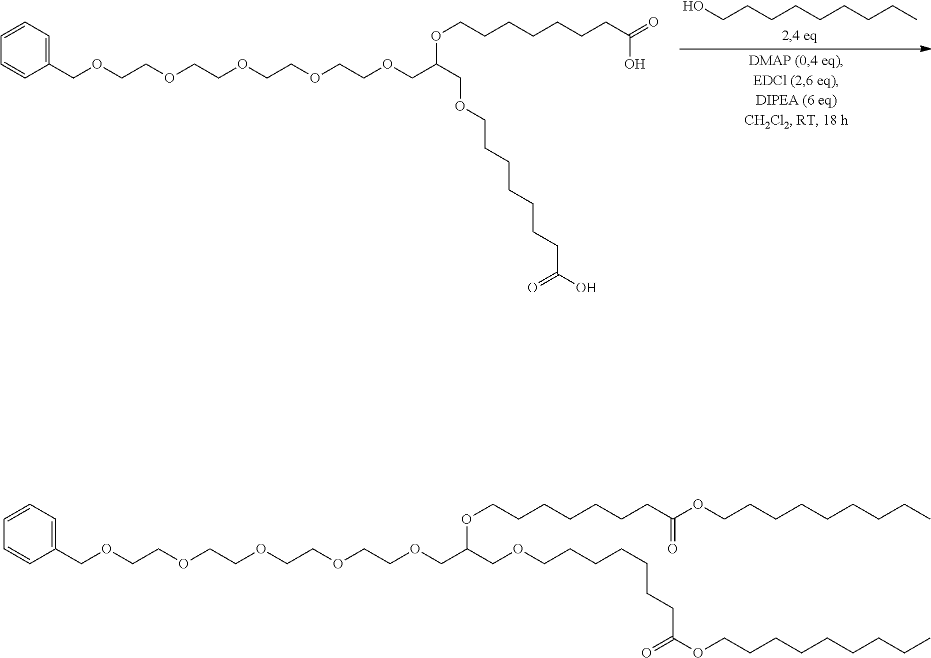

To the solution of 8-[3-[2-[2-[2-(2-benzyloxyethoxy)ethoxy]ethoxy]ethoxy]-2-(7-carboxyheptoxy)propoxy]octanoic acid (10 g, 14.8 mmol) and 1-Nonanol (5.12 g, 35.5 mmol) in dry dichloromethane (200 mL) under nitrogen were added N,N-Diisopropylethylamine (11.5 g, 88.7 mmol), DMAP (0.722 g, 5.91 mmol) and EDCI (7.37 g, 38.4 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 20% to 55% ethyl acetate in petroleum ether to give nonyl 8-[3-[2-[2-[2-(2-benzyloxyethoxy)ethoxy]ethoxy]ethoxy]-2-(8-nonoxy-8-oxo-octoxy)propoxy]octanoate (5 g, 35.9%) as colorless oil. n H NMR (400 MHz, CDC3) δ 7.38-7.27 (m, 5H), 4.57 (s, 2H), 4.05 (t, J=6.8 Hz, 4H), 3.70-3.61 (m, 16H), 3.59-3.39 (m, 9H), 2.28 (t, J=7.5 Hz, 4H), 1.67-1.50 (m, 12H), 1.37-1.21 (m, 36H), 0.88 (t, J=6.8 Hz, 6H).

Step (8)

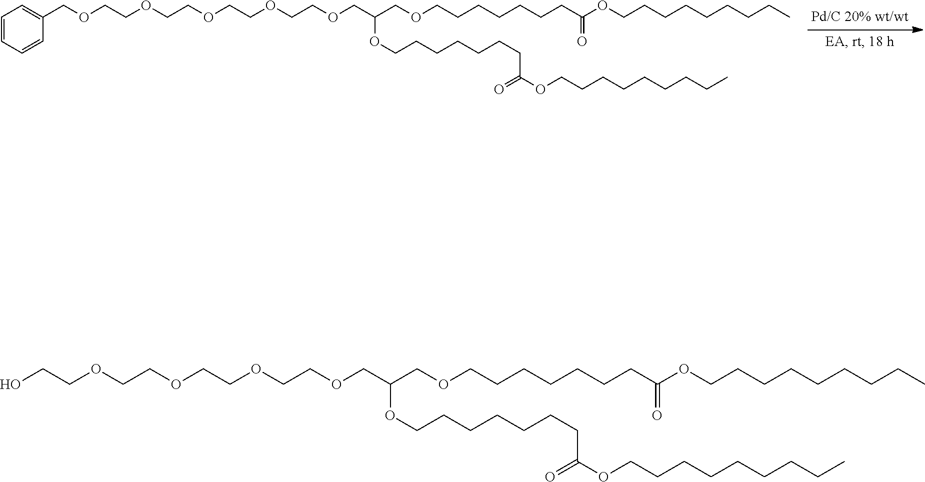 [SMILES: CCCCCCCCCOC(=O)CCCCCCCOCC(COCCOCCOCCOCCOCc1ccccc1)OCCCCCCCC(=O)OCCCCCCCCC ; CCCCCCCCCOC(=O)CCCCCCCOCC(COCCOCCOCCOCCO)OCCCCCCCC(=O)OCCCCCCCCC]

To the solution of nonyl 8-[3-[2-[2-[2-(2-benzyloxyethoxy)ethoxy]ethoxy]ethoxy]-2-(8-nonoxy-8-oxo-octoxy)propoxy]octanoate (5 g, 5.31 mmol) in ethyl acetate (100 mL) was added Pd/C (1.13 g, 20% wt/wt). The mixture was stirred at room temperature under hydrogen for 18 h.

TLC (ethyl acetate/petroleum ether 1/1) indicated that the starting material was consumed.

The reaction was filtered through celite and washed with ethyl acetate to give nonyl 8-[3-[2-[2-[2-(2-hydroxyethoxy)ethoxy]ethoxy]ethoxy]-2-(8-nonoxy-8-oxo-octoxy)propoxy]octanoate (4.22 g, 4.98 mmol, 93.8%) as colorless oil.

¹H NMR (400 MHz, CDCl₃) δ 4.05 (t, J=6.8 Hz, 4H), 3.74-3.38 (m, 27H), 2.28 (t, J=7.5 Hz, 4H), 1.68-1.50 (m, 12H), 1.39-1.21 (m, 37H), 0.88 (t, J=6.8 Hz, 6H).

Step (9)

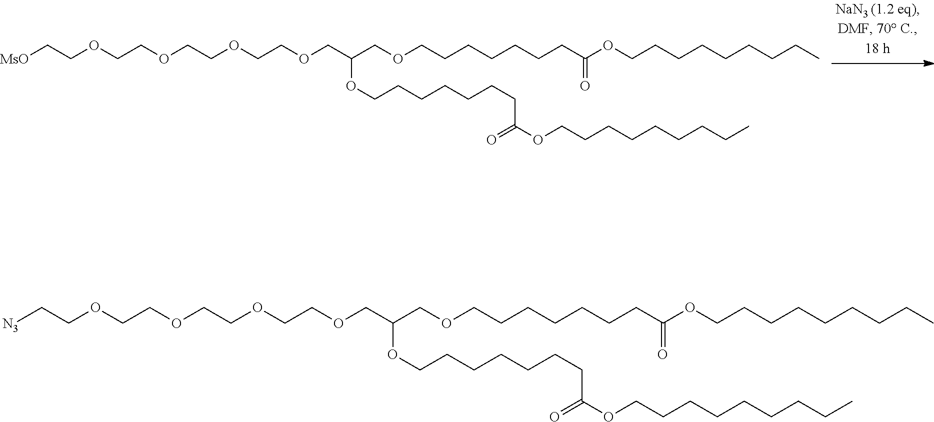 [SMILES: CCCCCCCCCOC(=O)CCCCCCCOCC(COCCOCCOCCOCCOS(C)(=O)=O)OCCCCCCCC(=O)OCCCCCCCCC ; CCCCCCCCCOC(=O)CCCCCCCOCC(COCCOCCOCCOCCN=[N+]=[N-])OCCCCCCCC(=O)OCCCCCCCCC]

To a solution of nonyl 8-[3-[2-[2-[2-(2-methylsulfonyloxyethoxy)ethoxy]ethoxy]ethoxy]-2-(8-nonoxy-8-oxo-octoxy)propoxy]octanoate (4.5 g, 4.8 mmol) in Dimethyl formamide (DMF, Volume: 30 ml) was added sodium azide (0.378 g, 5.81 mmol). Then the reaction mixture was stirred at 70° C. for 16 hr. Then water (200 mL) was added and the reaction mixture was extracted with ethyl acetate (100 mL*2). The combined organic phases were washed with brine (100 mL), dried over sodium sulfate, filtered and concentrated under reduced pressure to give a residue, which was purified by column chromatography eluted with petroleum ether:ethyl acetate=100:1 to 3:1 to afford nonyl 8-[3-[2-[2-[2-(2-azidoethoxy)ethoxy]ethoxy]ethoxy]-2-(8-nonoxy-8-oxo-octoxy)propoxy]octanoate (4 g, 4.58 mmol, 94% yield) as colorless oil.

¹H NMR (400 MHz, CDCl₃) δ 4.05 (t, J=6.8 Hz, 4H), 3.71-3.36 (m, 25H), 2.29 (t, J=7.5 Hz, 4H), 1.67-1.50 (m, 12H), 1.38-1.21 (m, 36H), 0.88 (t, J=6.8 Hz, 6H).

Step (10)

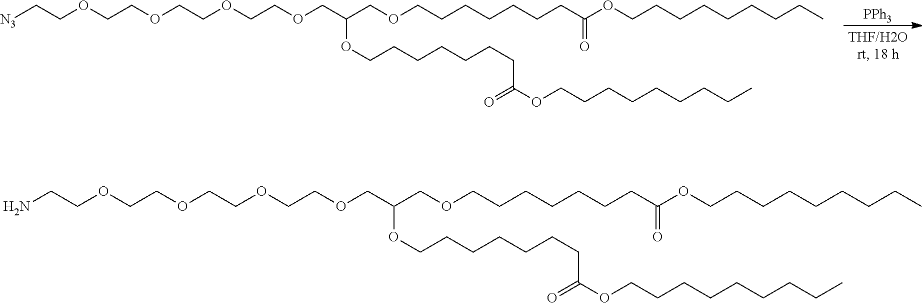 [SMILES: CCCCCCCCCOC(=O)CCCCCCCOCC(COCCOCCOCCOCCN=[N+]=[N-])OCCCCCCCC(=O)OCCCCCCCCC ; CCCCCCCCCOC(=O)CCCCCCCOCC(COCCOCCOCCOCCN)OCCCCCCCC(=O)OCCCCCCCCC ; "PPh3"]

A mixture of nonyl 8-[3-[2-[2-[2-(2-azidoethoxy)ethoxy]ethoxy]ethoxy]-2-(8-nonoxy-8-oxo-octoxy)propoxy]octanoate (1 g, 1.14 mmol) and triphenylphosphine (0.45 g, 1.72 mmol) in Tetrahydrofuran (THF), Ratio: 33, Volume: 10 ml)/Green (Name: Water, Ratio: 1, Volume: 0.3 ml) was stirred at 20° C. for 16 hr. TLC (5% methanol in dichloromethane) indicated the reaction was complete. The solvent was removed and the residue was loaded onto silica gel and purified by chromatography (silica, 1-10% methanol/ammonia in dichloromethane) to give nonyl 8-[3-[2-[2-[2-(2-aminoethoxy)ethoxy]ethoxy]ethoxy]-2-(8-nonoxy-8-oxo-octoxy)propoxy]octanoate (0.68 g, 0.8 mmol, 70% yield) as light yellow oil.

¹H NMR (400 MHz, CDCl₃) δ 4.05 (t, J=6.8 Hz, 4H), 3.70-3.39 (m, 23H), 2.91 (t, J=5.2 Hz, 2H), 2.47 (s, 2H), 2.32-2.25 (m, 4H), 1.66-1.50 (m, 12H), 1.38-1.21 (m, 36H), 0.88 (t, J=6.9 Hz, 6H).

Step (11)

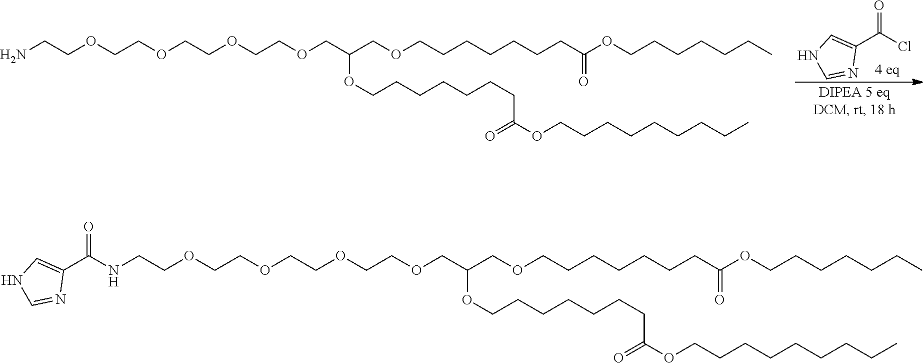 [SMILES: CCCCCCCCCOC(=O)CCCCCCCOC(COCCCCCCCC(=O)OCCCCCCC)COCCOCCOCCOCCN ; O=C(Cl)c1c[nH]cn1 ; CCCCCCCCCOC(=O)CCCCCCCOC(COCCCCCCCC(=O)OCCCCCCC)COCCOCCOCCOCCNC(=O)c1c[nH]cn1]

4-imidazolcarboxylic acid (2.5 g, 22.3 mmol) was dissolved in 60 mL oxalyl chloride and several drop of DMF was added to catalyse the reaction. The reaction was stirred at room temperature under nitrogen atmosphere overnight. The organic phase was evaporated and the remaining yellow solid was dried overnight under vacuum pump to obtain without purification the corresponding acid chloride (2.5 g, quant.). nonyl8-[3-[2-[2-[2-(2-aminoethoxy)ethoxy]ethoxy]ethoxy]-2-(8-nonoxy-8-oxo-octoxy)propoxy]octanoate (680 mg, 0.8 mmol) was dissolved in 30 mL anhydrous DCM and the acid chloride (419 mg, 3.2 mmol) in 3 mL anhydrous DMF and DIPEA (0.519 g, 4 mmol) were added. The mixture was stirred at room temperature overnight. The solvent was evaporated and the product purified by flash chromatography (40 g column, DCM/MeOH 20/1 to 10/1) followed by prep TLC (eluted with 10% methanol in dichloromethane) to give nonyl 8-[3-[2-[2-[2-[2-(1H-imidazole-4-carbonylamino)ethoxy]ethoxy]ethoxy]ethoxy]-2-(8-nonoxy-8-oxo-octoxy)propoxy]octanoate (302.3 mg, 0.326 mmol, 40.6% yield) as colorless oil.

MS (ESI) m/z=898.7 (M+H)+

¹H NMR (400 MHz, CDCl₃) δ 10.95 (s, 1H), 7.72-7.60 (m, 3H), 4.05 (t, J=6.7 Hz, 4H), 3.68-3.37 (m, 25H), 2.33-2.25 (m, 4H), 1.66-1.48 (m, 12H), 1.38-1.21 (m, 37H), 0.88 (t, J=6.8 Hz, 6H).

### Example 8: Synthesis of 1-octylnonyl 8-[3-[2-[2-[2-[2-(1H-imidazole-4-carbonylamino)ethoxy]ethoxy]ethoxy]ethoxy]-2-[8-(1-octylnonoxy)-8-oxo-octoxy]propoxy]octanoate (compound IX)

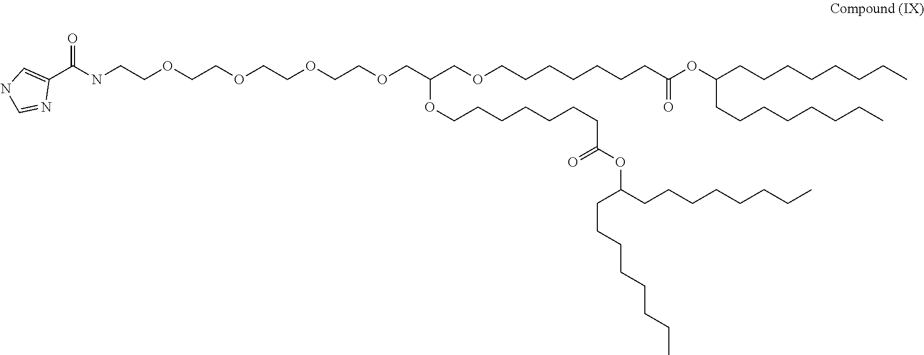 [SMILES: CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

Compound (IX) was synthesized based on the chemistry shown in Scheme (6).

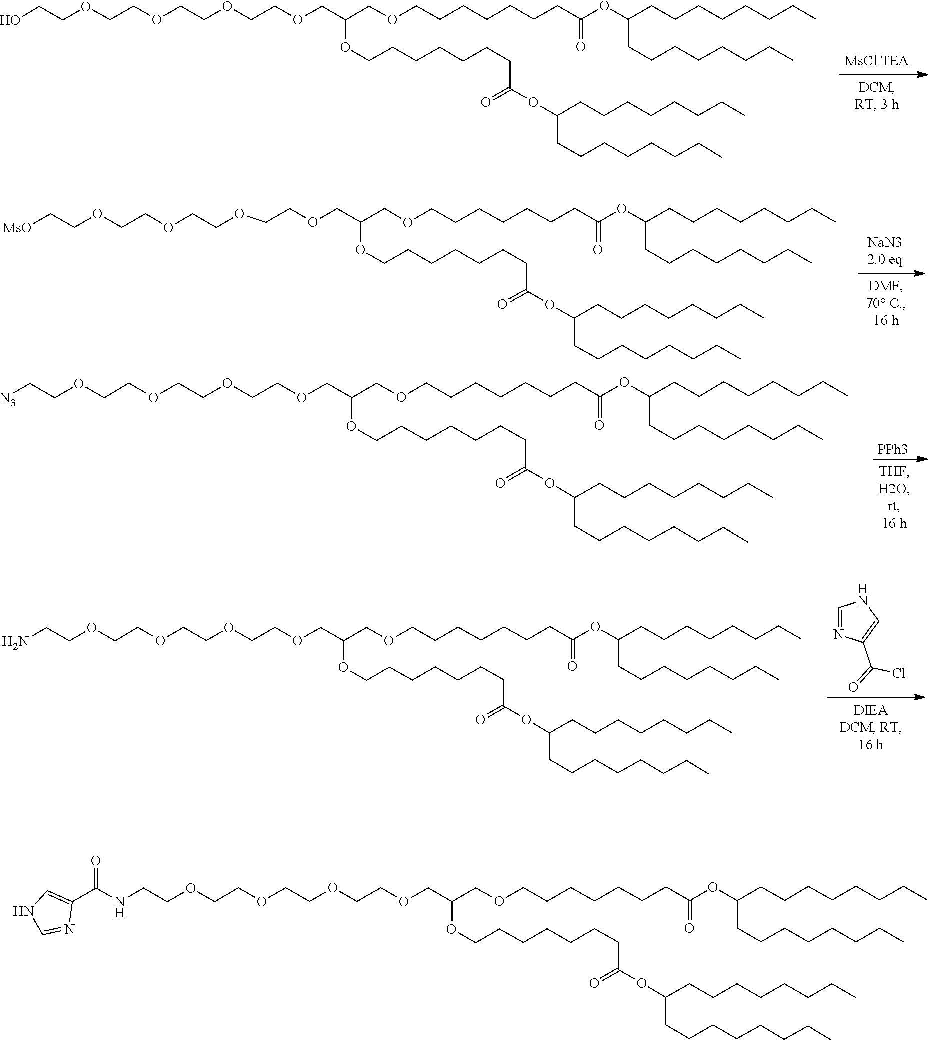

Synthesis

Step (1)

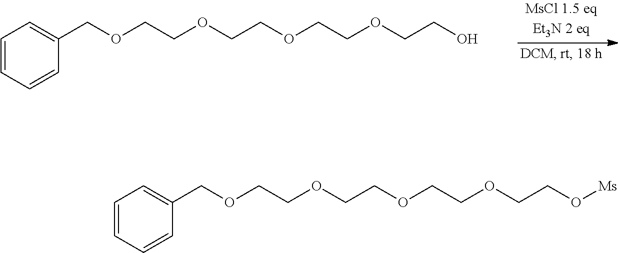 [SMILES: OCCOCCOCCOCCOCc1ccccc1 ; CS(=O)(=O)OCCOCCOCCOCCOCc1ccccc1]

2-[2-[2-(2-benzyloxyethoxy)ethoxy]ethoxy]ethanol (50 g, 0.176 mol) and triethylamine (36.2 g, 0.352 mol) in dry dichloromethane (600 mL) under nitrogen were cooled to 0° C.

Methanesulfonyl chloride (30.6 g, 0.264 mol) was added dropwise to this solution at 0° C. The mixture was allowed to warm to room temperature and stirred at room temperature for 18 h. Triethylamine hydrochloride was filtered off, and the DCM solution was washed with 0.1 N HCl and dried over sodium sulfate. Removing the solvent afforded 2-[2-[2-(2-benzyloxyethoxy)ethoxy]ethoxy]ethyl methanesulfonate (62 g, 92%) as light yellow oil which was used without further purification.

LCMS MS 363 (M+1)

Step (2)

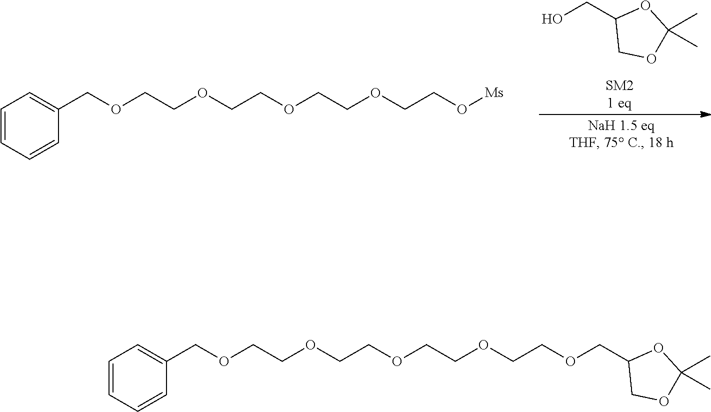

To the solution of (2,2-dimethyl-1,3-dioxolan-4-yl)methanol (62 g, 0.171 mol) in THF (600 mL) was added NaH (6.17 g, 0.257 mol) and the mixture was heated to reflux for 15 min. Then the reaction was cooled to room temperature and 2-[2-[2-(2-benzyloxyethoxy)ethoxy]ethoxy]ethyl methanesulfonate (25.0 g, 0.171 mol) was added under nitrogen and the reaction was heated at 80° C. for 18 h. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 20 to 50% ethyl acetate in petroleum ether to give 4-[2-[2-[2-(2-benzyloxyethoxy)ethoxy]ethoxy]ethoxymethyl]-2,2-dimethyl-1,3-dioxolane (43 g, 71% yield) as light yellow oil.

LCMS MS 421 (M+23)

Step (3)

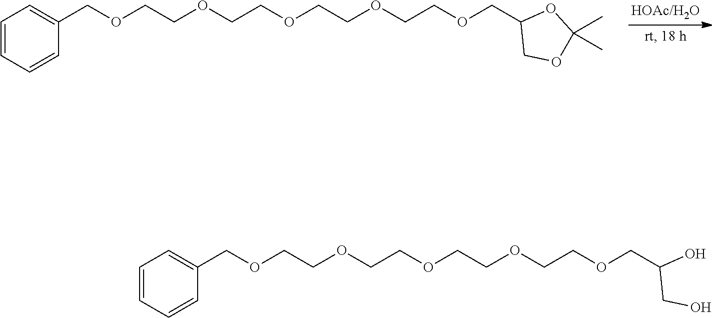 [SMILES: CC1(C)OCC(COCCOCCOCCOCCOCc2ccccc2)O1 ; OCC(O)COCCOCCOCCOCCOCc1ccccc1]

The mixture of 4-[2-[2-[2-(2-benzyloxyethoxy)ethoxy]ethoxy]ethoxymethyl]-2,2-dimethyl-1,3-dioxolane (43 g, 0.103 mol) in AcOH (200 mL) and water (200 mL). The mixture was stirred for 16 hrs at ambient temperature. Removing the solvent afforded 3-[2-[2-[2-(2-benzyloxyethoxy)ethoxy]ethoxy]ethoxy]propane-1,2-diol (36 g, 95%) as light yellow oil which was used without further purification.

LCMS MS 381 (M+23)

Step (4)

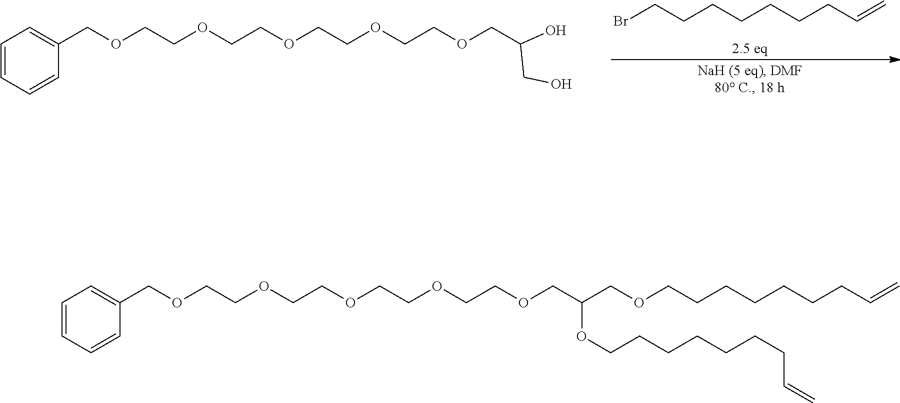

To the solution of 3-[2-[2-[2-(2-benzyloxyethoxy)ethoxy]ethoxy]ethoxy]propane-1,2-diol (20 g, 0.050 mol) in THF (200 mL) was added NaH (8.03 g, 0.201 mol) and the mixture was heated to reflux for 15 min. Then the reaction was cooled to room temperature and 9-bromonon-1-ene (26.6 g, 0.126 mol) was added under nitrogen and the reaction was heated at 80° C. for 18 hrs. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 10 to 30% ethyl acetate in petroleum ether to give 2-[2-[2-[2-[2,3-bis(non-8-enoxy)propoxy]ethoxy]ethoxy]ethoxy]ethoxymethylbenzene (8.8 g, 26% yield) as light yellow oil.

¹H NMR (400 MHz, CDCl₃) δ 7.37-7.27 (m, 5H), 5.87-5.73 (m, 2H), 5.04-4.87 (m, 4H), 4.57 (s, 2H), 3.71-3.59 (m, 16H), 3.59-3.38 (m, 9H), 2.03 (q, J=6.5 Hz, 4H), 1.60-1.49 (m, 4H), 1.41-1.28 (m, 16H).

Step (5)

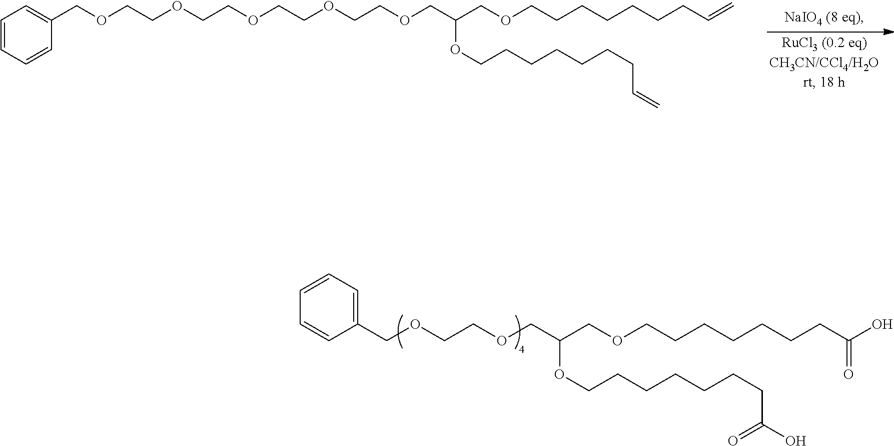

To a solution of 2-[2-[2-[2-[2,3-bis(non-8-enoxy)propoxy]ethoxy]ethoxy]ethoxy]ethoxymethylbenzene (8.5 g, 0.0140 mol) in MeCN (80 mL), CCl₄ (80 mL) and water (80 mL) was added NaIO₄ (24.9 g, 0.116 mol) and RuCl₃ (0.66 g, 2.93 mmol). The reaction mixture was stirred at room temperature for 24 h. LCMS indicated that the title compound was the major product. The reaction was filtered and the filtrate was diluted with ethyl acetate (600 mL) and washed with 1N aq. HCl (200 mL). The organic layer was dried over sodium sulfate, filtered and concentrated to give 8-[3-[2-[2-[2-(2-benzyloxyethoxy)ethoxy]ethoxy]ethoxy]-2-(7-carboxyheptoxy)propoxy]octanoic acid (8.7 g, 97% yield) as yellow oil which was used without further purification.

¹H NMR (400 MHz, CDCl₃) δ 7.31 (dd, J=22.6, 3.2 Hz, 5H), 4.57 (s, 2H), 3.71-3.61 (m, 19H), 3.59-3.38 (m, 11H), 2.32 (t, J=7.4 Hz, 4H), 1.68-1.47 (m, 10H), 1.32 (s, 14H).

Step (6)

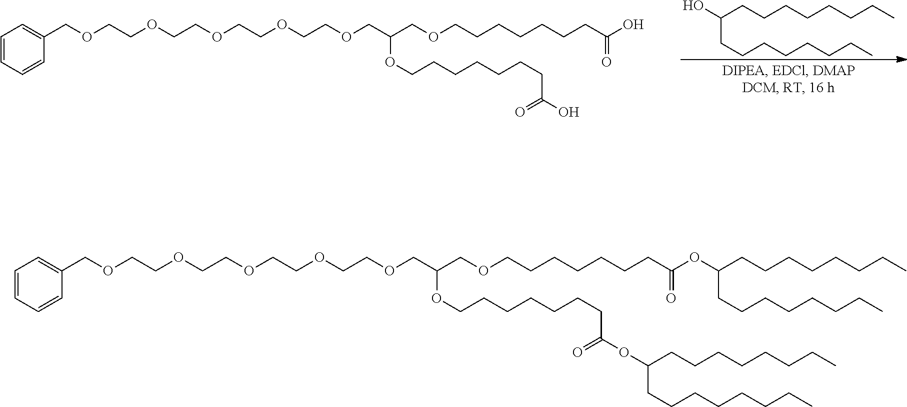 [SMILES: O=C(O)CCCCCCCOCC(COCCOCCOCCOCCOCc1ccccc1)OCCCCCCCC(=O)O ; CCCCCCCCC(O)CCCCCCCC ; CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC]

A mixture of 8-[3-[2-[2-[2-(2-benzyloxyethoxy)ethoxy]ethoxy]ethoxy]-2-(7-carboxyheptoxy)propoxy]octanoic acid (40 g, 49.8 mmol, purity: 80%), heptadecan-9-ol (25.5 g, 99.6 mmol), N,N-dimethylpyridin-4-amine (12.2 g, 99.6 mmol), EDC HCl (19.1 g, 99.6 mmol) and DIEA (19.3 g, 149 mmol) in DCM (500 mL). The mixture was stirred for 16 hrs at room temperature. The mixture was added DCM (500 mL) and washed with 1 N HCl, brine and concentrated. The residue was purified by flash column chromatography on silica gel eluting with 1:1 ethyl acetate/petroleum ether to give 1-octylnonyl 8-[3-[2-[2-[2-(2-benzyloxyethoxy)ethoxy]ethoxy]ethoxy]-2-[8-(1-octylnonoxy)-8-oxo-octoxy]propoxy]octanoate (15 g, 27%) as colourless oil.

¹H NMR (400 MHz, CDCl₃) δ 7.37-7.27 (m, 5H), 4.90-4.82 (m, 2H), 3.70-3.60 (m, 16H), 3.58-3.37 (m, 9H), 2.27 (t, J=7.5 Hz, 4H), 1.53 (dd, J=22.4, 6.0 Hz, 12H), 1.28 (d, J=22.7 Hz, 60H), 0.88 (t, J=6.8 Hz, 12H).

Step (7)

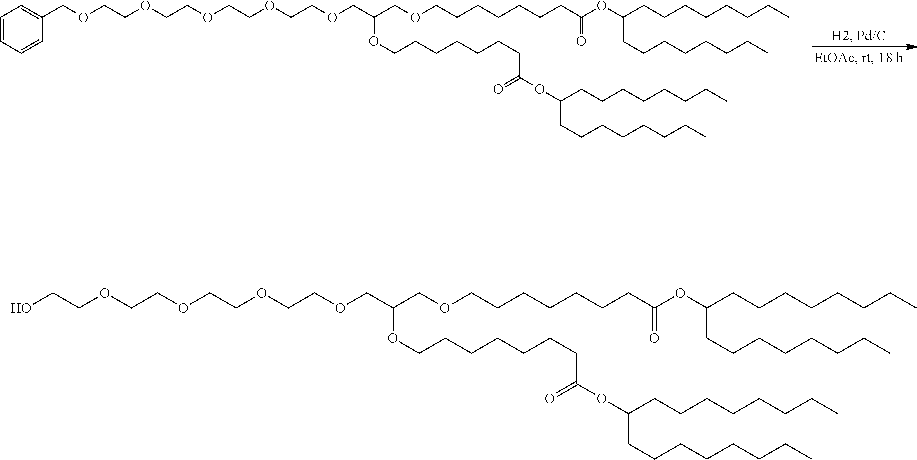 [SMILES: CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC ; CCCCCCCCC(CCCCCCCC)OC(=O)CCCCCCCOCC(COCCOCCOCCOCCO)OCCCCCCCC(=O)OC(CCCCCCCC)CCCCCCCC]

To the solution of 1-octylnonyl 8-[3-[2-[2-[2-(2-benzyloxyethoxy)ethoxy]ethoxy]ethoxy]-2-[8-(1-octylnonoxy)-8-oxo-octoxy]propoxy]octanoate (15 g, 13.4 mmol) in ethyl acetate (150 mL) was added Pd/C (2.85 g, 20% wt/wt). The mixture was stirred at room temperature under hydrogen for 18 hrs. TLC (ethyl acetate/petroleum ether 1/1) indicated that the starting material was consumed. The reaction was filtered through celite and washed with ethyl acetate to give 1-octylnonyl 8-[3-[2-[2-[2-(2-hydroxyethoxy)ethoxy]ethoxy]ethoxy]-2-[8-(1-octylnonoxy)-8-oxo-octoxy]propoxy]octanoate (11.1 g, 80.5%) as colorless oil.

¹H NMR (400 MHz, CDCl₃) δ 4.91-4.82 (m, 2H), 3.75-3.39 (m, 24H), 2.27 (t, J=7.2 Hz, 4H), 1.66-1.43 (m, 17H), 1.38-1.17 (m, 61H), 0.88 (t, J=6.8 Hz, 12H).

Step (8)

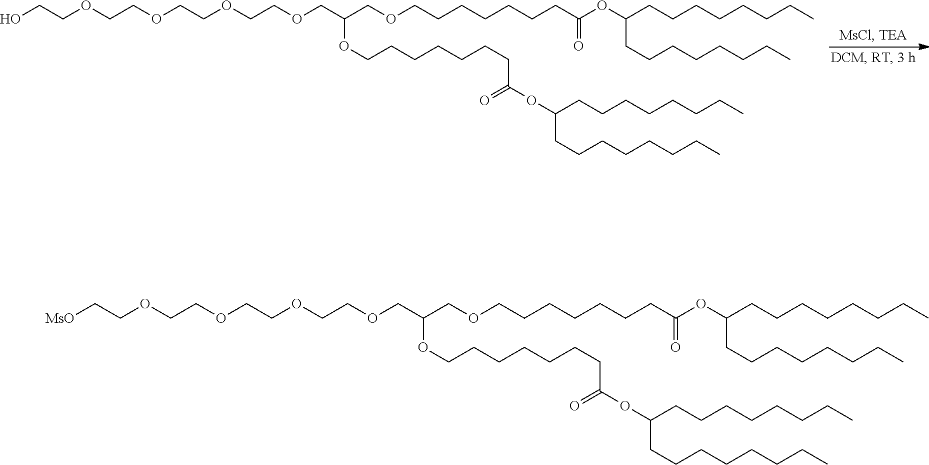 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC ; CCCCCCCCC(CCCCCCCC)OC(=O)CCCCCCCOCC(COCCOCCOCCOCCOS(C)(=O)=O)OCCCCCCCC(=O)OC(CCCCCCCC)CCCCCCCC]

A mixture of 1-octylnonyl 8-[3-[2-[2-[2-(2-hydroxyethoxy)ethoxy]ethoxy]ethoxy]-2-[8-(1-octylnonoxy)-8-oxo-octoxy]propoxy]octanoate (1.4 g, 1.36 mmol) and N,N-diethylethanamine (0.275 g, 2.72 mmol) in DCM (20 mL) was added methanesulfonyl chloride at 0° C. The mixture was stirred for 3 hrs at room temperature. The mixture was added DCM (100 mL) and washed with water, brine and concentrated to give 1-octylnonyl 8-[3-[2-[2-[2-(2-methylsulfonyloxyethoxy)ethoxy]ethoxy]ethoxy]-2-[8-(1-octylnonoxy)-8-oxo-octoxy]propoxy]octanoate (1.4 g, 93%) as colorless oil.

¹H NMR (400 MHz, CDCl₃) δ 7.37-7.27 (m, 5H), 4.90-4.82 (m, 2H), 3.70-3.60 (m, 16H), 3.58-3.37 (m, 9H), 2.27 (t, J=7.5 Hz, 4H), 1.53 (dd, J=22.4, 6.0 Hz, 12H), 1.28 (d, J=22.7 Hz, 60H), 0.88 (t, J=6.8 Hz, 12H).

Step (9)

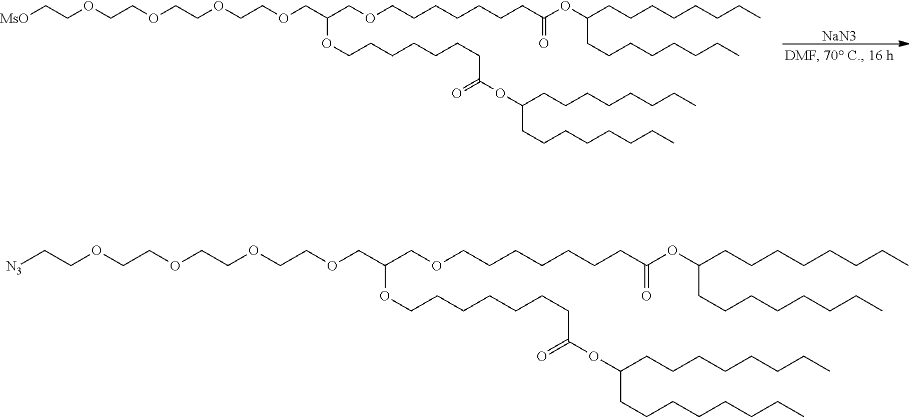 [SMILES: CCCCCCCCC(CCCCCCCC)OC(=O)CCCCCCCOCC(COCCOCCOCCOCCOS(C)(=O)=O)OCCCCCCCC(=O)OC(CCCCCCCC)CCCCCCCC ; CCCCCCCCC(CCCCCCCC)OC(=O)CCCCCCCOCC(COCCOCCOCCOCCN=[N+]=[N-])OCCCCCCCC(=O)OC(CCCCCCCC)CCCCCCCC ; "NaN3"]

To a mixture of 1-octylnonyl 8-[3-[2-[2-[2-(2-methylsulfonyloxyethoxy)ethoxy]ethoxy]ethoxy]-2-[8-(1-octylnonoxy)-8-oxo-octoxy]propoxy]octanoate (1.4 g, 1.26 mmol) in DMF (10 mL) at room temperature. The mixture was stirred for 16 hrs at 70° C. The mixture was added water (50 mL) and extracted with EtOAc (50 mL*3). The organic layers was washed with brine, dried over Na₂SO₄ and concentrated to give 1-octylnonyl 8-[3-[2-[2-[2-(2-azidoethoxy)ethoxy]ethoxy]ethoxy]-2-[8-(1-octylnonoxy)-8-oxo-octoxy]propoxy]octanoate (1.3 g, 97.5%) as colorless oil.

¹H NMR (400 MHz, CDCl₃) δ 4.86 (p, J=6.3 Hz, 2H), 3.72-3.58 (m, 15H), 3.60-3.35 (m, 11H), 2.27 (t, J=7.5 Hz, 4H), 1.56 (ddd, J=22.2, 14.3, 6.1 Hz, 18H), 1.38-1.19 (m, 64H), 0.88 (t, J=6.8 Hz, 12H).

Step (10)

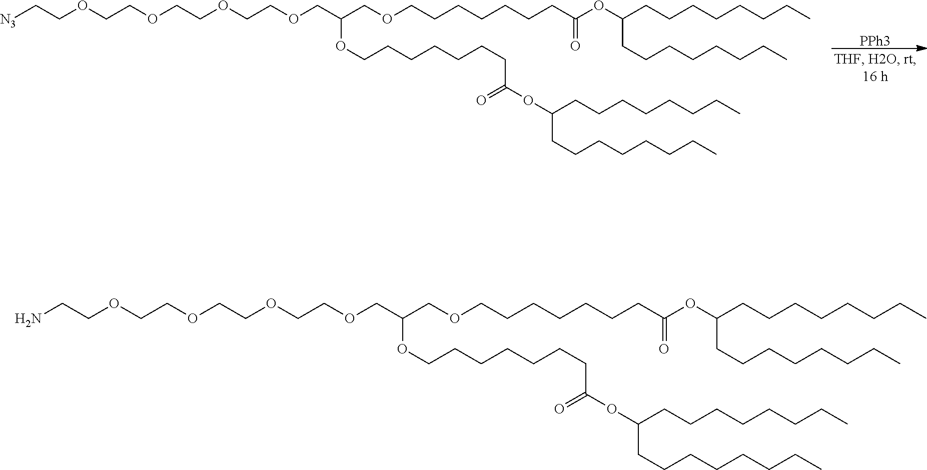 [SMILES: CCCCCCCCC(CCCCCCCC)OC(=O)CCCCCCCOCC(COCCOCCOCCOCCN=[N+]=[N-])OCCCCCCCC(=O)OC(CCCCCCCC)CCCCCCCC ; CCCCCCCCC(CCCCCCCC)OC(=O)CCCCCCCOCC(COCCOCCOCCOCCN)OCCCCCCCC(=O)OC(CCCCCCCC)CCCCCCCC ; "PPh3"]

A mixture of 1-octylnonyl 8-[3-[2-[2-[2-(2-azidoethoxy)ethoxy]ethoxy]ethoxy]-2-[8-(1-octylnonoxy)-8-oxo-octoxy]propoxy]octanoate (1.3 g, 1.23 mmol) and triphenylphosphane in THF (20 mL) and water (3 mL). The mixture was stirred for 16 hrs at room temperature. The mixture was concentrated to give 1-octylnonyl 8-[3-[2-[2-[2-(2-aminoethoxy)ethoxy]ethoxy]ethoxy]-2-[8-(1-octylnonoxy)-8-oxo-octoxy]propoxy]octanoate (1.1 g, 87%) as colorless oil.

¹H NMR (400 MHz, CDCl₃) δ 4.86 (p, J=6.2 Hz, 2H), 3.71-3.39 (m, 23H), 2.91 (t, J=5.1 Hz, 2H), 2.27 (t, J=7.4 Hz, 7H), 1.54 (dd, J=32.2, 15.1 Hz, 16H), 1.28 (d, J=23.3 Hz, 60H), 0.88 (t, J=6.8 Hz, 12H).

Step (11)

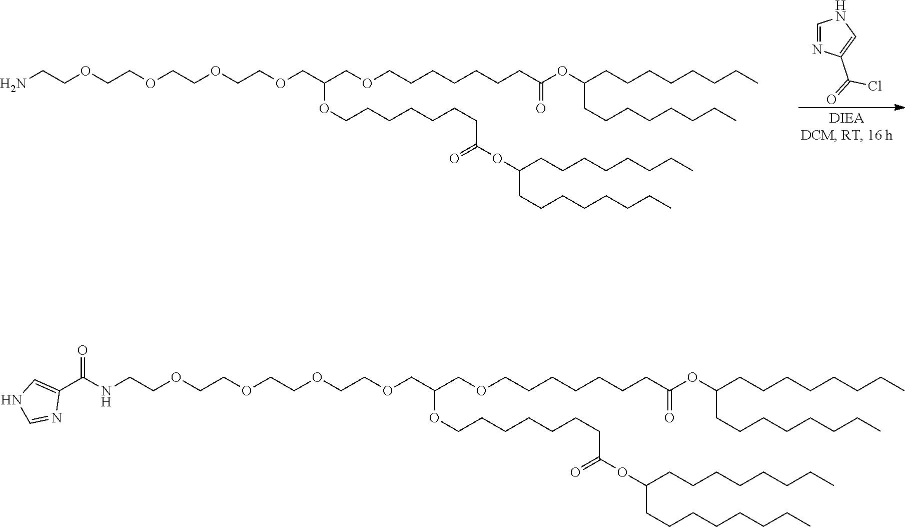 [SMILES: CCCCCCCCC(CCCCCCCC)OC(=O)CCCCCCCOCC(COCCOCCOCCOCCN)OCCCCCCCC(=O)OC(CCCCCCCC)CCCCCCCC ; O=C(Cl)c1c[nH]cn1 ; CCCCCCCCC(CCCCCCCC)OC(=O)CCCCCCCOCC(COCCOCCOCCOCCNC(=O)c1c[nH]cn1)OCCCCCCCC(=O)OC(CCCCCCCC)CCCCCCCC]

To a mixture of 1-octylnonyl 8-[3-[2-[2-[2-(2-azidoethoxy)ethoxy]ethoxy]ethoxy]-2-[8-(1-octylnonoxy)-8-oxo-octoxy]propoxy]octanoate (1.1 g, 1.04 mmol) and N-ethyl-N-isopropyl-propan-2-amine (1.35 g, 10.4 mmol) in DCM (40 mL) was added 1H-imidazole-4-carbonyl chloride (0.545 g, 4.17 mmol). The mixture was stirred for 16 hrs at room temperature. The mixture was concentrated and the residue was purified by column chromatography on silica gel eluting with 2%-15% MeOH in DCM to afford 1-octylnonyl 8-[3-[2-[2-[2-[2-(1H-imidazole-4-carbonylamino)ethoxy]ethoxy]ethoxy]ethoxy]-2-[8-(1-octylnonoxy)-8-oxo-octoxy]propoxy]octanoate (0.22 g, 18.8%) as yellow oil.

¹H NMR (400 MHz, CDCl₃) δ 10.78 (s, 1H), 7.63 (s, 3H), 4.86 (s, 2H), 3.70-3.36 (m, 25H), 2.28 (dd, J=10.6, 4.4 Hz, 4H), 1.94 (s, 3H), 1.66-1.43 (m, 17H), 1.36-1.16 (m, 62H), 0.88 (t, J=6.8 Hz, 12H).

### Example 9: Synthesis of 2-[2-[2-[2-(1H-imidazole-4-carbonylamino)ethoxy]ethoxy]ethoxy]ethyl 2,3-bis[(Z)-octadec-9-enoxy]propanoate (Compound X)

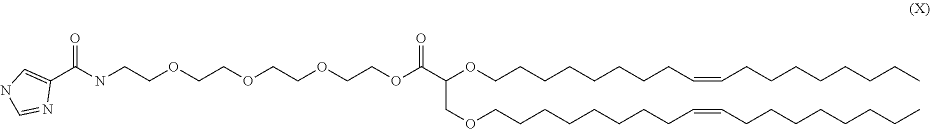 [SMILES: CCCCCCCC/C=C\CCCCCCCCOCC(OCCCCCCCC/C=C\CCCCCCCC)C(=O)OCCOCCOCCOCCNC(=O)c1c[nH]cn1]

Synthesis of Compound (X)

To a solution of 2-[2-[2-(2-aminoethoxy)ethoxy]ethoxy]ethyl 2,3-bis[(Z)-octadec-9-enoxy]propanoate (282 mg, 0.36 mmol) in DCM (10 ml) was added DIEA (233 mg, 1.8 mmol) and 1H-imidazole-4-carbonyl chloride (188 mg, 1.44 mmol). The mixture was stirred at 25° C. for 14 hr. After reaction, the mixture was dealt with EA (100 ml), washed with water (100 ml×2), NaCl sat·aq (100 ml). The organic was concentrated and purified by flash (10% MeOH in DCM), to give 2-[2-[2-[2-(1H-imidazole-4-carbonylamino) ethoxy] ethoxy] ethoxy] ethyl 2,3-bis[(Z)-octadec-9-enoxy]propanoate (192 mg, yield 59.6%) as a colourless oil.

¹H NMR (400 MHz, CDCl₃) δ 9.77-9.65 (m, 1H), 7.66 (s, 1H), 7.61 (s, 1H), 7.51-7.44 (m, 1H), 5.34 (t, J=5.4 Hz, 4H), 4.27 (d, J=4.5 Hz, 2H), 4.08-4.05 (m, 1H), 3.75-3.57 (m, 17H), 3.49-3.39 (m, 3H), 2.18-1.90 (m, 8H), 1.60 (s, 2H), 1.55-1.52 (m, 2H), 1.27 (s, 44H), 0.88 (t, J=6.8 Hz, 6H).

### Example 10: Synthesis of 2,3-bis[(Z)-octadec-9-enoxy]propyl 2-[2-[2-[2-(1H-imidazole-4-carbonylamino)ethoxy]ethoxy]ethoxy]acetate (compound XI)

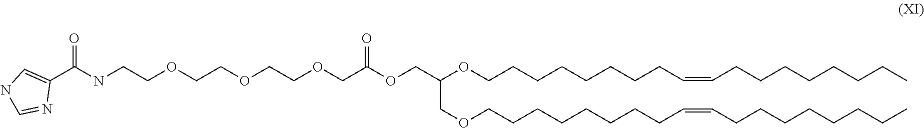 [SMILES: CCCCCCCC/C=C\CCCCCCCCOCC(COC(=O)COCCOCCOCCNC(=O)c1c[nH]cn1)OCCCCCCCC/C=C\CCCCCCCC]

Synthesis of Compound (XI)

To a solution of 2,3-bis[(Z)-octadec-9-enoxy]propyl 2-[2-[2-(2-aminoethoxy)ethoxy]ethoxy]acetate (623 mg, 0.79 mmol) in DCM (15 ml) was added DIEA (515 mg, 3.98 mmol), 1H-imidazole-4-carbonyl chloride (416 mg, 3.19 mmol) in DMF (5 ml). The mixture was stirred at 25° C. for 14 hr. The mixture was concentrated and dealt with EA (50 ml), washed with water (50 ml×2), NaCl sat.aq (50 ml) and dried over Na₂SO₄. The organic was concentrated and purified by flash (5% MeOH in DCM), to give 2,3-bis[(Z)-octadec-9-enoxy]propyl 2-[2-[2-[2-(1H-imidazole-4-carbonylamino)ethoxy]ethoxy]ethoxy]acetate (380 mg, yield 53.4 mmol) as a colorless oil.

¹H NMR (400 MHz, CDCl₃) δ 9.97 (s, 1H), 7.64 (d, J=15.8 Hz, 3H), 5.34 (t, J=5.4 Hz, 4H), 4.32 (dd, J=11.5, 4.0 Hz, 1H), 4.20-4.11 (m, 3H), 3.72-3.61 (m, 13H), 3.55 (t, J=6.7 Hz, 2H), 3.46 (dt, J=13.0, 5.5 Hz, 4H), 2.13-1.86 (m, 8H), 1.54 (d, J=6.2 Hz, 4H), 1.33-1.24 (m, 44H), 0.88 (t, J=6.8 Hz, 6H

### Example 11: Synthesis of 2,3-bis[(˜{Z})-octadec-9-enoxy]propyl˜{N}-[2-[2-[2-(1˜{H}-imidazole-4-carbonylamino)ethoxy]ethoxy]ethyl]carbamate (compound XII)

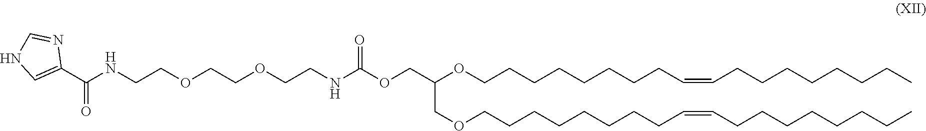 [SMILES: CCCCCCCC/C=C\CCCCCCCCOCC(COC(=O)NCCOCCOCCNC(=O)c1c[nH]cn1)OCCCCCCCC/C=C\CCCCCCCC]

Synthesis of Compound (XII)

Step (1)

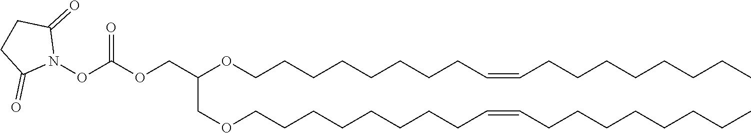 [SMILES: CCCCCCCC/C=C\CCCCCCCCOCC(COC(=O)ON1C(=O)CCC1=O)OCCCCCCCC/C=C\CCCCCCCC]

To a solution of 2,3-bis[(Z)-octadec-9-enoxy]propan-1-ol (1 g, 1.65 mmol) in DMF (10 ml) was added bis(2,5-dioxopyrrolidin-1-yl) carbonate (1.34 g, 4.96 mmol) and 4-Dimethylaminopyridine (202 mg, 1.65 mmol). The mixture was stirred at 25° C. The mixture was dealt with EA (50 ml), washed with water (50 ml×2), NaCl aq (50 ml) and dried over Na₂SO₄. The organic was concentrated and purified by flash (20% EA in PE), to give 2,3-bis[(Z)-octadec-9-enoxy]propyl (2,5-dioxopyrrolidin-1-yl) carbonate (937 mg, yield 75.7%) as a colourless oil.

¹H NMR (400 MHz, CDCl₃) δ 5.48-5.30 (m, 4H), 4.46 (dd, J=11.1, 3.9 Hz, 1H), 4.38-4.31 (m, 1H), 3.74-3.66 (m, 1H), 3.60-3.39 (m, 6H), 2.83 (s, 4H), 2.01 (dd, J=14.8, 9.2 Hz, 8H), 1.54 (d, J=6.6 Hz, 4H), 1.33-1.22 (m, 44H), 0.88 (t, J=6.8 Hz, 6H).

Step (2)

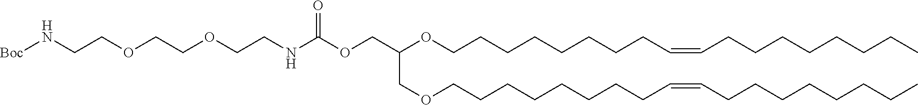 [SMILES: CCCCCCCC/C=C\CCCCCCCCOCC(COC(=O)NCCOCCOCCNC(=O)OC(C)(C)C)OCCCCCCCC/C=C\CCCCCCCC]

To a solution of 2,3-bis[(Z)-octadec-9-enoxy]propyl (2,5-dioxopyrrolidin-1-yl) carbonate (937 mg, 1.28 mmol) in DCM (10 ml) was added tert-butyl N-[2-[2-(2-aminoethoxy)ethoxy]ethyl]carbamate (327 mg, 1.28 mmol), TEA (194 mg, 1.91 mmol) and 4-Dimethylaminopyridine (15 mg, 0.128 mmol). The mixture was stirred at 25° C. for 14 hr. After reaction, the mixture was dealt with DCM (50 ml), washed with water (50 ml×2), NaCl aq (50 ml) and dried over Na2SO4. The organic was concentrated and purified by flash (0-50% EA in PE), to give tert-butyl N-[2-[2-[2-[2,3-bis[(Z)-octadec-9-enoxy]propoxycarbonylamino]ethoxy]ethoxy]ethyl]carbamate (802 mg, yield 71%) as a colourless oil.

¹H NMR (400 MHz, CDCl₃) δ 5.36 (dt, J=9.9, 5.0 Hz, 4H), 5.27 (s, 1H), 5.06 (s, 1H), 4.20 (dd, J=11.4, 3.8 Hz, 1H), 4.11 (dd, J=11.5, 5.3 Hz, 1H), 3.62-3.53 (m, 11H), 3.48 (d, J=5.4 Hz, 2H), 3.45-3.32 (m, 6H), 2.02 (dt, J=12.3, 6.3 Hz, 8H), 1.57-1.51 (m, 4H), 1.45 (s, 9H), 1.34-1.25 (m, 44H), 0.88 (t, J=6.7 Hz, 6H).

Step (3)

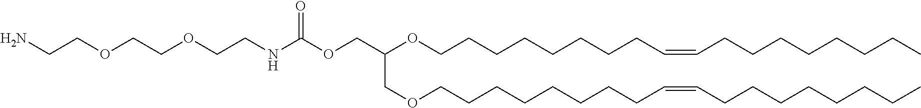 [SMILES: CCCCCCCC/C=C\CCCCCCCCOCC(COC(=O)NCCOCCOCCN)OCCCCCCCC/C=C\CCCCCCCC]

To a solution of tert-butyl N-[2-[2-[2-[2,3-bis[(Z)-octadec-9-enoxy]propoxycarbonylamino]ethoxy]ethoxy]ethyl]carbamate (802 mg, 0.925 mmol) in DCM (10 ml) was added TFA (1.3 ml). The mixture was stirred at 25° C. for 3 hr. The mixture was concentrated in vacuo, to give 2,3-bis[(Z)-octadec-9-enoxy]propyl N-[2-[2-(2-aminoethoxy)ethoxy]ethyl]carbamate (1.19 g, crude) as a yellow oil.

¹H NMR (400 MHz, CDCl₃) δ 7.52 (s, 2H), 5.39-5.31 (m, 4H), 4.26 (d, J=10.5 Hz, 1H), 4.14 (dd, J=11.7, 4.5 Hz, 1H), 3.76 (t, J=4.8 Hz, 2H), 3.70-3.51 (m, 12H), 3.46 (t, J=6.8 Hz, 2H), 3.39 (d, J=4.7 Hz, 2H), 3.25 (s, 2H), 2.01 (dd, J=12.5, 6.6 Hz, 8H), 1.56 (d, J=8.9 Hz, 4H), 1.31-1.23 (m, 44H), 0.88 (t, J=6.8 Hz, 6H).

Step (4)

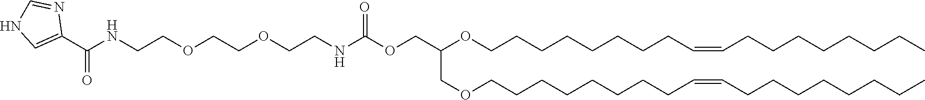 [SMILES: CCCCCCCC/C=C\CCCCCCCCOCC(COC(=O)NCCOCCOCCNC(=O)c1c[nH]cn1)OCCCCCCCC/C=C\CCCCCCCC]

To a solution of 2,3-bis[(Z)-octadec-9-enoxy]propyl N-[2-[2-(2-aminoethoxy)ethoxy]ethyl]carbamate (709 mg, 0.924 mmol) in DCM (15 ml) was added DIEA (717 mg, 5.54 mmol) and 1H-imidazole-4-carbonyl chloride (483 mg, 3.7 mmol). The mixture was stirred at 25° C. for 14 hr. The mixture was concentrated and dealt with EA (50 ml), washed with water (50 ml×2), NaCl sat.aq (50 ml) and dried over Na2SO4. The organic was concentrated and purified by flash (5%-10% MeOH in DCM), to give 2,3-bis[(Z)-octadec-9-enoxy]propyl N-[2-[2-[2-(1H-imidazole-4-carbonylamino)ethoxy]ethoxy]ethyl]carbamate (462 mg, yield 56.9%) as a colourless oil.

¹H NMR (400 MHz, CDCl₃) δ 9.85-9.75 (m, 1H), 7.66 (d, J=10.2 Hz, 2H), 7.52 (s, 1H), 5.75 (s, 1H), 5.34 (t, J=4.8 Hz, 4H), 4.19 (s, 1H), 4.12-4.07 (m, 1H), 3.71-3.59 (m, 9H), 3.56 (t, J=5.1 Hz, 4H), 3.50-3.42 (m, 4H), 3.37 (d, J=5.2 Hz, 2H), 2.10-1.87 (m, 8H), 1.54 (d, J=6.4 Hz, 4H), 1.26 (d, J=4.7 Hz, 44H), 0.88 (t, J=6.8 Hz, 6H).

### Example 12: Synthesis of [(Z)-non-2-enyl] 8-[3-[2-[2-[2-[2-(1H-imidazole-4-carbonylamino)ethoxy]ethoxy]ethoxy]ethoxy]-2-[8-[(Z)-non-2-enoxy]-8-oxo-octoxy]propoxy]octanoate (Compound XIII)

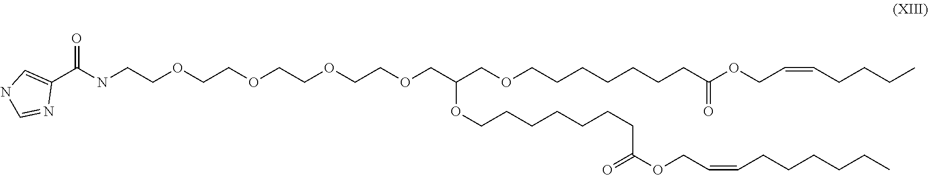 [SMILES: CCCC/C=C\COC(=O)CCCCCCCOCC(COCCOCCOCCOCCNC(=O)c1c[nH]cn1)OCCCCCCCC(=O)OC/C=C\CCCCCC]

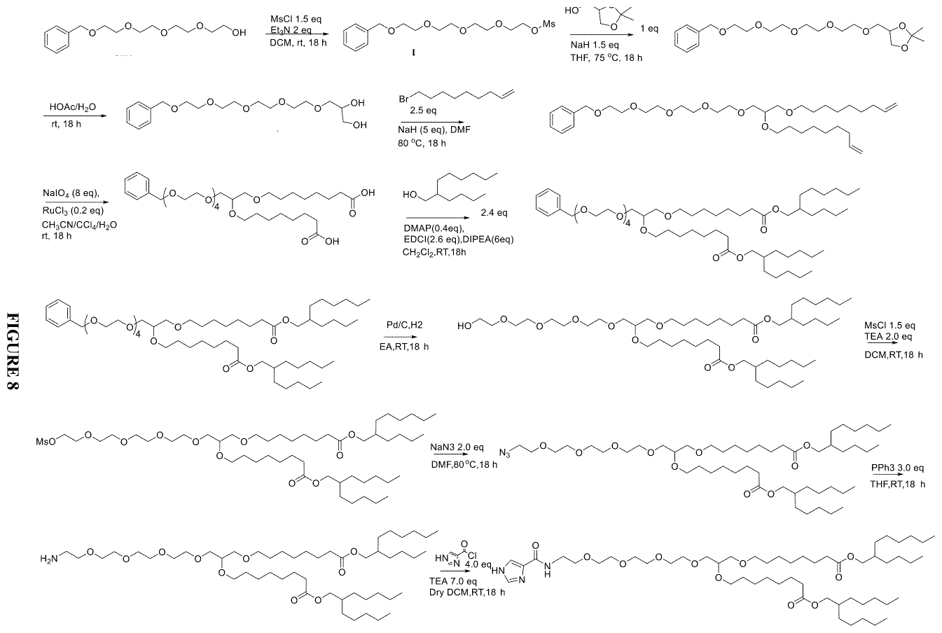

The compound was synthesized based on the chemistry shown in Scheme (7) as shown on FIG. 7.

Synthesis of Compound (XIII)

Step (1)

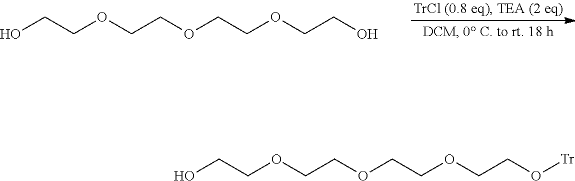

A mixture of 2-[2-[2-(2-hydroxyethoxy)ethoxy]ethoxy]ethanol (40 g, 0.206 mol), N,N-dimethylpyridin-4-amine (1.26 g, 0.0103 mol) and [chloro(diphenyl)methyl]benzene (45.9 g, 0.165 mol) in DCM (300 mL). The mixture was cooled to 0° C., then added N,N-diethylethanamine (41.7 g, 0.412 mol). The reaction mixture was stirred for 16 hrs at ambient temperature. LCMS showed a good reaction. The mixture was poured into water (600 mL) and extracted with DCM (2*400 mL). The organic layer was washed with water, NaCl, dried over Na₂SO₄ and concentrated. The residue was purified by flash column chromatography on silica gel eluting with 3:1 ethyl acetate/petroleum ether to give 2-[2-[2-(2-trityloxyethoxy)ethoxy]ethoxy]ethanol (33.0 g, 36.7%) as colorless oil.

LCMS 459 (M+23), 99% UV 214 nm

¹H NMR (400 MHz, CDCl₃) δ 7.49-7.44 (m, 6H), 7.32-7.26 (m, 6H), 7.25-7.19 (m, 3H), 3.72-3.64 (m, 12H), 3.61-3.57 (m, 2H), 3.27-3.22 (m, 2H), 2.55-2.50 (m, 1H).

Step (2)

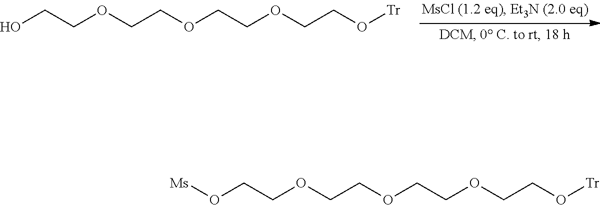

To a mixture of 2-[2-[2-(2-trityloxyethoxy)ethoxy]ethoxy]ethanol (33 g, 0.0756 mol) and N,N-diethylethanamine (15.3 g, 0.151 mol) in DCM (600 mL) was added methanesulfonyl chloride (10.4 g, 0.0907 mol) slowly at 0° C. The mixture was stirred overnight at room temperature. CH₂Cl₂ (400 mL) were added to the solution, and the mixture was washed with diluted HCl (1M, 1000 mL). The mixture was shaken, the layers were separated, and the organic layer was collected. The organic layer was further washed with water (1000 mL) and brine (1000 mL) and dried over Na₂SO₄. Solvent was removed to give 2-[2-[2-(2-trityloxyethoxy)ethoxy]ethoxy]ethyl methanesulfonate (38.8 g, 99.8%) as a yellow oil.

LCMS 537.2 (M+23) 98% UV (214 nm)

¹H NMR (400 MHz, CDCl₃) δ 7.48-7.44 (m, 6H), 7.32-7.27 (m, 5H), 7.26-7.20 (m, 4H), 4.35-4.30 (m, 2H), 3.75-3.71 (m, 2H), 3.70-3.63 (m, 10H), 3.26-3.20 (m, 2H), 2.98 (s, 3H).

Step (3)

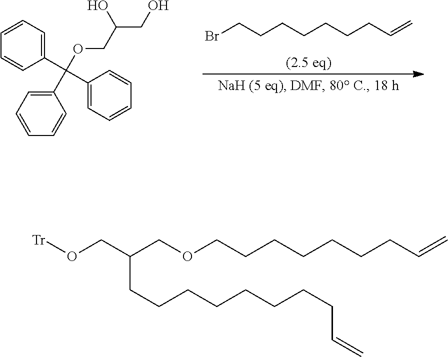

To a suspension of NaH (17.9 g) in 300 ml of anhydrous DMF was added 3-trityloxypropane-1,2-diol (30 g). The mixture was heated at 80° C. for 15 minutes and cooled to room temperature. 9-bromonon-1-ene (46 g) in 10 ml of anhydrous DMF was added dropwise to the mixture which was then heated under 80° C. for 18 h. After cooling to RT, 500 mL of H₂O were added to destroy remain NaH. The organic phase was extracted with 750 mL ethyl acetate. The extract was washed successively with 300 mL of 5% (w/v) NaHCO₃ and 150 ml of brine and dried on Na₂SO₄. The solvent was evaporated under reduced pressure and the resulting oil was purified on a silica gel column eluted with petroleum ether/ethyl acetate (6% to 25% ethyl acetate in petroleum ether) to yield a colorless oil (15.1 g, 28.9%).

¹H NMR (400 MHz, CDCl₃) δ 7.60-7.05 (m, 17H), 5.95-5.66 (m, 2H), 4.97 (ddd, J=21.1, 11.4, 5.9 Hz, 4H), 3.69-3.31 (m, 7H), 3.21-3.06 (m, 2H), 2.02 (dt, J=7.9, 3.7 Hz, 4H), 1.50-1.25 (m, 18H).

Step (4)

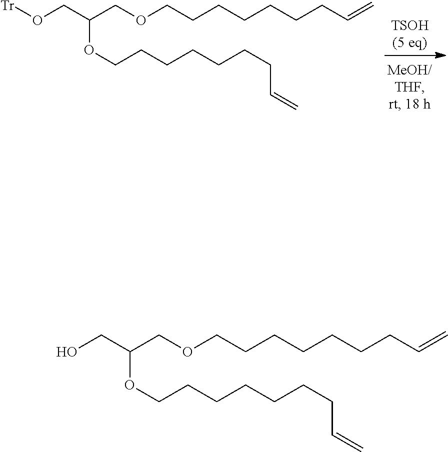

To a solution of [2,3-bis(non-8-enoxy)propoxy-diphenyl-methyl]benzene (30.7 g, 50 mmol) in methanol/THF (600 mL, 1/1 v/v) was added p-Toluenesulfonic acid (47.6 g, 250 mmol) in one portion at room temperature and the mixture was stirred at room temperature for 18 hrs. TLC (4% ethyl acetate in petroleum ether) indicated that the starting material was disappeared completely. 20 mL triethylamine was added to quench the reaction and the solvent was removed under vacuum. The residue was purified by flash chromatography eluted with 20% to 30% ethyl acetate in petroleum ether (21%) to give 2,3-bis(non-8-enoxy)propan-1-ol (12.33 g, 36.2 mmol 72.4% yield) as colorless oil.

¹H NMR (400 MHz, CDCl₃) δ 5.89-5.73 (m, 2H), 5.03-5.00 (m, 1H), 4.99-4.96 (m, 1H), 4.94 (d, J=0.9 Hz, 1H), 4.92 (d, J=0.9 Hz, 1H), 3.76-3.69 (m, 1H), 3.65-3.41 (m, 8H), 2.26-2.19 (m, 1H), 2.09-2.00 (m, 4H), 1.61-1.52 (m, 4H), 1.41-1.27 (m, 16H).

Step (5)

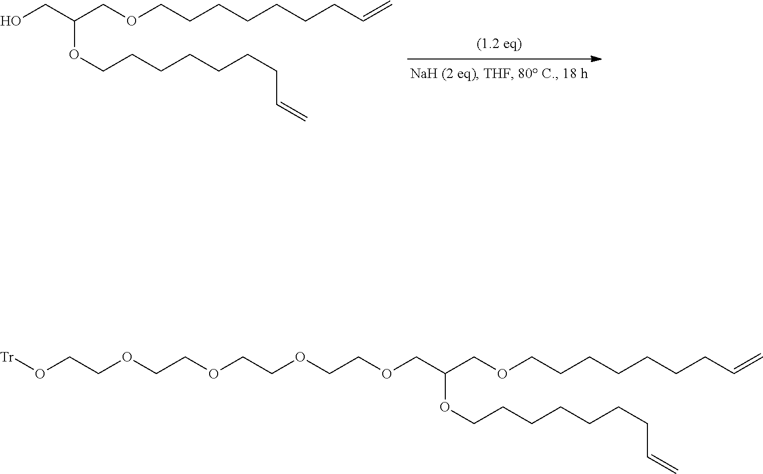

To a mixture of 2,3-bis(non-8-enoxy)propan-1-ol (12.33 g, 36.2 mmol) added NaH (60% mineral oil dispersion, 2.77 g, 72.4 mmol) in 200 mL of dry THF then stirred 15 min at 80° C. Then after the solvent was return at room temperature added 2-[2-[2-(2-trityloxyethoxy)ethoxy]ethoxy]ethyl methanesulfonate (22.4 g, 43.4 mmol) dissolved in 60 mL of dry THF. The reaction mixture was stirred reflux (80° C.) overnight. The reaction mixture was cooled to room temperature, and water (200 mL) was added. EtOAc (400 mL) was added, the mixture was shaken, the layers were separated, and the organic layer was collected. The aqueous layer was extracted with EtOAc (400 mL*2). The combined organic layers were washed with brine and dried over Na2SO4. The residue was purified by flash column chromatography on silica gel eluting with ethyl acetate in petroleum ether (0-15%)(14%) to give target product (23.17 g, 30.5 mmol, 84.3% yield) as pale yellow oil.

¹H NMR (400 MHz, CDCl₃) δ 7.48-7.44 (m, 6H), 7.31-7.26 (m, 6H), 7.25-7.19 (m, 3H), 5.88-5.72 (m, 2H), 5.02-4.99 (m, 1H), 4.98-4.95 (m, 1H), 4.93 (dd, J=2.0, 0.9 Hz, 1H), 4.92-4.89 (m, 1H), 3.70-3.64 (m, 10H), 3.63-3.59 (m, 4H), 3.59-3.40 (m, 9H), 3.26-3.21 (m, 2H), 2.07-1.99 (m, 4H), 1.60-1.50 (m, 4H), 1.41-1.25 (m, 16H).

Step (6)

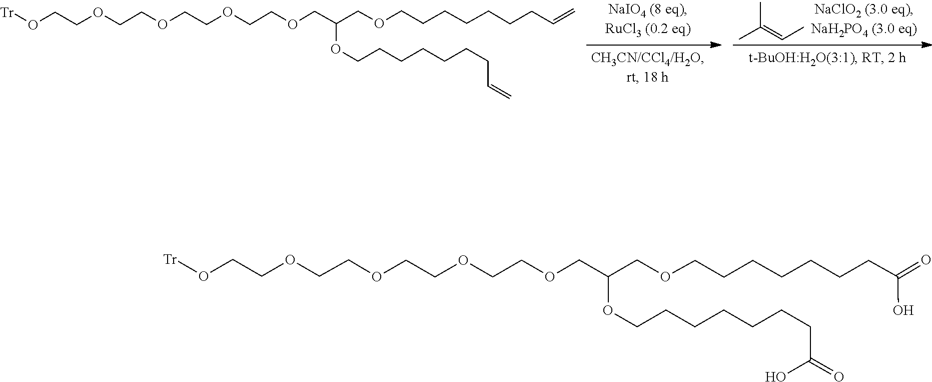

To a solution of [2-[2-[2-[2-[2,3-bis(non-8-enoxy)propoxy]ethoxy]ethoxy]ethoxy]ethoxy-diphenyl-methyl]benzene (23.17 g, 30.5 mmol) in MeCN (200 mL), CCl₄ (200 mL) and water (200 mL) was added NaIO₄ (52.2 g, 244 mmol) and RuCl₃ (1.27 g, 6.1 mmol). The reaction mixture was stirred at room temperature for 24 h. The reaction was filtered and the filtrate was diluted with ethyl acetate (800 mL) and washed with 1N aq. HCl (900 mL). The organic layer was washed with Na₂S2O₃ solution (700 mL*2) and then dried over sodium sulfate, filtered and concentrated to give 8-[2-(7-carboxyheptoxy)-3-[2-[2-[2-(2-trityloxyethoxy)ethoxy]ethoxy]ethoxy]propoxy]octanoic acid (23.28 g, 22 mmol, purity: 75%, 71.9% yield) as yellow oil which was used without further purification.

¹H NMR (400 MHz, CDCl₃) δ 9.75 (s, 1H), 7.46 (d, J=7.2 Hz, 2H), 7.34-7.27 (m, 12H), 7.25-7.19 (m, 1H), 3.75-3.72 (m, 1H), 3.69-3.40 (m, 23H), 3.26-3.20 (m, 1H), 2.45-2.28 (m, 4H), 1.68-1.50 (m, 8H), 1.32 (s, 12H).

Step (7)

8-[2-(8-oxooctoxy)-3-[2-[2-[2-(2-trityloxyethoxy)ethoxy]ethoxy]ethoxy]propoxy]octanoic acid (12.5 g, 4.81 mmol) was dissolved in t-BuOH:H₂O (3:1, 400 mL), containing NaH₂PO4 (1.72 g, 14.4 mmol), 2-methy-2-butene (15 mL) and sodium chlorite (1.3 g, 14.4 mmol). The reaction was stirred for 2 h at rt and LCMS indicated that the starting material was consumed. The reaction mixture was diluted with H₂O. The aqueous layer was extracted with ethyl acetate (800 mL*2). The residue was purified by flash column chromatography on silica gel eluting with CH₃₀H in DCM (0-6%)(3%) to give target product (4.663 g (EXP-20-IQ8160-P2: 0.576 g+EXP-20-IQ8160-2: 4.082 g), 19.2% yield (The combined yield of two-step oxidation)) as light yellow oil.

LCMS: Find peak: MS(ESI) m/z=818.5 (M+Na)+ at 2.300 min.

Multiplet Report

¹H NMR (400 MHz, CDCl₃) δ 7.49-7.43 (m, 6H), 7.32-7.26 (m, 6H), 7.25-7.19 (m, 3H), 4.23 (s, 1H), 3.69-3.39 (m, 23H), 3.26-3.21 (m, 2H), 2.37-2.29 (m, 4H), 1.67-1.50 (m, 8H), 1.33 (s, 12H).

Step (8)

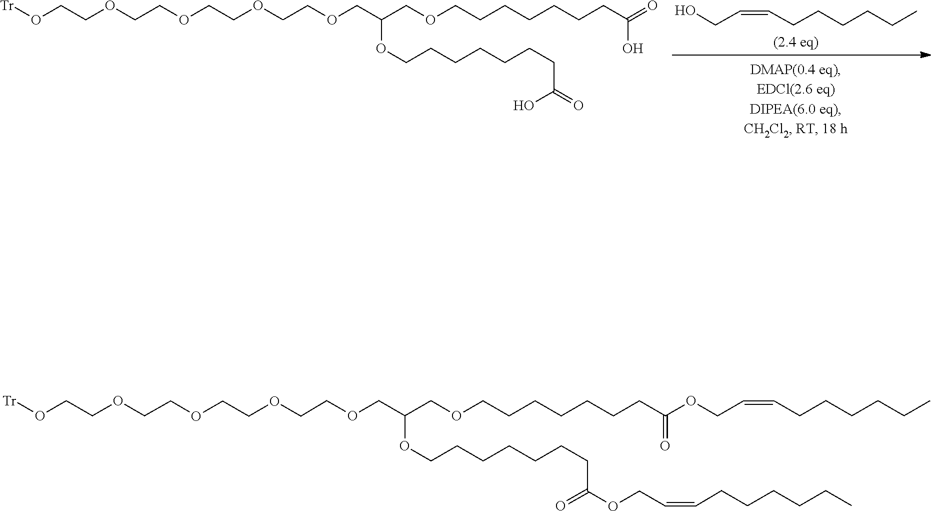

To the solution of 8-[2-(7-carboxyheptoxy)-3-[2-[2-[2-(2-trityloxyethoxy)ethoxy]ethoxy]ethoxy]propoxy]octanoic acid (4.087 g, 5.14 mmol) and (Z)-non-2-en-1-ol (1.75 g, 12.3 mmol) in dry dichloromethane (150 mL) then added DIPEA (3.99 g, 30.8 mmol), DMAP (0.251 mg, 2.06 mmol) and under ice bath added EDCI (2.56 g, 13.4 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 0% to 40%(25%) ethyl acetate in petroleum ether to give [(Z)-non-2-enyl] 8-[2-[8-[(Z)-non-2-enoxy]-8-oxo-octoxy]-3-[2-[2-[2-(2-trityloxyethoxy)ethoxy]ethoxy]ethoxy]propoxy]octanoate (1.646 g, 1.58 mmol, 30.7% yield) as colorless oil.

¹H NMR (400 MHz, CDCl₃) δ 7.50-7.44 (m, 6H), 7.32-7.27 (m, 6H), 7.25-7.18 (m, 3H), 5.69-5.59 (m, 2H), 5.57-5.47 (m, 2H), 4.62 (d, J=6.8 Hz, 4H), 3.70-3.38 (m, 23H), 3.26-3.21 (m, 2H), 2.33-2.26 (m, 4H), 2.13-2.05 (m, 4H), 1.65-1.50 (m, 8H), 1.39-1.25 (m, 28H), 0.92-0.84 (m, 6H).

Step (9)

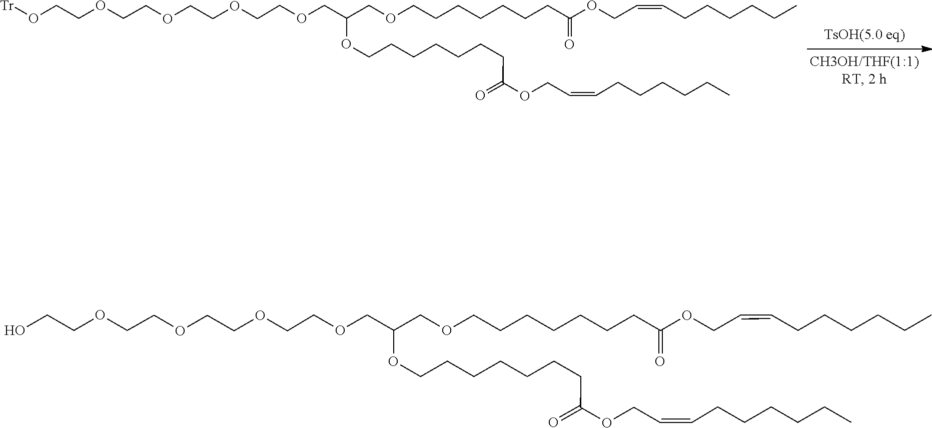

To a solution of[(Z)-non-2-enyl] 8-[2-[8-[(Z)-non-2-enoxy]-8-oxo-octoxy]-3-[2-[2-[2-(2-Trityloxyethoxy)ethoxy]ethoxy]ethoxy]propoxy]octanoate (1.881 g, 1.8 mmol) in methanol/THF (80 mL, 1/1 v/v) was added p-Toluenesulfonic acid (1.71 mg, 9.01 mmol) in one portion at room temperature and the mixture was stirred at room temperature for 2 h. TLC (30% ethyl acetate in petroleum ether) indicated that the starting material was disappeared completely. 5 mL triethylamine was added to quench the reaction and the solvent was removed under vacuum. The residue was purified by flash chromatography eluted with 0% to 10% (6%) CH₃₀H in DCM to give 2,3-bis(non-8-enoxy)propan-1-ol (1.32 g, 1.65 mmol, 91.4%) as colorless oil.

Multiplet Report

¹H NMR (400 MHz, CDCl₃) δ 5.69-5.58 (m, 2H), 5.57-5.47 (m, 2H), 4.62 (d, J=6.7 Hz, 4H), 3.75-3.71 (m, 2H), 3.68-3.60 (m, 14H), 3.58-3.40 (m, 9H), 2.76 (s, 1H), 2.33-2.27 (m, 4H), 2.14-2.03 (m, 4H), 1.66-1.51 (m, 8H), 1.39-1.21 (m, 28H), 0.96-0.80 (m, 6H).

Step (10)

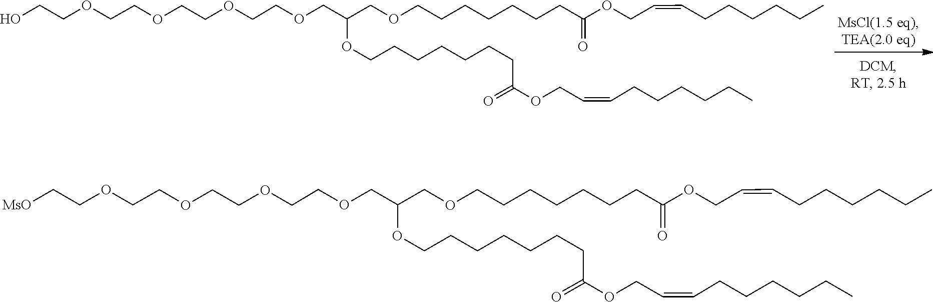 [SMILES: CCCCCC/C=C\COC(=O)CCCCCCCOCC(COCCOCCOCCOCCO)OCCCCCCCC(=O)OC/C=C\CCCCCC ; CCCCCC/C=C\COC(=O)CCCCCCCOCC(COCCOCCOCCOCCOS(C)(=O)=O)OCCCCCCCC(=O)OC/C=C\CCCCCC]

To a solution of [(Z)-non-2-enyl] 8-[3-[2-[2-[2-(2-hydroxyethoxy)ethoxy]ethoxy]ethoxy]-2-[8-[(Z)-non-2-enoxy]-8-oxo-octoxy]propoxy]octanoate (1.32 g, 1.65 mmol) and TEA (triethylamine) (0.333 g, 3.3 mmol) in 30 mL Dichloromethane (DCM), was added Ms-Cl (0.283 g, 2.47 mmol). The mixture was stirred at room temperature for 3 h. TLC (CH3OH/DCM 3%) indicated that the starting material was consumed. The reaction was diluted with dichloromethane and washed with water (10 mL). The organic layer was dried over sodium sulfate, filtered and concentrated to give nonyl to afford [(Z)-non-2-enyl] 8-[3-[2-[2-[2-(2-methylsulfonyloxyethoxy)ethoxy]ethoxy]ethoxy]-2-[8-[(Z)-non-2-enoxy]-8-oxo-octoxy]propoxy]octanoate (1.384 g, 1.57 mmol, 95.5% yield) as pale yellow liquid, which was used directly for next step.

¹H NMR (400 MHz, CDCl₃) δ 5.70-5.59 (m, 2H), 5.56-5.48 (m, 2H), 4.62 (d, J=6.7 Hz, 4H), 4.40-4.36 (m, 2H), 3.78-3.75 (m, 2H), 3.68-3.63 (m, 12H), 3.58-3.40 (m, 9H), 3.08 (s, 3H), 2.33-2.27 (m, 4H), 2.14-2.05 (m, 4H), 1.64-1.51 (m, 8H), 1.40-1.25 (m, 28H), 0.92-0.84 (m, 6H).

Step (11)

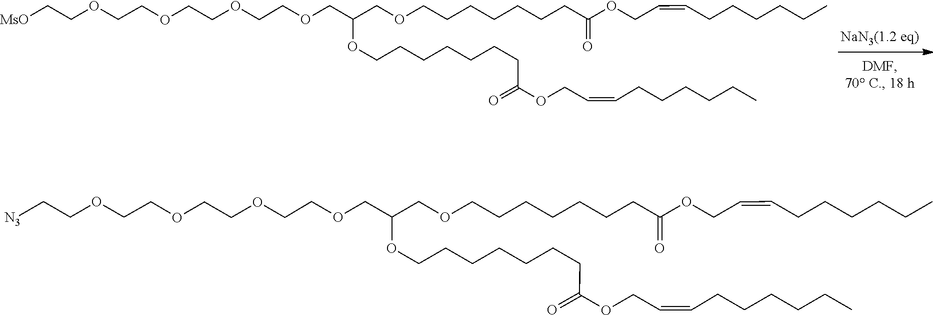 [SMILES: CCCCCC/C=C\COC(=O)CCCCCCCOCC(COCCOCCOCCOCCOS(C)(=O)=O)OCCCCCCCC(=O)OC/C=C\CCCCCC ; CCCCCC/C=C\COC(=O)CCCCCCCOCC(COCCOCCOCCOCCN=[N+]=[N-])OCCCCCCCC(=O)OC/C=C\CCCCCC]

To a solution of undecylundecyl[(Z)-non-2-enyl] 8-[3-[2-[2-[2-(2-methylsulfonyloxyethoxy)ethoxy]ethoxy]ethoxy]-2-[8-[(Z)-non-2-enoxy]-8-oxo-octoxy]propoxy]octanoate (1.38 g, 1.57 mmol) dissolved in DMF (25 mL) then added Na₃N (0.123 g, 1.89 mmol). Then the reaction mixture was stirred at 70° C. for 18 hr.

TCL showed that the starting material was disappeared, and a new spot was observed. Then water (100 mL) was added, and the reaction mixture was extracted with ethyl acetate (100 mL*2). The combined organic phases were washed with brine (100 mL), dried over Na₂SO₄, filtered and concentrated under reduced pressure to give a residue, which was purified by column chromatography eluted with CH₃OH in DCM (0-10%) (6%) (0.995 g, 1.2 mml, 76.5% yield) as colorless liquid.

¹H NMR (400 MHz, CDCl₃) δ 5.71-5.59 (m, 2H), 5.58-5.46 (m, 2H), 4.62 (d, J=6.8 Hz, 4H), 3.69-3.66 (m, 10H), 3.64 (s, 3H), 3.59-3.37 (m, 12H), 2.32-2.27 (m, 4H), 2.14-2.05 (m, 4H), 1.63-1.50 (m, 8H), 1.39-1.25 (m, 28H), 0.92-0.85 (m, 6H).

Step (11)

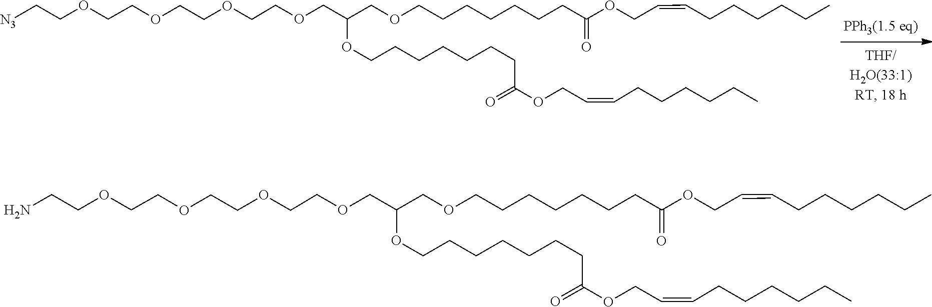 [SMILES: CCCCCC/C=C\COC(=O)CCCCCCCOCC(COCCOCCOCCOCCN=[N+]=[N-])OCCCCCCCC(=O)OC/C=C\CCCCCC ; CCCCCC/C=C\COC(=O)CCCCCCCOCC(COCCOCCOCCOCCN)OCCCCCCCC(=O)OC/C=C\CCCCCC]

A mixture of undecyl [(Z)-non-2-enyl] 8-[3-[2-[2-[2-(2-azidoethoxy)ethoxy]ethoxy]ethoxy]-2-[8-[(Z)-non-2-enoxy]-8-oxo-octoxy]propoxy]octanoate (0.995 g, 1.2 mmol) and triphenylphosphine (0.474 g, 1.81 mmol) in THF (20 mL)/Water (0.6 mL) was stirred at 20° C. for 16 hrs. TLC (ninhydrin, 3% methanol in dichloromethane) indicated the reaction was complete. The solvent was removed and added into DCM then concentrated under reduced pressure to give a residue, which was purified by column chromatography eluted with CH₃OH in CH₂Cl₂ (0-20%(14%)) to give [(Z)-non-2-enyl] 8-[3-[2-[2-[2-(2-aminoethoxy)ethoxy]ethoxy]ethoxy]-2-[8-[(Z)-non-2-enoxy]-8-oxo-octoxy]propoxy]octanoate (0.689 g, 0.861 mmol, 71.5% yield) as light yellow oil.

¹H NMR (400 MHz, CDCl₃) δ 5.70-5.60 (m, 2H), 5.57-5.48 (m, 2H), 4.62 (d, J=6.7 Hz, 4H), 3.72-3.38 (m, 25H), 2.94-2.87 (m, 2H), 2.32-2.28 (m, 4H), 2.14-2.06 (m, 4H), 1.65-1.51 (m, 8H), 1.38-1.26 (m, 28H), 0.91-0.85 (m, 6H).

Step (13)

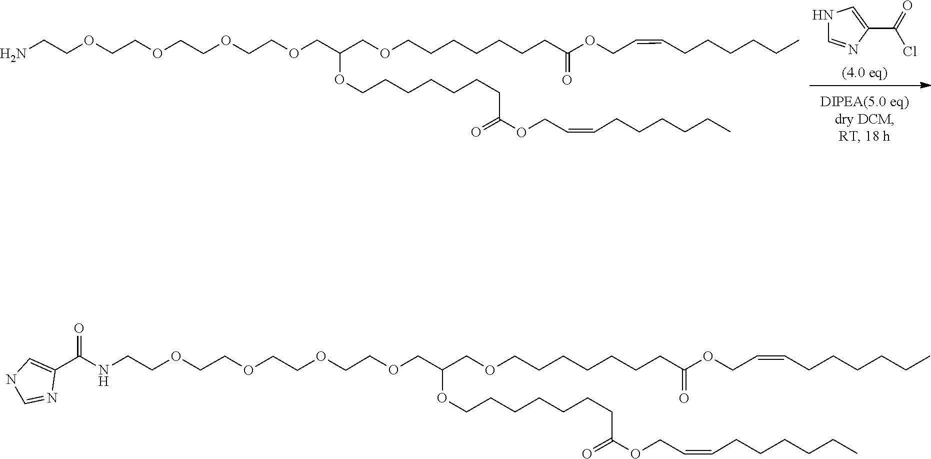

[(Z)-non-2-enyl] 8-[3-[2-[2-[2-(2-aminoethoxy)ethoxy]ethoxy]ethoxy]-2-[8-[(Z)-non-2-enoxy]-8-oxo-octoxy]propoxy]octanoate (190 mg, 0.237 mmol) was dissolved in 10 mL anhydrous DCM and the 1H-imidazole-4-carbonyl chloride (124 mg, 0.95 mmol) in 1 mL anhydrous DMF and DIPEA (153 mg, 1.19 mmol) were added. The mixture was stirred at room temperature overnight. The solvent was evaporated and the product purified by flash chromatography (40 g column, DCM/MeOH 0% to 15%) eluted with methanol in dichloromethane (4%) to give [(Z)-non-2-enyl] 8-[3-[2-[2-[2-[2-(1H-imidazole-4-carbonylamino)ethoxy]ethoxy]ethoxy]ethoxy]-2-[8-[(Z)-non-2-enoxy]-8-oxo-octoxy]propoxy]octanoate (112 mg, 0.119 mmol, 50.1% yield) as colorless oil.

¹H NMR (400 MHz, CDCl₃) δ 7.71-7.56 (m, 3H), 5.70-5.59 (m, 2H), 5.57-5.47 (m, 2H), 4.62 (d, J=6.8 Hz, 4H), 3.67-3.39 (m, 25H), 2.33-2.27 (m, 4H), 2.13-2.05 (m, 4H), 1.64-1.50 (m, 8H), 1.41-1.25 (m, 28H), 0.92-0.84 (m, 6H).

LCMS: MS (ESI) m/z=895.7 (M+H)⁺

### Example 13: Synthesis of 2-butyloctyl 8-[2-[8-(2-butyloctoxy)-8-oxo-octoxy]-3-[2-[2-[2-[2-(1H-imidazole-4-carbonylamino)ethoxy]ethoxy]ethoxy]ethoxy]propoxy]octanoate (compound XIV)

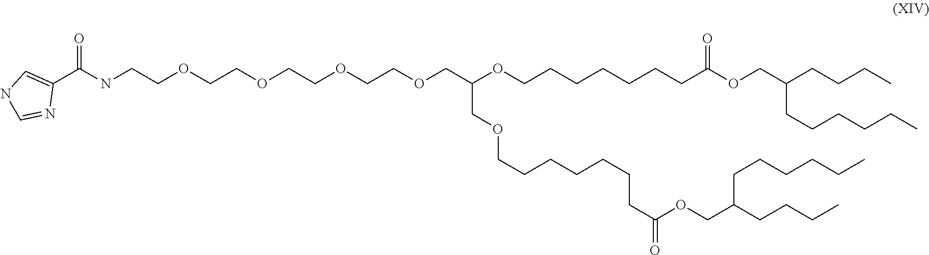 [SMILES: CCCCCCC(CCCC)COC(=O)CCCCCCCOCC(COCCOCCOCCOCCNC(=O)c1c[nH]cn1)OCCCCCCCC(=O)OCC(CCCC)CCCCCC]

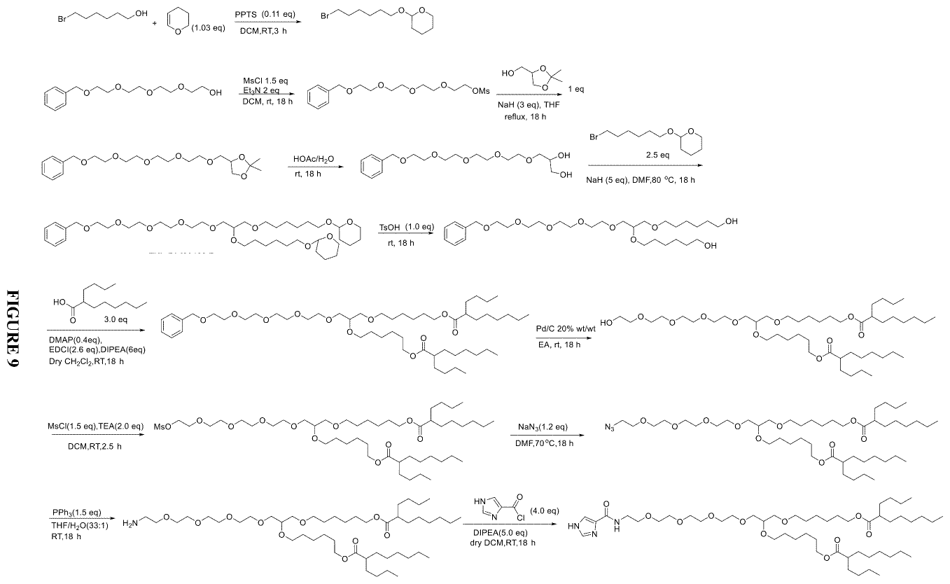

The compound (XIV) was synthesized based on the chemistry shown in Scheme (8) as shown on FIG. 8.

Synthesis of compound (XIV)

Step (1)

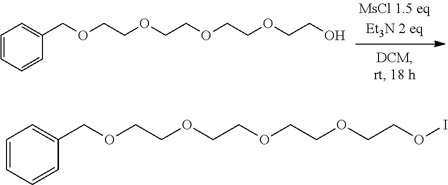 [SMILES: OCCOCCOCCOCCOCc1ccccc1 ; IOCCOCCOCCOCCOCc1ccccc1]

2-[2-[2-(2-benzyloxyethoxy)ethoxy]ethoxy]ethanol (50 g, 0.176 mol) and triethylamine (36.2 g, 0.352 mol) in dry dichloromethane (600 mL) under nitrogen were cooled to 0° C. Methanesulfonyl chloride (30.6 g, 0.264 mol) was added dropwise to this solution at 0° C. The mixture was allowed to warm to room temperature and stirred at room temperature for 18 h. Triethylamine hydrochloride was filtered off, and the DCM solution was washed with 0.1 N HCl and dried over sodium sulfate. Removing the solvent afforded 2-[2-[2-(2-benzyloxyethoxy)ethoxy]ethoxy]ethyl methanesulfonate (62 g, 92%) as light yellow oil which was used without further purification.

LCMS MS 363 (M+H)

Step (2)

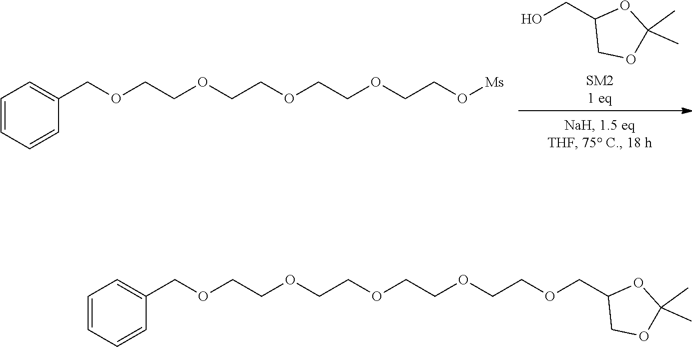

To the solution of (2,2-dimethyl-1,3-dioxolan-4-yl)methanol (62 g, 0.171 mol) in THF (600 mL) was added NaH (6.17 g, 0.257 mol) and the mixture was heated to reflux for 15 min. Then the reaction was cooled to room temperature and 2-[2-[2-(2-benzyloxyethoxy)ethoxy]ethoxy]ethyl methanesulfonate (25.0 g, 0.171 mol) was added under nitrogen and the reaction was heated at 80° C. for 18 hrs. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 20 to 50% ethyl acetate in petroleum ether to give 4-[2-[2-[2-(2-benzyloxyethoxy)ethoxy]ethoxy]ethoxymethyl]-2,2-dimethyl-1,3-dioxolane (43 g, 71% yield) as light yellow oil.

LCMS MS 421 (M+Na)

Step (3)

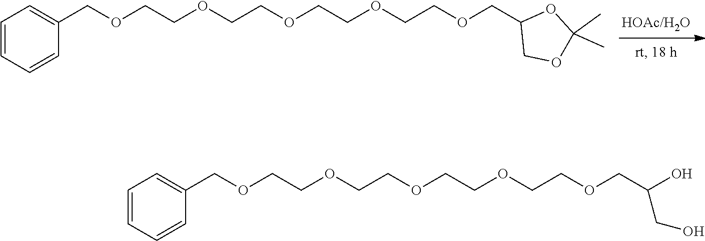 [SMILES: CC1(C)OCC(COCCOCCOCCOCCOCc2ccccc2)O1 ; OCC(O)COCCOCCOCCOCCOCc1ccccc1]

The mixture of 4-[2-[2-[2-(2-benzyloxyethoxy)ethoxy]ethoxy]ethoxymethyl]-2,2-dimethyl-1,3-dioxolane (43 g, 0.103 mol) in AcOH (200 mL) and water (200 mL). The mixture was stirred for 16 h at ambient temperature. Removing the solvent afforded 3-[2-[2-[2-(2-benzyloxyethoxy)ethoxy]ethoxy]ethoxy]propane-1,2-diol (36 g, 95% yield) as light yellow oil which was used without further purification.

LCMS MS 381 (M+Na)

Step (4)

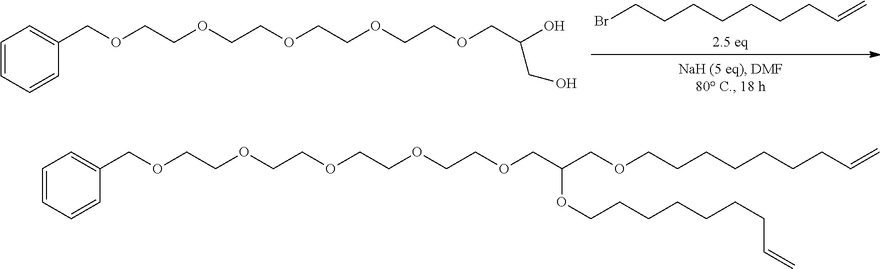

To the solution of 3-[2-[2-[2-(2-benzyloxyethoxy)ethoxy]ethoxy]ethoxy]propane-1,2-diol (20 g, 0.050 mol) in THF (200 mL) was added NaH (8.03 g, 0.201 mol) and the mixture was heated to reflux for 15 min. Then the reaction was cooled to room temperature and 9-bromonon-1-ene (26.6 g, 0.126 mol) was added under nitrogen and the reaction was heated at 80° C. for 18 hrs. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 10 to 30% ethyl acetate in petroleum ether to give 2-[2-[2-[2-[2,3-bis(non-8-enoxy)propoxy]ethoxy]ethoxy]ethoxy]ethoxymethylbenzene (8.8 g, 26% yield) as light yellow oil.

¹H NMR (400 MHz, CDCl₃) δ 7.37-7.27 (m, 5H), 5.87-5.73 (m, 2H), 5.04-4.87 (m, 4H), 4.57 (s, 2H), 3.71-3.59 (m, 16H), 3.59-3.38 (m, 9H), 2.03 (q, J=6.5 Hz, 4H), 1.60-1.49 (m, 4H), 1.41-1.28 (m, 16H).

Step (5)

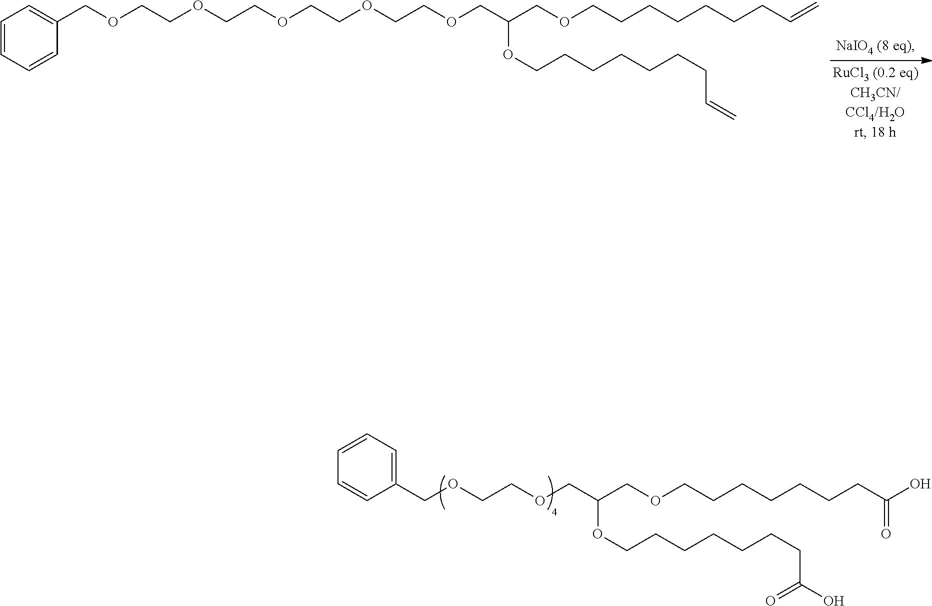

To a solution of 2-[2-[2-[2-[2,3-bis(non-8-enoxy)propoxy]ethoxy]ethoxy]ethoxy]ethoxymethylbenzene (8.5 g, 0.0140 mol) in MeCN (80 mL), CCl₄ (80 mL) and water (80 mL) was added NaIO₄ (24.9 g, 0.116 mol) and RuCl₃ (0.66 g, 2.93 mmol). The reaction mixture was stirred at room temperature for 24 h. LCMS indicated that the title compound was the major product. The reaction was filtered, and the filtrate was diluted with ethyl acetate (600 mL) and washed with 1N aq. HCl (200 mL). The organic layer was dried over sodium sulfate, filtered and concentrated to give 8-[3-[2-[2-[2-(2-benzyloxyethoxy)ethoxy]ethoxy]ethoxy]-2-(7-carboxyheptoxy)propoxy]octanoic acid (8.7 g, 97% yield) as yellow oil which was used without further purification.

¹H NMR (400 MHz, CDCl₃) δ 7.31 (dd, J=22.6, 3.2 Hz, 5H), 4.57 (s, 2H), 3.71-3.61 (m, 19H), 3.59-3.38 (m, 11H), 2.32 (t, J=7.4 Hz, 4H), 1.68-1.47 (m, 10H), 1.32 (s, 14H).

Step (6)

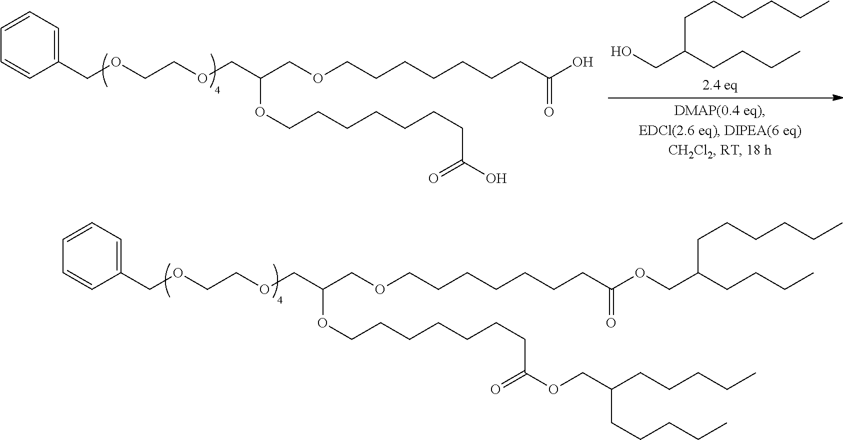

To the solution of 8-[3-[2-[2-[2-(2-benzyloxyethoxy)ethoxy]ethoxy]ethoxy]-2-(7-carboxyheptoxy)propoxy]octanoic acid (0.0135 mol, 8.7 g) and 2-butyloctan-1-ol (0.0324 mol, 6.04 g) in dichloromethane (500 mL) under nitrogen were added N,N-Diisopropylethylamine (0.081 mol, 10.47 g), 4-Dimethylaminopyridine (5.4 mmol, 0.66 g) and 1-(3-Dimethylaminopropyl)-3-ethylcarbodiimide hydrochloride (0.0351 mol, 6.73 g). The mixture was stirred at room temperature for 18 h. The reaction was diluted with dichloromethane, the organic layer was washed with 1 N HCl. Then the organic layer was washed with brine then dried over sodium sulfate, filtered and concentrated. The residue was purified by flash chromatography eluted with 0 to 60% ethyl acetate in petroleum ether to give 2-butyloctyl8-[3-[2-[2-[2-(2-benzyloxyethoxy)ethoxy]ethoxy]ethoxy]-2-[8-(2-butyloctoxy)-8-oxo-octoxy]propoxy] (2.3 g, 16.5% yield) octanoate as colorless oil.

¹H NMR (400 MHz, CDCl₃) δ 7.37-7.28 (m, 5H), 4.57 (s, 2H), 3.96 (d, J=5.8 Hz, 4H), 3.74-3.60 (m, 18H), 3.59-3.38 (m, 11H), 2.29 (t, 4H), 1.84 (s, 1H), 1.67-1.50 (m, 12H), 1.40-1.19 (m, 54H), 0.89 (t, 12H).

Step (7)

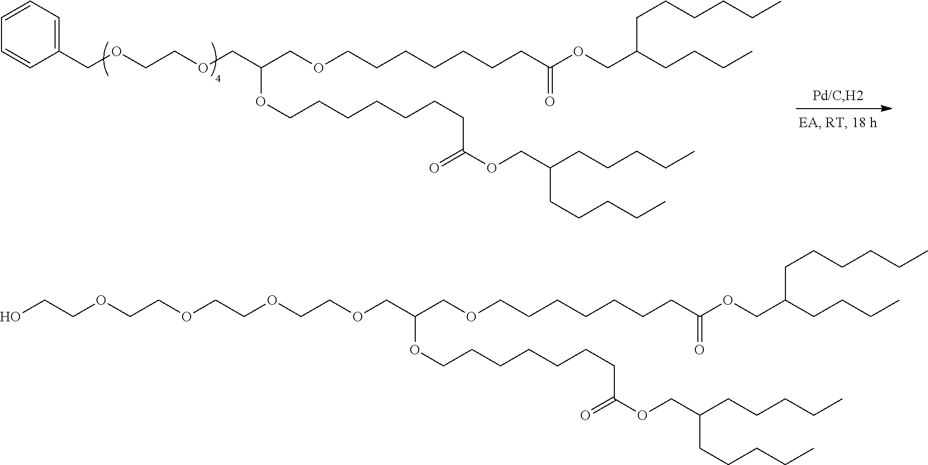

2-butyloctyl 8-[3-[2-[2-[2-(2-benzyloxyethoxy)ethoxy]ethoxy]ethoxy]-2-[8-(2-butyloctoxy)-8-oxo-octoxy]propoxy]octanoate (2.15 g, 2.2 mmol) in ethyl acetate (50 mL) was added Pd/C (500 mg, 20% wt/wt). The mixture was stirred at room temperature under hydrogen for 18 h. TLC (ethyl acetate/petroleum ether=1/1) indicated that the starting material was consumed. The reaction was filtered through celite and washed with ethyl acetate to give 2-butyloctyl 8-[2-[8-(2-butyloctoxy)-8-oxo-octoxy]-3-[2-[2-[2-(2-hydroxyethoxy)ethoxy]ethoxy]ethoxy]propoxy]octanoate (1.51 g, 77.1% yield) as colorless oil.

¹H NMR (400 MHz, CDCl₃) δ 3.97 (t, J=5.8 Hz, 4H), 3.74-3.40 (m, 27H), 2.29 (t, J=7.5 Hz, 4H), 1.69-1.48 (m, 11H), 1.37-1.21 (m, 48H), 0.88 (t, J=5.3 Hz, 12H).

Step (8)

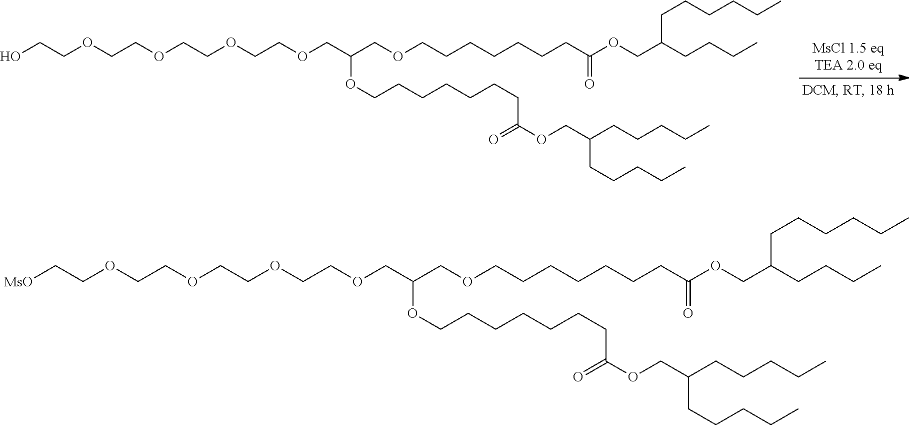 [SMILES: CCCCCCC(CCCC)COC(=O)CCCCCCCOCC(COCCOCCOCCOCCO)OCCCCCCCC(=O)OCC(CCCCC)CCCCC ; CCCCCCC(CCCC)COC(=O)CCCCCCCOCC(COCCOCCOCCOCCOS(C)(=O)=O)OCCCCCCCC(=O)OCC(CCCCC)CCCCC]

2-butyloctyl 8-[2-[8-(2-butyloctoxy)-8-oxo-octoxy]-3-[2-[2-[2-(2-hydroxyethoxy) ethoxy]ethoxy]ethoxy]propoxy]octanoate (1.0 g, 1.12 mmol) and triethylamine (228 mg, 2.25 mmol) in dry dichloromethane (10 mL) under nitrogen were cooled to −5° C. Methanesulfonyl chloride (193 mg, 1.69 mmol) in dry dichloromethane (10 mL) was added dropwise to this solution at 0° C. The mixture was allowed to warm to room temperature and stirred at room temperature for 1 h. TLC (EA:PE=1:1, Rf=0.6) indicated that the starting material was consumed. Triethylamine hydrochloride was filtered off, and the DCM solution was washed with 1N HCl and dried over sodium sulfate. Removing the solvent afforded 2-butyloctyl 8-[2-[8-(2-butyloctoxy)-8-oxo-octoxy]-3-[2-[2-[2-(2-methylsulfonyloxyethoxy)ethoxy]ethoxy]ethoxy]propoxy]octanoate (1.03 g, 94.7% yield) as colorless oil was used without further purification.

¹H NMR (400 MHz, CDCl₃) δ 4.41-4.36 (m, 2H), 3.96 (t, J=5.8 Hz, 4H), 3.79-3.75 (m, 2H), 3.70-3.39 (m, 22H), 3.08 (s, 3H), 2.29 (t, J=7.5 Hz, 4H), 1.67-1.49 (m, 11H), 1.39-1.18 (m, 48H), 0.89 (t, J=6.6, 3.8 Hz, 12H).

Step (9)

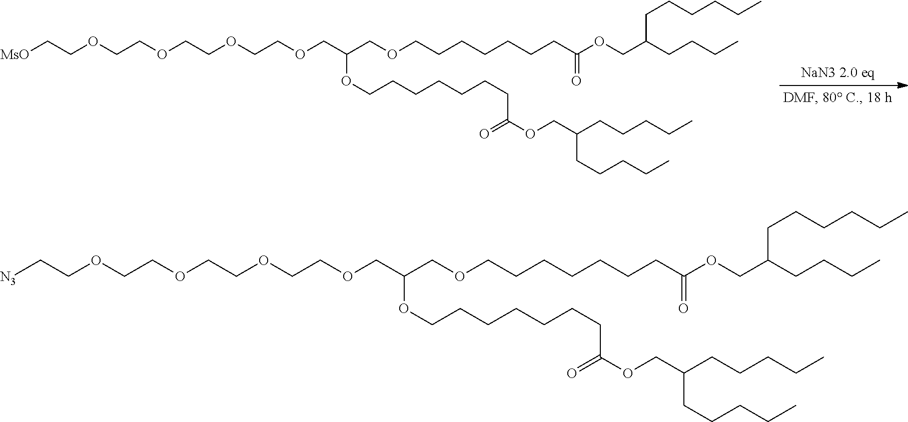 [SMILES: CCCCCCC(CCCC)COC(=O)CCCCCCCOCC(COCCOCCOCCOCCOS(C)(=O)=O)OCCCCCCCC(=O)OCC(CCCCC)CCCCC ; CCCCCCC(CCCC)COC(=O)CCCCCCCOCC(COCCOCCOCCOCCN=[N+]=[N-])OCCCCCCCC(=O)OCC(CCCCC)CCCCC]

2-butyloctyl 8-[2-[8-(2-butyloctoxy)-8-oxo-octoxy]-3-[2-[2-[2-(2-methylsulfonyl oxyethoxy)ethoxy]ethoxy]ethoxy]propoxy]octanoate (1.0 g, 1.0 eq) in N,N-Dimethylformamide (20 mL) was added NaN₃ (0.134 g, 2.0 eq) and the mixture was heated at 80° C. for 18 hrs. TLC indicated that the starting material was consumed. The reaction was quenched with water (200 mL) and then extracted with ethyl acetate (3*100 mL). The combined organic layers were dried over sodium sulfate, filtered and concentrated to obtain 2-butyloctyl 8-[3-[2-[2-[2-(2-azidoethoxy)ethoxy]ethoxy]ethoxy]-2-[8-(2-butyloctoxy)-8-oxo-octoxy]propoxy]octanoate (900 mg, 95.2% yield) without purification.

¹H NMR (400 MHz, CDCl₃) δ 3.97 (t, J=5.8 Hz, 4H), 3.72-3.37 (m, 26H), 2.30 (t, 4H), 1.66-1.50 (m, 11H), 1.37-1.23 (m, 48H), 0.89 (t, 12H).

Step (10)

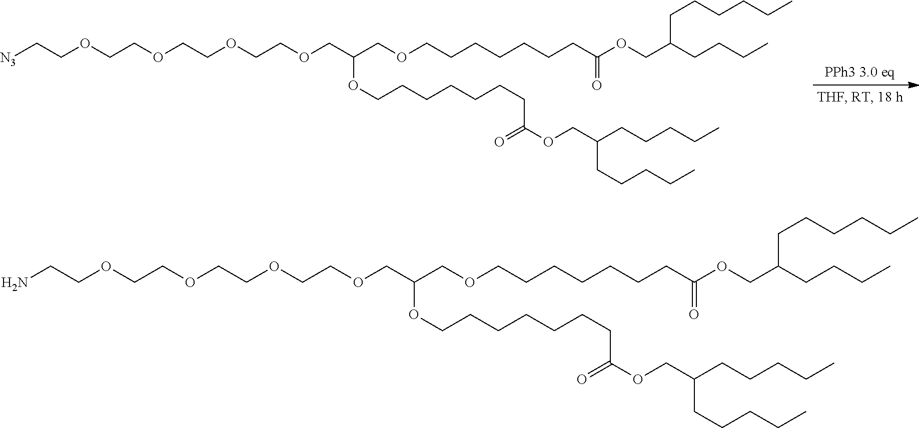 [SMILES: CCCCCCC(CCCC)COC(=O)CCCCCCCOCC(COCCOCCOCCOCCN=[N+]=[N-])OCCCCCCCC(=O)OCC(CCCCC)CCCCC ; CCCCCCC(CCCC)COC(=O)CCCCCCCOCC(COCCOCCOCCOCCN)OCCCCCCCC(=O)OCC(CCCCC)CCCCC]

2-butyloctyl 8-[3-[2-[2-[2-(2-azidoethoxy)ethoxy]ethoxy]ethoxy]-2-[8-(2-butyloctoxy)-8-oxo-octoxy]propoxy]octanoate (0.900 g, 1.0 eq) and triphenylphosphine (0.775 g, 3.0 eq) were dissolved in THF (30 mL) and water (3 mL). The reaction was stirred overnight at room temperature. The reaction was concentrated and purified by flash column chromatography on silica gel eluting with 5% to 25% MeOH in DCM to give 2-butyloctyl 8-[3-[2-[2-[2-(2-aminoethoxy)ethoxy]ethoxy]ethoxy]-2-[8-(2-butyloctoxy)-8-oxo-octoxy]propoxy]octanoate (643 mg, 74% yield) as colorless oil.

¹H NMR (400 MHz, CDCl₃) δ 3.98-3.95 (m, 4H), 3.68-3.40 (m, 26H), 2.94-2.90 (m, 2H), 2.36-2.32 (m, 4H), 1.66-1.51 (m, 11H), 1.35-1.23 (m, 48H), 0.89 (t, J=6.6, 3.9 Hz, 12H).

Step (11)

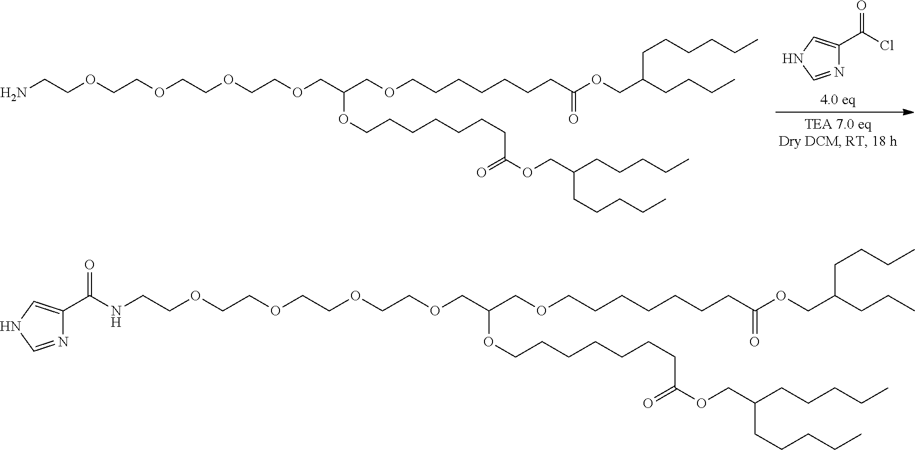 [SMILES: CCCCCCC(CCCC)COC(=O)CCCCCCCOCC(COCCOCCOCCOCCN)OCCCCCCCC(=O)OCC(CCCCC)CCCCC ; O=C(Cl)c1c[nH]cn1 ; CCCCCC(CCCCC)COC(=O)CCCCCCCOC(COCCCCCCCC(=O)OCC(CCC)CCCC)COCCOCCOCCOCCNC(=O)c1c[nH]cn1]

2-butyloctyl 8-[3-[2-[2-[2-(2-aminoethoxy)ethoxy]ethoxy]ethoxy]-2-[8-(2-butyloctoxy)-8-oxo-octoxy]propoxy]octanoate (600 mg, 0.675 mmol) in dry DCM (70 mL) was added N,N-diethylethanamine (0.478 g, 7.0 eq) and 1H-imidazole-4-carbonyl chloride (0.353 g, 4.0 eq), the mixture was stirred at RT for 18 h.

TLC (DCM/MeOH=10:1) and LCMS indicated that the starting material was disappeared. The mixture was concentrated then purified by flash column chromatography on silica gel eluting with 0% to 20% methanol in dichloromethane to give 2-butyloctyl 8-[2-[8-(2-butyloctoxy)-8-oxo-octoxy]-3-[2-[2-[2-[2-(1H-imidazole-4-carbonylamino)ethoxy]ethoxy]ethoxy]ethoxy]propoxy]octanoate (310 mg, 47% yield).

¹H NMR (400 MHz, CDCl₃) δ 7.62 (t, J=14.2 Hz, 3H), 3.97 (t, J=5.8 Hz, 4H), 3.68-3.39 (m, 26H), 2.30 (t, J=7.6, 1.5 Hz, 4H), 1.66-1.51 (m, 11H), 1.44-1.14 (m, 48H), 0.88 (t, J=6.9, 4.0 Hz, 12H).

### Example 14: Synthesis of N-[2-[2-[2-[2-[3-[2,3-bis[(Z)-octadec-9-enoxy]propyl-octyl-amino]-3-oxopropoxy]ethoxy]ethoxy]ethoxy]ethyl]-1H-imidazole-4-carboxamide (compound XV)

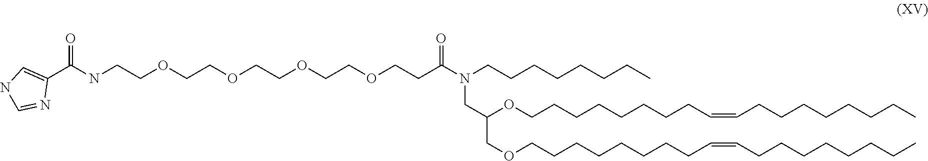 [SMILES: CCCCCCCC/C=C\CCCCCCCCOCC(CN(CCCCCCCC)C(=O)CCOCCOCCOCCOCCNC(=O)c1c[nH]cn1)OCCCCCCCC/C=C\CCCCCCCC]

The compound (XV) was synthesized based on the chemistry shown in Scheme (13).

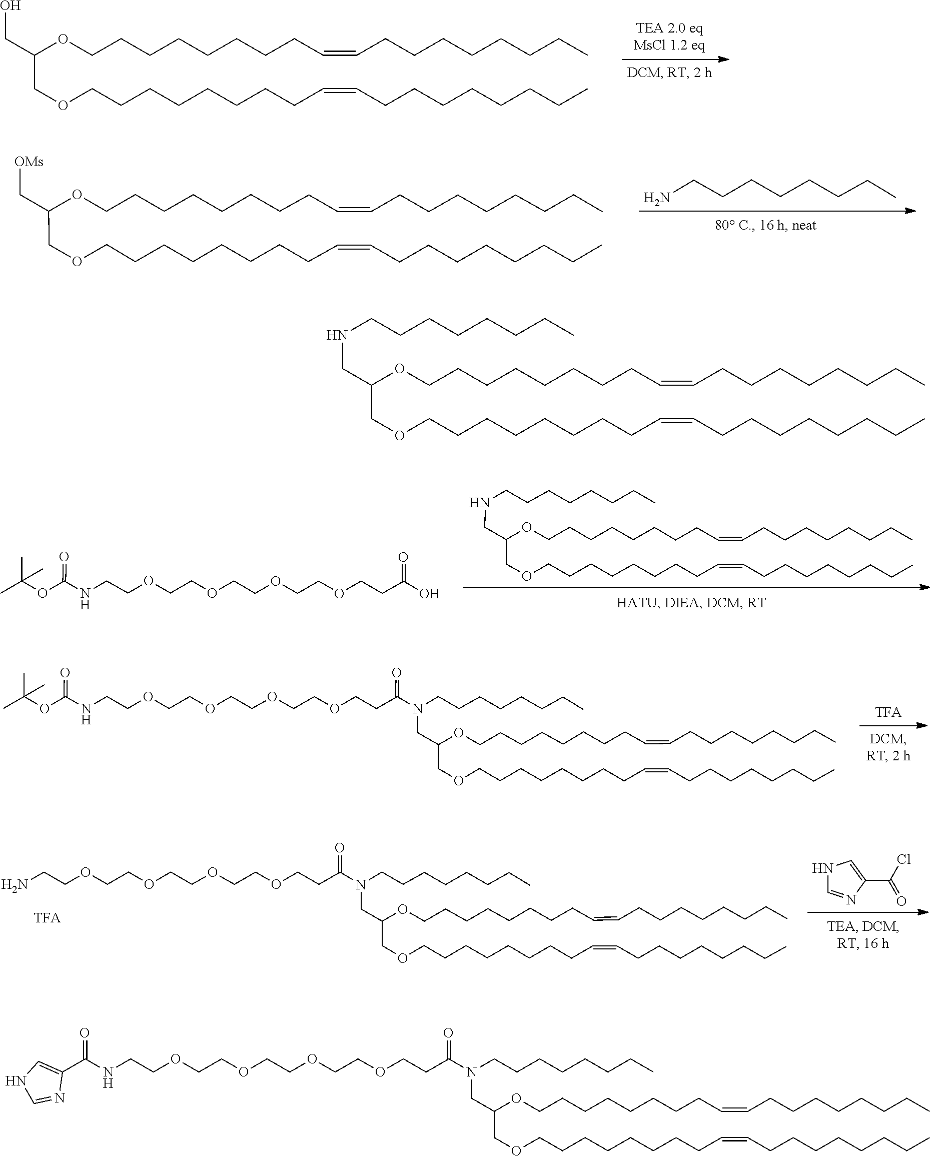

Synthesis of Compound (XV)

Step (1)

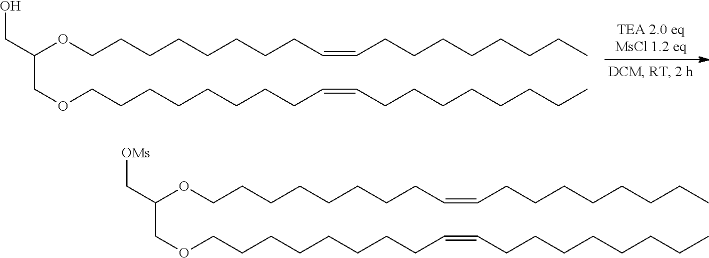 [SMILES: CCCCCCCC/C=C\CCCCCCCCOCC(CO)OCCCCCCCC/C=C\CCCCCCCC ; CCCCCCCC/C=C\CCCCCCCCOCC(COS(C)(=O)=O)OCCCCCCCC/C=C\CCCCCCCC]

To the solution of 2,3-bis[(Z)-octadec-9-enoxy]propan-1-ol (1.0 g, 1.69 mmol), triethylamine (0.512 g, 5.06 mmol) in DCM (20 mL) was added Methanesulfonyl chloride (0.386 g, 3.37 mmol) and the mixture was stirred at rt for 2 h. TLC indicated that the starting material was consumed. The reaction was quenched with water and extracted with DCM. The aqueous layer was extracted with DCM again. The combined organic layers were washed with brine, dried over sodium sulfate, filtered and concentrated to give 2,3-bis[(Z)-octadec-9-enoxy]propyl methanesulfonate (1.1 g, 97%) as colorless oil.

¹H NMR (400 MHz, CDCl₃) δ 5.37-5.32 (m, 3H), 4.25 (dd, J=10.9, 5.7 Hz, 1H), 3.68 (s, 2H), 3.59-3.39 (m, 7H), 3.17-3.07 (m, 7H), 3.04 (s, 3H), 2.01 (dd, J=12.4, 6.6 Hz, 6H), 1.56 (d, J=4.5 Hz, 4H), 1.37-1.22 (m, 45H), 0.88 (t, J=6.8 Hz, 6H).

Step (2)

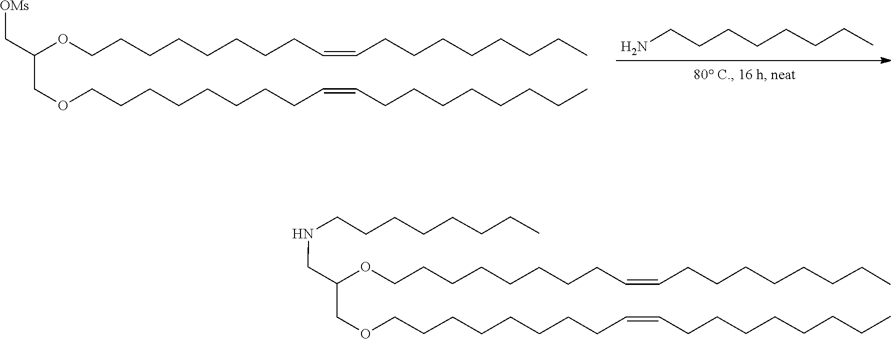

The mixture of 3-2,3-bis[(Z)-octadec-9-enoxy]propyl methanesulfonate (4.5 g, 6.71 mmol) and octan-1-amine in (17.3 g, 134 mmol) was heated at 80° C. for 18 h. The reaction mixture was purified through flash chromatography eluted with 10 to 50% ethyl acetate in petroleum ether to give N-[2,3-bis[(Z)-octadec-9-enoxy]propyl]octan-1-amine (4.2 g, 89% yield) as light yellow oil.

¹H NMR (400 MHz, CDCl₃) δ 5.45-5.27 (m, 3H), 3.68-3.38 (m, 7H), 2.78-2.52 (m, 4H), 2.08-1.90 (m, 7H), 1.68-1.43 (m, 9H), 1.39-1.19 (m, 55H), 0.88 (t, J=6.6 Hz, 9H).

Step (3)

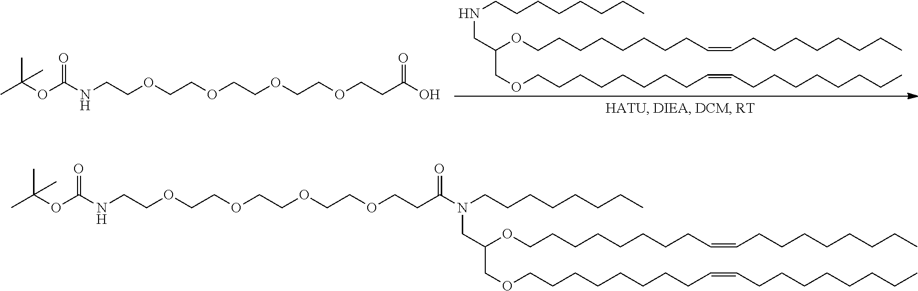 [SMILES: CC(C)(C)OC(=O)NCCOCCOCCOCCOCCC(=O)O ; CCCCCCCC/C=C\CCCCCCCCOCC(CNCCCCCCCC)OCCCCCCCC/C=C\CCCCCCCC ; CCCCCCCC/C=C\CCCCCCCCOCC(CN(CCCCCCCC)C(=O)CCOCCOCCOCCOCCNC(=O)OC(C)(C)C)OCCCCCCCC/C=C\CCCCCCCC]

A mixture of 3-[2-[2-[2-[2-(tert-butoxycarbonylamino)ethoxy]ethoxy]ethoxy]ethoxy]propanoic acid (0.4 g, 1.09 mmol), bis(dimethylamino)methylene-(triazolo[4,5-b]pyridin-3-yl)oxonium; hexafluorophosphate (0.624 g, 1.64 mmol), DIEA (0.283 g, 2.19 mmol) and N-[2,3-bis[(Z)-octadec-9-enoxy]propyl]octan-1-amine (0.771 g, 1.09 mmol) in DCM (10 mL) was stirred for 16 h at ambient temperature. The mixture was poured into DCM (100 mL). The organic layer was washed with 1 N HCl, sat NaCl, dried over NaSO4 and concentrated. The residue was purified by flash column chromatography on silica gel eluting with 1:81ethyl acetate/petroleum ether to give tert-butyl N-[2-[2-[2-[2-[3-[2,3-bis[(Z)-octadec-9-enoxy]propyl-octyl-amino]-3-oxo-propoxy]ethoxy]ethoxy] ethoxylethyllcarbamate (0.95 g, 82.5%) as colourless oil.

¹H NMR (400 MHz, CDCl₃) δ 5.36 (dt, J=10.6, 4.7 Hz, 3H), 3.84-3.16 (m, 31H), 2.69-2.61 (m, 2H), 2.07-1.93 (m, 6H), 1.60-1.47 (m, 6H), 1.44 (s, 9H), 1.23 (d, J=33.4 Hz, 56H), 0.91-0.85 (m, 9H).

Step (4)

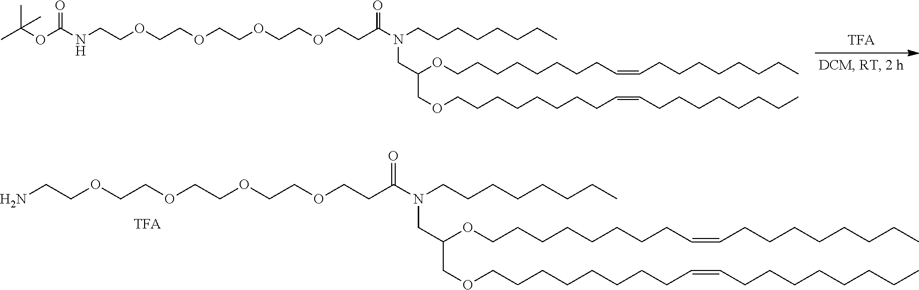 [SMILES: CCCCCCCC/C=C\CCCCCCCCOCC(CN(CCCCCCCC)C(=O)CCOCCOCCOCCOCCNC(=O)OC(C)(C)C)OCCCCCCCC/C=C\CCCCCCCC ; CCCCCCCC/C=C\CCCCCCCCOCC(CN(CCCCCCCC)C(=O)CCOCCOCCOCCOCCN)OCCCCCCCC/C=C\CCCCCCCC]

A mixture of tert-butyl N-[2-[2-[2-[2-[3-[2,3-bis[(Z)-octadec-9-enoxy]propyl-octyl-amino]-3-oxo-propoxy]ethoxy]ethoxy]ethoxy]ethyl]carbamate (0.95 g, 0.903 mmol) in DCM (5 mL) was added TFA (2.06 g, 18.1 mmol) at rt. The mixture was stirred for 3 h at ambient temperature. The mixture was concentrated to give 3-[2-[2-[2-(2-aminoethoxy)ethoxy]ethoxy]ethoxy]-N-[2,3-bis[(Z)-octadec-9-enoxy]propyl]-N-octyl-propanamide; 2,2,2-trifluoroacetic acid (0.94 g, 97.7%) as colourless oil.

¹H NMR (400 MHz, CDCl₃) δ 5.46-5.27 (m, 3H), 3.87-3.79 (m, 2H), 3.77-3.15 (m, 28H), 2.84-2.58 (m, 2H), 2.10-1.88 (m, 6H), 1.63-1.44 (m, 6H), 1.27 (s, 54H), 0.88 (t, J=6.7 Hz, 9H).

Step (5)

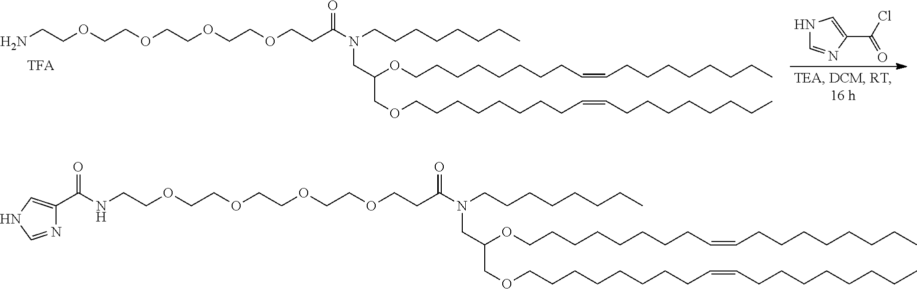 [SMILES: CCCCCCCC/C=C\CCCCCCCCOCC(CN(CCCCCCCC)C(=O)CCOCCOCCOCCOCCN)OCCCCCCCC/C=C\CCCCCCCC ; O=C(Cl)c1c[nH]cn1 ; CCCCCCCC/C=C\CCCCCCCCOCC(CN(CCCCCCCC)C(=O)CCOCCOCCOCCOCCNC(=O)c1c[nH]cn1)OCCCCCCCC/C=C\CCCCCCCC]

A mixture of 3-[2-[2-[2-(2-aminoethoxy)ethoxy]ethoxy]ethoxy]-N-[2,3-bis[(Z)-octadec-9-enoxy]propyl]-N-octyl-propanamide; 2,2,2-trifluoroacetaldehyde (0.5 g, 0.476 mmol) and N,N-diethylethanamine (0.289 g, 2.86 mmol) in DCM (40 mL) was added 1H-imidazole-4-carbonyl chloride (0.249 g, 191 mmol). The mixture was stirred for 16 h at room temperature. The mixture was concentrated and the residue was purified by column chromatography on silica gel eluting with 2%-8% MeOH in DCM to afford N-[2-[2-[2-[2-[3-[2,3-bis[(Z)-octadec-9-enoxy]propyl-octyl-amino]-3-oxo-propoxy]ethoxy]ethoxy]ethoxy]ethyl]-1H-imidazole-4-carboxamide (0.305 g, 61%) as yellow oil.

¹H NMR (400 MHz, CDCl₃) δ 7.64 (d, J=5.8 Hz, 2H), 5.46-5.23 (m, 3H), 3.83-3.20 (m, 29H), 2.77-2.57 (m, 2H), 2.14-1.86 (m, 8H), 1.63-1.42 (m, 7H), 1.27 (s, 55H), 0.93-0.82 (m, 9H).

### Example 15: Synthesis of N-[2-[2-[2-[2-[2-[2,3-bis[(Z)-octadec-9-enoxy]propanoyl-octylamino]ethoxy]ethoxy]ethoxy]ethoxy]ethyl]-1H-imidazole-4-carboxamide (compound XVI)

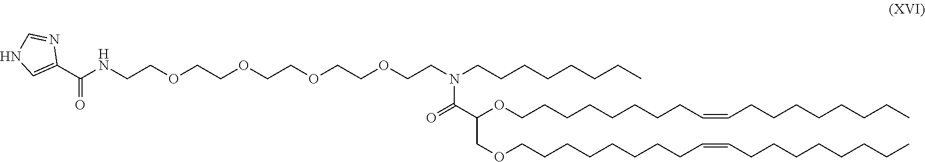 [SMILES: CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1]

Synthesis of Compound (XVI)

Step (1)

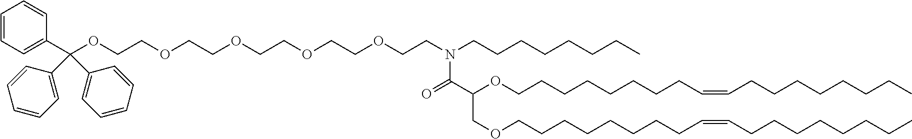 [SMILES: CCCCCCCC/C=C\CCCCCCCCOCC(OCCCCCCCC/C=C\CCCCCCCC)C(=O)N(CCCCCCCC)CCOCCOCCOCCOCCOC(c1ccccc1)(c1ccccc1)c1ccccc1]

To a solution of N-[2-[2-[2-[2-(2-trityloxyethoxy)ethoxy]ethoxy]ethoxy]ethyl]octan-1-amine (2 g, 2.87 mmol) in DCM (50 ml) was added 2,3-bis[(Z)-octadec-9-enoxy]propanoic acid (2.09 g, 3.45 mmol), 4-Dimethylaminopyridine (35 mg), 0-(7-Azabenzotriazol-1-yl)-N,N,N′,N′-tetramethyluronium hexafluorophosphate (1.64 g, 4.3 mmol) and TEA (581 mg, 5.74 mmol). The mixture was stirred at 25° C. for 18 hrs. Then the mixture was dealt with DCM (50 ml), washed with water (250 ml×2), NaCl sat.aq (250 ml) and dried over Na₂SO₄. The organic was purified by flash (5% MeOH in DCM), to give 2,3-bis[(Z)-octadec-9-enoxy]-N-octyl-N-[2-[2-[2-[2-(2-trityloxyethoxy)ethoxy]ethoxy]ethoxy]ethyl]propanamide (2.7 g, 2.17 mmol, yield 75.6%) as a yellow oil.

¹H NMR (400 MHz, CDCl₃) δ 7.46 (d, J=7.6 Hz, 6H), 7.29 (t, J=6.4 Hz, 6H), 7.25-7.20 (m, 3H), 5.34 (s, 3H), 4.36 (d, J=36.5 Hz, 1H), 3.85-3.17 (m, 29H), 2.20-1.89 (m, 7H), 1.60-1.14 (m, 60H), 0.87 (d, J=6.8 Hz, 9H)

Step (2)

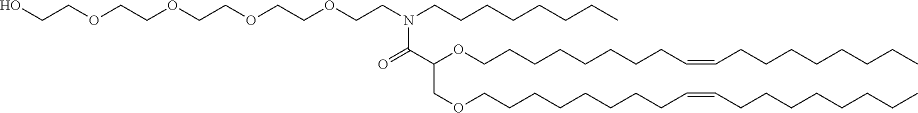 [SMILES: CCCCCCCC/C=C\CCCCCCCCOCC(OCCCCCCCC/C=C\CCCCCCCC)C(=O)N(CCCCCCCC)CCOCCOCCOCCOCCO]

To a solution of 2,3-bis[(Z)-octadec-9-enoxy]-N-octyl-N-[2-[2-[2-[2-(2-trityloxyethoxy)ethoxy]ethoxy]ethoxy]ethyl]propanamide (1020 mg, 0.86 mmol) in THF/MeOH (20 ml, 1/1) was added Toluene-4-sulfonic acid (822 mg, 4.32 mmol). The mixture was stirred at 25° C. for 2 hrs. TEA (1.5 ml) was added to this mixture and the mixture was concentrated and purified by flash (10 MeOH % in DCM), to give N-[2-[2-[2-[2-(2-hydroxyethoxy)ethoxy]ethoxy]ethoxy]ethyl]-2,3-bis[(Z)-octadec-9-enoxy]-N-octyl-propanamide (766 mg, 0.8 mmol, yield 92.6%) as a colorless oil.

¹H NMR (500 MHz, CDCl₃) δ 5.39-5.31 (m, 4H), 3.75-3.56 (m, 27H), 3.46-3.43 (m, 2H), 1.99 (dd, J=14.3, 7.9 Hz, 8H), 1.55 (dd, J=11.6, 6.7 Hz, 4H), 1.26 (d, J=7.0 Hz, 56H), 0.90-0.87 (m, 9H).

Step (3)

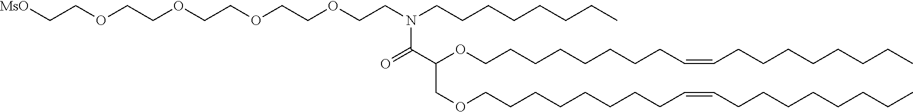 [SMILES: CCCCCCCC/C=C\CCCCCCCCOCC(OCCCCCCCC/C=C\CCCCCCCC)C(=O)N(CCCCCCCC)CCOCCOCCOCCOCCOS(C)(=O)=O]

To a solution of N-[2-[2-[2-[2-(2-hydroxyethoxy)ethoxy]ethoxy]ethoxy]ethyl]-2,3-bis[(Z)-octadec-9-enoxy]-N-octylpropanamide (860 mg, 0.92 mmol) in DCM (10 ml) was added TEA (185 mg, 1.83 mmol) and Methanesulfonyl chloride (157 mg, 1.37 mmol). The mixture was stirred at 25° C. for 18 hrs. Then the mixture was dealt with DCM (50 ml), washed with water (50 ml), 1N HCl (50 ml), NaHCO₄ sat.aq (50 ml), NaCl sat.aq (50 ml) and dried over Na2SO4. The organic was concentrated to give 2-[2-[2-[2-[2-[2,3-bis[(Z)-octadec-9-enoxy]propanoyl-octylamino]ethoxy]ethoxy]ethoxy]ethoxy]ethyl methanesulfonate (806 mg, 0.75 mmol) as a colorless oil. EXP-21-TV4334-N2 (621 mg)

¹H NMR (400 MHz, CDCl₃) δ 5.34 (s, 4H), 4.38 (d, J=3.7 Hz, 1H), 3.76 (d, J=4.5 Hz, 2H), 3.70-3.55 (m, 22H), 3.43 (d, J=18.9 Hz, 4H), 3.08 (s, 3H), 2.01 (d, J=5.2 Hz, 8H), 1.56-1.50 (m, 4H), 1.27 (s, 56H), 0.88 (t, J=5.0 Hz, 9H).

Step (4)

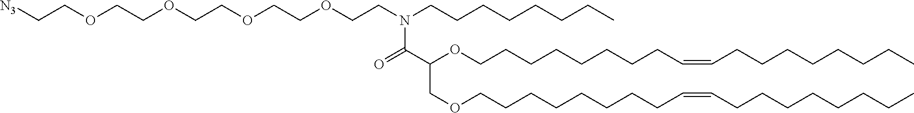 [SMILES: CCCCCCCC/C=C\CCCCCCCCOCC(OCCCCCCCC/C=C\CCCCCCCC)C(=O)N(CCCCCCCC)CCOCCOCCOCCOCCN=[N+]=[N-]]

To a solution of 2-[2-[2-[2-[2-[2,3-bis[(Z)-octadec-9-enoxy]propanoyl-octylamino]ethoxy]ethoxy]ethoxy]ethoxy]ethyl methanesulfonate (806 mg, 0.79 mmol) in DMF (10 ml) was added NaN3 (155 mg, 2.38 mmol). The mixture was stirred at 70° C. for 14 hr. The mixture was dealt with EA (50 ml), washed with water (50 ml), NaCl sat.aq (50 ml) and dried over Na2SO4. The organic was concentrated and purified by flash (50% EA in PE), to give N-[2-[2-[2-[2-(2-azidoethoxy)ethoxy]ethoxy]ethoxy]ethyl]-2,3-bis[(Z)-octadec-9-enoxy]-N-octyl-propanamide (400 mg, 0.4 mmol, yield 50.3%) as a colorless oil.

¹H NMR (400 MHz, CDCl₃) δ 5.42-5.29 (m, 4H), 4.37 (dt, J=39.0, 5.4 Hz, 1H), 3.71-3.57 (m, 22H), 3.47-3.39 (m, 6H), 2.12-1.91 (m, 8H), 1.57-1.23 (m, 70H), 0.88 (t, J=5.0 Hz, 9H).

Step (5)

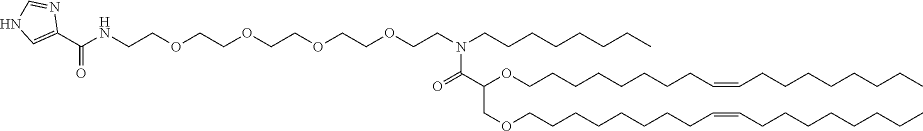 [SMILES: CCCCCCCC/C=C\CCCCCCCCOCC(OCCCCCCCC/C=C\CCCCCCCC)C(=O)N(CCCCCCCC)CCOCCOCCOCCOCCNC(=O)c1c[nH]cn1]

To a solution of N-[2-[2-[2-[2-(2-aminoethoxy)ethoxy]ethoxy]ethoxy]ethyl]-2,3-bis[(Z)-octadec-9-enoxy]-N-octylpropanamide (420 mg, 0.45 mmol) in DCM (5 ml) was added DIEA (290 mg, 2.24 mmol) and 1H-imidazole-4-carbonyl chloride (234 mg, 1.79 mmol). The mixture was stirred at 25° C. for 18 hrs. The mixture was dealt with DCM (50 ml), washed with water (50 ml), NaCl sat.aq (50 ml) and dried over Na₂SO₄. The organic was concentrated and purified by flash (0-10% MeOH in DCM), to give N-[2-[2-[2-[2-[2-[2,3-bis[(Z)-octadec-9-enoxy]propanoyl-octyl-amino]ethoxy]ethoxy]ethoxy]ethoxy]ethyl]-1H-imidazole-4-carboxamide (278 mg, 0.26 mmol, yield 58.8%) as a colourless oil.

¹H NMR (400 MHz, CDCl₃) δ 7.64 (s, 1H), 7.59 (d, J=17.0 Hz, 1H), 7.52 (s, 1H), 5.34 (s, 4H), 4.33 (s, 1H), 3.71-3.36 (m, 28H), 2.01 (d, J=5.4 Hz, 8H), 1.27 (s, 60H), 0.88 (t, J=5.2 Hz, 9H)

### Example 16: Synthesis of 6-[2-[6-(2-butyloctanoyloxy)hexoxy]-3-[2-[2-[2-[2-(1H-imidazole-4-carbonylamino)ethoxy]ethoxy]ethoxy]ethoxy]propoxy]hexyl 2-butyloctanoate (Compound XVII)

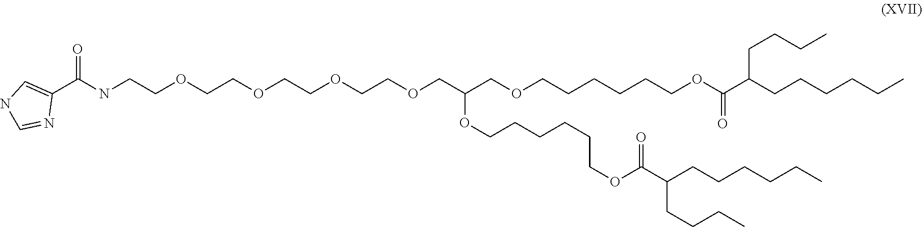 [SMILES: CCCCCCC(CCCC)C(=O)OCCCCCCOCC(COCCOCCOCCOCCNC(=O)c1c[nH]cn1)OCCCCCCOC(=O)C(CCCC)CCCCCC]

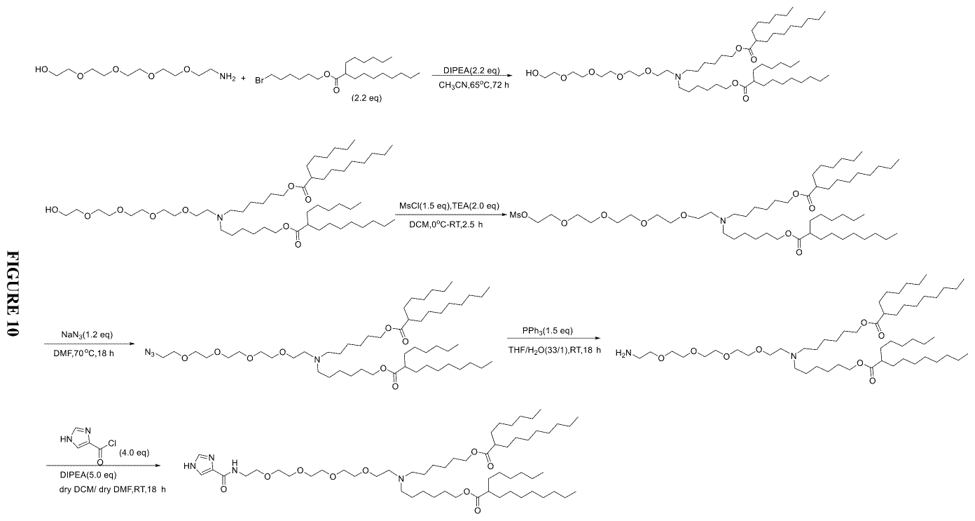

The compound (XVII) was synthesized based on the chemistry shown in Scheme (9) as shown on FIG. 9.

Synthesis of Compound (XVII)

Step (1)

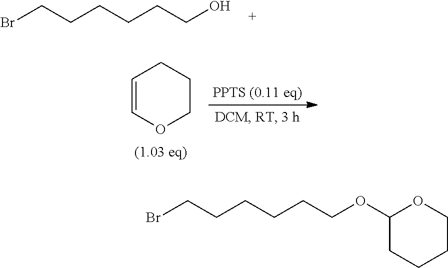 [SMILES: OCCCCCCBr ; C1=COCCC1 ; BrCCCCCCOC1CCCCO1]

To a solution of 6-bromohexan-1-ol (10 g, 55.2 mmol) and 3,4-dihydro-2H-pyran (4.78 g, 56.9 mmol) in DCM (150 mL) was added PPTS (1.61 g, 6.41 mmol) then stirred at room temperature for 3 h. TLC (EA/PE 9/1, SM R_{f}: 0.2; product, R_{f}: 0.7) indicated that all the starting materials was consumed. The solvent was concentrated and purified by flash chromatography column (0-10% EA in PE (5%) to give 2-(6-bromohexoxy)tetrahydropyran (12.72 g, 48 mmol, 86.9% yield) as colorless oil.

¹H NMR (500 MHz, CDCl₃) δ 4.60-4.53 (m, 1H), 3.90-3.83 (m, 1H), 3.77-3.71 (m, 1H), 3.53-3.47 (m, 1H), 3.44-3.35 (m, 3H), 1.93-1.79 (m, 3H), 1.76-1.67 (m, 1H), 1.65-1.36 (m, 10H).

Step (2)

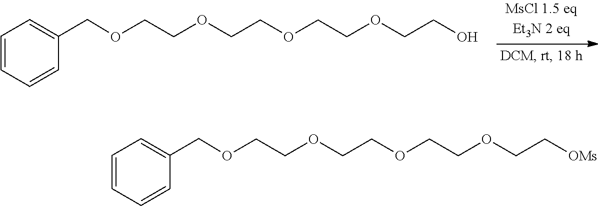 [SMILES: OCCOCCOCCOCCOCc1ccccc1 ; CS(=O)(=O)OCCOCCOCCOCCOCc1ccccc1]

To the solution of 2-[2-[2-(2-benzyloxyethoxy)ethoxy]ethoxy]ethanol (10 g, 35.2 mmol) and triethylamine (7.12 g, 70.3 mmol) in dry dichloromethane (100 mL) at 0° C. was added methanesulfonyl chloride (6.04 g, 52.8 mmol) in dry DCM (10 mL) dropwise. The mixture was warmed to room temperature and stirred at room temperature for 18 h. Triethylamine hydrochloride was filtered off, and the DCM solution was washed with 0.1 N HCl and dried over sodium sulfate. Removing the solvent afforded 2-[2-[2-(2-benzyloxyethoxy)ethoxy]ethoxy]ethyl methanesulfonate (12.7 g, 35 mmol, quant.) as light yellow oil which was used without further purification.

¹H NMR (500 MHz, CDCl₃) δ 7.35-7.26 (m, 5H), 4.56 (s, 2H), 4.38-4.34 (m, 2H), 3.77-3.73 (m, 2H), 3.69-3.61 (m, 12H), 3.06 (s, 3H).

Step (3)

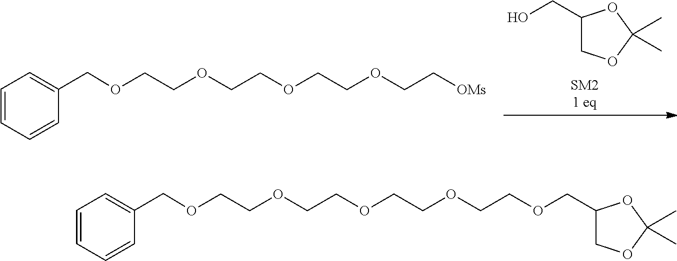

To the solution of (2,2-dimethyl-1,3-dioxolan-4-yl)methanol (4.63 g, 35 mmol) in dry THF (90 mL) was added NaH (4.2 g, 105 mmol) portionwise at 0° C. then the mixture was heated to reflux for 30 min. Then the reaction was cooled to room temperature and 2-[2-[2-(2-benzyloxyethoxy)ethoxy]ethoxy]ethyl methanesulfonate (12.7 g, 35 mmol) in dry THF (30 mL) was added under nitrogen and the reaction was heated at 80° C. for 24 h. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 0 to 5% CH₃OH in DCM to give 4-[2-[2-[2-(2-benzyloxyethoxy)ethoxy]ethoxy]ethoxymethyl]-2,2-dimethyl-1,3-dioxolane (7.217 g, 18.1 mmol, 51.7% yield) as light yellow oil.

¹H NMR (400 MHz, CDCl₃) δ 7.37-7.27 (m, 5H), 4.57 (s, 2H), 4.32-4.23 (m, 1H), 4.07-4.02 (m, 1H), 3.75-3.70 (m, 1H), 3.69-3.61 (m, 16H), 3.60-3.54 (m, 1H), 3.52-3.46 (m, 1H), 1.42 (s, 3H), 1.35 (s, 3H).

Step (4)

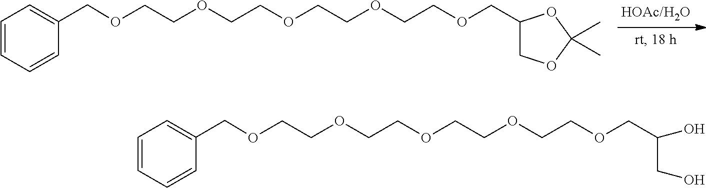 [SMILES: CC1(C)OCC(COCCOCCOCCOCCOCc2ccccc2)O1 ; OCC(O)COCCOCCOCCOCCOCc1ccccc1]

The mixture of 4-[2-[2-[2-(2-benzyloxyethoxy)ethoxy]ethoxy]ethoxymethyl]-2,2-dimethyl-1,3-dioxolane (7.217 g, 18.1 mmol) in AcOH (30 mL) and H₂O (30 mL) was stirred at room temperature for 18 hrs. TLC (EA/PE 1/1, SM R_{f}: 0.5; product, R_{f}: 0.1) indicated that all the starting materials was consumed. The solvent was removed under vacuum and azeotroped with toluene several times. 2-[2-[2-(2-methylsulfonyloxyethoxy)ethoxy]ethoxy]ethyl methanesulfonate (6.48 g, 18.1 mmol, quant.) as light yellow oil was obtained which was used without further purification.

¹H NMR (500 MHz, CDCl₃) δ 7.36-7.26 (m, 5H), 4.57 (s, 2H), 3.88-3.81 (m, 1H), 3.71-3.49 (m, 20H).

Step (5)

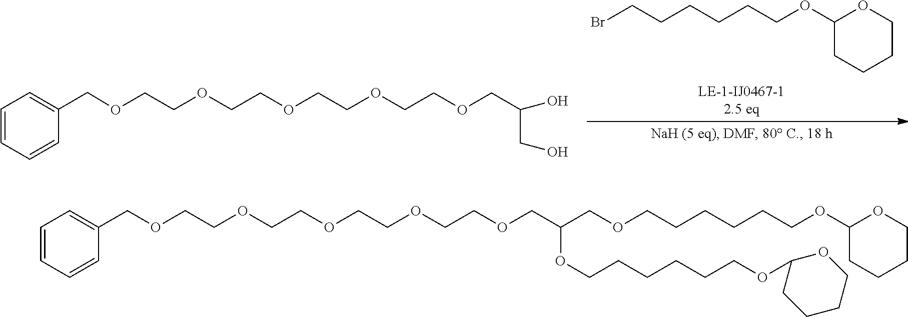 [SMILES: BrCCCCCCOC1CCCCO1 ; OCC(O)COCCOCCOCCOCCOCc1ccccc1 ; c1ccc(COCCOCCOCCOCCOCC(COCCCCCCOC2CCCCO2)OCCCCCCOC2CCCCO2)cc1]

To the solution of 3-[2-[2-[2-(2-benzyloxyethoxy)ethoxy]ethoxy]ethoxy]propane-1,2-diol (3.3 g, 9.21 mmol) in dry DMF (40 mL) was added NaH (1.84 g, 46 mmol) several times at 0° C. then the mixture was heated to 80° C. for 30 min. Then the reaction was cooled to room temperature and 2-(6-bromohexoxy)tetrahydropyran (6.1 g, 23 mmol) in dry DMF (20 mL) was added under nitrogen and the reaction was heated at 80° C. for 18 hrs. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 0 to 5% CH₃OH in DCM to give 2-[6-[1-[2-[2-[2-(2-benzyloxyethoxy)ethoxy]ethoxy]ethoxymethyl]-2-(6-tetrahydropyran-2-yloxyhexoxy)ethoxy]hexoxy]tetrahydropyran (2.834 g, 3.9 mmol, 42.3% yield) as colorless oil.

¹H NMR (500 MHz, CDCl₃) δ 7.35-7.27 (m, 5H), 4.57 (d, J=1.0 Hz, 4H), 3.90-3.83 (m, 2H), 3.78-3.30 (m, 31H), 1.75-1.65 (m, 3H), 1.63-1.49 (m, 17H), 1.41-1.34 (m, 8H).

Step (6)

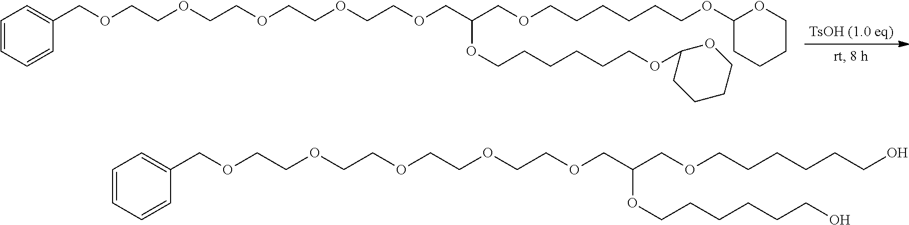 [SMILES: c1ccc(COCCOCCOCCOCCOCC(COCCCCCCOC2CCCCO2)OCCCCCCOC2CCCCO2)cc1 ; OCCCCCCOCC(COCCOCCOCCOCCOCc1ccccc1)OCCCCCCO]

To a solution of 2-[6-[1-[2-[2-[2-(2-benzyloxyethoxy)ethoxy]ethoxy]ethoxymethyl]-2-(6-tetrahydropyran-2-yloxyhexoxy)ethoxy]hexoxy]tetrahydropyran (2.834 g, 3.9 mmol) in EtOH (70 mL) was added p-Toluenesulfonic acid (0.742 g, 3.9 mmol) in one portion at room temperature and the mixture was stirred at room temperature for 24 h. TLC (4% CH₃OH in DCM) indicated that the starting material was disappeared completely. After the reaction was quenched with dilute sodium bicarbonate solution (150 mL), the solvent was extracted with EA (2*100 mL). The organic layers were washed with brine, dried over sodium sulfate, filtered and concentrated. The residue was purified by flash chromatography eluted with 0% to 5% CH₃OH in DCM (4%) to give 6-[3-[2-[2-[2-(2-benzyloxyethoxy)ethoxy]ethoxy]ethoxy]-2-(6-hydroxyhexoxy)propoxy]hexan-1-ol (1.435 g, 2.57 mmol, 65.9% yield) as colorless oil.

¹H NMR (500 MHz, CDCl₃) δ 7.36-7.27 (m, 5H), 4.57 (s, 2H), 3.70-3.39 (m, 29H), 1.67-1.53 (m, 9H), 1.40-1.35 (m, 7H).

Step (7)

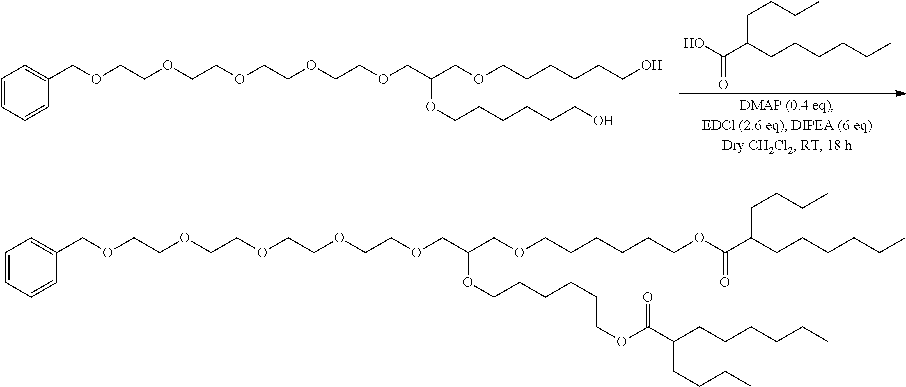 [SMILES: OCCCCCCOCC(COCCOCCOCCOCCOCc1ccccc1)OCCCCCCO ; CCCCCCC(CCCC)C(=O)O ; CCCCCCC(CCCC)C(=O)OCCCCCCOCC(COCCOCCOCCOCCOCc1ccccc1)OCCCCCCOC(=O)C(CCCC)CCCCCC]

To the solution of 6-[3-[2-[2-[2-(2-benzyloxyethoxy)ethoxy]ethoxy]ethoxy]-2-(6-hydroxyhexoxy)propoxy]hexan-1-ol (1.435 g, 2.57 mmol) and 2-butyloctanoic acid (1.54 g, 7.7 mmol) in dry dichloromethane (30 mL) were added DIPEA (1.99 g, 15.4 mmol), DMAP (0.126 g, 1.03 mmol) and under ice bath added EDCI (1.28 g, 6.68 mmol). The mixture was stirred at room temperature for 18 hrs. The reaction was quenched with NaHCO₃ (30 mL) and washed with brine. The organic layer was dried over sodium sulfate, filtered and concentrated. The residue was purified by flash chromatography eluted with 0% to 5% (3%)CH₃OH in DCM to give 6-[3-[2-[2-[2-(2-benzyloxyethoxy)ethoxy]ethoxy]ethoxy]-2-[6-(2-butyloctanoyloxy)hexoxy]propoxy]hexyl 2-butyloctanoate (1.816 g (P: 1.15 g+NP: 0.666 g), 1.97 mmol, 76.6% yield) as colorless oil.

¹H NMR (500 MHz, CDCl₃) δ 7.36-7.27 (m, 5H), 4.57 (s, 2H), 4.10-4.01 (m, 4H), 3.72-3.61 (m, 16H), 3.60-3.40 (m, 9H), 2.35-2.26 (m, 2H), 1.66-1.52 (m, 12H), 1.47-1.20 (m, 36H), 0.92-0.83 (m, 12H).

Step (8)

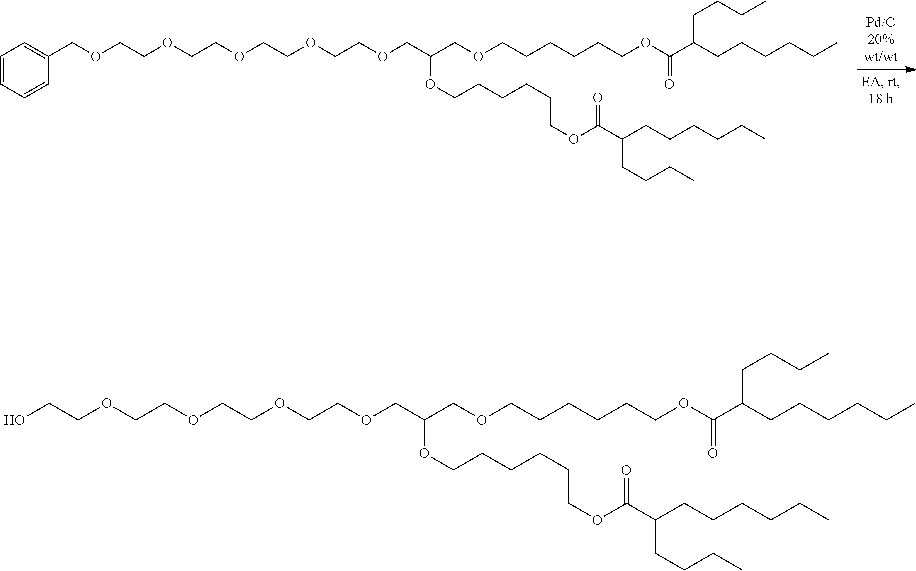 [SMILES: CCCCCCC(CCCC)C(=O)OCCCCCCOCC(COCCOCCOCCOCCOCc1ccccc1)OCCCCCCOC(=O)C(CCCC)CCCCCC ; CCCCCCC(CCCC)C(=O)OCCCCCCOCC(COCCOCCOCCOCCO)OCCCCCCOC(=O)C(CCCC)CCCCCC]

A solution of 6-[3-[2-[2-[2-(2-benzyloxyethoxy)ethoxy]ethoxy]ethoxy]-2-[6-(2-butyloctanoyloxy)hexoxy]propoxy]hexyl 2-butyloctanoate (1.15 g, 1.25 mmol) in EtOAc (20 mL) was purged for 10 minutes with N₂ followed by addition of Pd/C (230 mg) and the reaction continued purging with N₂. The reaction was next evacuated under vacuum and backfilled with H₂ 3 times. The reaction was next stirred overnight at room temperature under an atmosphere of H₂. TLC (5% CH3OH in DCM) shows that the reaction was finished. The slurry filtered through celite and the celite was rinsed with EtOAc several times. The combined organics were next concentrated in vacuo to give 6-[2-[6-(2-butyloctanoyloxy)hexoxy]-3-[2-[2-[2-(2-hydroxyethoxy)ethoxy]ethoxy]ethoxy]propoxy]hexyl 2-butyloctano-ate (1.0 g, 1.2 mmol, 96.4% yield) as colorless liquid.

¹H NMR (500 MHz, CDCl₃) δ 4.09-4.03 (m, 4H), 3.76-3.39 (m, 25H), 2.82 (s, 1H), 2.35-2.26 (m, 2H), 1.67-1.52 (m, 12H), 1.47-1.21 (m, 36H), 0.92-0.83 (m, 12H).

Step (9)

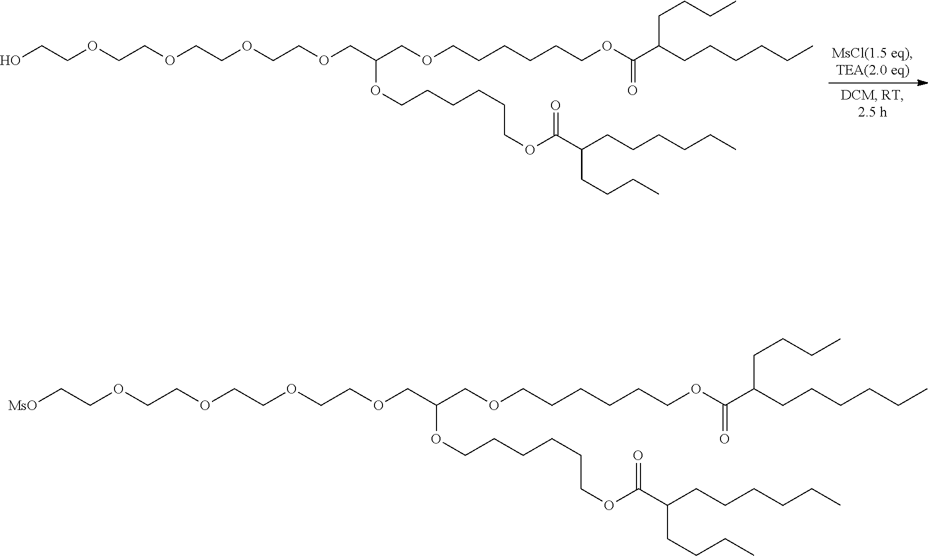 [SMILES: CCCCCCC(CCCC)C(=O)OCCCCCCOCC(COCCOCCOCCOCCO)OCCCCCCOC(=O)C(CCCC)CCCCCC ; CCCCCCC(CCCC)C(=O)OCCCCCCOCC(COCCOCCOCCOCCOS(C)(=O)=O)OCCCCCCOC(=O)C(CCCC)CCCCCC]

To a solution of 6-[2-[6-(2-butyloctanoyloxy)hexoxy]-3-[2-[2-[2-(2-hydroxyethoxy)ethoxy]ethoxy]ethoxy]propoxy]hexyl 2-butyloctanoate (0.6 g, 0.72 mmol) and TEA (triethylamine) (0.146 g, 1.44 mmol) in 10 mL Dichloromethane (DCM), was added Ms-Cl (0.124 g, 1.08 mmol). The mixture was stirred at room temperature for 3 h. TLC (CH₃OH/DCM 3%) indicated that the starting material was consumed. The reaction was diluted with dichloromethane and washed with water (10 mL). The organic layer was dried over sodium sulfate, filtered and concentrated to give nonyl to afford 6-[2-[6-(2-butyloctanoyloxy)hexoxy]-3-[2-[2-[2-(2-hydroxyethoxy)ethoxy]ethoxy]ethoxy]propoxy]hexyl 2-butyloctanoate (0.582 g, 0.639 mmol, 88.7% yield) as pale yellow liquid, which was used directly for next step.

¹H NMR (500 MHz, CDCl₃) δ 4.41-4.36 (m, 2H), 4.08-4.02 (m, 4H), 3.78-3.74 (m, 2H), 3.71-3.39 (m, 21H), 3.08 (s, 3H), 2.35-2.25 (m, 2H), 1.64-1.55 (m, 12H), 1.47-1.20 (m, 36H), 0.91-0.84 (m, 12H).

Step (10)

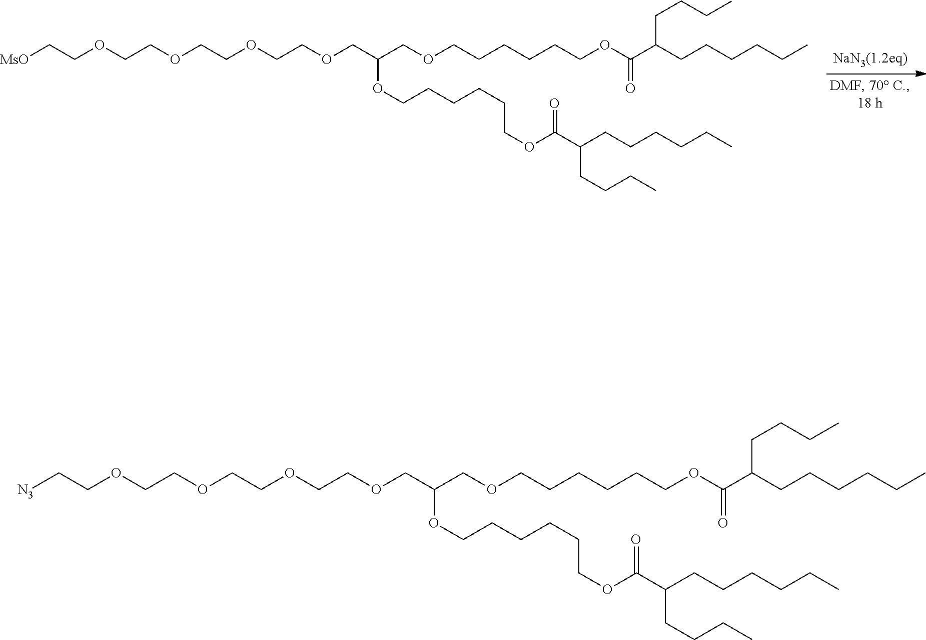 [SMILES: CCCCCCC(CCCC)C(=O)OCCCCCCOCC(COCCOCCOCCOCCOS(C)(=O)=O)OCCCCCCOC(=O)C(CCCC)CCCCCC ; CCCCCCC(CCCC)C(=O)OCCCCCCOCC(COCCOCCOCCOCCN=[N+]=[N-])OCCCCCCOC(=O)C(CCCC)CCCCCC]

To a solution of 6-[2-[6-(2-butyloctanoyloxy)hexoxy]-3-[12-[2-[2-(2-methylsulfonyloxyethoxy)ethoxy]ethoxy]ethoxy]propoxy]hexyl 2-butyloctanoate (0.582 g, 0.639 mmol) dissolved in DMF (10 mL) then added Na₃N (50 m g, 0.766 mmol). Then the reaction mixture was stirred at 70° C. for 18 hr. TCL showed that the starting material was disappeared and a new spot was observed. Then water (100 mL) was added and the reaction mixture was extracted with ethyl acetate (100 mL*2). The combined organic phases were washed with brine (100 mL), dried over Na₂SO₄, filtered and concentrated under reduced pressure to give a residue, which was purified by column chromatography eluted with CH₃OH in DCM (0-10%)(4%) to give 6-[3-[2-[2-[2-(2-azidoethoxy)ethoxy]ethoxy]ethoxy]-2-[6-(2-butyloctanoyloxy)hexoxy]propoxy]hexyl 2-butyloctanoate (0.409 g, 0.477 mmol, 74.6% yield) as yellow oil.

¹H NMR (500 MHz, CDCl₃) δ 4.09-4.03 (m, 4H), 3.70-3.62 (m, 14H), 3.60-3.37 (m, 11H), 2.34-2.26 (m, 2H), 1.63-1.53 (m, 12H), 1.46-1.21 (m, 36H), 0.91-0.84 (m, 12H).

Step (11)

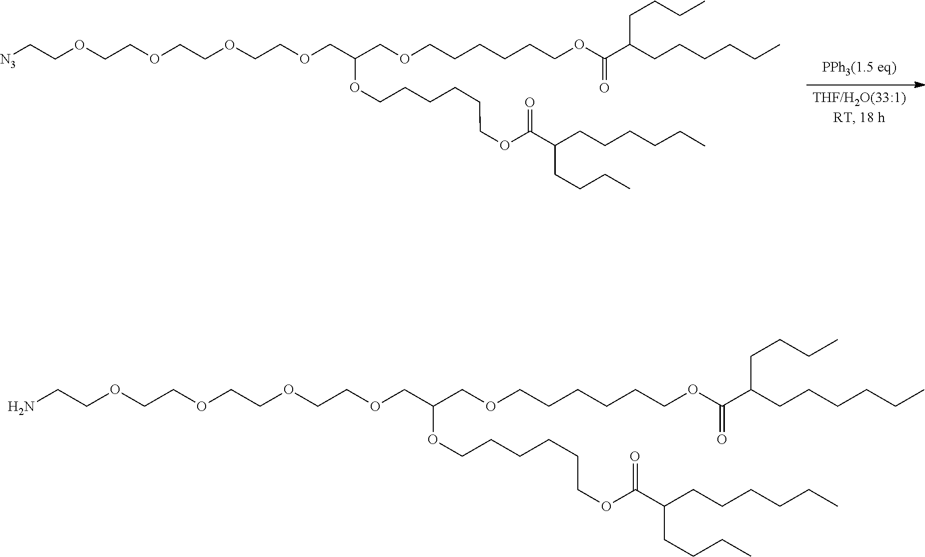 [SMILES: CCCCCCC(CCCC)C(=O)OCCCCCCOCC(COCCOCCOCCOCCN=[N+]=[N-])OCCCCCCOC(=O)C(CCCC)CCCCCC ; CCCCCCC(CCCC)C(=O)OCCCCCCOCC(COCCOCCOCCOCCN)OCCCCCCOC(=O)C(CCCC)CCCCCC]

A mixture of 6-[3-[2-[2-[2-(2-azidoethoxy)ethoxy]ethoxy]ethoxy]-2-[6-(2-butyloctanoyloxy)hexoxy]propoxy]hexyl 2-butyloctanoate (0.409 g, 0.477 mmol) and triphenylphosphine (0.187 g, 0.715 mmol) in THF (10 mL)/Water (0.3 mL) was stirred at 20° C. for 16 hrs. TLC (ninhydrin, 3% methanol in dichloromethane) indicated the reaction was complete. The solvent was removed and added into DCM then concentrated under reduced pressure to give a residue, which was purified by column chromatography eluted with CH₃OH in CH₂Cl₂ (0-20%(14%)) to give 6-[3-[2-[2-[2-(2-aminoethoxy)ethoxy]ethoxy]ethoxy]-2-[6-(2-butyloctanoyloxy)hexoxy]propoxy]hexyl 2-butyloctanoate (0.300 g, 0.237 mmol, 75.6% yield) as light yellow oil.

¹H NMR (500 MHz, CDCl₃) δ 4.09-4.03 (m, 4H), 3.70-3.41 (m, 23H), 2.91 (t, J=5.1 Hz, 2H), 2.46 (s, 2H), 2.34-2.27 (m, 2H), 1.68-1.51 (m, 12H), 1.46-1.21 (m, 36H), 0.92-0.84 (m, 12H).

Step (12)

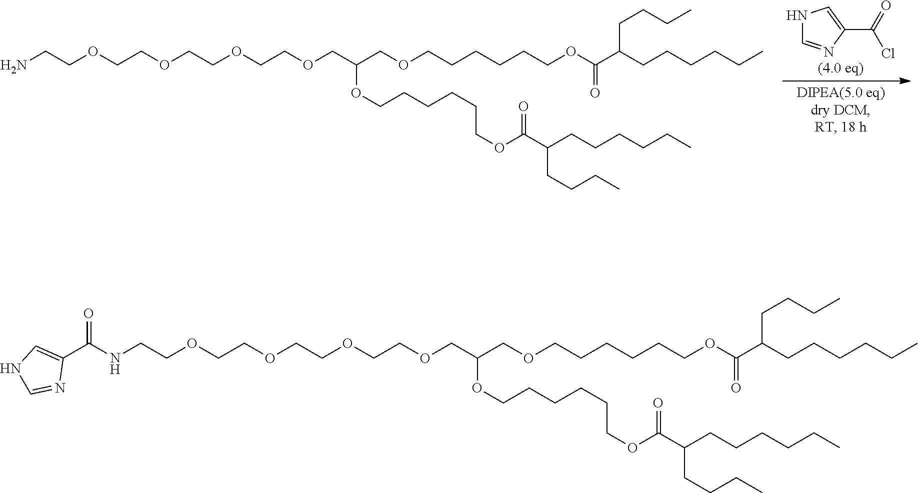 [SMILES: CCCCCCC(CCCC)C(=O)OCCCCCCOCC(COCCOCCOCCOCCN)OCCCCCCOC(=O)C(CCCC)CCCCCC ; O=C(Cl)c1c[nH]cn1 ; CCCCCCC(CCCC)C(=O)OCCCCCCOCC(COCCOCCOCCOCCNC(=O)c1c[nH]cn1)OCCCCCCOC(=O)C(CCCC)CCCCCC]

6-[3-[2-[2-[2-(2-aminoethoxy)ethoxy]ethoxy]ethoxy]-2-[6-(2-butyloctanoyloxy) hexoxy]propoxy]hexyl 2-butyloctanoate (300 mg, 0.36 mmol) was dissolved in 10 mL anhydrous DCM and the 1H-imidazole-4-carbonyl chloride (188 mg, 1.44 mmol) in 1 mL anhydrous DMF and DIPEA (233 mg, 1.80 mmol) were added. The mixture was stirred at room temperature overnight. The solvent was evaporated and the product purified by flash chromatography (25 g column, DCM/MeOH 0% to 5%) eluted with methanol in dichloromethane (4%) to give 6-[2-[6-(2-butyloctanoyloxy)hexoxy]-3-[2-[2-[2-[2-(1H-imidazole-4-carbonylamino)ethoxy]ethoxy]ethoxy]ethoxy]propoxy]hexyl 2-butyloctanoate (259 mg, 0.266 mmol, 73.7% yield) as colorless oil.

LCMS: EXP-21-IX3021-29498-LCMSA020 Find peak: MS(ESI) m/z=926.8/927.8 (M+H)+ at 2.854 min ¹H NMR (500 MHz, CDCl₃) δ 7.77-7.56 (m, 3H), 4.09-4.02 (m, 4H), 3.69-3.39 (m, 25H), 2.34-2.27 (m, 2H), 1.65-1.53 (m, 12H), 1.46-1.22 (m, 36H), 0.91-0.84 (m, 12H).

### Example 17: Synthesis of N-[2-[2-[2-[2-[1-[1,2-bis[(Z)-octadec-9-enoxy]ethyl]tridecoxy]ethoxy]ethoxy]ethoxy]ethyl]-1Himidazole-4-carboxamide (Compound XVIII)

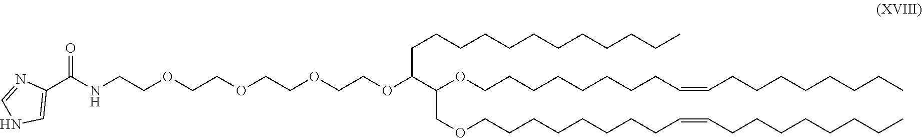 [SMILES: CCCCCCCC/C=C\CCCCCCCCOCC(OCCCCCCCC/C=C\CCCCCCCC)C(CCCCCCCCCCCC)OCCOCCOCCOCCNC(=O)c1c[nH]cn1]

Synthesis of Compound (XVIII)

Step (1)

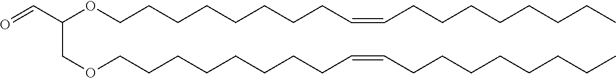 [SMILES: CCCCCCCC/C=C\CCCCCCCCOCC(C=O)OCCCCCCCC/C=C\CCCCCCCC]

To a solution of 2,3-bis[(Z)-octadec-9-enoxy]propan-1-ol (5 g, 8.43 mmol) in DCM (40 ml) was added Dess-Martin Periodinane (5.05 g, 10.1 mmol) at 0° C. for 5 min. Then the mixture was stirred at 25° C. for 2 hrs under N₂. After reaction, the mixture was dealt DCM (40 ml), washed with NaHCO₃/Na2S203 (1/1) (50 ml×3), NaCl sat.aq (50 ml), dried over Na2SO4. The organic was concentrated and purified by flash (10% EA in PE), to give 2,3-bis[(Z)-octadec-9-enoxy]propanal (3.04 g, 5.04 mmol, yield 59.8%) as a colorless oil.

¹H NMR (500 MHz, CDCl₃) δ 9.73 (d, J=1.3 Hz, 1H), 5.41-5.31 (m, 4H), 3.85-3.77 (m, 1H), 3.75-3.64 (m, 2H), 3.58 (tt, J=9.3, 4.6 Hz, 2H), 3.49-3.40 (m, 2H), 2.21-1.91 (m, 8H), 1.64 (dd, J=14.1, 7.0 Hz, 2H), 1.57-1.48 (m, 2H), 1.26 (d, J=4.4 Hz, 44H), 0.88 (dd, J=8.7, 5.0 Hz, 6H).

Step (2)

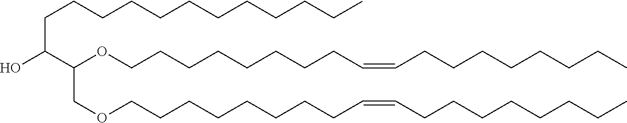 [SMILES: CCCCCCCC/C=C\CCCCCCCCOCC(OCCCCCCCC/C=C\CCCCCCCC)C(O)CCCCCCCCCCCC]

To a solution of Mg (3.75 g) and 12 (1.31 g) in anhydrous THF (5 ml) was added 1-bromododecane (2.56 g, 10.28 mmol). The mixture was stirred at 70° C. under N2 until the mixture as the colorless one. 1-bromododecane (10.24 g, 41.12 mmol) was added to the reaction. The mixture was stirred at 70° C. for 3 hr. Then the mixture was added to the solution of 2,3-bis[(Z)-octadec-9-enoxy]propanal (3.04 g, 5.14 mmol) in anhydrous THF (45 ml). The mixture was stirred at 7° C. for 14 hr. The mixture was dealt with EA (150 ml), washed with water (150 ml×2), NaCl sat.aq (150 ml). The organic was purified by flash (5% EA in PE), to give 1,2-bis[(Z)-octadec-9-enoxy]pentadecan-3-ol (3.97 g, 5.21 mmol, yield 100%) as a yellow oil.

¹H NMR (500 MHz, CDCl₃) δ 5.40-5.31 (m, 3H), 3.76-3.36 (m, 7H), 3.31-3.23 (m, 1H), 2.03-1.94 (m, 6H), 1.64-1.57 (m, 2H), 1.54-1.50 (m, 2H), 1.27 (d, J=12.1 Hz, 66H), 0.87 (d, J=6.7 Hz, 9H).

Step (3)

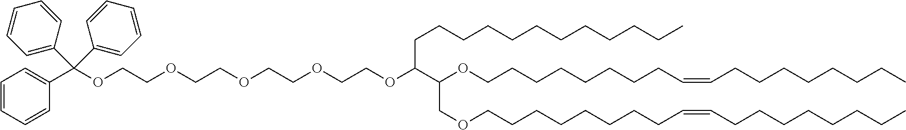 [SMILES: CCCCCCCC/C=C\CCCCCCCCOCC(OCCCCCCCC/C=C\CCCCCCCC)C(CCCCCCCCCCCC)OCCOCCOCCOCCOC(c1ccccc1)(c1ccccc1)c1ccccc1]

To a solution of 1,2-bis[(Z)-octadec-9-enoxy]pentadecan-3-ol (1 g, 1.31 mmol) in THF (20 ml) was added NaH (210 mg, 5.25 mmol). The mixture was stirred at 70° C. for 1 hr. 2-[2-[2-(2-trityloxyethoxy)ethoxy]ethoxy]ethyl methanesulfonate (1.01 g, 1.97 mmol) was added to this mixture and the mixture was stirred at 70° C. for 18 hr. The mixture was dealt with EA (150 ml), washed with water (150 ml×2), NaCl sat.aq (150 ml) and dried over Na2SO4. The organic was concentrated and purified by flash (10% EA in PE), to give [2-[2-[2-[2-[l-[1,2-bis[(Z)-octadec-9-enoxy]ethyl]tridecoxy]ethoxy]ethoxy]ethoxy]ethoxy-diphenyl-methyl]benzene (1.08 g, 0.9 mmol, yield 68.3%) as a colorless oil.

¹H NMR (500 MHz, CDCl₃) δ 7.48-7.44 (m, 6H), 7.28 (t, J=7.6 Hz, 6H), 7.22 (t, J=7.3 Hz, 3H), 5.39-5.31 (m, 3H), 3.73-3.54 (m, 16H), 3.50-3.32 (m, 6H), 3.23 (t, J=5.3 Hz, 2H), 2.04-1.93 (m, 7H), 1.53 (s, 4H), 1.37-1.18 (m, 66H), 0.89-0.85 (m, 9H). EXP-21-TV4361-N2 (571 mg, yield 24%)

Step (4)

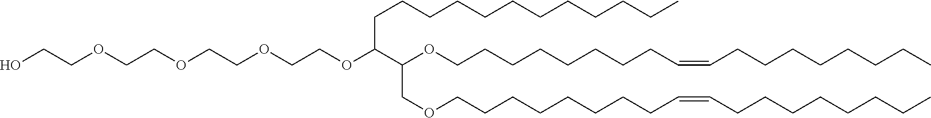 [SMILES: CCCCCCCC/C=C\CCCCCCCCOCC(OCCCCCCCC/C=C\CCCCCCCC)C(CCCCCCCCCCCC)OCCOCCOCCOCCO]

To a solution of [2-[2-[2-[2-[l-[1,2-bis[(Z)-octadec-9-enoxy]ethyl]tridecoxy]ethoxy]ethoxy]ethoxy]ethoxy-diphenylmethyl]benzene (1.65 g, 1.4 mmol) in THF/MeOH (20 ml 1/1) was added Toluene-4-sulfonic acid (1.33 g, 6.99 mmol). The mixture was stirred at 25° C. for 18 hr. The mixture was concentrated and dealt with EA (150 ml), washed with NaHCO₃ sat.aq (150 ml×2), NaCl sat.aq (150 ml) and dried over Na₂SO₄. The organic was concentrated and purified by flash (50% EA in PE), to give 2-[2-[2-[2-[1-[1,2-bis[(Z)-octadec-9-enoxy]ethyl]tridecoxy]ethoxy]ethoxy]ethoxy]ethanol (1.18 g, 1.23 mmol, yield 87.8%) as a colourless oil.

¹H NMR (500 MHz, CDCl₃) δ 5.41-5.31 (m, 3H), 3.74-3.54 (m, 18H), 3.50-3.33 (m, 6H), 2.59 (dd, J=9.7, 6.0 Hz, 1H), 2.04-1.93 (m, 7H), 1.57-1.22 (m, 70H), 0.88 (t, J=6.9 Hz, 9H).

Step (5)

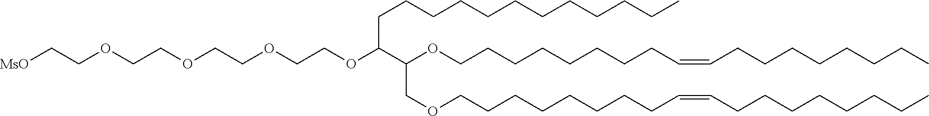 [SMILES: CCCCCCCC/C=C\CCCCCCCCOCC(OCCCCCCCC/C=C\CCCCCCCC)C(CCCCCCCCCCCC)OCCOCCOCCOCCOS(C)(=O)=O]

To a solution of 2-[2-[2-[2-[1-[1,2-bis[(Z)-octadec-9-enoxy]ethyl]tridecoxy]ethoxy]ethoxy]ethoxy]ethanol (1.1 g, 1.17 mmol) in DCM (15 ml) was added TEA (297 mg, 2.93 mmol) and Methanesulfonyl chloride (269 mg, 2.35 mmol) at 0° C. for 5 min. The mixture was stirred at 25° C. for 18 hr. Then the mixture was dealt with DCM (50 ml), washed with water (50 ml×2), 1N HCl (50 ml), NaHCO₃ sat.aq (50 ml) and dried over Na2SO4. The organic was concentrated to give 2-[2-[2-[2-[1-[1,2-bis[(Z)-octadec-9-enoxy]ethyl]tridecoxy]ethoxy]ethoxy]ethoxy]ethyl methanesulfonate (860 mg, 0.8 mmol, yield 70.7%) as a yellow oil.

¹H NMR (500 MHz, CDCl₃) δ 5.39-5.31 (m, 3H), 4.40-4.36 (m, 2H), 3.77-3.56 (m, 16H), 3.49-3.34 (m, 6H), 3.07 (s, 3H), 2.06-1.91 (m, 7H), 1.56-1.51 (m, 4H), 1.35-1.21 (m, 66H), 0.88 (t, J=6.9 Hz, 9H).

Step (6)

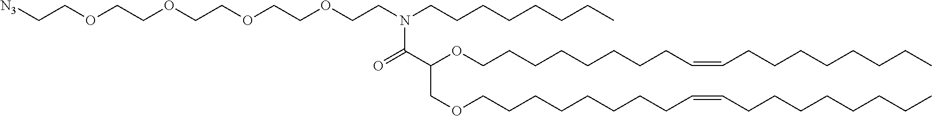 [SMILES: CCCCCCCC/C=C\CCCCCCCCOCC(OCCCCCCCC/C=C\CCCCCCCC)C(=O)N(CCCCCCCC)CCOCCOCCOCCOCCN=[N+]=[N-]]

To a solution of 2-[2-[2-[2-[2-[2,3-bis[(Z)-octadec-9-enoxy]propanoyl-octylamino]ethoxy]ethoxy]ethoxy]ethoxy]ethyl methanesulfonate (860 mg, 0.85 mmol) in DMF (10 ml) was added NaN3 (165 mg, 2.54 mmol). The mixture was stirred at 70° C. for 14 hr. The mixture was dealt with EA (50 ml), washed with water (50 ml), NaCl sat.aq (50 ml) and dried over Na2SO4. The organic was concentrated and purified by flash (50% EA in PE), to give N-[2-[2-[2-[2-(2-azidoethoxy)ethoxy]ethoxy]ethoxy]ethyl]-2,3-bis[(Z)-octadec-9-enoxy]-N-octyl-propanamide (657 mg, 0.65 mmol, yield 77.4%%) as a colorless oil.

¹H NMR (500 MHz, CDCl₃) δ 5.40-5.30 (m, 3H), 3.78-3.28 (m, 24H), 2.04-1.92 (m, 7H), 1.56-1.20 (m, 70H), 0.88 (t, J=6.9 Hz, 9H).

Step (7)

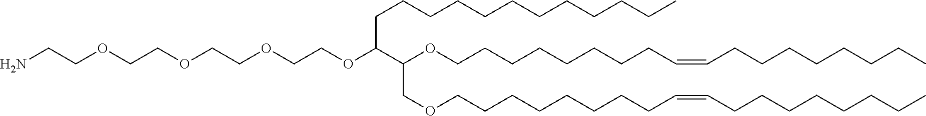 [SMILES: CCCCCCCC/C=C\CCCCCCCCOCC(OCCCCCCCC/C=C\CCCCCCCC)C(CCCCCCCCCCCC)OCCOCCOCCOCCN]

To a solution of (Z)-1-[3-[2-[2-[2-(2-azidoethoxy)ethoxy]ethoxy]ethoxy]-2-[(Z)-octadec-9-enoxy]pentadecoxy]octadec-9-ene (657 mg, 0.68 mmol) in THF/water (20 ml/0.6 ml) was added Triphenyl phosphine (269 mg, 1 mmol). The mixture was stirred at 25° C. for 18 hrs. The mixture was concentrated and purified by flash (10%-20% MeOH in DCM), to give 2-[2-[2-[2-[1-[1,2-bis[(Z)-octadec-9-enoxy]ethyl]tridecoxy]ethoxy]ethoxy]ethoxy]ethanamine (560 mg, 0.59 mmol, yield 85.8%) as a colorless oil.

¹H NMR (500 MHz, CDCl₃) δ 5.41-5.31 (m, 3H), 3.77-3.33 (m, 22H), 2.89 (dt, J=12.6, 5.0 Hz, 2H), 2.06-1.90 (m, 7H), 1.57-1.51 (m, 4H), 1.48-1.15 (m, 66H), 0.88 (t, J=6.9 Hz, 9H).

Step (8)

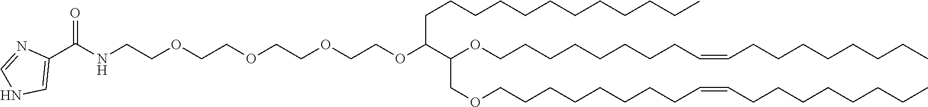 [SMILES: CCCCCCCC/C=C\CCCCCCCCOCC(OCCCCCCCC/C=C\CCCCCCCC)C(CCCCCCCCCCCC)OCCOCCOCCOCCNC(=O)c1c[nH]cn1]

To a solution of 2-[2-[2-[2-[l-[1,2-bis[(Z)-octadec-9-enoxy]ethyl]tridecoxy]ethoxy]ethoxy]ethoxy]ethanamine (560 mg, 0.6 mmol) in DCM (20 ml) was added DIEA (386 mg, 3 mmol) and 1H-imidazole-4-carbonyl chloride (312 mg, 2.39 mmol). The mixture was stirred at 25° C. for 18 hr. The mixture was dealt with DCM (50 ml), washed with water (50 ml), brine (50 ml×2) and dried over Na₂SO₄. The organic was concentrated and purified by flash (10% MeOH in DCM), to give N-[2-[2-[2-[2-[l-[1,2-bis[(Z)-octadec-9-enoxy]ethyl]tridecoxy]ethoxy]ethoxy]ethoxy]ethyl]-1H-imidazole-4-carboxamide (348 mg, 0.32 mmol) as a colorless oil.

¹H NMR (500 MHz, CDCl₃) δ 7.65 (d, J=8.7 Hz, 1H), 7.61 (d, J=11.0 Hz, 1H), 7.54 (s, 1H), 5.40-5.31 (m, 3H), 3.80-3.30 (m, 24H), 2.06-1.93 (m, 7H), 1.60-1.17 (m, 70H), 0.88 (t, J=6.9 Hz, 9H).

### Example 18: Synthesis of bis(2-butyloctyl) 10-[2-[2-[2-(1H-imidazole-4-carbonylamino)ethoxy]ethoxy]ethyl-nonyl-amino]nonadecanedioate; hydrochloride (Compound XIX)

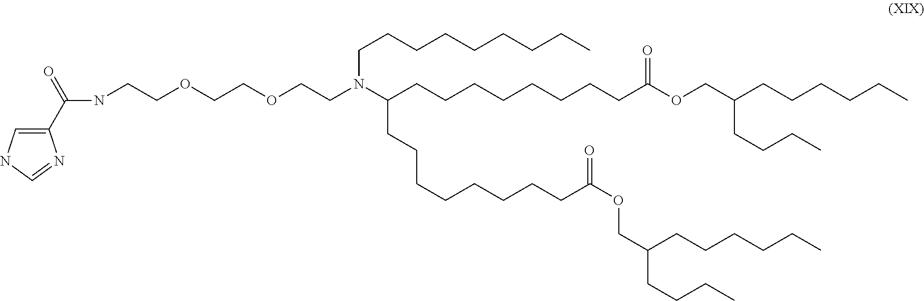 [SMILES: CCCCCCCCCN(CCOCCOCCNC(=O)c1c[nH]cn1)C(CCCCCCCCC(=O)OCC(CCCC)CCCCCC)CCCCCCCCC(=O)OCC(CCCC)CCCCCC]

The synthesis of compound (XIX) was carried according to the following scheme (10).

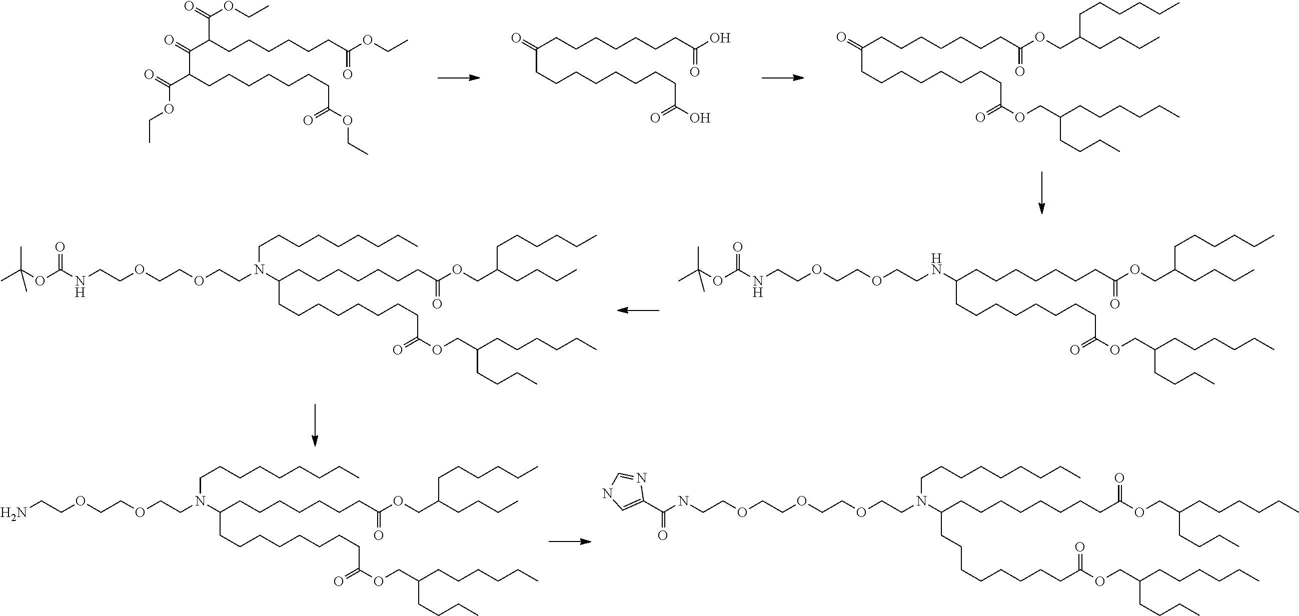

Synthesis of Compound (XIX)

Step (1)

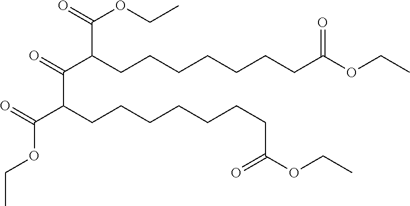 [SMILES: CCOC(=O)CCCCCCCC(C(=O)OCC)C(=O)C(CCCCCCCC(=O)OCC)C(=O)OCC]

A mixture of diethyl 3-oxopentanedioate (20 g) and a 20% sodium ethoxide-ethanol solution (33.5 g) was stirred at 80° C. for 20 minutes, ethyl 8-bromooctanoate (25 g) was then added thereto, and the mixture was stirred for 4 hours. A 20% sodium ethoxide-ethanol solution (33.5 g) was added to the reaction mixture, the reaction mixture was stirred for 5 minutes, ethyl 8-bromooctanoate (25 g) was then added thereto, and the mixture was stirred for 3 hours. The reaction mixture was cooled to room temperature, hexane and a 20% aqueous ammonium chloride solution (110 mL) were then added thereto, the organic layer was separated, and the solvent was distilled away under reduced pressure, thereby obtaining tetraethyl 9-oxoheptadecane-1,8,10,17-tetracarboxylate (51.5 g) as a crude product.

LCMS Rt=2.194

Step (2)

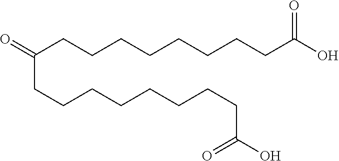 [SMILES: O=C(O)CCCCCCCCC(=O)CCCCCCCCC(=O)O]

A mixture of the obtained tetraethyl 9-oxoheptadecane-1,8,10,17-tetracarboxylate (25 g), acetic acid (40 mL), and a 30% aqueous hydrochloric acid solution (80 mL) was stirred at 115° C. for 6 hours. The reaction mixture was cooled to room temperature, the solvent was then distilled away under reduced pressure, and water and acetone were added to the residue. Solids were collected by filtration, washed with water and acetone, and then dried under reduced pressure, thereby obtaining 10-oxononane decanedioic acid (0.6 g) as white solids.

¹H NMR (400 MHz, DMSO) δ 11.97 (s, 2H), 2.38 (t, J=7.3 Hz, 4H), 2.18 (t, J=7.4 Hz, 4H), 1.54-1.35 (m, 8H), 1.23 (s, 16H).

Step (3)

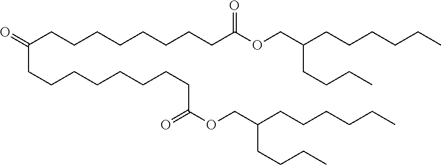 [SMILES: CCCCCCC(CCCC)COC(=O)CCCCCCCCC(=O)CCCCCCCCC(=O)OCC(CCCC)CCCCCC]

1-Ethyl-3-(3-dimethylaminopropyl)carbodiimide hydrochloride (8.53 g) was added to a mixture of 10-oxononane decanedioic acid (6.10 g), 2-butyloctan-1-ol (6.63 g), triethylamine (12.5 mL), 4-dimethylaminopyridine (2.17 g), and dichloromethane (60 mL), and the mixture was stirred at room temperature for 2 days. A 10% aqueous potassium hydrogen sulfate solution (120 mL), hexane (60 mL), and ethyl acetate (60 mL) were added to the reaction mixture, the organic layer was separated and then dried over anhydrous sodium sulfate, and the solvent was distilled away under reduced pressure. The obtained residue was purified by silica gel column chromatography (ethyl acetate-hexane), thereby obtaining bis(2-butyloctyl)IO-oxononane decanedioate (6 mg) as a colorless oily substance

¹H NMR (400 MHz, CDCl₃) δ 3.97 (d, J=5.8 Hz, 4H), 2.37 (t, J=7.5 Hz, 4H), 2.29 (t, J=7.5 Hz, 4H), 1.71-1.43 (m, 11H), 1.28 (d, J=1.2 Hz, 49H), 0.89 (tt, J=6.6, 4.1 Hz, 12H).

Step (4)

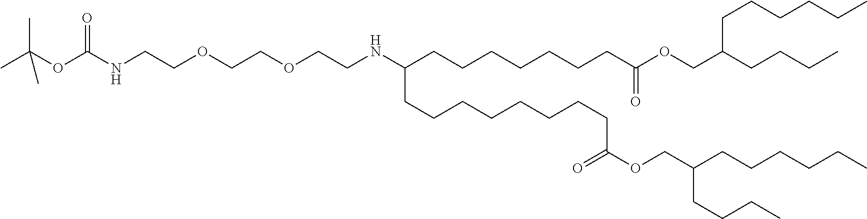 [SMILES: CCCCCCC(CCCC)COC(=O)CCCCCCCCC(CCCCCCCC(=O)OCC(CCCC)CCCCCC)NCCOCCOCCNC(=O)OC(C)(C)C]

A mixture of bis(2-butyloctyl)IO-oxononane decanedioate (2.3 g) and Boc-1-amino-3,6-8-octanediaminedioxa diamine (1.27 g) was stirred in dichloromethane at room temperature for 15 min. Then sodium triacetoborohydride (0.76 g) and acetic acid (0.21 ml) were added. The reaction mixture was stirred 5 h at room temperature. After dilution with dichloromethane (25 mL), the reaction mixture was washed with saturated sodium bicarbonate (NaHCO₃). The organic layer was washed with water and brine, dried over Na₂SO₄. After removal of the solvent, the residue was purified by flash chromatography (DCM/MeOH/TEA, 85/15/1, (v,v,v)) thereby obtaining bis(2-butyloctyl)-10-[2-[2-[2-(ter-butoxycarbonylamino]ethoxy]ethylamino]nonadecanedioate as a colorless oily substance.

Step (5)

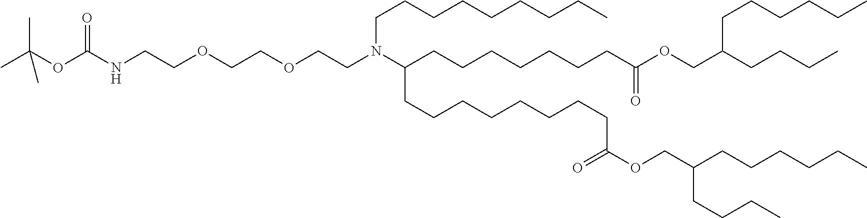 [SMILES: CCCCCCCCCN(CCOCCOCCNC(=O)OC(C)(C)C)C(CCCCCCCCC(=O)OCC(CCCC)CCCCCC)CCCCCCCC(=O)OCC(CCCC)CCCCCC]

A mixture of bis(2-butyloctyl)-10-[2-[2-[2-(ter-butoxycarbonylamino]ethoxy]ethylamino]nonadecanedioate (0.5 g) and nonanal (0.18 g) was stirred in dichloromethane at room temperature for 15 min. Then sodium triacetoborohydride (0.174 g) and acetic acid (0.05 ml) were added. The reaction mixture was stirred 8 h at room temperature. After dilution with dichloromethane (20 mL), the reaction mixture was washed with saturated sodium bicarbonate (NaHCO₃). The organic layer was washed with brine, dried over Na₂SO₄. After removal of the solvent, the residue was purified by flash chromatography (Heptane/AcOEt (a gradient 0 to 50%)) thereby obtaining bis(2-butyloctyl) 10-[2-[2-[2-(tert-butoxycarbonylamino)ethoxy]ethoxy]ethyl-nonyl-amino]nonadecanedioate as a colorless oily substance.

¹H NMR (400 MHz, DMSO-d6) δ ppm 0.83-0.88 (m, 15H), 1.14-1.43 (m, 82H), 1.46-1.59 (m, 6H), 2.23-2.29 (m, 4H), 2.34-2.36 (m, 2H), 3.01-3.07 (m, 2H), 3.30-3.34 (m, 4H), 3.46 (s, 4H), 3.91 (dd, J=6, 2 Hz, 4H), 6.47-6.75 (m, 1H), pseudo-molecular ion m/z=1038, retention time (min)=2.37

Step (6)

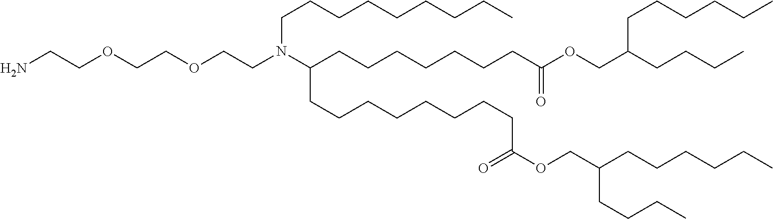 [SMILES: CCCCCCCCCN(CCOCCOCCN)C(CCCCCCCCC(=O)OCC(CCCC)CCCCCC)CCCCCCCC(=O)OCC(CCCC)CCCCCC]

A bis(2-butyloctyl) 10-[2-[2-[2-(tert-butoxycarbonylamino)ethoxy]ethoxy]ethyl-nonyl-amino]nonadecanedioate (0.080 g) was diluted in dichloromethane (5 mL). Then a solution of chlorohydric acid (4M in dioxane, 5 equiv.) was added. The mixture was stirred at room temperature for 2 hours. After removal of the solvent, the residue was stirred with isopropyl ether (3 mL) and filtered and dried thereby obtaining bis(2-butyloctyl) 10-[2-[2-(2-aminoethoxy)ethoxy]ethyl-nonyl-amino]nonadecanedioate; hydrochloride as a hygroscopic white solid.

¹H NMR (400 MHz, DMSO-d6) δ ppm 0.79-0.92 (m, 15H), 1.14-1.59 (m, 72H), 1.62-1.88 (m, 3H), 2.27 (t, J=7 Hz, 5H), 2.90-3.01 (m, 2H), 3.01-3.12 (m, 2H), 3.13-3.29 (m, 3H), 3.54-3.66 (m, 6H), 3.77-3.87 (m, 2H), 3.92 (d, J=6 Hz, 4H), 7.89-8.21 (m, 3H), 9.51 (br s, 1H). pseudo-molecular ion m/z=937, retention time (min)=2.17

Step (7)

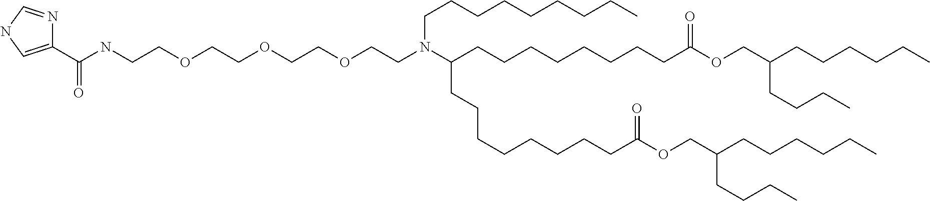 [SMILES: CCCCCCCCCN(CCOCCOCCOCCNC(=O)c1c[nH]cn1)C(CCCCCCCCC(=O)OCC(CCCC)CCCCCC)CCCCCCCCC(=O)OCC(CCCC)CCCCCC]

A mixture of bis(2-butyloctyl) 10-[2-[2-[2-(1H-imidazole-4-carbonylamino)ethoxy]ethoxy]ethyl-nonyl-amino]nonadecanedioate; hydrochloride (0.011 g), 1H-imidazole-4-carbonyl chloride (0.023 g) in solution with dichloromethane (3 mL) was stirred at room temperature. Then triethylamine (0.038 mL) was slowly added. The mixture was stirred 16 hours at room temperature. After dilution with dichloromethane (20 mL), the reaction mixture was washed with water. The organic layer was dried over Na2SO4. After removal of the solvent, the residue was purified by flash chromatography (DCM/MeOH=92/2 and 95/5 (v/v)) thereby obtaining bis(2-butyloctyl) 10-[2-[2-[2-(1H-imidazole-4-carbonylamino)ethoxy]ethoxy]ethyl-nonyl-amino]nonadecanedioate as a yellow oil.

¹H NMR (500 MHz, DMSO-d6) δ ppm 0.73-0.91 (m, 15H), 1.03-1.38 (m, 68H), 1.41-1.63 (m, 6H), 2.26 (t, J=7 Hz, 4H), 2.77-3.18 (m, 9H), 3.34-3.40 (m, 2H), 3.43-3.57 (m, 6H), 3.91 (dd, J=6, 3 Hz, 4H), 7.58 (dd), pseudo-molecular ion m/z=1031, retention time (min)=2.45.

### Example 19: Synthesis of nonyl 8-[2-[3-[2-[2-[2-[2-(1H-imidazole-4-carbonylamino)ethoxy]ethoxy]ethoxy]ethoxy]propanoyloxy]ethyl-[8-(1-octylnonoxy)-8-oxooctyl]amino]octanoate (compound XX)

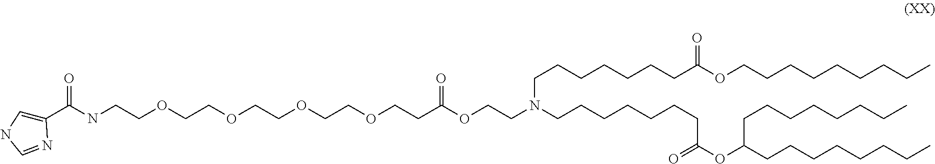 [SMILES: CCCCCCCCCOC(=O)CCCCCCCN(CCCCCCCC(=O)OC(CCCCCCCC)CCCCCCCC)CCOC(=O)CCOCCOCCOCCOCCNC(=O)c1c[nH]cn1]

The compound (XX) was synthesized based on the chemistry shown in Scheme (11).

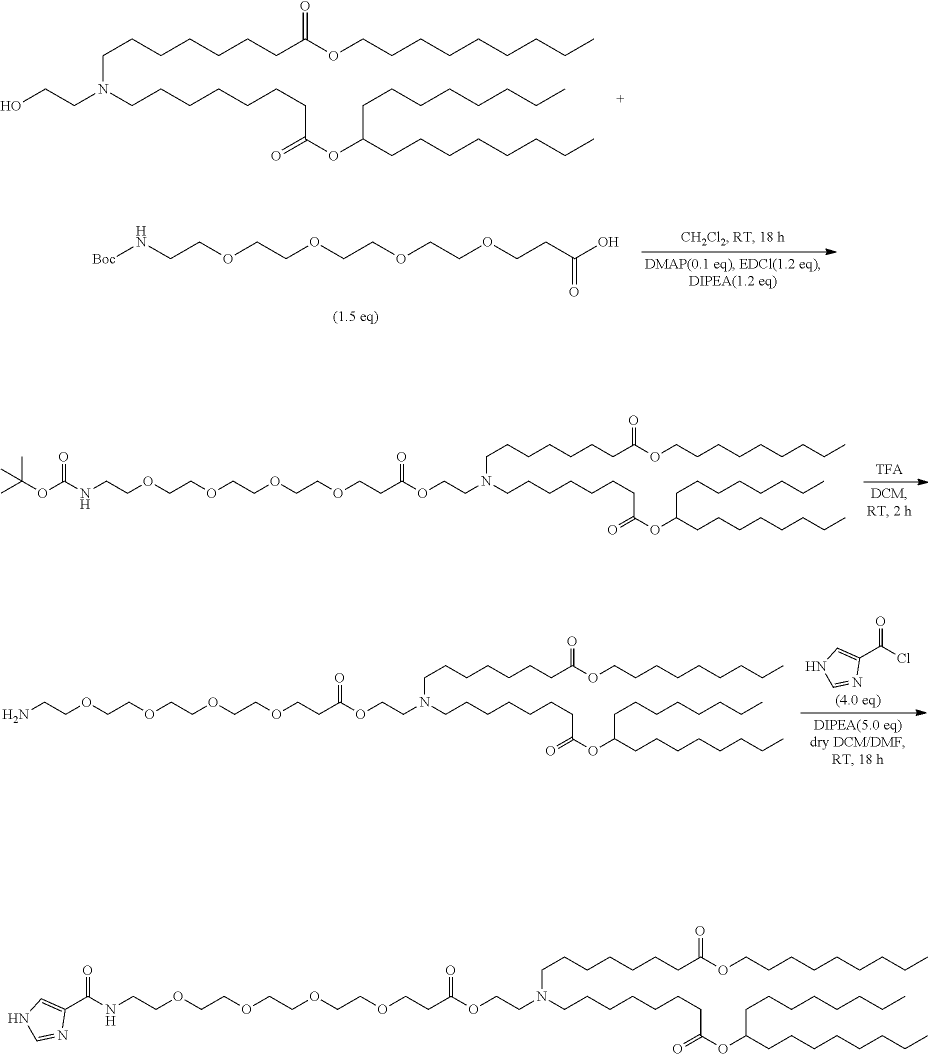

Synthesis of Compound (XX)

Step (1)

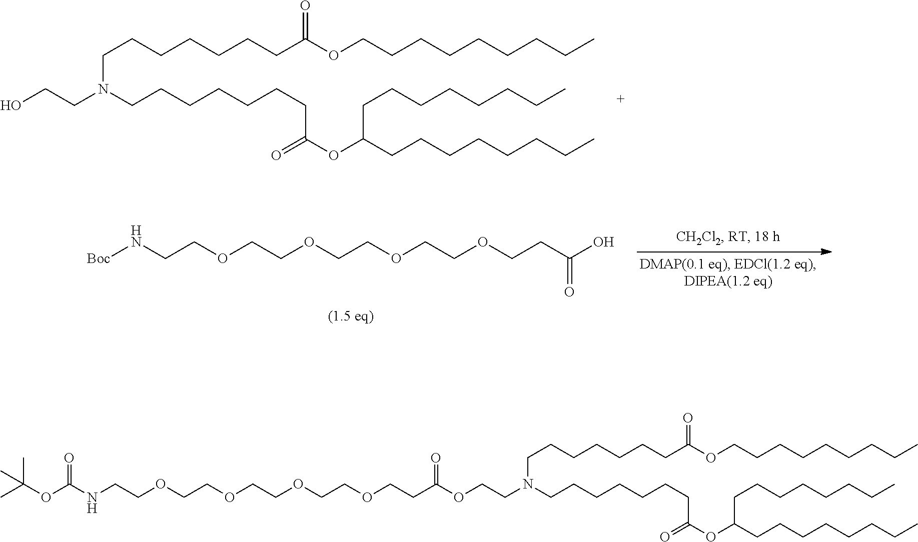

To a solution of nonyl 8-[2-hydroxyethyl-[8-(1-octylnonoxy)-8-oxo-octyl]amino]octanoate (1.00 g, 1.41 mmol) and 3-[2-[2-[2-[2-(tert-butoxycarbonylamino)ethoxy]ethoxy]ethoxy]ethoxy]propanoic acid (0.772 g, 2.11 mmol) in dry DCM (10 mL) was added DMAP (17.2 mg, 0.141 mmol), DIPEA (0.218 g, 1.69 mmol) and then added EDCI (0.324 g, 1.69 mmol) at 0° C. The reaction was stirred at room temperature for 18 hrs. TLC indicated that the starting material was disappeared and a new spot formed. Water (20 mL) was added to quench the reaction and the mixture was extracted with DCM (50 mL). The organic layer was washed with saturated sodium bicarbonate and dried over Na₂SO₄, filtered and concentrated. The residue was purified by silica gel chromatography (0-5% CH₃OH in DCM (3%)) to give nonyl 8-[2-[3-[2-[2-[2-[2-(tert-butoxycarbonylamino)ethoxy]ethoxy]ethoxy]ethoxy]propanoyloxy]ethyl-[8-(1-octylnonoxy)-8-oxo-octyl]amino]octanoate (1.126 g, 1.06 mmol, 75.6% yield) as colorless oil.

¹H NMR (400 MHz, CDCl₃) δ 5.07 (s, 1H), 4.92-4.81 (m, 1H), 4.16-4.02 (m, 4H), 3.78-3.72 (m, 2H), 3.68-3.58 (m, 12H), 3.58-3.50 (m, 2H), 3.37-3.26 (m, 2H), 2.71-2.57 (m, 4H), 2.47-2.38 (m, 4H), 2.33-2.24 (m, 4H), 1.66-1.56 (m, 6H), 1.53-1.22 (m, 65H), 0.92-0.83 (m, 9H).

Step (2)

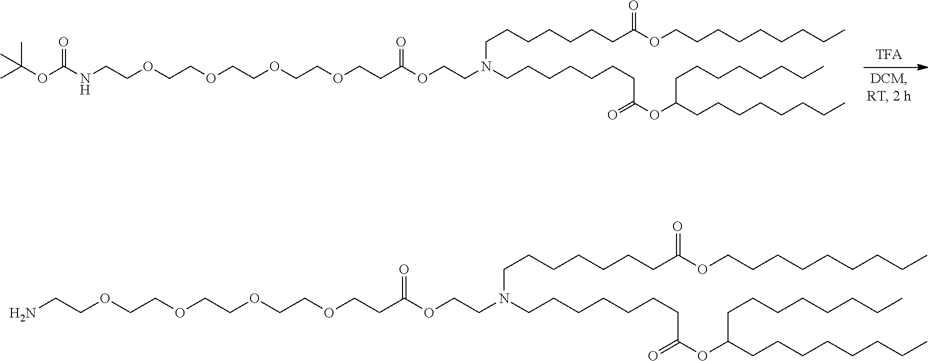 [SMILES: CCCCCCCCCOC(=O)CCCCCCCN(CCCCCCCC(=O)OC(CCCCCCCC)CCCCCCCC)CCOC(=O)CCOCCOCCOCCOCCNC(=O)OC(C)(C)C ; CCCCCCCCCOC(=O)CCCCCCCN(CCCCCCCC(=O)OC(CCCCCCCC)CCCCCCCC)CCOC(=O)CCOCCOCCOCCOCCN]

Nonyl 8-[2-[3-[2-[2-[2-[2-(tert-butoxycarbonylamino)ethoxy]ethoxy]ethoxy]ethoxy]propanoyloxy]ethyl-[8-(1-octylnonoxy)-8-oxo-octyl]amino]octanoate (1.13 g, 1.06 mmol) was dissolved in 10 mL DCM and then added TFA (4 mL). The mixture was stirred at room temperature 2 h. TLC (3% CH₃OH in DCM) shows that the reaction was done. The solvent was evaporated (50 mL DCM*2) and then dissolved in DCM (100 mL) and washed with saturated NaHCO₃ (10 mL), organic layer was separated and dried over Na₂SO₄, filtrated and solvent was removed to give nonyl 8-[2-[3-[2-[2-[2-(2-aminoethoxy)ethoxy]ethoxy]ethoxy]propanoyloxy]ethyl-[8-(1-octylnonoxy)-8-oxo-octyl]amino]octanoate (1.0 g, 1.04 mmol, 98.1% yield) as colorless oil.

¹H NMR (500 MHz, CDCl₃) δ 4.90-4.82 (m, 1H), 4.15 (t, J=6.2 Hz, 2H), 4.05 (t, J=6.8 Hz, 2H), 3.76 (dd, J=10.8, 5.0 Hz, 4H), 3.72-3.61 (m, 12H), 3.14-3.09 (m, 2H), 2.73 (t, J=6.2 Hz, 2H), 2.62 (t, J=6.0 Hz, 2H), 2.51-2.42 (m, 4H), 2.28 (dd, J=14.1, 7.3 Hz, 4H), 1.67-1.36 (m, 14H), 1.35-1.22 (m, 48H), 0.90-0.84 (m, 9H).

Step (3)

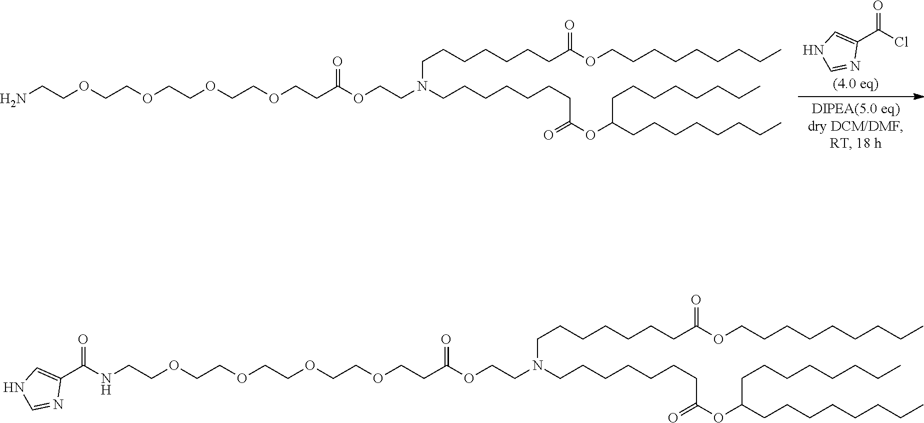 [SMILES: CCCCCCCCCOC(=O)CCCCCCCN(CCCCCCCC(=O)OC(CCCCCCCC)CCCCCCCC)CCOC(=O)CCOCCOCCOCCOCCN ; O=C(Cl)c1c[nH]cn1 ; CCCCCCCCCOC(=O)CCCCCCCN(CCCCCCCC(=O)OC(CCCCCCCC)CCCCCCCC)CCOC(=O)CCOCCOCCOCCOCCNC(=O)c1c[nH]cn1]

Nonyl 8-[2-[3-[2-[2-[2-(2-aminoethoxy)ethoxy]ethoxy]ethoxy]propanoyloxy]ethyl-[8-(1-octylnonoxy)-8-oxo-octyl]amino]octanoate (1.0 g, 1.04 mmol) was dissolved in 15 mL anhydrous DCM and the 1H-imidazole-4-carbonyl chloride (545 mg, 4.18 mmol) in 1 mL anhydrous DMF and DIPEA (675 mg, 5.22 mmol) were added. The mixture was stirred at room temperature overnight. The solvent was evaporated and the product purified by flash chromatography (25 g column, DCM/MeOH 0% to 5%) eluted with 0-5% methanol in dichloromethane (4%) to give nonyl 8-[2-[3-[2-[2-[2-[2-(1H-imidazole-4-carbonylamino)ethoxy]ethoxy]ethoxy]ethoxy]propanoyloxy]ethyl-[8-(1-octylnonoxy)-8-oxo-octyl]amino]octanoate (542.5 mg, 0.495 mmol, 47.4% yield) as pale yellow oil.

¹H NMR (500 MHz, CDCl₃) δ 11.22 (s, 1H), 7.63 (s, 3H), 4.86 (p, J=6.2 Hz, 1H), 4.16 (d, J=5.5 Hz, 2H), 4.05 (t, J=6.8 Hz, 2H), 3.75-3.57 (m, 18H), 2.72 (s, 2H), 2.54 (dd, J=24.0, 17.5 Hz, 6H), 2.28 (dd, J=13.5, 7.4 Hz, 4H), 1.61 (dd, J=13.0, 6.5 Hz, 6H), 1.53-1.22 (m, 56H), 0.91-0.84 (m, 9H). LCMS: Find peak: MS(ESI) m/z=1052.9 (M+H)+ at 2.278 min

### Example 20: Synthesis of 6-[6-(2-hexyldecanoyloxy)hexyl-[2-[2-[2-[2-[2-(1H-imidazole-4-carbonylamino)ethoxy]ethoxy]ethoxy]ethoxy]ethyl]amino]hexyl 2-hexyldecanoate (compound XXI)

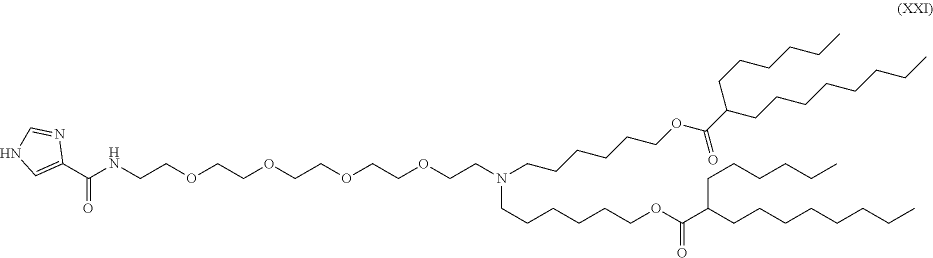 [SMILES: CCCCCCCCC(CCCCCC)C(=O)OCCCCCCN(CCCCCCOC(=O)C(CCCCCC)CCCCCCCC)CCOCCOCCOCCOCCNC(=O)c1c[nH]cn1]

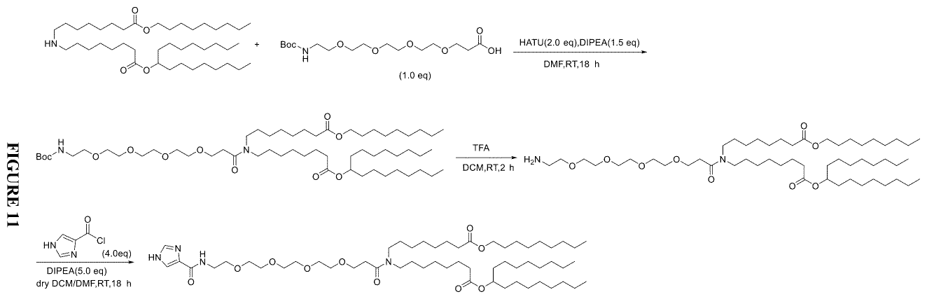

The compound (XXI) was synthesized based on the chemistry shown in Scheme (11) as shown on FIG. 10.

Synthesis

Step (1)

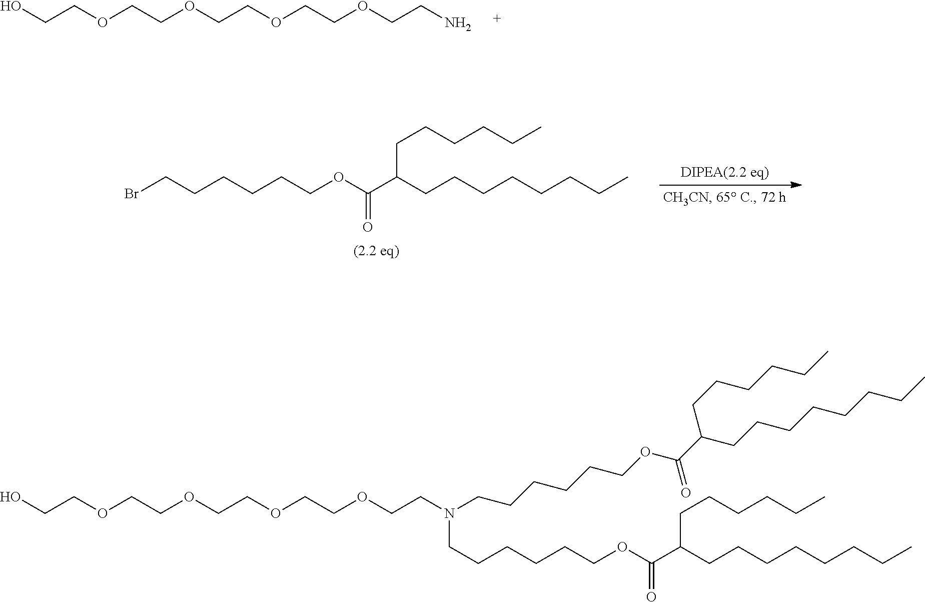 [SMILES: NCCOCCOCCOCCOCCO ; CCCCCCCCC(CCCCCC)C(=O)OCCCCCCBr ; CCCCCCCCC(CCCCCC)C(=O)OCCCCCCN(CCCCCCOC(=O)C(CCCCCC)CCCCCCCC)CCOCCOCCOCCOCCO]

A solution of 2-[2-[2-[2-(2-aminoethoxy)ethoxy]ethoxy]ethoxy]ethanol (0.450 g, 1.90 mmol) and 6-bromohexyl 2-hexyldecanoate (1.75 g, 4.17 mmol) and DIPEA (0.539 g, 4.17 mmol) in CH₃CN (10 mL) and cyclopentyl methyl ether (3 mL) was stirred at 65° C. for 72 hours. The reaction was cooled to rt and solvents were evaporated in vacuo. The residue was taken-up in EtOAc (50 mL*2) and H₂O (20 mL). The organic layer was separated, dried over Na₂SO₄ and evaporated in vacuo. The residue was purified by silica gel chromatography (0-5% MeOH in dichloromethane (3%)) to obtain 6-[6-(2-hexyldecanoyloxy)hexyl-[2-[2-[2-[2-(2-hydroxyethoxy)ethoxy]ethoxy]ethoxy]ethyl]amino]hexyl 2-hexyldecanoate (1.082 g, 1.18 mmol, 56.2% yield) as pale yellow oil.

¹H NMR (500 MHz, CDCl₃) δ 4.09-4.02 (m, 4H), 3.91-3.55 (m, 18H), 2.84 (s, 6H), 2.34-2.26 (m, 2H), 1.68-1.51 (m, 10H), 1.47-1.20 (m, 54H), 0.90-0.84 (m, 12H).

Step (2)

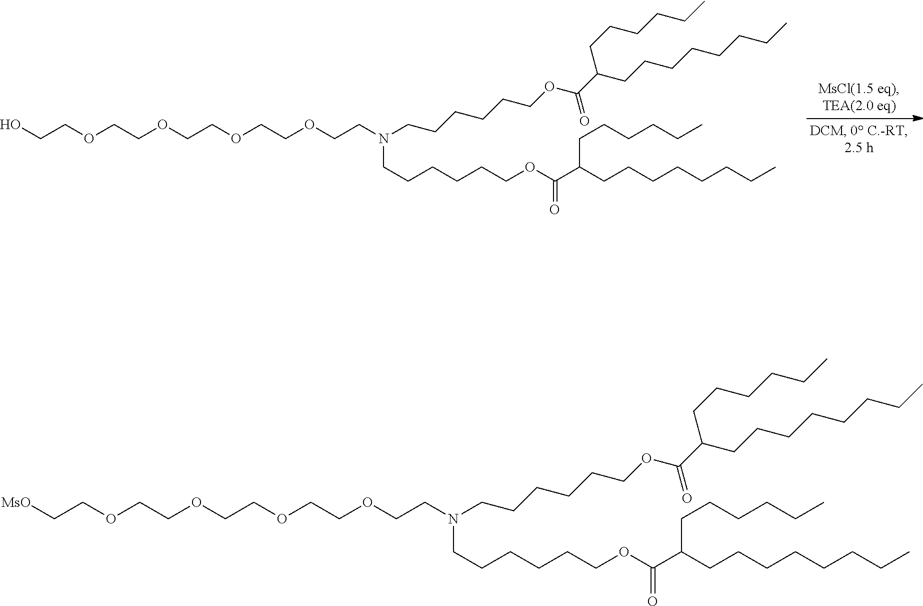 [SMILES: CCCCCCCCC(CCCCCC)C(=O)OCCCCCCN(CCCCCCOC(=O)C(CCCCCC)CCCCCCCC)CCOCCOCCOCCOCCO ; CCCCCCCCC(CCCCCC)C(=O)OCCCCCCN(CCCCCCOC(=O)C(CCCCCC)CCCCCCCC)CCOCCOCCOCCOCCOS(C)(=O)=O]

To a solution of 6-[6-(2-hexyldecanoyloxy)hexyl-[2-[2-[2-[2-(2-hydroxyethoxy)ethoxy]ethoxy] ethoxy]ethyl]amino]hexyl 2-hexyldecanoate (1.55 g, 1.70 mmol) and TEA (triethylamine) (0.343 g, 3.39 mmol) in 15 mL Dichloromethane (DCM) was added Ms-Cl (291 mg, 2.54 mmol). The mixture was stirred at room temperature for 3 hrs. TLC (CH3OH/DCM 4%) indicated that the starting material was consumed. The reaction was diluted with dichloromethane and washed with water (10 mL). The organic layer was dried over sodium sulfate, filtered and concentrated to afford 6-[2-[6-(2-butyloctanoyloxy)hexoxy]-3-[2-[2-[2-(2-hydroxyethoxy)ethoxy]ethoxy]ethoxy] propoxy]hexyl 2-butyloctanoate (1.592 g, 1.60 mmol, 94.6% yield) as yellow oil, which was used directly for next step.

¹H NMR (400 MHz, CDCl3) δ 4.44-4.32 (m, 2H), 4.05 (t, J=6.7 Hz, 4H), 3.91-3.52 (m, 16H), 3.17-2.36 (m, 9H), 2.35-2.25 (m, 2H), 1.67-1.52 (m, 10H), 1.48-1.19 (m, 54H), 0.88 (t, J=6.7 Hz, 12H).

Step (3)

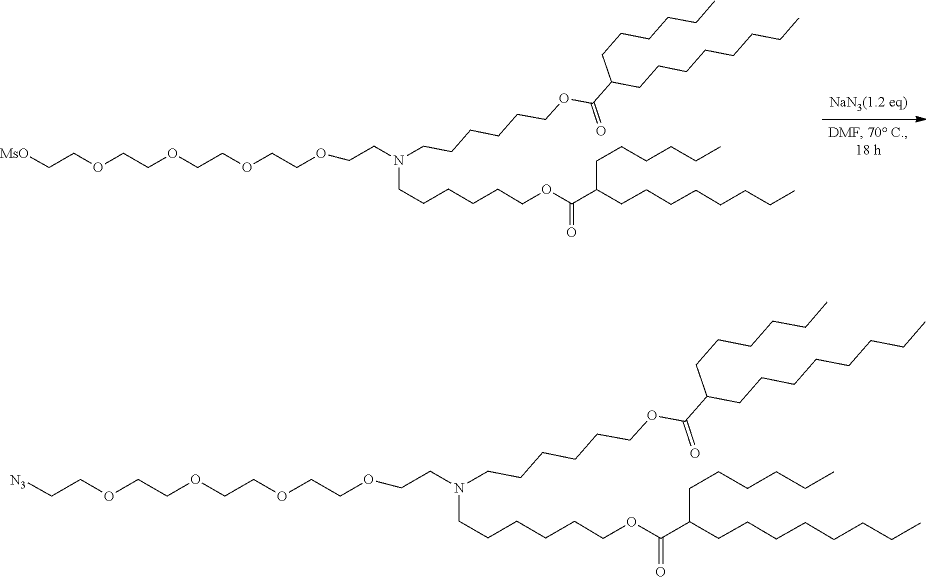 [SMILES: CCCCCCCCC(CCCCCC)C(=O)OCCCCCCN(CCCCCCOC(=O)C(CCCCCC)CCCCCCCC)CCOCCOCCOCCOCCOS(C)(=O)=O ; CCCCCCCCC(CCCCCC)C(=O)OCCCCCCN(CCCCCCOC(=O)C(CCCCCC)CCCCCCCC)CCOCCOCCOCCOCCN=[N+]=[N-]]

To a solution of 6-[6-(2-hexyldecanoyloxy)hexyl-[2-[2-[2-[2-(2-methylsulfonyloxyethoxy)ethoxy]ethoxy]ethoxy]ethyl]amino]hexyl 2-hexyldecanoate (1.592 g, 1.6 mmol) dissolved in DMF (10 mL) then added NaN₃ (125 m g, 1.92 mmol). Then the reaction mixture was stirred at 70° C. for 18 hr. TLC indicated that the starting material was disappeared and a new spot was formed (CH₃OH in DCM (3%)). The DMF was removed under vacuum and the residue was diluted with H₂O (20 mL) then extracted with EA (2*50 mL). The organic layer was washed with brine (50 mL*3), dried over Na₂SO₄, filtered and concentrated. The residue was purified by flash chromatography column eluted with 0-4% CH₃OH in DCM (2%) to give 6-[2-[2-[2-[2-(2-azidoethoxy)ethoxy]ethoxy]ethoxy]ethyl-[6-(2-hexyldecanoyloxy)hexyl]amino]hexyl 2-hexyldecanoate (1.059 g, 1.13 mmol, 70.3% yield) as yellow oil.

¹H NMR (400 MHz, CDCl₃) δ 4.09-4.01 (m, 4H), 3.71-3.46 (m, 16H), 3.42-3.36 (m, 2H), 2.54 (d, J=83.2 Hz, 6H), 2.36-2.25 (m, 2H), 1.68-1.51 (m, 8H), 1.49-1.20 (m, 56H), 0.92-0.83 (m, 12H).

Step (4)

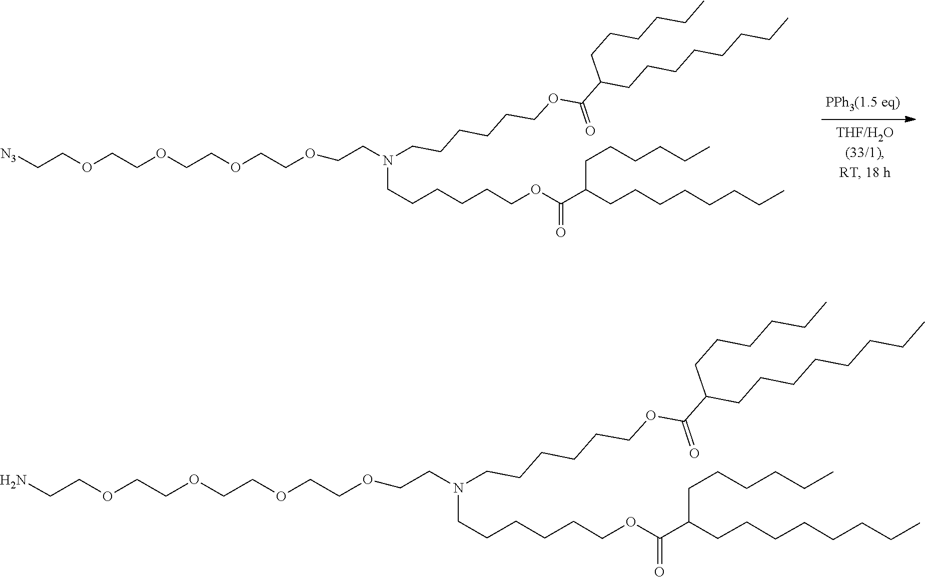 [SMILES: CCCCCCCCC(CCCCCC)C(=O)OCCCCCCN(CCCCCCOC(=O)C(CCCCCC)CCCCCCCC)CCOCCOCCOCCOCCN=[N+]=[N-] ; CCCCCCCCC(CCCCCC)C(=O)OCCCCCCN(CCCCCCOC(=O)C(CCCCCC)CCCCCCCC)CCOCCOCCOCCOCCN]

A mixture of 6-[2-[2-[2-[2-(2-azidoethoxy)ethoxy]ethoxy]ethoxy]ethyl-[6-(2-hexyldecanoyloxy)hexyl]amino]hexyl 2-hexyldecanoate (1.059 g, 1.13 mmol) and triphenylphosphine (0.443 g, 1.69 mmol) in THF (20 mL)/Water (0.6 mL) was stirred at 20° C. for 16 hrs. TLC (3% methanol in dichloromethane) indicated the reaction was complete. The solvent was removed and the residue was purified by column chromatography eluted with 0-20% CH₃OH in CH₂Cl₂ ((14%)) to give 6-[2-[2-[2-[2-(2-aminoethoxy)ethoxy]ethoxy]ethoxy]ethyl-[6-(2-hexyldecanoyloxy)hexyl]amino]hexyl 2-hexyldecanoate (0.843 g, 0.923 mmol, 81.9% yield) as light yellow oil.

¹H NMR (400 MHz, CDCl₃) δ 4.10-4.01 (m, 4H), 3.71-3.50 (m, 16H), 2.93-2.86 (m, 2H), 2.70-2.62 (m, 2H), 2.51-2.41 (m, 4H), 2.34-2.27 (m, 2H), 1.67-1.51 (m, 8H), 1.48-1.21 (m, 56H), 0.92-0.83 (m, 12H).

Step (5)

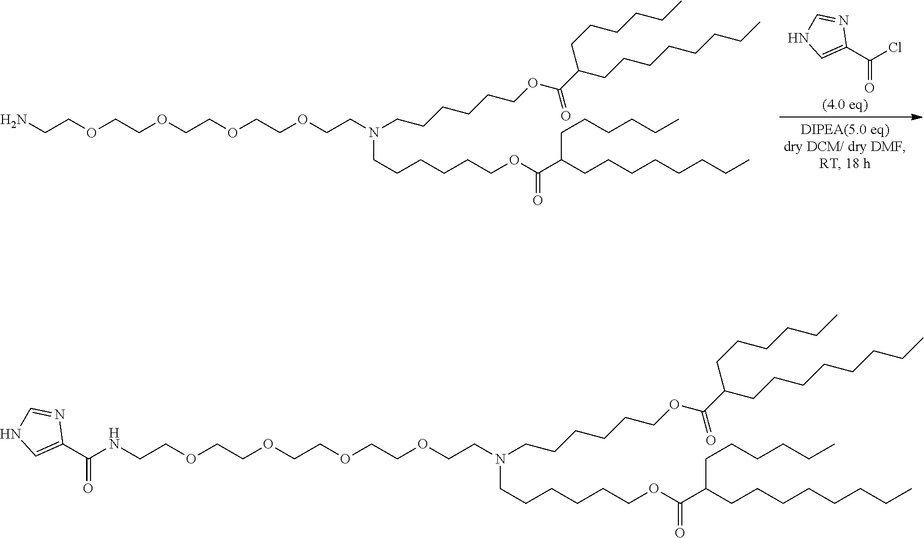 [SMILES: CCCCCCCCC(CCCCCC)C(=O)OCCCCCCN(CCCCCCOC(=O)C(CCCCCC)CCCCCCCC)CCOCCOCCOCCOCCN ; O=C(Cl)c1c[nH]cn1 ; CCCCCCCCC(CCCCCC)C(=O)OCCCCCCN(CCCCCCOC(=O)C(CCCCCC)CCCCCCCC)CCOCCOCCOCCOCCNC(=O)c1c[nH]cn1]

6-[2-[2-[2-[2-(2-aminoethoxy)ethoxy]ethoxy]ethoxy]ethyl-[6-(2-hexyldecanoyloxy)hexyl]amino]hexyl 2-hexyldecanoate (840 mg, 0.92 mmol) was dissolved in 10 mL anhydrous DCM and 1H-imidazole-4-carbonyl chloride (480 mg, 3.68 mmol) and DIPEA (594 mg, 4.60 mmol) were added. The mixture was stirred at room temperature overnight. The solution was added water (10 mL) then extracted with DCM, the organic was washed with brine then dried with Na₂SO4. The residue was purified by flash chromatography (40 g column, DCM/MeOH 0% to 6%) eluted with methanol in dichloromethane (6%) to give 6-[6-(2-hexyldecanoyloxy)hexyl-[2-[2-[2-[2-[2-(1H-imidazole-4-carbonylamino)ethoxy]ethoxy]ethoxy]ethoxy]ethyl]amino]hexyl 2-hexyldecanoate (488.6 mg, 0.461 mmol, 50.1% yield) as yellow oil.

¹H NMR (400 MHz, CDCl₃) δ 7.62 (d, J=7.7 Hz, 3H), 4.10-4.00 (m, 4H), 3.71-3.52 (m, 18H), 2.72 (d, J=73.5 Hz, 6H), 2.36-2.26 (m, 2H), 1.66-1.21 (m, 64H), 0.87 (t, J=6.6 Hz, 12H).

LCMS: EXP-21-IX3047-32609-LCMSA020

### Example 21: Synthesis of 6 nonyl 8-[3-[2-[2-[2-[2-(1H-imidazole-4-carbonylamino)ethoxy]ethoxy]ethoxy]ethoxy]propanoyl-[8-(1-octylnonoxy)-8-oxo-octyl]amino]octanoate (Compound XXII)

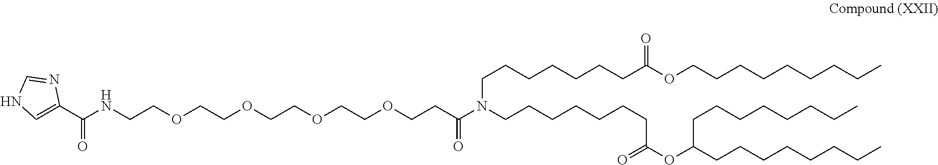 [SMILES: CCCCCCCCCOC(=O)CCCCCCCN(CCCCCCCC(=O)OC(CCCCCCCC)CCCCCCCC)C(=O)CCOCCOCCOCCOCCNC(=O)c1c[nH]cn1]

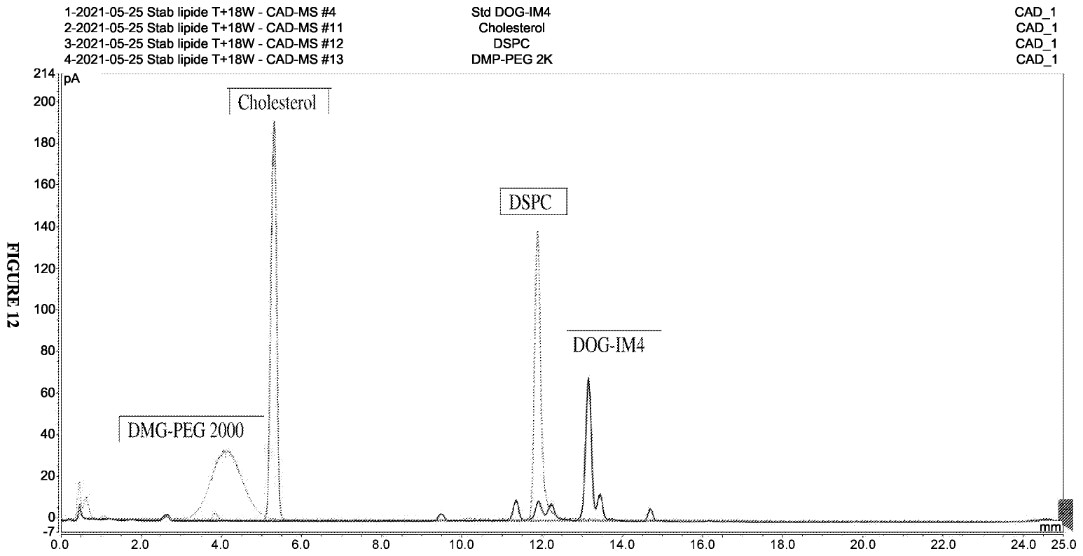

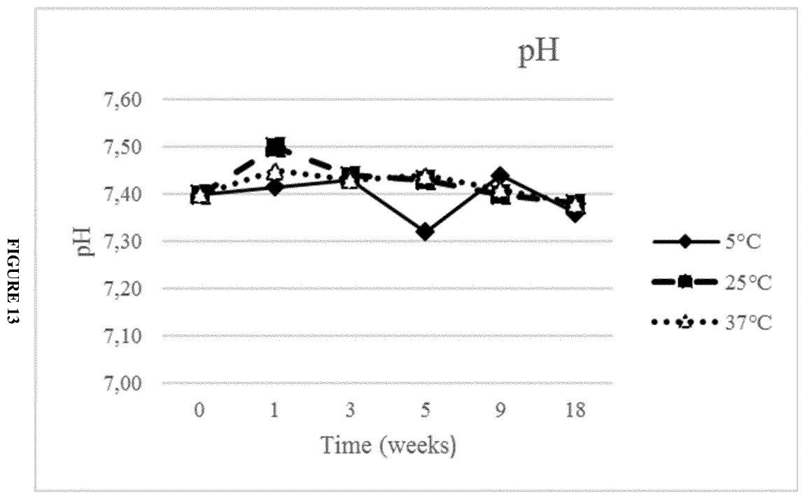

The compound (XXII) was synthesized based on the chemistry shown in Scheme (12) as shown on FIG. 11.

Synthesis of Compound (XXII)

Step (1)

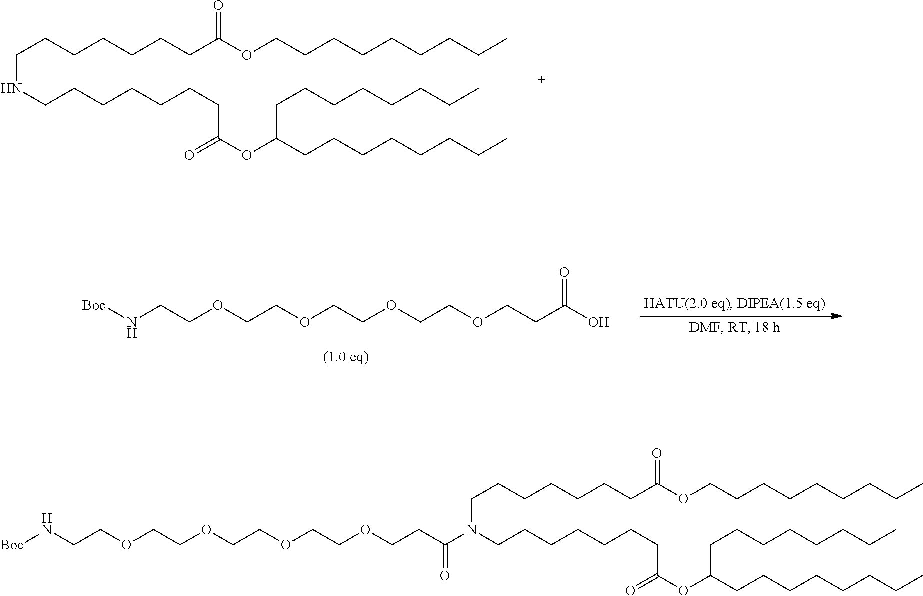 [SMILES: CCCCCCCCCOC(=O)CCCCCCCNCCCCCCCC(=O)OC(CCCCCCCC)CCCCCCCC ; CC(C)(C)OC(=O)NCCOCCOCCOCCOCCC(=O)O ; CCCCCCCCCOC(=O)CCCCCCCN(CCCCCCCC(=O)OC(CCCCCCCC)CCCCCCCC)C(=O)CCOCCOCCOCCOCCNC(=O)OC(C)(C)C]

To the solution of nonyl 8-[[8-(1-octylnonoxy)-8-oxo-octyl]amino]octanoate (1.00 g, 1.51 mmol) in anhydrous DMF (10 mL) and anhydrous DCM (2 mL) was added 3-[2-[2-[2-[2-(tert-butoxycarbonylamino)ethoxy]ethoxy]ethoxy]ethoxy]propanoic acid (551 mg, 1.51 mmol), HATU (0.859 g, 2.26 mmol) and DIPEA (0.389 g, 3.01 mmol). The mixture was stirred at room temperature for 18 h. TLC (4% methanol in DCM) indicated that the reaction was finished. The solvent was removed under vacuum and the residue was partitioned between H₂O (20 mL) and ethyl acetate (2*50 mL). The organic layer was washed with brine (50 mL*3), dried over Na₂SO₄. The residue was purified by flash chromatography column eluted with 0% to 3%(2%) methanol in dichloromethane to give nonyl 8-[3-[2-[2-[2-[2-(tert-butoxycarbonylamino)ethoxy]ethoxy]ethoxy]ethoxy]propanoyl-[8-(1-octylnonoxy)-8-oxo-octyl]amino]octanoate (1.319 g, 1.3 mmol, 86.4% yield).

¹H NMR (500 MHz, CDCl₃) δ 5.12 (s, 1H), 4.90-4.82 (m, 1H), 4.08-4.01 (m, 2H), 3.78 (t, J=6.8 Hz, 2H), 3.69-3.60 (m, 12H), 3.55 (t, J=5.0 Hz, 2H), 3.36-3.16 (m, 6H), 2.62 (t, J=6.7 Hz, 2H), 2.33-2.23 (m, 4H), 1.65-1.47 (m, 14H), 1.44 (s, 9H), 1.35-1.23 (m, 48H), 0.90-0.85 (m, 9H).

Step (2)

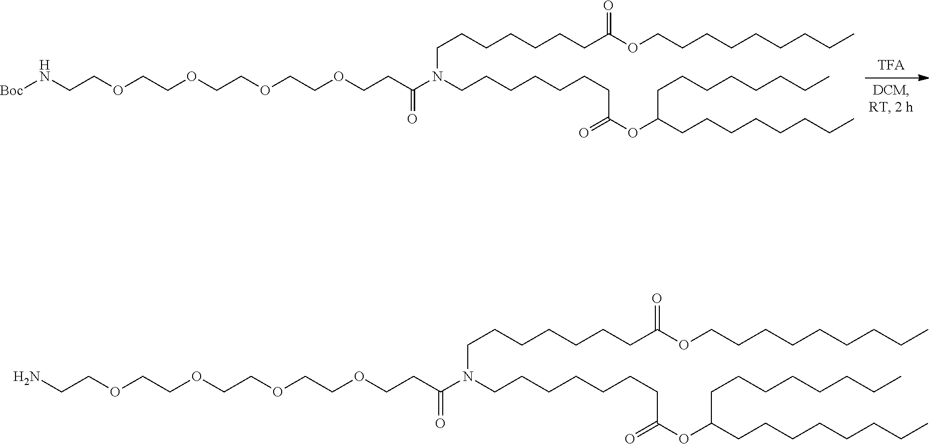 [SMILES: CCCCCCCCCOC(=O)CCCCCCCN(CCCCCCCC(=O)OC(CCCCCCCC)CCCCCCCC)C(=O)CCOCCOCCOCCOCCNC(=O)OC(C)(C)C ; CCCCCCCCCOC(=O)CCCCCCCN(CCCCCCCC(=O)OC(CCCCCCCC)CCCCCCCC)C(=O)CCOCCOCCOCCOCCN]

To the solution of nonyl 8-[3-[2-[2-[2-[2-(tert-butoxycarbonylamino)ethoxy]ethoxy]ethoxy]ethoxy]propanoyl-[8-(1-octylnonoxy)-8-oxo-octyl]amino]octanoate (1.319 g, 1.3 mmol) in 10 mL DCM was added TFA (4 mL) and the mixture was stirred at room temperature for 2 h. TLC (4% CH₃OH in DCM) indicated that the reaction was finished. The solvent was removed and azeotroped with dichloromethane (50 mL DCM*2) and then dissolved in DCM (100 mL) and washed with saturated NaHCO₃ (10 mL). The organic layer was dried over Na₂SO₄, filtrated and concentrated to give nonyl 8-[3-[2-[2-[2-(2-aminoethoxy)ethoxy]ethoxy]ethoxy]propanoyl-[8-(1-octylnonoxy)-8-oxo-octyl]amino]octanoate (1.19 g, 1.24 mmol, 95.1% yield) as yellow oil.

¹H NMR (400 MHz, CDCl₃) δ 4.92-4.80 (m, 1H), 4.09-4.02 (m, 2H), 3.84-3.71 (m, 4H), 3.71-3.52 (m, 12H), 3.33-3.04 (m, 6H), 2.60 (t, J=5.8 Hz, 2H), 2.33-2.22 (m, 4H), 1.57 (dd, J=32.9, 14.6 Hz, 14H), 1.38-1.21 (m, 48H), 0.92-0.83 (m, 9H).

Step (3)

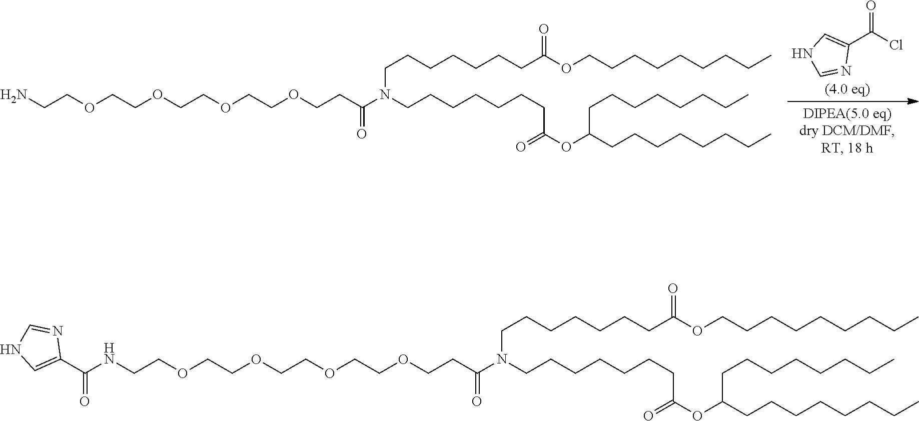

To the solution of nonyl 8-[3-[2-[2-[2-(2-aminoethoxy)ethoxy]ethoxy]ethoxy]propanoyl-[8-(1-octylnonoxy)-8-oxo-octyl]amino]octanoate (600 mg, 0.657 mmol) in 10 mL anhydrous DCM were added 1H-imidazole-4-carbonyl chloride (343 mg, 2.63 mmol) in 1 mL anhydrous DMF and DIPEA (424 mg, 3.28 mmol). The mixture was stirred at room temperature overnight. The solvent was removed under vacuum. Then the residue was purified by flash chromatography column (eluted with 0% to 5% methanol in dichloromethane (4%) to give nonyl 8-[3-[2-[2-[2-[2-(1H-imidazole-4-carbonylamino)ethoxy]ethoxy]ethoxy]ethoxy]propanoyl-[8-(1-octylnonoxy)-8-oxo-octyl]amino]octanoate (312.3 mg, 0.294 mmol, 44.8% yield) as yellow oil.

¹H NMR (500 MHz, CDCl₃) δ 7.60 (t, J=23.3 Hz, 2H), 4.91-4.83 (m, 1H), 4.09-4.02 (m, 2H), 3.80-3.51 (m, 18H), 3.31-3.16 (m, 4H), 2.60 (t, J=6.7 Hz, 2H), 2.33-2.24 (m, 4H), 1.66-1.45 (m, 14H), 1.34-1.23 (m, 48H), 0.90-0.85 (m, 9H).

LCMS: Find peak: MS(ESI) m/z=1008.8 (M+H)+ at 4.616 min

### Example 22: Synthesis of nonyl 8-[2-[3-[2-[2-[2-[2-(1H-imidazole-4-carbonylamino)ethoxy]ethoxy]ethoxy]ethoxy]propanoylamino]ethyl-[8-(1-octylnonoxy)-8-oxooctyl]amino]octanoate (Compound XXIII)

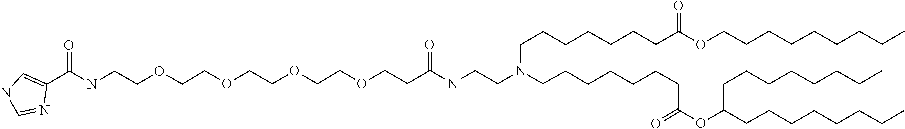

Synthesis of Compound (XXIII)

Step (1)

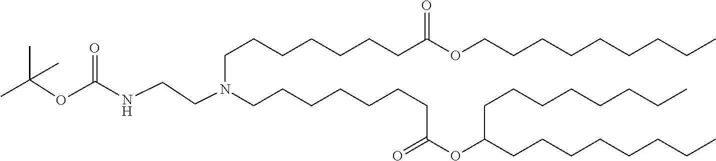 [SMILES: CCCCCCCCCOC(=O)CCCCCCCN(CCCCCCCC(=O)OC(CCCCCCCC)CCCCCCCC)CCNC(=O)OC(C)(C)C]

A solution of nonyl 8-bromooctanoate (4.65 g, 0.0133 mol), 1-octylnonyl 8-[2-(tertbutoxycarbonylamino)ethylamino]octanoate (6 g, 0.0111 mol) and N-ethyl-N-isopropyl-propan-2-amine (1.72 g, 0.0133 mol) in acetonitrile was allowed to stir at 65° C. for 72 h. The reaction was cooled to RT and solvents were evaporated in vacuo. The residue was taken-up in ethyl acetate and saturated sodium bicarbonate. The organic layer was separated, dried over Na₂SO₄ and evaporated in vacuo. The residue was purified by silica gel chromatography (mixture of 1% NH₄OH, 20% MeOH in dichloromethane) to obtain nonyl 8-[2-(tertbutoxycarbonylamino)ethyl-[8-(1-octylnonoxy)-8-oxo-octyl]amino]octanoate (7.24 g, 80.7% yield).

¹H NMR (500 MHz, CDCl₃) δ 4.98 (s, 1H), 4.86 (s, 1H), 4.05 (t, J=6.8 Hz, 2H), 3.14 (s, 2H), 2.49 (s, 2H), 2.38 (s, 4H), 2.31-2.24 (m, 4H), 1.61 (dd, J=14.0, 6.8 Hz, 6H), 1.50 (d, J=6.0 Hz, 4H), 1.45 (d, J=7.2 Hz, 9H), 1.40 (d, J=6.3 Hz, 4H), 1.35-1.22 (m, 48H), 0.88 (td, J=6.8, 2.0 Hz, 9H).

Step (2)

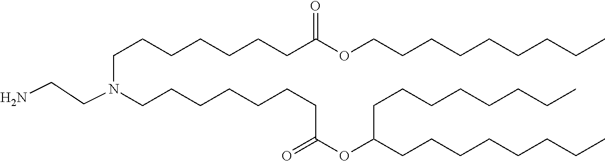 [SMILES: CCCCCCCCCOC(=O)CCCCCCCN(CCN)CCCCCCCC(=O)OC(CCCCCCCC)CCCCCCCC]

To the solution of [(Z)-non-2-enyl] 8-[2-(tert-butoxycarbonylamino)ethyl-[(7R,11R)-3,7,11,15-tetramethylhexadecyl]amino]octanoate (3.22 g, 0.00456 mol) in CH₂Cl₂ (30 mL) under ice bath, was TFA (10.4 g) dropwise and the mixture was stirred at room temperature for 10 h. The reaction was quenched with a saturated NaHCO₃ at 0° C. The organic layer was washed with a saturated NaHCO₃, 0.1 M NaOH and brine, dried over sodium sulfate. The solvent was remove under vacuum to give [(Z)-non-2-enyl] 8-[2-aminoethyl-[(7R,11R)-3,7,11,15-tetramethylhexadecyl]amino]octanoate (2.03 g, yield: 73.3%) as colorless oil.

¹H NMR (500 MHz, CDCl₃) δ 4.90-4.82 (m, 1H), 4.10-4.02 (m, 2H), 2.79-2.69 (m, 2H), 2.50-2.45 (m, 2H), 2.43-2.38 (m, 3H), 2.32-2.25 (m, 4H), 2.11-1.99 (m, 4H), 1.66-1.57 (m, 6H), 1.55-1.47 (m, 4H), 1.46-1.38 (m, 4H), 1.37-1.19 (m, 51H), 0.95-0.81 (m, 9H).

Step (3)

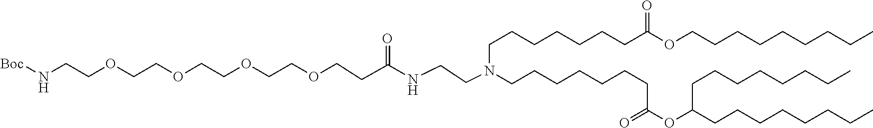 [SMILES: CCCCCCCCCOC(=O)CCCCCCCN(CCCCCCCC(=O)OC(CCCCCCCC)CCCCCCCC)CCNC(=O)CCOCCOCCOCCOCCNC(=O)OC(C)(C)C]

To a solution of nonyl 8-[2-aminoethyl-[8-(1-octylnonoxy)-8-oxo-octyl]amino]octanoate (1.33 g, 0.00187 mol) in dichloromethane (10 ml) was added 3-[2-[2-[2-[2-(tertbutoxycarbonylamino)ethoxy]ethoxy]ethoxy]ethoxy]propanoic acid (0.684 g, 0.00187 mol), DIPEA (0.484 g, 0.00375 mol), HATU (1.07 g, 0.00281 mol). The mixture was stirred at 25° C. for 18 h. After reaction, the mixture was diluted with DCM (100 ml), washed with water (300 ml×2), brine (300 ml), dried over Na2SO₄. The organic was concentrated and purified by flash chromatography column eluted with 20% ethyl acetate in petroleum ether to give nonyl 8-[2-[3-[2-[2-[2-[2-(tert-butoxycarbonylamino)ethoxy]ethoxy]ethoxy]ethoxy]propanoylamino]ethyl-[8-(1-octylnonoxy)-8-oxo-octyl]amino]octanoate (1.28 g, yield 64.8%) as a colourless oil.

¹H NMR (500 MHz, CDCl₃) δ 5.14-5.06 (m, 1H), 4.91-4.81 (m, 1H), 4.09-4.01 (m, 2H), 3.80-3.70 (m, 3H), 3.68-3.59 (m, 14H), 3.56-3.50 (m, 2H), 3.41-3.22 (m, 4H), 2.51-2.41 (m, 4H), 2.32-2.24 (m, 4H), 2.05-1.85 (m, 2H), 1.65-1.57 (m, 6H), 1.53-1.40 (m, 17H), 1.37-1.22 (m, 48H), 0.92-0.83 (m, 9H).

Step (4)

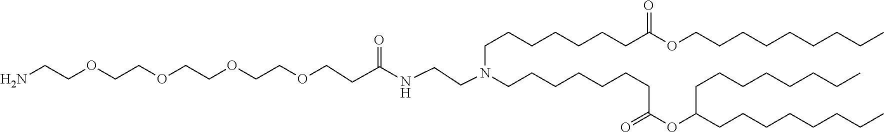 [SMILES: CCCCCCCCCOC(=O)CCCCCCCN(CCCCCCCC(=O)OC(CCCCCCCC)CCCCCCCC)CCNC(=O)CCOCCOCCOCCOCCN]

To a solution of nonyl 8-[2-[3-[2-[2-[2-[2-(tertbutoxycarbonylamino)ethoxy]ethoxy]ethoxy]ethoxy]propanoylamino]ethyl-[8-(1-octylnonoxy)-8-oxooctyl]amino]octanoate (0.275 g, 0.247 mmol) in dichloromethane (6 mL) was added TFA (0.5 mL) and the mixture was stirred at 25° C. for 18 h. TLC (5% methanol in DCM) indicated that the starting material was consumed. The solvent was removed and the residue was diluted with DCM (50 ml), washed with 0.2 N NaOH solution (10 ml), NaHCO₃ solution (10 ml), dried over Na₂SO₄, filtered and concentrated to give nonyl 8-[2-[3-[2-[2-[2-(2-aminoethoxy)ethoxy]ethoxy]ethoxy]propanoylamino]ethyl-[8-(1-octylnonoxy)-8-oxo-octyl]amino]octanoate (0.21 g, 0.198 mmol, yield 84.4%) as a light yellow oil.

¹H NMR (500 MHz, CDCl₃) δ 4.09-4.00 (m, 2H), 3.82-3.49 (m, 17H), 3.21-3.04 (m, 4H), 2.94-2.84 (m, 3H), 2.81-2.80 (m, 8H), 2.54-2.48 (m, 2H), 2.33-2.22 (m, 4H), 1.65-1.46 (m, 12H), 1.37-1.18 (m, 48H), 0.92-0.82 (m, 9H).

Step (5)

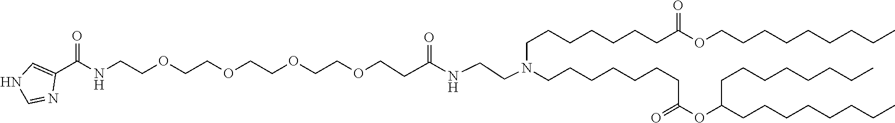 [SMILES: CCCCCCCCCOC(=O)CCCCCCCN(CCCCCCCC(=O)OC(CCCCCCCC)CCCCCCCC)CCNC(=O)CCOCCOCCOCCOCCNC(=O)c1c[nH]cn1]

The nonyl 8-[2-[3-[2-[2-[2-(2-aminoethoxy)ethoxy]ethoxy]ethoxy]propanoylamino]ethyl-[8-(1-octylnonoxy)-8-oxooctyl]amino]octanoate (580 mg, 0.546 mmol) and the 1H-imidazole-4-carbonyl chloride (285 mg, 2.18 mmol) in was dissolved in 15 mL anhydrous DCM and DIPEA (353 mg, 2.73 mmol) were added. The mixture was stirred at room temperature overnight. The solvent was evaporated and the product purified by flash chromatography (25 g column, DCM/MeOH 0% to 5%) eluted with methanol in dichloromethane (4%) to give nonyl 8-[2-[3-[2-[2-[2-[2-(1H-imidazole-4-carbonylamino)ethoxy]ethoxy]ethoxy]ethoxy]propanoylamino]ethyl-[8-(1-octylnonoxy)-8-oxooctyl]amino]octanoate (308 mg, 51.6% yield) as pale yellow oil. The product of this step was combined with [(Z)-non-2-enyl] 8-[2-aminoethyl-[(7R,11R)-3,7,11,15-tetramethylhexadecyl]amino]octanoate (obtained step 2)

¹H NMR (500 MHz, CDCl₃) δ 7.72-7.56 (m, 3H), 4.93-4.77 (m, 1H), 4.11-3.99 (m, 2H), 3.78-3.42 (m, 20H), 2.94-2.59 (m, 6H), 2.53-2.43 (m, 2H), 2.31-2.24 (m, 4H), 1.64-1.56 (m, 9H), 1.53-1.48 (m, 4H), 1.34-1.23 (m, 49H), 0.90-0.85 (m, 9H). LSMC: 526 (M+1) 98% UV (214 nm)

¹H-NMR (500 MHz, CDCl3) δ 7.72-7.56 (m, 3H), 4.93-4.77 (m, 1H), 4.11-3.99 (m, 2H), 3.78-3.42 (m, 20H), 2.94-2.59 (m, 6H), 2.53-2.43 (m, 2H), 2.31-2.24 (m, 4H), 1.64-1.56 (m, 9H), 1.53-1.48 (m, 4H), 1.34-1.23 (m, 49H), 0.90-0.85 (m, 9H).

LSMC: 526 (M+1) 98% UV (214 nm).

### Example 23: Synthesis of 6-[2-[6-(2-hexyldecanoyloxy)hexoxy]-3-[2-[2-[2-[2-[2-(1H-imidazole-4-carbonylamino)ethoxy]ethoxy]ethoxy]ethoxy]ethyl-octyl-amino]-3-oxo-propoxy]hexyl 2-hexyldecanoate (compound XXIV)

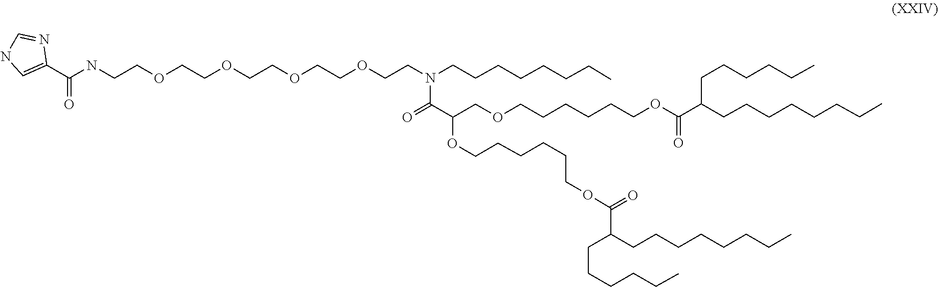

Synthesis of Compound (XXIV)

6-[3-[2-[2-[2-[2-(2-aminoethoxy)ethoxy]ethoxy]ethoxy]ethyl-octyl-amino]-2-[6-(2-hexyldecanoyloxy)hexoxy]-3-oxopropoxy]hexyl 2-hexyldecanoate (790 mg, 0.71 mmol) in dry DCM (20 mL) was added N,N-diethylethanamine (0.72 g, 7.10 mmol) and 1H-imidazole-4-carbonyl chloride (0.74 g, 5.67 mmol), the mixture was stirred at room temperature for 18 hrs. TLC indicated that the starting material was disappeared. The mixture was concentrated then purified by flash column chromatography on silica gel eluting with 3% to 6% (5%) methanol in dichloromethane to give 6-[2-[6-(2-hexyldecanoyloxy)hexoxy]-3-[2-[2-[2-[2-[2-(1H-imidazole-4-carbonylamino)ethoxy]ethoxy]ethoxy]ethoxy]ethyloctyl-amino]-3-oxo-propoxy]hexyl 2-hexyldecanoate (570 mg, 66.5% yield).

Multiplet Report

¹H-NMR (400 MHz, CDCl₃) δ 7.65 (s, 1H), 7.57 (s, 1H), 4.47-4.30 (m, 1H), 4.09-4.01 (m, 4H), 3.73-3.36 (m, 28H), 2.35-2.27 (m, 2H), 1.65-1.32 (m, 28H), 1.25 (s, 50H), 0.90-0.85 (m, 15H).

### Example 24: Synthesis of 1-hexylnonyl 8-[[8-(1-hexylnonoxy)-8-oxo-octyl]-[2-[2-[2-[2-[2-(1H-imidazole-4-carbonylamino)ethoxy]ethoxy]ethoxy]ethoxy]ethyl]amino]octanoate (Compound XXV)

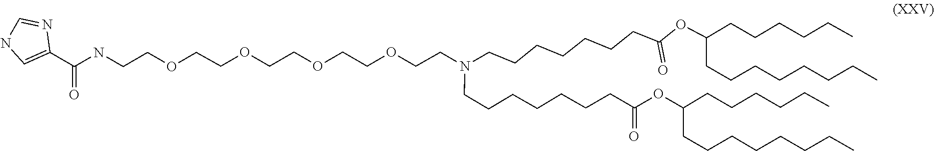

Synthesis of Compound (XXV)

1-hexylnonyl 8-[2-[2-[2-[2-(2-aminoethoxy)ethoxy]ethoxy]ethoxy]ethyl-[8-(1-hexylnonoxy)-8-oxooctyl]amino]octanoate (780 mg, 0.828 mmol) was dissolved in 10 mL anhydrous DCM and the 1H-imidazole-4-carbonyl chloride (433 mg, 3.31 mmol) and DIPEA (535 mg, 4.14 mmol) were added. The mixture was stirred at room temperature overnight. The solution was added water (10 mL) then extracted with DCM, the organic was washed with brine then dried with Na₂SO₄. The residue purified by flash chromatography (40 g column, DCM/MeOH 0% to 6%) eluted with methanol in dichloromethane (4%) to give 1-hexylnonyl 8-[[8-(1-hexylnonoxy)-8-oxo-octyl]-[2-[2-[2-[2-[2-(1H-imidazole-4-carbonylamino)ethoxy]ethoxy]ethoxy]ethoxy]ethyl]amino]octanoate (533.4 mg, 0.518 mmol, 62.6% yield) as yellow oil.

¹H-NMR (500 MHz, CDCl3) δ 7.65-7.57 (m, 2H), 7.54 (s, 1H), 4.90-4.83 (m, 2H), 3.69-3.49 (m, 18H), 2.73 (d, J=90.4 Hz, 6H), 2.28 (t, J=7.5 Hz, 4H), 1.67-1.17 (m, 68H), 0.88 (t, J=6.9 Hz, 12H).

### Example 25: Synthesis of 1-octylnonyl 8-[3-[2-[2-[2-(1H-imidazole-4-carbonylamino)ethoxy]ethoxy]ethylcarbamoyloxy]-2-[8-(1-octylnonoxy)-8-oxo-octoxy]propoxy]octanoate (Compound XXVI)

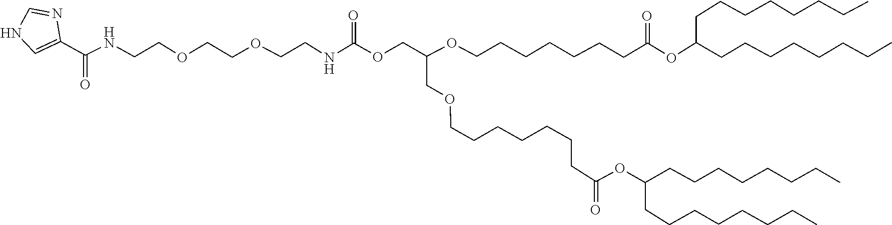 [SMILES: CCCCCCCCC(CCCCCCCC)OC(=O)CCCCCCCOCC(COC(=O)NCCOCCOCCNC(=O)c1c[nH]cn1)OCCCCCCCC(=O)OC(CCCCCCCC)CCCCCCCC]

Synthesis

Step (1)

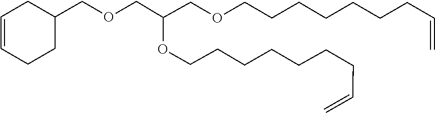 [SMILES: C=CCCCCCCCOCC(COCC1CC=CCC1)OCCCCCCCC=C]

To a solution of 3-benzyloxypropane-1,2-diol (5 g, 27.4 mmol) in DMF (150 ml) was added NaH (5.40 g, 137 mmol). The mixture was stirred at 80° C. for 1 hr. 9-bromonon-1-ene (14.1 g, 68.6 mmol) in DMF (10 ml) was added to the mixture at 25° C. and then the mixture was stirred at 80° C. for 18 hrs. The mixture was dealt with EA (300 ml), washed with water (300 ml×2), LiCl aq (300 ml), NaCl sat.aq (300 ml) and dried over Na₂SO₄. The organic was concentrated and purified by flash (5% EA in PE), to give 2,3-bis(non-8-enoxy)propoxymethylbenzene (3.74 g, 8.51 mmol, yield 31%) as a colourless oil.

¹H NMR (500 MHz, CDCl₃) δ 7.37-7.26 (m, 5H), 5.81 (ddt, J=16.9, 10.2, 6.7 Hz, 2H), 4.94 (ddd, J=17.4, 10.2, 9.3 Hz, 4H), 4.55 (s, 2H), 3.63-3.39 (m, 9H), 2.03 (td, J=7.9, 1.3 Hz, 4H), 1.58-1.26 (m, 20H).

Step (2)

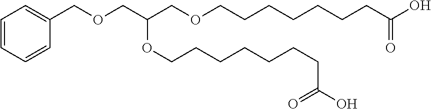 [SMILES: O=C(O)CCCCCCCOCC(COCc1ccccc1)OCCCCCCCC(=O)O]

To a solution of 2,3-bis(oct-7-enoxy)-N-octyl-N-[2-[2-[2-[2-(2-trityloxyethoxy)ethoxy]ethoxy]ethoxy]ethyl]propanamide (3.74 g, 8.68 mmol) in ACN/CCl₄/H₂O (80 ml/80 ml/80 ml) was added NaIO₄ (14.9 g, 69.5 mmol) and Ruthenium (III) chloride hydrate (392 mg, 1.74 mmol). The mixture was stirred at 25° C. for 18 hr. The mixture was filtered and dealt with EA (500 ml), washed Na₂S2O₃ aq (500 ml), brine (500 ml) and dried over Na₂SO₄. The organic was concentrated and dealt with tert-butyl alcohol/water (90 ml/30 ml). Sodium chlorite (2.36 g, 26.1 mmol), 2-Methyl-2-butene (15.2 g, 217 mmol) and sodium dihydrogen phosphate (3.13 g, 26.1 mmol) was added to the mixture. The mixture was stirred at 25° C. for 2 hrs. Then the mixture was dealt with EA (500 ml), washed with water (500 ml), brine (500 ml) and dried over Na2SO4. The organic was concentrated and purified by flash (10% MeOH in DCM), to give 7-[2-(6-carboxyhexoxy)-3-[octyl-[2-[2-[2-[2-(2-trityloxyethoxy)ethoxy]ethoxy]ethoxy]ethyl]amino]-3-oxo-propoxy]heptanoic acid (2.58 g, 5.25 mmol, yield 60.5%) as a grey oil.

¹H NMR (500 MHz, CDCl₃) δ 7.38-7.27 (m, 5H), 4.55 (s, 2H), 3.62-3.41 (m, 9H), 2.44-2.29 (m, 4H), 1.59 (dt, J=44.8, 6.8 Hz, 8H), 1.33 (s, 12H).

Step (3)

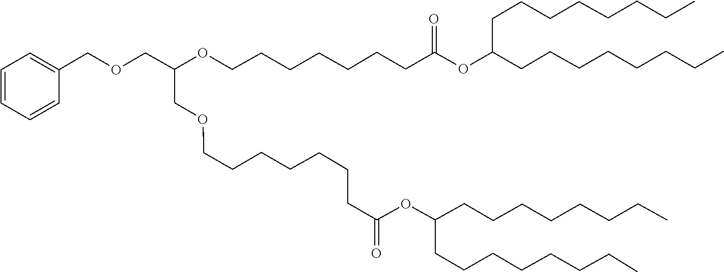

To a solution of 8-[3-benzyloxy-2-(7-carboxyheptoxy)propoxy]octanoic acid (2.58 g, 5.53 mmol) in DCM (20 ml) was added heptadecan-9-ol (3.12 g, 12.2 mmol) was added 3-(ethyliminomethyleneamino)-N,N-dimethyl-propan-1-amine; hydrochloride (3.18 g, 16.6 mmol), N-ethyl-N-isopropyl-propan-2-amine (2.5 g, 19.4 mmol) and N,Ndimethylpyridin-4-amine (338 mg). The mixture was stirred at 25° C. for 18 hrs. The mixture was dealt with EA (300 ml). washed with water (300 ml×2), NaCl sat.aq (300 ml) and dried over Na₂SO₄. The organic was concentrated and purified by flash (5% EA in PE), to give 1-octylnonyl 8-[3-benzyloxy-2-[8-(1-octylnonoxy)-8-oxooctoxy]propoxy]octanoate (2 g, 2.08 mmol) as a colourless oil.

¹H NMR (400 MHz, CDCl₃) δ 7.37-7.27 (m, 5H), 4.90-4.82 (m, 2H), 4.55 (s, 2H), 3.59-3.40 (m, 9H), 2.30-2.24 (m, 4H), 1.52-1.23 (m, 76H), 0.88 (t, J=6.8 Hz, 12H).

Step (4)

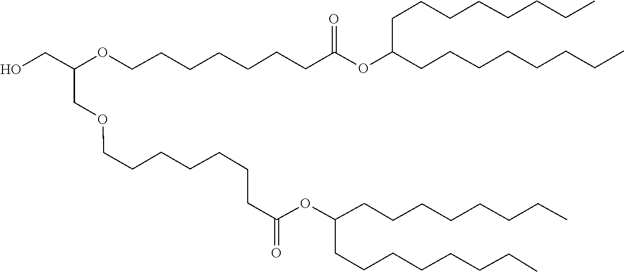

To a solution of 1-octylnonyl 8-[3-benzyloxy-2-[8-(1-octylnonoxy)-8-oxo-octoxy]propoxy]octanoate (2.28 g, 2.42 mmol) in EA (50 ml) was added pd/c (514 mg). The mixture was stirred at 25° C. under H₂ for 18 hrs. Then the mixture was filtered and concentrated, to give 1-octylnonyl 8-[3-hydroxy-2-[8-(1-octylnonoxy)-8-oxooctoxy]propoxy]octanoate (1.93 g, 2.22 mmol, yield 91.7%) as a colorless oil.

¹H NMR (400 MHz, CDCl₃) δ 4.95-4.80 (m, 2H), 3.81-3.38 (m, 9H), 2.27 (t, J=7.5 Hz, 4H), 1.72-1.14 (m, 76H), 0.88 (t, J=6.8 Hz, 12H).

Step (5)

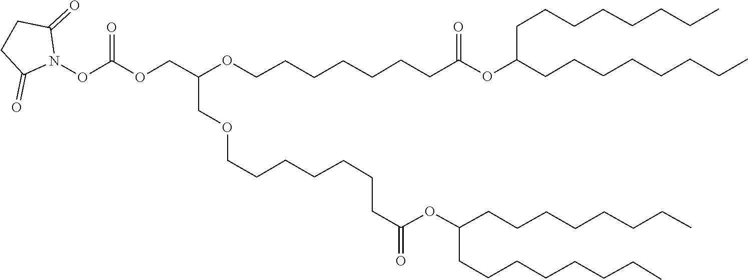

To a solution of 1-octylnonyl 8-[3-hydroxy-2-[8-(1-octylnonoxy)-8-oxo-octoxy]propoxy]octanoate (1.93 g, 2.26 mmol) in DMF (50 ml) was added bis(2,5-dioxopyrrolidin-1-yl) carbonate (2.32 g, 9.05 mmol) and N,N-dimethyl pyridin-4-amine (1.11 g, 9.05 mmol). The mixture was stirred at 25° C. for 18 hr. The mixture was dealt with EA (300 ml), washed with water (300 ml×2), LiCl aq (300 ml), NaCl sat.aq (300 ml) and dried over Na2SO4. The organic was concentrated and purified by flash (10-20% EA in PE), to give 1-octylnonyl 8-[3-(2,5-dioxopyrrolidin-1-yl)oxycarbonyloxy-2-[8-(1-octylnonoxy)-8-oxo-octoxy]propoxy]octanoate (1.66 g, 1.64 mmol, yield 72.3%) as a colorless oil.

Step (6)

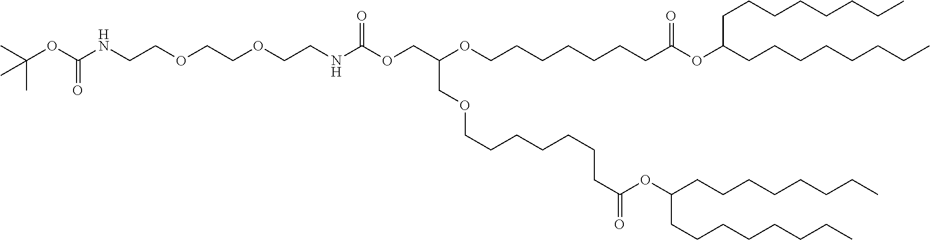 [SMILES: CCCCCCCCC(CCCCCCCC)OC(=O)CCCCCCCOCC(COC(=O)NCCOCCOCCNC(=O)OC(C)(C)C)OCCCCCCCC(=O)OC(CCCCCCCC)CCCCCCCC]

To a solution of tert-butyl N-[2-[2-(2-aminoethoxy)ethoxy]ethyl]carbamate (300 mg, 1.21 mmol) in DCM (15 ml) was added 1-octylnonyl 8-[3-(2,5-dioxopyrrolidin-1-yl)oxycarbonyloxy-2-[8-(1-octylnonoxy)-8-oxooctoxy]propoxy]octanoate (1 g, 1.01 mmol), N,N-diethylethanamine (153 mg, 1.51 mmol) and N,Ndimethylpyridin-4-amine (12 mg). The mixture was stirred at 25° C. for 18 hr. Then the mixture was dealt with EA (50 ml), washed with water (50 ml×2), NaCl sat.aq (50 ml) and dried over Na2SO4. The organic was concentrated and purified by flash (20% EA in PE), to give 1-octylnonyl 8-[3-[2-[2-[2-(tertbutoxycarbonylamino)ethoxy]ethoxy]ethylcarbamoyloxy]-2-[8-(1-octylnonoxy)-8-oxo-octoxy]propoxy]octanoate (763 mg, 0.67 mmol, yield 65.9%) as a colorless oil.

¹H NMR (400 MHz, CDCl₃) δ 5.26 (s, 1H), 5.05 (s, 1H), 4.91-4.80 (m, 2H), 4.14 (ddd, J=16.8, 11.4, 6.5 Hz, 2H), 3.63-3.22 (m, 19H), 2.33-2.20 (m, 4H), 1.60-1.14 (m, 85H), 0.88 (t, J=6.8 Hz, 12H).

Step (7)

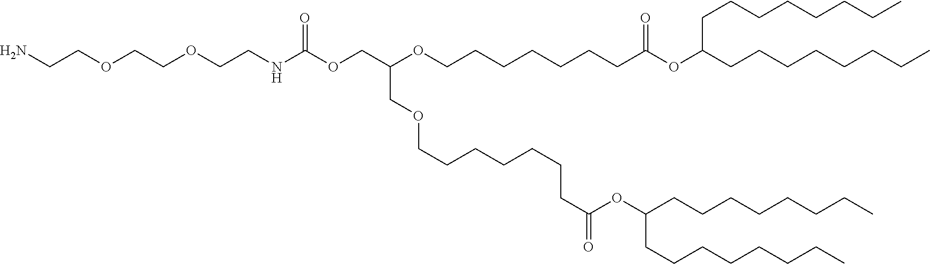 [SMILES: CCCCCCCCC(CCCCCCCC)OC(=O)CCCCCCCOCC(COC(=O)NCCOCCOCCN)OCCCCCCCC(=O)OC(CCCCCCCC)CCCCCCCC]

To a solution of 1-octylnonyl 8-[3-[2-[2-[2-(tert-butoxycarbonylamino)ethoxy]ethoxy]ethylcarbamoyloxy]-2-[8-(1-octylnonoxy)-8-oxo-octoxy]propoxy]octanoate (763 mg, 0.68 mmol) in DCM (10 ml) was added 2,2,2-trifluoroacetic acid (0.5 ml). The mixture was stirred at 25° C. for 2 hr. The mixture was dealt with EA (50 ml), washed with NaHCO₃ aq (50 ml), NaCl sat.aq (50 ml) and dried over Na2SO4. The organic was concentrated and purified by flash (5-10% EA in PE), to give 1-octylnonyl 8-[3-[2-[2-(2-aminoethoxy)ethoxy]ethylcarbamoyloxy]-2-[8-(1-octylnonoxy)-8-oxo-octoxy]propoxy]octanoate (310 mg, 0.3 mmol, yield 43.7%) as a colorless oil.

¹H NMR (500 MHz, CDCl₃) δ 5.73 (s, 1H), 4.86 (p, J=6.3 Hz, 2H), 4.16 (ddd, J=60.4, 11.6, 4.6 Hz, 2H), 3.72-3.29 (m, 19H), 2.98 (s, 2H), 2.27 (t, J=7.5 Hz, 4H), 1.56 (ddd, J=28.8, 18.1, 6.3 Hz, 16H), 1.33-1.24 (m, 60H), 0.88 (t, J=6.9 Hz, 12H).

Step (8)

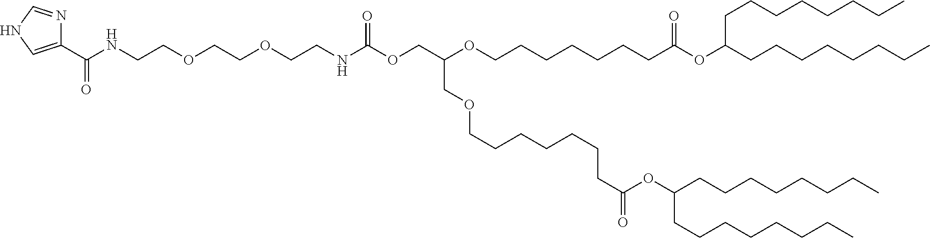 [SMILES: CCCCCCCCC(CCCCCCCC)OC(=O)CCCCCCCOCC(COC(=O)NCCOCCOCCNC(=O)c1c[nH]cn1)OCCCCCCCC(=O)OC(CCCCCCCC)CCCCCCCC]

To a solution of 1-octylnonyl 8-[3-[2-[2-(2-aminoethoxy)ethoxy]ethylcarbamoyloxy]-2-[8-(1-octylnonoxy)-8-oxooctoxy]propoxy]octanoate (310 mg, 0.3 mmol) in DCM (5 ml) was added N-ethyl-N-isopropyl-propan-2-amine (195 mg, 1.51 mmol) and 1H-imidazole-4-carbonyl chloride (158 mg, 1.21 mmol). The mixture was stirred at 25° C. for 18 hrs. The mixture was concentrated and purified by flash (7% MeOH in DCM), to give 1-octylnonyl 8-[3-[2-[2-[2-(1H-imidazole-4-carbonylamino)ethoxy]ethoxy]ethylcarbamoyloxy]-2-[8-(1-octylnonoxy)-8-oxooctoxy]propoxy]octanoate (259 mg, 0.23 mmol, yield 75%) as a colourless oil.

¹H NMR (400 MHz, CDCl₃) δ 7.66 (d, J=4.9 Hz, 2H), 7.52 (s, 1H), 6.02 (s, 1H), 4.91-4.82 (m, 2H), 4.21 (d, J=7.6 Hz, 1H), 4.08 (dd, J=11.6, 5.3 Hz, 1H), 3.70-3.34 (m, 19H), 2.28 (t, J=7.5 Hz, 4H), 1.61-1.13 (m, 76H), 0.87 (t, J=6.8 Hz, 12H)

### Example 26: Synthesis of [(Z)-non-2-enyl] 8-[3-[2-[2-[2-[2-[2-(1H-imidazole-4-carbonylamino)ethoxy]ethoxy]ethoxy]ethoxy]ethyl-octyl-amino]-2-[8-[(Z)-non-2-enoxy]-8-oxooctoxy]-3-oxo-propoxy]octanoate (Compound XXVII)

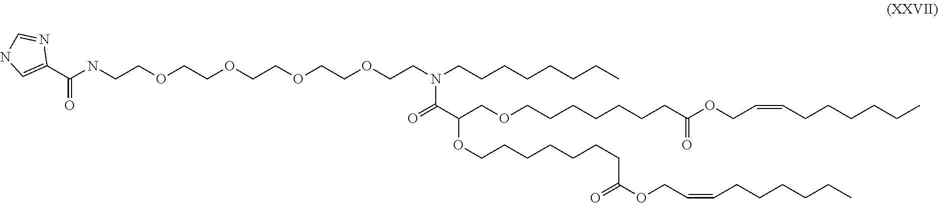

Synthesis of Compound (XXVII)

Step (1)

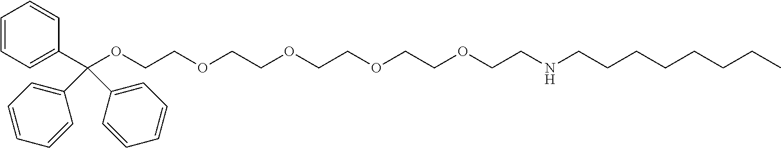 [SMILES: CCCCCCCCNCCOCCOCCOCCOCCOC(c1ccccc1)(c1ccccc1)c1ccccc1]

2-[2-[2-[2-(2-trityloxyethoxy)ethoxy]ethoxy]ethoxy]ethyl methanesulfonate (11 g, 17.7 mmol) was added to octan 1-amine (44 ml), and the mixture was stirred at 80° C. for 18 hr. LCMS showed the SM was consumed and the product was formed. The mixture was dealt with EA (500 ml), washed with water (500 ml×2), NaCl sat.aq (500 ml) and dried over Na₂SO₄. The organic was concentrated and purified by flash (10% MeOH in DCM), to give N-[2-[2-[2-[2-(2-trityloxyethoxy)ethoxy]ethoxy]ethoxy]ethyl]octan-1-amine (9.5 g, 15.7 mmol, yield 88.4%) as a yellow oil.

¹H NMR (500 MHz, CDCl₃) δ 7.49-7.42 (m, 6H), 7.29 (dd, J=10.4, 4.8 Hz, 6H), 7.25-7.20 (m, 3H), 3.71-3.54 (m, 16H), 3.23 (t, J=5.2 Hz, 2H), 2.76 (t, J=5.3 Hz, 2H), 2.61-2.55 (m, 2H), 1.27 (d, J=4.2 Hz, 12H), 0.87 (d, J=7.1 Hz, 3H).

Step (2)

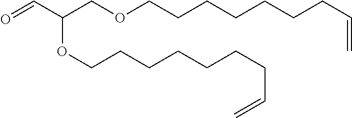 [SMILES: C=CCCCCCCCOCC(C=O)OCCCCCCCC=C]

To a solution of 2,3-bis(non-8-enoxy)propan-1-ol (12.8 g, 37.6 mmol) in DCM (200 ml) was added Dess-Martin Periodinane (23.9 g, 56.4 mmol) at 0° C. for 5 min. Then the mixture was stirred at 25° C. for 2 hr. The mixture was concentrated and dealt with EA (500 ml), washed with Na2S2O3 aq/NaHCO₃ aq (500 ml/500 ml), brine (500 ml) and dried over Na2SO4. The organic was concentrated to give 2,3-bis(non-8-enoxy)propanal (11.7 g, crude, yield 90.1%) as a colourless oil.

¹H NMR (500 MHz, CDCl₃) δ 9.72 (d, J=1.3 Hz, 1H), 5.81 (ddt, J=16.9, 10.2, 6.7 Hz, 2H), 5.05-4.87 (m, 4H), 3.86-3.33 (m, 7H), 2.09-1.97 (m, 4H), 1.61-1.20 (m, 20H)

Step (3)

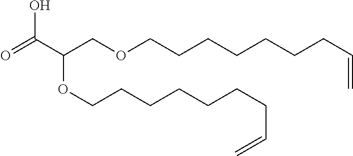 [SMILES: C=CCCCCCCCOCC(OCCCCCCCC=C)C(=O)O]

To a solution of 2,3-bis(non-8-enoxy)propanal (11.7 g, 34.6 mmol) in tert-butyl ALCOHOL/water (180 ml/60 ml) was added sodium chlorite (9.38 g, 104 mmol, 2-Methyl-2-butene (60.6 g, 864 mmol) and sodium dihydrogen phosphate (9.38 g, 104 mmol). The mixture was stirred at 25° C. for 2 hr. Then, the mixture was dealt with EA (500 ml), washed with water (500 ml×2), brine (300 ml) and dried over Na₂SO₄. The organic was concentrated to give 2,3-bis(non-8-enoxy)propanoic acid (9.75 g, 27 mmol, yield 78%) as a colorless oil.

¹H NMR (400 MHz, CDCl₃) δ 5.81 (ddt, J=16.9, 10.2, 6.7 Hz, 2H), 5.07-4.87 (m, 4H), 4.04 (dd, J=5.1, 3.3 Hz, 1H), 3.81-3.44 (m, 6H), 2.04 (dd, J=13.1, 6.5 Hz, 4H), 1.66-1.54 (m, 4H), 1.32 (ddd, J=12.7, 9.1, 5.4 Hz, 16H)

Step (4)

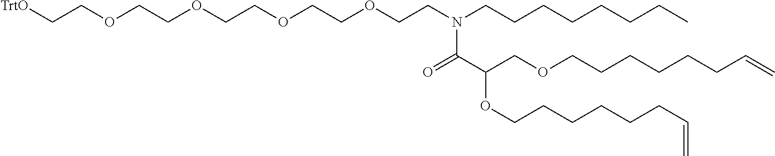

To a solution of N-[2-[2-[2-[2-(2-trityloxyethoxy)ethoxy]ethoxy]ethoxy]ethyl]octan-1-amine (9.52 g, 16.1 mmol) in DCM (150 ml) was added 2,3-bis(non-8-enoxy)propanoic acid (5 g, 14.1 mmol), [dimethylamino(triazolo[4,5-b]pyridin-3-yloxy)methylene]-dimethyl-ammonium; hexafluorophosphate (8.04 g, 21.2 mmol), N,N-diethylethan amine (2.85 g, 28.2 mmol). The mixture was stirred at 25° C. for 18 hr. Then the mixture was dealt with EA (300 ml), washed with water (300 ml×2), NaCl sat.aq (300 ml) and dried over Na2SO4. The organic was concentrated and purified by flash (25% EA in PE), to give 2,3-bis(non-8-enoxy)-N-octyl-N-[2-[2-[2-[2-(2-trityloxyethoxy)ethoxy]ethoxy]ethoxy]ethyl]propanamide (8.22 g, 8.69 mmol, yield 61.5%) as a colorless oil.

¹H NMR (500 MHz, CDCl₃) δ 7.46 (d, J=7.5 Hz, 6H), 7.29 (t, J=7.6 Hz, 6H), 7.22 (t, J=7.3 Hz, 3H), 5.86-5.74 (m, 2H), 5.03-4.89 (m, 4H), 4.43-4.29 (m, 1H), 3.70-3.22 (m, 29H), 2.03 (dd, J=13.5, 6.5 Hz, 4H), 1.57-1.51 (m, 4H), 1.40-1.23 (m, 28H), 0.88 (q, J=6.9 Hz, 3H)

Step (5)

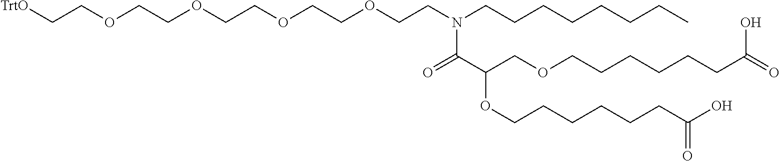

To a solution of 2,3-bis(oct-7-enoxy)-N-octyl-N-[2-[2-[2-[2-(2-trityloxyethoxy)ethoxy]ethoxy]ethoxy]ethyl]propanamide (8.22 g, 9.13 mmol) in ACN/CCl₄/H₂O (80 ml/80 ml/80 ml) was added NaIO4 (15.6 g, 73 mmol) ruthenium (III) chloride hydrate (412 mg, 1.83 mmol). The mixture was stirred at 25° C. for 18 hrs. The mixture was filtered and dealt with EA (500 ml), washed with Na₂S2O₃ aq (300 ml), brine (300 ml) and dried over Na2SO4. The organic was concentrated and dealt with tert-butyl alcohol/water (120 ml/40 ml). sodium chlorite (2.48 g, 27.4 mmol), 2-Methyl-2-butene (16 g, 228 mmol) and sodium dihydrogen phosphate (3.29 g, 27.4 mmol) was added to the mixture. The mixture was stirred at 25° C. for 2 hr. Then the mixture was dealt with EA (500 ml), washed with water (500 ml), brine (300 ml) and dried over Na₂SO₄. The organic was concentrated and purified by flash (10% MeOH in DCM), to give 7-[2-(6-carboxyhexoxy)-3-[octyl-[2-[2-[2-[2-(2-trityloxyethoxy)ethoxy]ethoxy]ethoxy]ethyl]amino]-3-oxo-propoxy]heptanoic acid (5.11 g, 5.19 mmol, yield 56.8%) as a grey oil.

¹H NMR (400 MHz, CDCl₃) δ 7.48 (dd, J=15.2, 13.8 Hz, 6H), 7.29 (dd, J=10.1, 4.8 Hz, 6H), 7.22 (dd, J=8.3, 6.1 Hz, 3H), 4.38 (ddd, J=35.0, 7.3, 4.4 Hz, 1H), 3.80-3.33 (m, 26H), 3.23 (t, J=5.2 Hz, 2H), 2.50-2.24 (m, 4H), 1.56-1.18 (m, 28H), 0.87 (q, J=6.8 Hz, 3H).

Step (6)

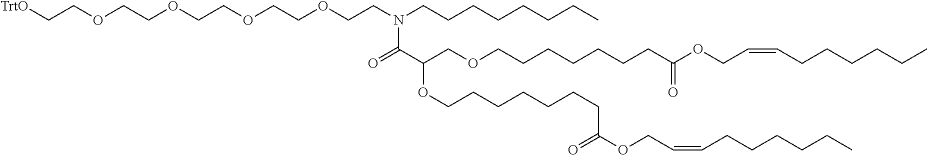

To a solution of 7-[2-(6-carboxyhexoxy)-3-[octyl-[2-[2-[2-[2-(2-trityloxyethoxy)ethoxy]ethoxy]ethoxy]ethyl]amino]-3-oxo-propoxylheptanoic acid (5.1 g, 5.45 mmol) in DCM (80 ml) was added (Z)-non-2-en-1-ol (1.86 mg, 13.1 mmol), EDC HCl (3.13 g, 16.3 mmol), DIEA (2.46 g, 19.1 mmol) and DMAP (333 mg). The mixture was stirred at 25° C. for 18 hr. Then the mixture was concentrated and purified by flash (25% EA in PE), to give [(Z)-non-2-enyl] 8-[2-[8-[(Z)-non-2-enoxy]-8-oxo-octoxy]-3-[octyl-[2-[2-[2-[2-(2-trityloxyethoxy)ethoxy]ethoxy]ethoxy]ethyl]amino]-3-oxopropoxy]octanoate (2.27 g, 1.83 mmol, yield 33.7%) as a colorless oil.

¹H NMR (500 MHz, CDCl₃) δ 7.46 (d, J=7.4 Hz, 6H), 7.29 (t, J=7.5 Hz, 6H), 7.22 (t, J=7.3 Hz, 3H), 5.64 (dd, J=18.3, 7.5 Hz, 2H), 5.55-5.47 (m, 2H), 4.61 (d, J=6.9 Hz, 4H), 4.42-4.28 (m, 1H), 3.69-3.38 (m, 26H), 3.23 (t, J=5.2 Hz, 2H), 2.29 (t, J=7.5 Hz, 4H), 2.09 (q, J=7.3 Hz, 4H), 1.52-1.24 (m, 48H), 0.88 (t, J=6.8 Hz, 9H)

Step (7)

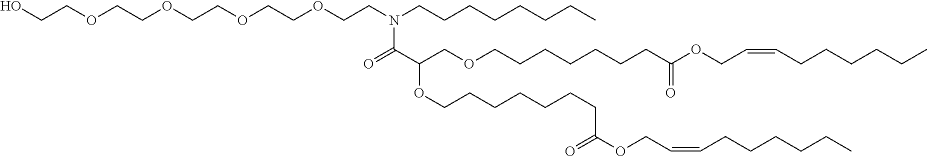 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCO]

To a solution of [(Z)-non-2-enyl] 8-[2-[8-[(Z)-non-2-enoxy]-8-oxo-octoxy]-3-[octyl-[2-[2-[2-[2-(2-trityloxyethoxy)ethoxy]ethoxy]ethoxy]ethyl]amino]-3-oxo-propoxy]octanoate (2.38 g, 1.96 mmol) in THF/MeOH (15 ml/15 ml) was added Toluene-4-sulfonic acid (560 mg, 2.94 mmol). The mixture was stirred at 25° C. for 2 hr. Then the mixture was dealt with EA (150 ml), washed with water (150 ml), brine (150 ml) and dried over Na₂SO₄. The mixture was concentrated and purified by flash (5% MeOH in DCM), to give [(Z)-non-2-enyl] 8-[3-[2-[2-[2-[2-(2-hydroxyethoxy)ethoxy]ethoxy]ethoxy]ethyl-octyl-amino]-2-[8-[(Z)-non-2-enoxy]-8-oxo-octoxy]-3-oxopropoxy]octanoate (1.51 g, 1.52 mmol, yield 77.7%) as a colorless oil.

¹H NMR (400 MHz, CDCl₃) δ 5.69-5.46 (m, 4H), 4.62 (d, J=6.8 Hz, 4H), 4.43-4.29 (m, 1H), 3.74-3.40 (m, 28H), 2.29 (td, J=7.7, 1.5 Hz, 4H), 2.10 (dd, J=14.1, 7.0 Hz, 4H), 1.61-1.21 (m, 48H), 0.88 (td, J=6.7, 4.3 Hz, 9H).

Step (8)

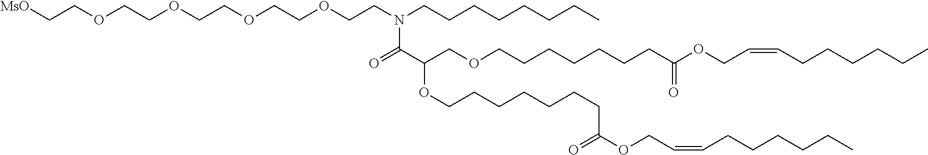 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCOS(C)(=O)=O]

To a solution of [(Z)-non-2-enyl] 8-[3-[2-[2-[2-[2-(2-hydroxyethoxy)ethoxy]ethoxy]ethoxy]ethyl-octyl-amino]-2-[8-[(Z)-non-2-enoxy]-8-oxo-octoxy]-3-oxo-propoxy]octanoate (742 mg) was added N,N-diethylethanamine (155 mg, 1.53 mmol) and methanesulfonyl chloride (131 mg, 1.15 mmol). The mixture was stirred at 25° C. for 2 hr. The mixture was dealt with EA (100 ml), washed with water (100 ml×2), 1N HCl (50 ml×2), NaHCO₃ aq (100 ml), NaCl sat.aq (100 ml) and dried over Na₂SO₄. The organic was concentrated to give [(Z)-non-2-enyl] 8-[3-[2-[2-[2-[2-(2-methylsulfonyloxyethoxy)ethoxy]ethoxy]ethoxy]ethyl-octyl-amino]-2-[8-[(Z)-non-2-enoxy]-8-oxo-octoxy]-3-oxopropoxy]octanoate (753 mg, 0.7 mmol, yield 92%) as a yellow oil.

¹H NMR (400 MHz, CDCl₃) δ 5.77-5.44 (m, 4H), 4.62 (d, J=6.8 Hz, 4H), 4.44-4.29 (m, 3H), 3.79-3.39 (m, 26H), 3.08 (s, 3H), 2.34-2.26 (m, 4H), 2.14-2.02 (m, 4H), 1.62-1.23 (m, 48H), 0.88 (td, J=6.7, 4.2 Hz, 9H).

Step (9)

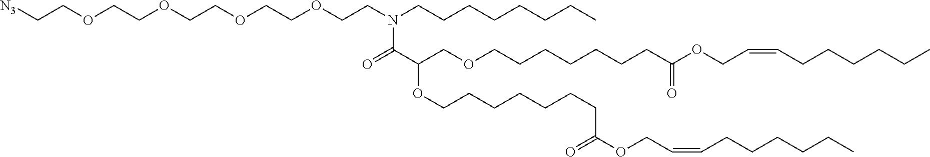 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCN=[N+]=[N-]]

To a solution of [(Z)-non-2-enyl] 8-[3-[2-[2-[2-[2-(2-methylsulfonyloxyethoxy)ethoxy]ethoxy]ethoxy]ethyl-octylamino]-2-[8-[(Z)-non-2-enoxy]-8-oxo-octoxy]-3-oxo-propoxy]octanoate (753 mg, 0.72 mmol) in DMF (10 ml) was added sodium; azide (70 mg, 1.08 mmol). The mixture was stirred at 80° C. for 3 hr. The mixture was dealt with EA (100 ml), washed with water (100 ml), NaCl sat.aq (100 ml) and dried over Na2SO4. The organic was concentrated and purified by flash (30% EA in PE), to give [(Z)-non-2-enyl] 8-[3-[2-[2-[2-[2-(2-azidoethoxy)ethoxy]ethoxy]ethoxy]ethyl-octyl-amino]-2-[8-[(Z)-non-2-enoxy]-8-oxo-octoxy]-3-oxopropoxy]octanoate (364 mg, 0.36 mmol) as a colorless oil.

¹H NMR (400 MHz, CDCl₃) δ 5.58 (dtd, J=17.7, 10.9, 7.1 Hz, 4H), 4.62 (d, J=6.8 Hz, 4H), 4.44-4.30 (m, 1H), 3.72-3.35 (m, 28H), 2.30 (td, J=7.7, 1.6 Hz, 4H), 2.10 (q, J=7.0 Hz, 4H), 1.61-1.22 (m, 48H), 0.88 (td, J=6.8, 4.1 Hz, 9H)

Step (10)

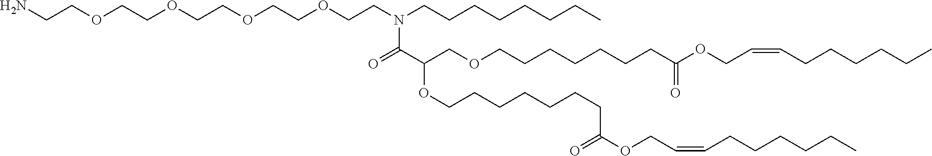 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCN]

To a solution of [(Z)-non-2-enyl] 8-[3-[2-[2-[2-[2-(2-azidoethoxy)ethoxy]ethoxy]ethoxy]ethyl-octyl-amino]-2-[8-[(Z)-non-2-enoxy]-8-oxo-octoxy]-3-oxo-propoxy]octanoate (364 mg, 0.366 mmol) in THF/water (10 ml/0.3 ml) was added triphenylphosphane (288 mg, 1.1 mmol). The mixture was stirred at 25° C. for 18 hr. The mixture was concentrated and purified by flash (5% MeOH in DCM), to give [(Z)-non-2-enyl] 8-[3-[2-[2-[2-[2-(2-aminoethoxy)ethoxy]ethoxy]ethoxy]ethyl-octyl-amino]-2-[8-[(Z)-non-2-enoxy]-8-oxo-octoxy]-3-oxopropoxy]octanoate (300 mg, 0.3 mmol, yield 82.9%) as a colorless oil.

¹H NMR (400 MHz, CDCl₃) δ 5.69-5.59 (m, 2H), 5.56-5.47 (m, 2H), 4.62 (d, J=6.8 Hz, 4H), 4.44-4.30 (m, 1H), 3.69-3.35 (m, 26H), 2.96 (dt, J=44.2, 5.2 Hz, 2H), 2.30 (t, J=7.1 Hz, 4H), 2.12-2.07 (m, 4H), 1.58 (dd, J=16.2, 7.3 Hz, 8H), 1.37-1.22 (m, 40H), 0.88 (dd, J=8.7, 4.9 Hz, 9H)

Step (11)

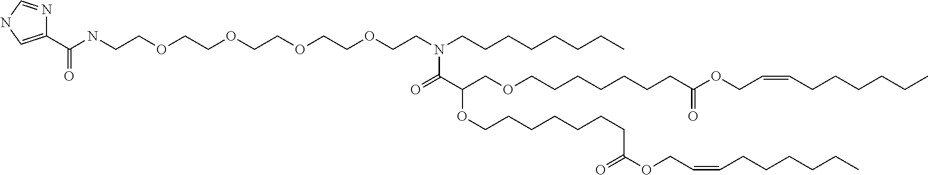 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCNC(=O)c1c[nH]cn1]

To a solution of [(Z)-non-2-enyl] 8-[3-[2-[2-[2-[2-(2-aminoethoxy)ethoxy]ethoxy]ethoxy]ethyl-octyl-amino]-2-[8-[(Z)-non-2-enoxy]-8-oxo-octoxy]-3-oxo-propoxy]octanoate (300 mg, 0.31 mmol) in DCM (5 ml) was added N-ethyl-Nisopropyl-propan-2-amine (240 mg, 1.86 mmol) and 1H-imidazole-4-carbonyl chloride (202 mg, 1.55 mmol). The mixture was stirred at 25° C. for 18 hrs. The mixture was concentrated and purified by flash (5% MeOH in DCM), to give [(Z)-non-2-enyl] 8-[3-[2-[2-[2-[2-[2-(1H-imidazole-4-carbonylamino)ethoxy]ethoxy]ethoxy]ethoxy]ethyl-octylamino]-2-[8-[(Z)-non-2-enoxy]-8-oxo-octoxy]-3-oxo-propoxy]octanoate (213 mg, 0.20 mmol, yield 63.5%) as a yellow oil.

¹H NMR (400 MHz, CDCl₃) δ 7.65 (s, 1H), 7.60 (d, J=13.2 Hz, 2H), 5.64 (dd, J=18.3, 7.5 Hz, 2H), 5.55-5.48 (m, 2H), 4.62 (d, J=6.8 Hz, 4H), 4.32 (s, 1H), 3.74-3.34 (m, 28H), 2.29 (dd, J=10.7, 4.4 Hz, 4H), 2.09 (q, J=7.2 Hz, 4H), 1.59-1.50 (m, 8H), 1.43-1.16 (m, 40H), 0.92-0.84 (m, 9H)

### Example 27: Lipid Nanoparticles (LNPs) Preparation

Preparation of the Organic Phase

Lipids were dissolved in ethanol at molar ratios of 50:10:38.5:1.5 (ionizable lipid:DSPC:cholesterol:PEG2000-PE).

PEG2000-PE can also be replaced by DMG-PEG2000.

DSPC can also be replaced with DOPE (obtained from Avanti Polar Lipids)

Four ionizable cationic lipidic compounds were used to manufacture the LNPs: L319 (DLin-MC3-DMA) and lipidic compounds of formula (III), (IV) and (V).

Preparation of 1.5 mL of Organic Phase for L319-Containing LNPs (LNPs L-319) Formulation

3.9 mg of DSPC (1,2-distearoyl-sn-glycero-3-phosphocholine—Avanti Polar Lipids: 850365), 2 mg of PEG2000-PE (1,2-dimyristoyl-sn-glycero-3-phosphoethanolamine-N-[methoxy(polyethylene glycol)-2000] (ammonium salt)—Avanti Polar Lipids: 880150P) and 7.4 mg of cholesterol (Sigma Aldrich: C3045) were dissolved with 1319 μL of ethanol. Then 167 μL of L319 stock solution (100 mg/mL in ethanol—DLin-MC3-DMA—Maier et al., Molecular Therapy, 2013, 21 (8): 1570-1578) was added to obtain 20 mg/mL of lipid phase solution.

Preparation of 1.7 mL of Organic Phase for Lipidic Compound of Formula (III)-Containing LNPs (LNPs Lip. (III))

3.8 mg of DSPC, 2 mg of PEG2000-PE and 7.2 mg of cholesterol were dissolved in 1477 μL of ethanol. Then 210 μL of lipidic compound of formula (III) stock solution (100 mg/mL in ethanol) was added to obtain 20 mg/mL of lipid phase solution.

Preparation of 1.7 mL of Organic Phase for Lipidic Compound of Formula (IV)-Containing LNPs (LNPs Lip. (IV))

3.8 mg of DSPC, 2 mg of PEG2000-PE and 7.2 mg of cholesterol were dissolved in 1477 μL of ethanol. Then 210 μL of lipidic compound of formula (IV) stock solution (100 mg/mL in ethanol) was added to obtain 20 mg/mL of lipid phase solution.

Preparation of 1.7 mL of Organic Phase for Lipidic Compound of Formula (V)-Containing LNPs (LNPs Lip. (V))

3.9 mg of DSPC, 2 mg of PEG2000-PE and 7.4 mg of cholesterol were dissolved in 1561 μL of ethanol. Then 226 μL of lipidic compound of formula (V) stock solution (100 mg/mL in ethanol) was added to obtain 20 mg/mL of lipid phase solution.

Preparation of the Aqueous Phase

Preparation of 1.8 mL of aqueous phase for L319 LNPs

A non-replicative mRNA encoding A/Netherlands HA (SEQ ID NO: 1) was used. The influenza HA mRNA was produced by in vitro transcription (IVT) as an unmodified mRNA transcript from a linear DNA template generated by PCR, using wild type bases and T7 RNA polymerase (Avci-Adali et al (J. Vis. Exp. (93), e51943, doi:10.3791/51943 (2014) and Kwon et al., Biomaterials 156 (2018) 172e193). The mRNA was 3′ polyadenylated (A120) and 5′ capped (Cap 1).

After Dnase and phosphatase treatment, the mRNA was purified to high degree of purity by silica membrane filtration followed by HPLC. mRNA was packaged as 1 mL aliquots of 2 mg/mL solution in 1 mM Sodium Citrate, pH 6.4.

mRNA concentration to be used in aqueous phase was calculated to obtain a cationic amino group/anionic phosphate group ratio of 6 (N/P=6). This concentration was determined from the cationic lipid concentration assuming 1 μg mRNA corresponds to 0.003 μmol of phosphate. Since 1.5 mL of aqueous solution is needed to make 2 mL of LNPs when using a ratio of aqueous solution to ethanol solution of 3:1 with the NanoAssemblR® (Nanoassemblr Benchtop from Precision Nanosystem; Belliveau et al., Molecular Therapy-Nucleic Acids (2012)), the required mRNA concentration was calculated to be 305 μg/mL.

The mRNA solution was prepared in 50 mM citrate buffer pH 4.0.

Preparation of 1.8 mL of Aqueous Phase for LNPs Lip. (III), LNPs Lip. (IV) and LNPs Lip. (V)

mRNA was prepared as above described and the calculated concentration was 0.265 mg/ml to prepare LNPs with N/P=6.

LNPs Preparation

LNPs were prepared using a NanoAssemblR equipment according to manufacturer recommendations.

The aqueous and organic phases were each loaded in a syringe suitable for NanoAssemblR according to manufacturer recommendations. The flow rate was set up at a ratio: 3:1 and total flow rate: 4 ml/min. The aqueous and lipid phases were then mixed to obtain the LNPs.

LNPs Purification and Harvest

The obtained LNPs were dialyzed against a citrate buffer (50 mM-pH 4.0) to remove residual ethanol and then twice against a PBS buffer (pH 7.4). Each dialysis was carried out at least during 12 hours at 4° C. The LNPs were then filtered through a 0.22 μm filter and stored under nitrogen at +4° C.

### Example 28: Characterization of LNPs

RNA Titration/Encapsulation Rate

The percentage of encapsulated mRNA and concentration of mRNA in LNPs were measured using the Quant-iT Ribogreen RNA reagent kit according to manufacturer recommendations (Invitrogen Detection Technologies) and quantified with a fluorescent microplate reader or a standard spectrophotometer using fluorescein excitation and emission wavelength.

For quantification of non-encapsulated RNA, LNPs were diluted in Tris/EDTA assay buffer (10 mM Tris-HCl, 1 mM EDTA, pH 7.5).

For quantification of total amount of RNA, LNPs were diluted in Tris/EDTA assay buffer (10 mM Tris-HCl, 1 mM EDTA, pH 7.5) containing 0.5% (v/v) Triton X100.

Ribogreen dye (200× diluted) was added to the samples (50/50 mix; sample/Ribogreen reagent), mixed thoroughly and incubated 5 min at room temperature in the dark. Fluorescence was measured on the plate reader.

Lipid Quantification

It was assumed that there was no loss of lipids during the formulation process. The total lipid concentration before dialysis was then assumed to be 5 mg/mL. The final lipid concentration was defined by taking into account the dilution factor occurring during the dialysis step.

Particle Size Distribution, Polydispersity Index Zeta Potential, and Osmolarity,

Zeta potential and particle size distribution of LNPs were measured by using a zeta sizer Nano ZS light scattering instrument (Malvern Instruments) according to manufacturer recommendations. Particle sizes were reported as the Z-average size (harmonic intensity averaged particle diameter) along with the Polydispersity Index (PDI), an indicator of the “broadness” of the particle size distribution. Samples were diluted to 1/100 in phosphate buffered saline (PBS) before measurement. For accurate particle sizing with the Nano ZS, the viscosity of the buffer and the refractive index of the material had to be provided to the equipment software (PBS: v=1.02 cP, RI=1.45). For zeta potential measurements, samples were diluted in water (v=0.8872 cP). Data were analyzed using the Zetasizer Software V 7.11 from Malvern Instruments.

The osmolarity and the pH of formulations were measured by using respectively a micro-sample Osmometer (Fiske Associates model) and a pHmeter (Mettler Toledo) according to the equipment manufacturer instructions.

Results

The characterizations of LNPs L319, LNPs Lip. (III), LNPs Lip. (IV), and LNPs Lip. (V) are described in the TABLE 1 below.

TABLE 1
Characterization of the LNPs

| | | | | | | | Total | |
|---|---|---|---|---|---|---|---|---|
| | | | Zeta | | | Encapsulated | mRNA | |
| Ionizable | Size | | Potential | | Osmolarity | mRNA conc. | conc. | Encapsulat |
| lipid | (d · nm) | PDI | (mV) | pH | mOsmol/kg | (μg/mL) | (μg/mL) | rate (%) |
| L319 | 130 | 0.03 | −13.9 | 7.3 | 301 | 114 | 114 | 100 |
| Lip. (III) | 129 | 0.09 | −16.6 | 7.4 | 287 | 59 | 119 | 46 |
| Lip. (IV) | 226 | 0.02 | −12.3 | 7.4 | 288 | 48 | 103 | 46 |
| Lip. (V) | 133 | 0.1 | −21.6 | 7.4 | 291 | 49 | 105 | 46 |

### Example 29: Immunogenicity of LNPs Comprising Influenza HA mRNA in Mice

The aim of the study was to compare the immunogenicity of different doses of natural, non-replicative mRNA encoding full-length hemagglutinin (HA) of influenza virus strain A/Netherlands/602/2009 (H1N1) formulated in 4 different lipid nanoparticles, LNPs L319, LNPs Lip. (III), LNPs Lip. (IV), and LNPs Lip. (V).

LNPs were prepared as described in Example 27.

Mouse Immunization Procedure

Ten groups of 8 BALBc/ByJ mice (8 weeks old at D0) received two intramuscular (IM) injections, given three weeks apart, of either 1 or 5 μg of mRNA with each the 4 LNPs formulations.

The LNPs L319 was used as benchmark and tested at 4 doses of 0.5 μg, 1, 2.5 and 5 μg of total mRNA to potentially evidence a dose-range effect. The 3 LNPs formulations, LNPs (III), LNPs (IV), and LNPs (V), were tested at 2 doses of 5 and 1 μg of total mRNA.

A positive control group of 8 mice, received 10 μg of monovalent A/California/07/2009 (H1N1) split vaccine Vaxigrip® following the same immunization schedule.

A negative control group of 4 mice was immunized with PBS.

Blood samples were collected on D20 (post-1) and D42 (post-2) for antibody response analysis by hemagglutination inhibition assay (HI).

Determination of Hemagglutination Inhibiting Antibody Titers (HI Titers)

This technique is used to titrate the functional anti-HA antibodies present in the sera of influenza immunized animals, on the basis of the ability of a serum containing specific functional antibodies directed against HA to inhibit the influenza virus hemagglutination activity.

Serial dilutions of virus (clarified allantoic fluid) A/H1N1/California/7/2009 strain were performed in PBS in order to calibrate the viral suspension and to obtain 4 HAU (Hemagglutination Unit) in presence of cRBCs (0.5% in PBS). Calibrated virus (50 μL) was then added to the V shaped well of a 96 well plate on 50 μL of serum serial dilutions (2-fold) in PBS starting from 1:10 and incubated one hour at room temperature.

In order to eliminate serum non-specific inhibitors directed against the HA, each serum was treated with a receptor-destroying enzyme (RDE) (neuraminidase from Vibrio cholerae—Type III—Sigma Aldrich N7885) and with chicken red blood cells (cRBCs). Briefly, 10 mU/mL of RDE was added to each serum. The mix was then incubated 18 h at 37° C., followed by 1 h inactivation at 56° C. To cool, the mixture “serum-RDE” was placed for a time ranging from 30 min to 4 hours at 4° C. The “serum-RDE” mixture was then absorbed on 10% cRBCs in PBS for 30 min, at room temperature, and then centrifuged at 5° C., 10 min at 700 g. The supernatant corresponding to 10-fold diluted serum was collected to perform the HI assay.

Chicken red blood cells (0.5% in PBS) (50 μL) were then added to each well and inhibition of hemagglutination or hemagglutination was visually read after one hour at room temperature.

The titer in HI antibody is the reciprocal of the last dilution giving no hemagglutination. A value of 5 corresponding to half of the initial dilution (1:10) was arbitrary given to all sera determined negative in order to perform statistical analysis.

Statistical Analysis

HI titers were log 10 transformed prior to statistical analyses.

To compare the 4 LNP formulations (LNPs L319, LNPs Lip. (III), LNPs Lip. (IV), and LNPs Lip. (V)), a model of analysis of variances with two factors (LNPs and doses) was applied with a Turkey adjustment for multiple comparisons Statistical analyses were performed on groups with more than 50% of responding mice (more than 4 out of 8). The model's residuals were studied to test the model's validity (normality, extreme individuals, etc.). All analyses were done on SAS v9.4®. A margin of error of 5% was used for effects of the main factors.

Results

The antibody responses elicited against A/California/7/2009 (H1N1) were measured by HI assay in individual sera collected from all animals at D20 and D42.

Mean HI Titers Measured in Mice after One and Two IM Injections of LNPs

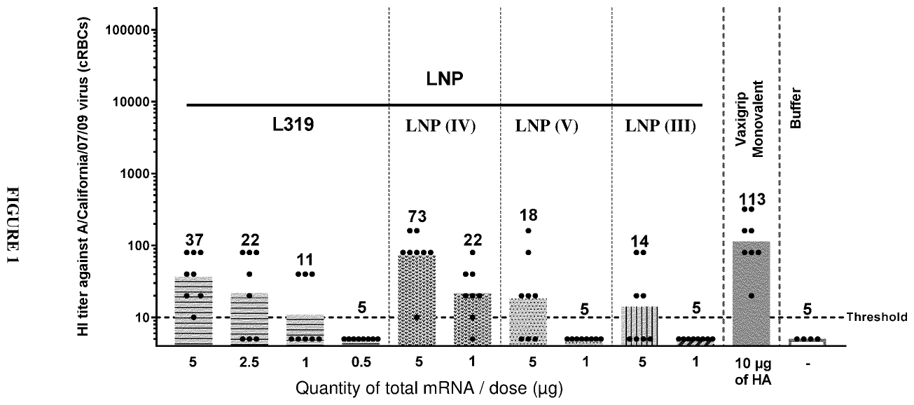

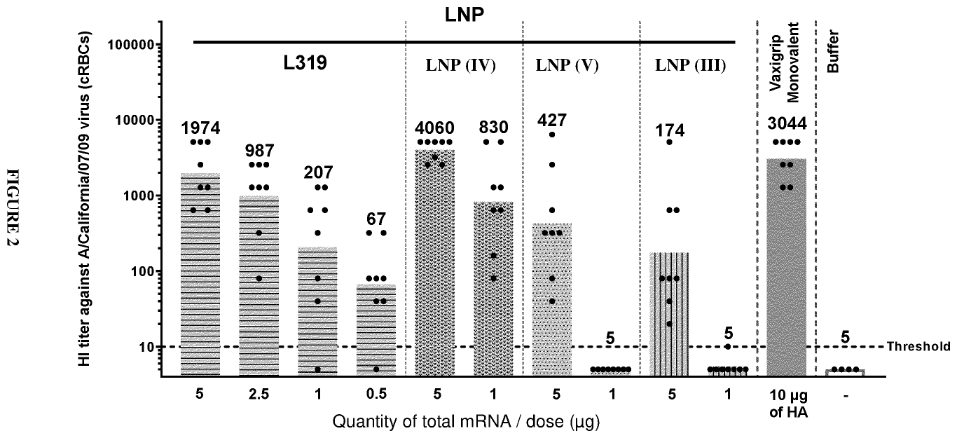

Results analyzed according the total mRNA content per dose showed the following: After a single injection of mRNA (post-1 immunization (D20), doses below 5 μg induced very low or undetectable HI responses, whatever the tested LNP. At the highest dose of 5 μg of total mRNA (D20), part of the animals administered with LNPs Lip. (III) or LNPs Lip. (V) showed detectable HI responses (4 to 5 out of 8), achieving modest mean HI responses (below 20). Conversely, all the animals injected with 5 μg of total mRNA formulated either with LNPs (IV) or L319 LNPs seroconverted with mean HI titers of 73 and 37, respectively (see TABLE 2: Post-one injection titers—FIG. 1).

TABLE 2
Post-one injection titers

| Ionizable | | | HI titer | |
|---|---|---|---|---|
| Lipid | Total | Encaps. | (D20) Mean | Nb |
| LNP | mRNA | mRNA | 95% CI | responders |
| L319 | 5 | 5 | 37 [19; 70] | 8/8 |
| | 2.5 | 2.5 | 22 [7; 65] | 5/8 |
| | 1 | 1 | 11 [4; 27] | 3/8 |
| | 0.5 | 0.5 | 5 [.;.] | 0/8 |
| Lip. (III) | 5 | 2.5 | 14 [5; 39] | 4/8 |
| | 1 | 0.5 | 5 [.;.] | 0/8 |
| Lip. (IV) | 5 | 2.5 | 73 [36; 151] | 8/8 |
| | 1 | 0.5 | 22 [11; 45] | 7/8 |
| Lip. (V) | 5 | 2.5 | 18 [6:55] | 5/8 |
| | 1 | 0.5 | 5 [.;.] | 0/8 |
| Vaxigrip | N.A. | N.A. | 113 [53; 242] | 8/8 |
| (10 μg) | | | | |
| Buffer | N.A. | N.A. | 5 [.;.] | 0/4 |

The second dose of mRNA-LNP boosted the response in all groups (post-2 immunization (D42).

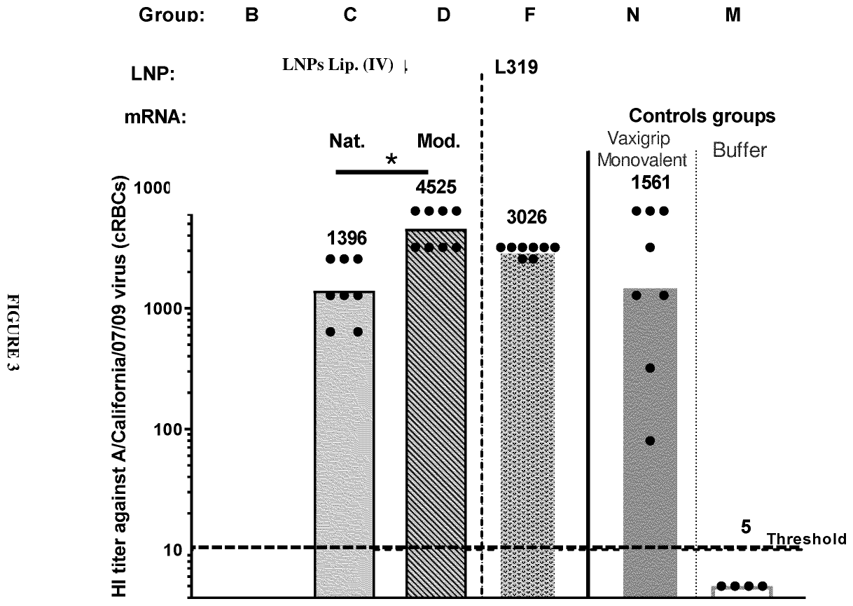

Following two injections with the mRNA/LNPs L319 formulation, a significant dose-effect was evidenced with mean HI titers ranging from 67 to 1974 from 0.5 to 5 μg of total mRNA, respectively (p-value <0.001). Following the booster injection, at the dose of 5 μg of total mRNA, the 4 LNPs, LNPs L319, LNPs Lip. (IV), LNPs Lip. (III) or LNPs Lip. (V), induced HI responses in all mice with mean HI titers of 4060, 1974, 427 and 174, respectively (see FIG. 2 and TABLE 3: Post-two injection titers).

The mean HI response obtained with LNPs (IV) formulation was 2-fold higher than that elicited by LNPs L319 formulation, statistical significance was not demonstrated.

LNPs Lip. (IV) was able to induce significantly higher HI responses than that elicited by the LNPs (III) formulation (respective p-value of 0.007 and 0.001). Moreover, the LNPs Lip. (IV) formulation induced significantly higher HI responses than those elicited by LNPs Lip. (V) (p-value=0.013).

At the dose of 1 μg of total mRNA, responses were found too low to be statistically analyzed except for two groups formulated with either LNPs L319 or LNPs Lip. (IV). Once again, the mean HI titer elicited by LNPs Lip. (IV) tended to be higher to that induced by LNPs L319 (830 versus 207, close to significance, p=0.057).

TABLE 3
Post-two injection titers

| Ionizable | | | HI titer | |
|---|---|---|---|---|
| Lipid | Total | Encaps. | (D42) Mean | Nb |
| LNP | mRNA | mRNA | 95% CI | responders |
| L319 | 5 | 5 | 1974 [928; 4199] | 8/8 |
| | 2.5 | 2.5 | 987 [354; 2749] | 8/8 |
| | 1 | 1 | 207 [40; 1067] | 7/8 |
| | 0.5 | 0.5 | 67 [22; 203] | 7/8 |
| Lip. (III) | 5 | 2.5 | 174 [38; 807] | 8/8 |
| | 1 | 0.5 | 5 [4; 7] | 1/8 |
| Lip. (IV) | 5 | 2.5 | 4060 [3087; 5339] | 8/8 |
| | 1 | 0.5 | 830 [241; 2858] | 8/8 |
| Lip. (V) | 5 | 2.5 | 427 [106:1714] | 8/8 |
| | 1 | 0.5 | 5 [.;.] | 0/8 |
| Vaxigrip | N.A. | N.A. | 3044 [1822; 5088] | 8/8 |
| (10 μg) | | | | |
| Buffer | N.A. | N.A. | 5 [.;.] | 0/4 |

Reactogenicity Observed in Mice Injected with LNPs

After one immunization, no clinical signs were observed in any of the animals. After two immunizations, a slight inflammation was observed in 8 mice which received 5 μg of total mRNA in LNP (IV) and in 4 mice which received 5 μg of total mRNA in LNP Lip. (III) at the injection site on D22, D23 or D24, i.e. 1 to 3 days following the booster injection.

### Example 30: Evaluation of LNPs Lip. (IV) and Influenza HA mRNA in BALBc/ByJ Mice

Purposes and Design of the Study

The aims of the study were:
To test LNPs Lip. (IV) with a non-replicative mRNA encoding full-length hemagglutinin (HA) of influenza virus strain A/Netherlands/602/2009 (H1N1) either in: i) all-natural bases mRNA (Nat.) or ii) with 1-methyl pseudo-uridine (instead of uridine) modified mRNA (Mod.).
To evaluate the replacement of DSPC with DOPE in the LNP composition in terms of immunogenicity and injection site reactogenicity assessed in terms of hind leg swelling after each injection.
The test the stability and immunogenicity of LNPs-Lip. (IV)/HA mRNA after one year of storage at 4° C. as a liquid formulation in PBS pH 7.4. A group of mice was injected with the LNPs batch of LNPs Lip. (IV)/HA mRNA with 2.5 μg of mRNA. This testing was performed as a “potency test” to assess the long stability of LNPs Lip. (IV) when stored as a liquid formulation at 4° C.

TABLE 4
Groups of mice tested

| | | Quantity | | Vol. |
|---|---|---|---|---|
| | | mRNA | | (μl)/ |
| Group | LNPs/mRNA | (μg) | N/P | Route |
| B (8 mice) | (IV)/DSPC-mRNA Nat. | 5 | 6 | 50/IM |
| C (8 mice) | (IV)/DSPC-mRNA Nat. | 5 | 6 | 50/IM |
| D (8 mice) | (IV)/DSPC-mRNA Mod. | 5 | 6 | 50/IM |
| E (8 mice) | (IV)/DOPE-mRNA Nat. | 5 | 6 | 50/IM |
| F (8 mice) | L319/DSPC-mRNA Nat. | 5 | 6 | 50/IM |
| M (4 mice) | Buffer | | | 50/IM |
| N (8 mice) | VaxigripTM Monovalent | (10 μg of | | 50/IM |
| | H1N1 | HA) | | |

Preparation of the LNPs

The LNPs were prepared as described in Example 27 with varying either the cationic lipid ((IV) or L319), the neutral lipid (DSPC or DOPE) or the mRNA (all natural bases mRNA or uridine-modified bases mRNA). The steroid alcohol (cholesterol) and the PEGylated lipid (PEG2000-PE) were the same.

The amounts of mRNA and cationic lipid were adjusted to reach the indicated cationic (N)/anionic (P) charges ratio.

The molar ratio of the LNPs-Lip. (IV)/DSPC was (IV):DSPC:PEG200-PE:Cholesterol=50:10:1.5:38.5.

The molar ratio of the LNPs-Lip. (IV)/DOPE was (IV):DOPE:PEG200-PE:Cholesterol=50:10:1.5:38.5.

The molar ratio of the LNPs-L319 was L319:DSPC:PEG200-PE:Cholesterol=50:10:1.5:38.5.

For stability testing a batch of LNPs-(IV)/HA mRNA was stored for one year at 4° C. as a liquid formulation in PBS pH 7.4, and was tested at t=0, t=6 months and t=lyear.

Mouse Immunization Procedure and Readouts

Immunization procedure and readouts were as follows.

In brief, groups of 8 BALBc/ByJ mice (8-week-old at D0) received two intramuscular (IM) injections, given three weeks apart (D0 and D21) of the indicated LNPs. A negative control group of 4 mice received buffer and a positive control group of 8 mice received 10 μg of monovalent A/California/07/2009 (H1N1) split vaccine Vaxigrip® following the same immunization schedule.

Blood samples were collected on D42 (3 weeks post-dose 2) for antibody response analysis by hemagglutination inhibition assay (HI) as described in Example 29.

Local reactogenicity was assessed through by observation of injection site after each injection. Swellings were observed in part of the animals and classified from no swelling, low swelling, medium low swelling, medium high swelling to high swelling.

Results

Comparison of the Different mRNA: HI Responses & Local Reactogenicity

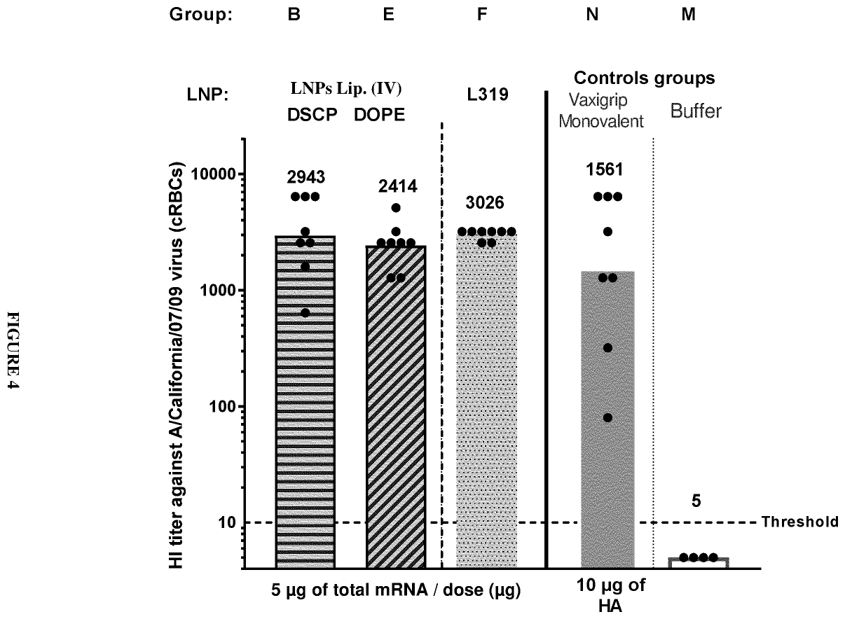

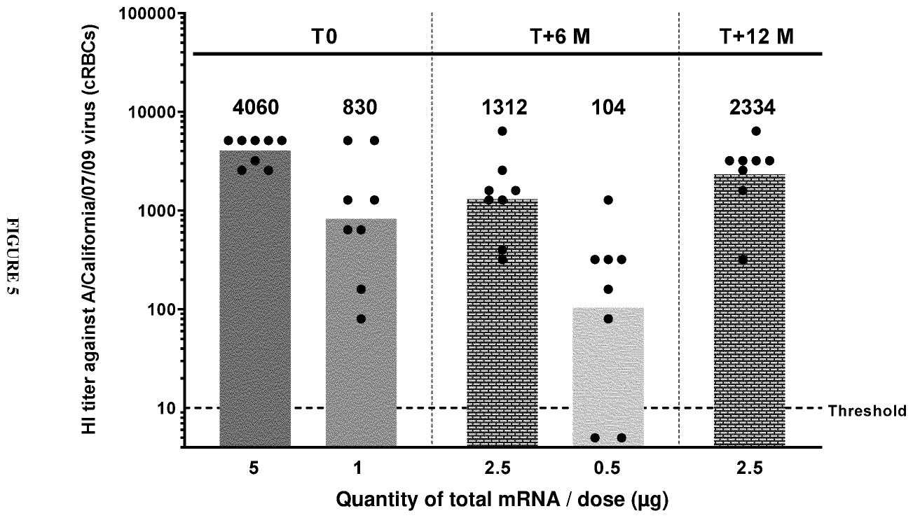

The obtained HI results are shown on FIG. 3 and FIG. 4.

mRNA whether all-natural or 1-methyl pseudo-uridine-modified was as effective at inducing HI responses when formulated in LNPs Lip. (IV)/DSPC (FIG. 3).

The 1-methyl pseudo-uridine-modified mRNA was more effective at inducing HI responses than natural mRNA when formulated in LNPs Lip. (IV)/DSPC (p value=0.044) (FIG. 3).

Overall, LNPs Lip. (IV)/DSPC induced more local swelling than LNPs L319/DSPC but swelling decreased 3 days following each injection (see Groups B, C, D vs F—TABLE 5).

TABLE 5
Local reactogenicity

| | | Site observations post-1 | | | Site observations post-2 | | |
|---|---|---|---|---|---|---|---|
| | | (swelling) | | | (swelling) | | |
| | Mouse | Day | Day | Day | Day | Day | Day |
| Group | # | 1 | 2 | 3 | 1 | 2 | 3 |
| B | 1 | ++ | + | | ++ | ++ | |
| | 2 | + | + | | ++ | ++ | |
| | 3 | + | + | | ++ | ++ | |
| | 4 | + | + | | ++ | ++ | |
| | 5 | + | | | + | + | + |
| | 6 | | + | | + | + | + |
| | 7 | | + | | | | |
| | 8 | + | + | | + | + | + |
| C | 1 | | | | + | + | + |
| | 2 | | + | +/− | + | + | |
| | 3 | | | | + | + | |
| | 4 | | | +/− | + | + | |
| | 5 | | + | | ++ | + | |
| | 6 | | + | | ++ | +/− | |
| | 7 | | | | ++ | +/− | |
| | 8 | | | | ++ | ++ | + |
| D | 1 | ++ | ++ | + | + | + | + |
| | 2 | | | | ++ | +++ | +++ |
| | 3 | ++ | ++ | + | +++ | ++ | ++ |
| | 4 | ++ | ++ | + | +++ | + | + |
| | 5 | ++ | ++ | | + | + | |
| | 6 | ++ | ++ | | | | |
| | 7 | | ++ | | | | |
| | 8 | ++ | ++ | | + | + | + |
| F | 1 | | | | | | |
| | 2 | | | | | +/− | +/− |
| | 3 | | | | | | |
| | 4 | | | | +/− | +/− | +/− |
| | 5 | | | | | | |
| | 6 | | | | | | |
| | 7 | | | | | | |
| | 8 | | | | | | |

Blank cell: no swelling
+/−: low swelling
+: medium low swelling
++: medium high swelling
+++: high swelling

Replacement of DSPC with DOPE in LNPs Composition: HI Responses & Local Reactogenicity

Replacement of DSPC with DOPE had no significant impact on the HI responses induced by LNPs Lip. (IV) or LNPs L319 (see results shown on FIG. 4).

There was a marked reduction of local swelling when DSPC was replaced with DOPE in Lip. (IV) LNPs (see Group E—TABLE 6).

The Lip. (IV)/DOPE/Chol/PEG-PE (50/10:38.5/1.5) LNP formulation seems to ideally combine effectiveness in terms of immunogenicity with tolerability at the injection site.

TABLE 6
Local reactogenicity

| | | Site observations post-1 | | | Site observations post-2 | | |
|---|---|---|---|---|---|---|---|
| | | (swelling) | | | (swelling) | | |
| | Mouse | Day | Day | Day | Day | Day | Day |
| Group | # | 1 | 2 | 3 | 1 | 2 | 3 |
| B | 1 | ++ | + | | ++ | ++ | |
| | 2 | + | + | | ++ | ++ | |
| | 3 | + | + | | ++ | ++ | |
| | 4 | + | + | | ++ | ++ | |
| | 5 | + | | | + | + | + |
| | 6 | | + | | + | + | + |
| | 7 | | + | | | | |
| | 8 | + | + | | + | + | + |
| E | 1 | | | | | | |
| | 2 | | | | | | |
| | 3 | | | | | | |
| | 4 | | | | | | |
| | 5 | | | | | | |
| | 6 | | | | | | |
| | 7 | | | | | | |
| | 8 | | | | | +/− | |
| F | 1 | | | | | | |
| | 2 | | | | | +/− | +/− |
| | 3 | | | | | | |
| | 4 | | | | +/− | +/− | +/− |
| | 5 | | | | | | |
| | 6 | | | | | | |
| | 7 | | | | | | |
| | 8 | | | | | | |

Blank cell: no swelling
+/−: low swelling
+: medium low swelling
++: medium high swelling
+++: high swelling

Stability

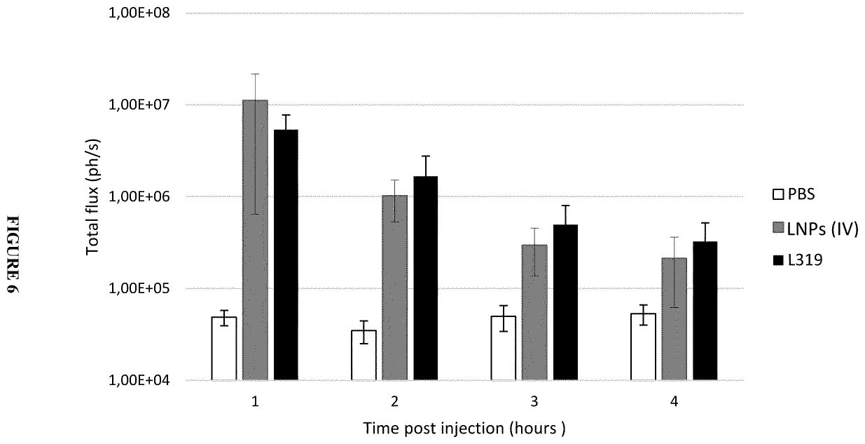

The same preparations of LNPs Lip. (IV)/DSPC at different mRNA concentration were tested in 3 independent experiments performed at t=0, t=6 months and t=1 year. Results are shown on FIG. 5.

Because of the different dose level tested throughout the experiment no statistical conclusion can be drawn. However, from the trends of results presented on FIG. 5, one can conclude that LNPs Lip. (IV)/DSPC display a remarkable stability upon storage as a liquid formulation at 4° C. since the immunogenicity did not vary for a time ranging from 6 months to 12 months of storage.

### Example 31: LNP Delivered Luciferase mRNA Imaging

The purpose of the study was to determine the liver transduction efficiency and tissue biodistribution of different LNP formulations in normal mice.

Materials and Methods

LNPs Reagents

1,3-dioleoyl-sn-glycero-3-phosphocholine (DOPC), 1,2-distearoyl-sn-glycero-3-phosphocholine (DSPC), 1,2-dimyristoyl-rac-glycero-3-methoxypolyethylene glycol-2000 (DMG-PEG) were purchased from Avanti Polar Lipids (Alabaster, Albany). 30-Hydroxy-5-cholestene, 5-Cholesten-30-ol (Cholesterol) and Acrodisc Syringe Filters with Supor Membrane, Sterile-0.2 μm, 13 mm, were purchased Sigma Aldrich (St. Louis, Missouri). Coatsome SS-OP (SS-OP) was purchased from NOF America Corporation (White Plains, NY headquartered in Japan). Nuclease-free water, 10×PBS Buffer pH 7.4, Sodium Citrate, Dihydrate, Citric Acid, Sodium Chloride, Sucrose, Ethyl Alcohol, BD Vacutainer General Use Syringe Needles (BD Blunt Fill Needle 18G), BD Slip Tip Sterile Syringes (3 ml & 1 mL), Amicon Ultra Centifugal Filter Units, DNA-free microcentrifuge tubes (1.5 mL), Invitrogen RNase-free Microfuge Tubes (0.5 mL), Invitrogen Conical Tubes (15 mL) (DNase-RNase-free), Fisher Brand Semi-Micro Cuvette, Quant-iT™ RiboGreen® RNA Assay Kit and RnaseZap™ were purchased from Thermo Fisher Scientific (Waltham, Massachusetts). mRNA encoding luciferase was purchased from TriLink™ Biotechnologies (mRNA-Luc—Ref.: L-7602).

All LNPs were manufactured using the NanoAssemblr Benchtop from Precision Nanosystems (British Columbia, Canada).

LNPs Manufacturing

The following buffer systems were used:
Phosphate Buffer Saline (PBS): 8 mM Na₂HPO₄, and 2 mM KH₂PO₄, 137 mM NaCl, 2.7 mM KCl, pH 7.4
Citrate Buffer-10 mM: 5 mM Sodium citrate, 5 mM citric acid, 150 mM Sodium Chloride, pH 4.5
Citrate Buffer-50 mM: 25 mM Sodium citrate, 25 mM citric acid, pH 4.0
Citrate-Sucrose Buffer: 5 mM Sodium citrate, 5 mM citric acid, 8% (w/v) sucrose, pH 6.3

The following LNPs were prepared according to the procedure described in Example 27.

Lipid Phase

LNPs SS-OP: SS-OP/DOPC/Chol=52.5/7.5/40, DMG-PEG2000 1.5 mol %

533 μL of mixed lipids solution were prepared (total lipid concentration 9 mM) in a 1.5 mL conical tube. (total lipid=SS lipid+DOPC+Chol) by mixing the following solutions:

TABLE 7
LNP mix working solution for LNP SS-OP

| | Concentration in | Volume |
|---|---|---|
| Lipid | Ethanol (mM) | (μL) |
| SS-OP | 8.52 | 222 |
| DOPC | 10 | 27 |
| Cholesterol | 20 | 72 |
| DMG-PEG 2000 | 1 | 54 |
| Ethanol | — | 158 |
| Total | 4.58 | 533 |

LNPs Lip. (IV): lipidic compound (IV)/DSPC/Chol/DMG-PEG=50/10/38.5/1.5

533 μL of mixed lipids solution (total lipid concentration 5 mM) was prepared in a 1.5 mL conical tube by mixing the following solutions:

TABLE 8
LNP mix working solution for LNPs Lip. (IV)

| | Concentration in | Volume |
|---|---|---|
| Lipid | Ethanol (mM) | (μL) |
| Lipidic comp'd (IV) | 6.13 | 127 |
| DSPC | 31.6 | 8 |
| Cholesterol | 20 | 51 |
| DMG-PEG 2000 | 1 | 40 |
| Ethanol | — | 217 |
| Total | 5 | 533 |

Aqueous Nucleic Acid Containing Phase

The following mRNA containing aqueous solutions were used for manufacturing the LNPs.

TABLE 9
mRNA working solution

| | | Target | Volume | | Volume of | |
|---|---|---|---|---|---|---|
| | | mRNA | from | Citrate | citrate | Total |
| | | concentration | stock | buffer | buffer | Volume |
| | N/P | (μg/mL) | (μL) | type | (μL) | (μL) |
| LNP | 6 | 90 | 43.5 | Citrate | 1306.5 | 1350 |
| SSOP | | | | Buffer- | | |
| | | | | 10 mM | | |
| LNP | 6 | 45 | 21.8 | Citrate | 1328.2 | 1350 |
| Lip. | | | | Buffer- | | |
| (IV) | | | | 50 mM | | |

Procedure

The LNPs were manufactured with the NanoAssemblr™ according to manufacturer's recommendations the following parameters:

TABLE 10
NanoAssemblr ™ parameters

| | |
|---|---|
| Volume | 1.37 mL |
| Flow Rate Ratio | 3:1 |
| Total Flow Rate | 4 mL/min |
| Left Syringe Size | 3 mL |
| Right Syringe Size | 1 mL |
| Start Waste Volume | 0.25 mL |
| End Waste Volume | 0.05 mL |

The LNPs were formulated to encapsulate 4.25 μg of mRNA (final content)

LNPs Harvest and Purification

The harvested LNPs SS-OP were three-time washed by dilution in PBS and filtration (100 KD MWCO). The resuspended LNPs were then filtered on 0.2 μm filter.

The harvested LNPs Lip. (IV) were three-time washed by dilution in PBS, pH 7.4 and filtration (100 KD MWCO). The resuspended LNPs were then filtered on 0.2 μm filter.

Animals Study

2 groups, each of 4 of SKH1 hairless mice, female 10-12 weeks old, were tested with LNPs SS-OP and LNPs Lip. (IV) encapsulating a luciferase encoding mRNA. A last group of 2 mice was injected with PBS and used as control.

The LNPs were formulated to encapsulate 4.25 μg of mRNA (final content).

TABLE 11
Design of experiment

| | | Animal | |
|---|---|---|---|
| Group | LNPs | per group | Timepoints |
| A | LNPs (IV) | 4 | Radiance measured 24 h |
| | | | post-injection |
| B | LNPs SS-OP | 4 | Radiance measured 24 h |
| | | | post-injection |
| C | PBS, IV | 2 | Radiance measured 24 h |
| | | | post-injection |

Radiance of liver, spleen, kidney, lung and hear were taken at 24 h, ex vivo according to the following procedure.

Ex vivo bioluminescence of isolated organs was performed immediately after euthanasia of the animals by CO₂, around 15 min after subcutaneous injection of luciferin (200 mg/kg). Dissected organs were placed on a black sheet and imaged with IVIS Spectrum CT (PerkinElmer, Hopkinton, MA). To quantify bioluminescence emission signal, identical regions of interest (ROI) were positioned to encircle each organ region, the imaging signal was quantitated as average radiance (photons/s/cm2/steradian).

Results

Ex vivo organ analysis was performed twenty-four hours post injection. The organ data is summarized in Table 12.

Significant transduction and luciferase expression were observed in the spleen for LNPs (IV) (10 to 50-folds higher) compared to other organs (spleen, kidney, heart and lungs). The specificity of LNPs (IV) targeting spleen compared to liver was 10-folds higher than the LNPs SS-OP. The LNPs SS-OP although demonstrated higher absolute expression in the spleen compared to LNPs (IV), the relative expression in the liver was significantly higher showing that the LNPs SS-OP formulation is better suited for targeting liver than spleen. The findings thus support the claim that LNPs (IV) can be effectively used for delivery of the nucleic acid specifically to the spleen via intravenous administration.

TABLE 12
Radiance per organ

| | Liver | | Spleen | | Kidney | |
|---|---|---|---|---|---|---|
| GROUP | Average | SD | Average | SD | Average | SD |
| A | 2.32E+04 | 5.73E+03 | 2.12E+05 | 9.77E+04 | 2.08E+03 | 5.65E+02 |
| B | 1.29E+07 | 1.34E+06 | 1.23E+06 | 1.52E+05 | 8.01E+03 | 4.30E+02 |
| C | 1.11E+03 | 1.02E+03 | 6.05E+02 | 4.52E+01 | 5.79E+02 | 1.50E+02 |

| | Lung | | Heart | |
|---|---|---|---|---|
| GROUP | Average | SD | Average | SD |
| A | 1.05E+04 | 6.98E+03 | 8.45E+02 | 2.77E+02 |
| B | 1.36E+04 | 1.29E+04 | 1.95E+04 | 9.25E+03 |
| C | 4.62E+02 | 4.72E+02 | 4.94E+02 | 4.36E+01 |

### Example 32: BioImaging Following IM Injection

The purpose of the study was to determine in vivo protein expression after injection of LNP formulations in normal mice.

Materials and Methods:

Ten weeks-old female BALB/c ByJ mice were obtained from Charles River lab (Les Oncins, Saint-Germain-Nuelles, 69210, France). At T0, animals were injected by intramuscular (IM) route with 50 μl of LNPs 319 or LNPs Lip. (IV) (prepared as described in Example 27) containing 5 μg of mRNA encoding luciferase (mRNA-Luc—Ref.: L-7602 TriLink™ Biotechnologies). Luciferin potassium salt (D-luciferin, K+ salt Fluoprobes, Interchim) diluted in PBS was injected through intraperitoneal (i.p) route at 3 mg per mouse which is in large excess relative to the luciferase amount.

Optical imaging was performed using the IVIS Spectrum CT device (PerkinElmer Inc., Paris, France). Bioluminescence acquisition was initiated 15 min after the injection of the substrate. The luminescence level was evaluated by an ROI applied to the injection site zone (Living Image software, PerkinElmer Inc., Paris, France). Results are expressed as total flux (ph/s) in function of time (hours) post the injection of LNPs/mRNA-Luc.

Results

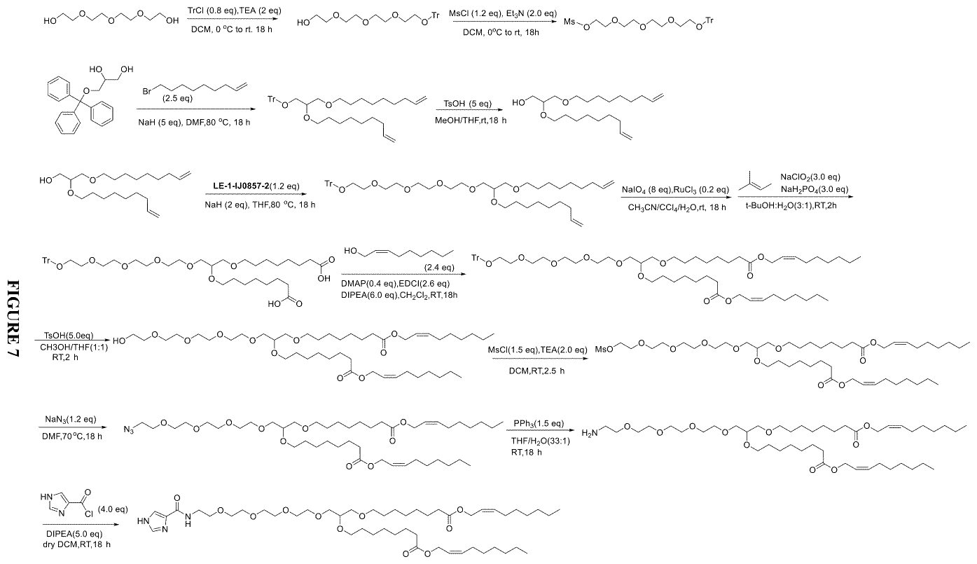

The results are presented on FIG. 6. The protein expression (luciferase) was measured with bioluminescence imaging at 6 h, 24 h, 48 h and 78 h post LNPs/mRNA-Luc injected in the quadriceps muscle. Mice were injected by i.p. route with 3 mg of Luciferin and bioluminescence signal acquisition was performed with IVIS CT camera. LNPs Lip. (IV) (n=5) was tested in comparison with L319 LNP (n=3); a positive control. PBS (n=2) was the negative control.

Results are expressed as total flux (ph/s) in function of time (hours). Mean±SD The protein expression was observed in the two groups as compared to the PBS (white box) with an expression peak at 6 h post IM injection of LNPs/mRNA-Luc. The bioluminescence signal decreased over time with a similar profile for all LNP. LNPs (IV) (grey box) demonstrated a similar bioluminescence signal pattern than the positive control LNP L319 (black box).

### Example 33: Formulation of hEPO mRNA in LNPs Comprising Ionizable Cationic Lipids of the Invention

CleanCap® EPO mRNA (5moU), a non-replicative, highly purified, mRNA encoding the human erythropoietin was obtained from TriLink Biotechnologies, San Diego, CA (catalogue number L7209; hEPO mRNA). This mRNA is capped using CleanCap, TriLink's proprietary co-transcriptional capping method, which results in the naturally occurring Cap 1 structure with high capping efficiency. It is polyadenylated, modified with 5-methoxyuridine and optimized for mammalian systems. It mimics a fully processed mature mRNA.

LNPs comprising ionizable lipid of formula IV (DOG-IM4) or a given analogue of compounds (VIII), (IX), (XII), (XVI), (XIX) or (XXII), DSPC, Chol and DMG-PEG2000 at a molar ratio of 50:10:3.5:1.5, and hEPO mRNA at a N/P charge ratio=6, were prepared as described in Example 27 by using the NanoassemblR. An Aqueous/Ethanolic phase volume ratio of 3/1 and at total flow rate of 4 mL/min was used. LNPs were prepared at a concentration of 60 μg of hEPO mRNA/mL in PBS 1×.

### Example 34: Intramuscular Injection of Mice with LNPs Lip. (IV)/DSPC, or a Given Analogue, Containing hEPO mRNA and Detection of hEPO Expression in the Serum

Animals

Female Balb/c ByJ mice (7 weeks of age at receipt) were purchased from Charles River Laboratories (Saint-Germain-Nuelles, France) and housed for one-week acclimation before starting the study. Mice were identified individually by fur coloration. Experiments were approved by Sanofi Pasteur's animal ethics committee and followed European guidelines for standards of animal care.

Study Schedule

Four 8-week-old mice per group were injected on D0 via intramuscular route in the quadriceps with 1 μg-dose of hEPO mRNA formulated in LNPs Lip. (IV)/DSPC under a final volume of 50 μL. As negative control, 4 mice received the same volume of PBS (for accelerated stability study and lipid screening) or Citrate Buffer (lipid screening) Blood samples were collected 6 hours post-injection to measure the expression of hEPO in serum using a specific ELISA assay.

Blood Samples

Blood samples were collected 6 hours post-injection by carotid section under deep anesthesia with Imalgene/Rompun (1.6 mg of Ketamine/0.32 mg of Xylazine) in serum-separation tubes (BD Vacutainer #BD367957). Sera were aliquoted and stored at −20° C. until hEPO determination.

hEPO Determination in Mouse Serum

hEPO expression in mouse sera was assessed using human Erythropoietin Quantikine IVD ELISA kit (R&D Systems #DEP00). The ELISA was performed following supplier's instructions. Briefly, sera were added in pre-coated plates and incubated for one hour at room temperature under orbital shaking. After sera removal, Erythropoietin conjugate was added for one hour at room temperature under orbital shaking. Plates were washed and Substrate solution was added for 20-25 minutes at room temperature before stopping the reaction with Stop solution. Absorbance at 450 nm with 650 nm signal subtraction was determined in a microplate reader. Data were analyzed using SoftmaxPro software and expressed in log 10 of the concentration of hEPO measured in mouse sera in μg/mL.

TABLE 13
hEPO expression in mice having received intramuscular
injection of LNPs Lip. (IV)/DSPC, or a
given analogue, containing hEPO mRNA

| LNPs WITH | EPO secretion IM |
|---|---|
| COMPOUND No. | (Log10 pg/ml) |
| (IV) | 2.3 |
| (VIII) | 2.6 |
| (IX) | 2.5 |
| (XII) | 2.3 |
| (XVI) | 2.5 |
| (XIX) | 3.3 |
| (XXII) | 2 |

### Example 35: Stability of LNPs Comprising Lipid of Formula IV (DOG-IM4) and hEPO mRNA

A stability study was performed in order to confirm the long-term stability observed in Example 30 with the LNPs Lip. (IV)/DSPC.

The LNPs Lip. (IV)/DSPC formulated with hEPO mRNA as described above, were first diluted with PBS 1× to a concentration of 20 μg/ml. The product stability was then studied by storing the LNP suspension at 5° C., 25° C. and 37° C. into temperature-controlled incubators for up to 18 weeks. The following physico-chemical characteristics of the formulation were monitored at different time points (TABLE 14):

TABLE 14

| | Time (week) | | | | | | | | | | | | | | |
|---|---|---|---|---|---|---|---|---|---|---|---|---|---|---|---|
| | 0 | 1 | | | 3 | | | 5 | | | 9 | | 18 | | |
| Temp. ° C. | 4 | 4 | 25 | 37 | 4 | 25 | 37 | 4 | 25 | 37 | 4 | 37 | 4 | 25 | 37 |
| Size, pH, | x | | x | x | x | x | x | x | x | x | x | x | x | x | x |
| osmolality | | | | | | | | | | | | | | | |
| % | x | | x | x | x | x | x | x | x | x | x | x | x | x | x |
| encapsulation | | | | | | | | | | | | | | | |
| mRNA | x | | x | x | x | x | x | x | x | x | x | x | x | x | x |
| integrity | | | | | | | | | | | | | | | |
| (fragment | | | | | | | | | | | | | | | |
| analyser) | | | | | | | | | | | | | | | |
| Lipids | | | x | x | x | x | x | x | x | x | x | x | x | x | x |
| integrity | | | | | | | | | | | | | | | |
| (HPLC) | | | | | | | | | | | | | | | |
| EPO assay | | x | x | x | x | x | x | | | | x | | x | | |
| (number of | | (8) | (4) | (4) | (8) | (4) | (4) | | | | (8) | | (8) | | |
| mice) | | | | | | | | | | | | | | | |

RNA titration, encapsulation rate, LNP particle sizing, polydispersity indexes and the pH and osmolality of the LNP suspension were determined as described in Example 27.

For the determination of mRNA integrity in the LNPs, the mRNA was first extracted by using an extraction procedure (see below), and its integrity was determined by using a Fragment Analyzer 5200 (Agilent Technologies, Santa Clara, CA).

LNP Lipid integrity was determined by using an HPLC method with CAD detection (see below).

Extraction of mRNA from the LNPs

In brief, the procedure combines LNP Triton X100 treatment with a phenol/chloroform/isoamyl alcohol lipid extraction as described hereafter.

In a RNase-free microfuge tube, 192 μL of LNP and 8 μL of the 25% solution Triton X100 (Acros Organics) are added in order to obtain a final concentration of 1% Triton X100. The suspension is then vortexed and heated for 10 minutes at 50° C. with stirring at 700 rpm in an Eppendorf thermocycler. After cooling to room temperature, 200 μL of Phenol/Chloroform/Isoamyl alcohol 25:24:1 vol/vol (Sigma Ref. 77617) are added. The mixture is vortexed and then centrifuged for 5 min. at 12000 g by using an Eppendorf tabletop microcentrifuge. After centrifugation, the upper aqueous phase containing the mRNA is transferred to new RNAse free microfuge tube, and 200 μL of Chloroform/Isoamyl alcohol 24:1 vol/vol (Acros Organics Ref. 327155000) are added. The mixture is vortexed and then centrifuged for 5 min. at 12 000 g in the Eppendorf microcentrifuge. The upper aqueous phase is collected and mixed with 0.1 volume of 3M sodium acetate solution, pH 5.2 (Molecular biologics Ref. R1181) and 2.5 volumes of 100% ethanol to precipitate the mRNA. The mixture is stored for 12 h at −20° C. and centrifuged for 10 min at 12000 g. After centrifugation, the supernatant is discarded, and the mRNA pellet is rinsed with 200 μL of 70% ethanol. The suspension is centrifuged again for 5 minutes at 12000 g. The supernatant is eliminated and the remaining solid is dried ina SpeedVac vacuum concentrator.

Finally, the dry pellet is resuspended with 30 μL of RNAse free water and mRNA content of the resulting solution is determined by UV Spectrophotometry (absorbance at 260 nm) by using a NanoDrop 2000c UV Spectrophotometer (Thermo Scientific) and its integrity is analyzed by percentage of main peak analysis using a Fragment Analyzer 5200 (Agilent Technologies, Santa Clara, CA).

HPLC-CAD Method for LNP Lipid Content and Integrity Analysis

For the separation and analysis of the different lipid components in LNPs, a RP-HPLC method with Charged Aerosol Detection (CAD) was used. The equipment and chromatographic conditions are described in the tables below.

TABLE 15

| Equipment | UHPLC/CAD from Thermo vanquish |
|---|---|
| Solvent system A | Acetonitrile/Water (60:40) |
| | with 10 mM ammonium |
| | formate and 0.1% formic acid |
| Solvent system B | Isopropanol/acetonitrile (90:10) |
| | with 10 mM ammonium |
| | formate and 0.1% formic acid |
| Column | Waters Acquity Premier CSH |
| | C18 Column 130 Å, 1.7 |
| | μm, 2.1 mm × 50 mm |
| | (Part Number: 186009460) |
| Flow rate | 0.4 mL/min |
| Column temperature | 55° C. |
| Autosampler temperature | 5° C. |
| Injection Volume | 4 μL |
| Detector Wavelength | 210 nm |
| CAD parameters | Temperature: 50° C. |
| | Power Function: 1 |
| Run time | 25 minutes |

The column was eluted with a gradient of solvent system B in A according to the following steps:

TABLE 16

| No | Time (min) | % B | Curve |
|---|---|---|---|
| 1 | 0.0 | 40 | 5 |
| 2 | 15.0 | 70 | 5 |
| 3 | 23.0 | 99 | 5 |
| 4 | 23.1 | 40 | 5 |
| 5 | 25.0 | 40 | 5 |

By using this method, DOG-IM4, DSPC, Chol and DMG-PEG2000 are well separated on the C18-HPLC column as shown on a typical chromatogram below.

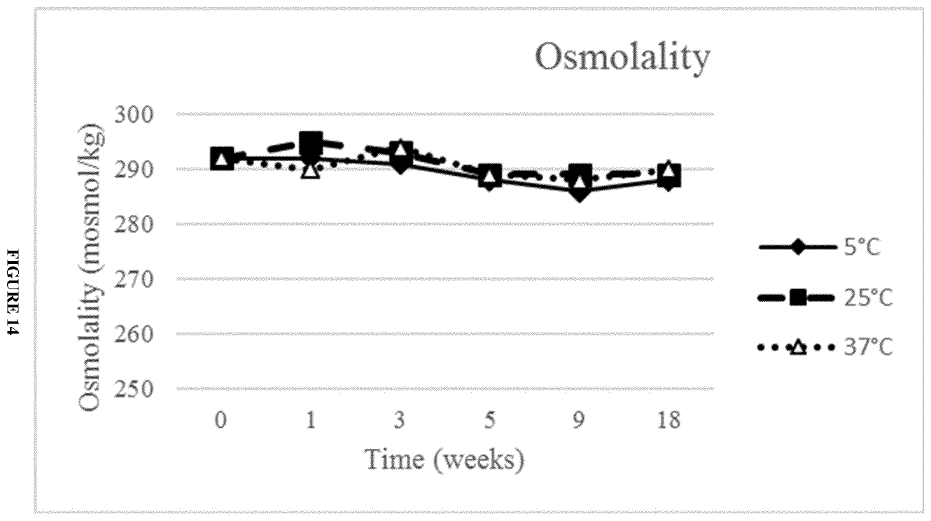

Four microliters of LNPs Lip. (IV)/DSPC containing hEPO mRNA (prepared as described in the stability study) were injected into the chromatographic system and the chromatogram recorded as a function of time is shown on FIG. 12.

Results

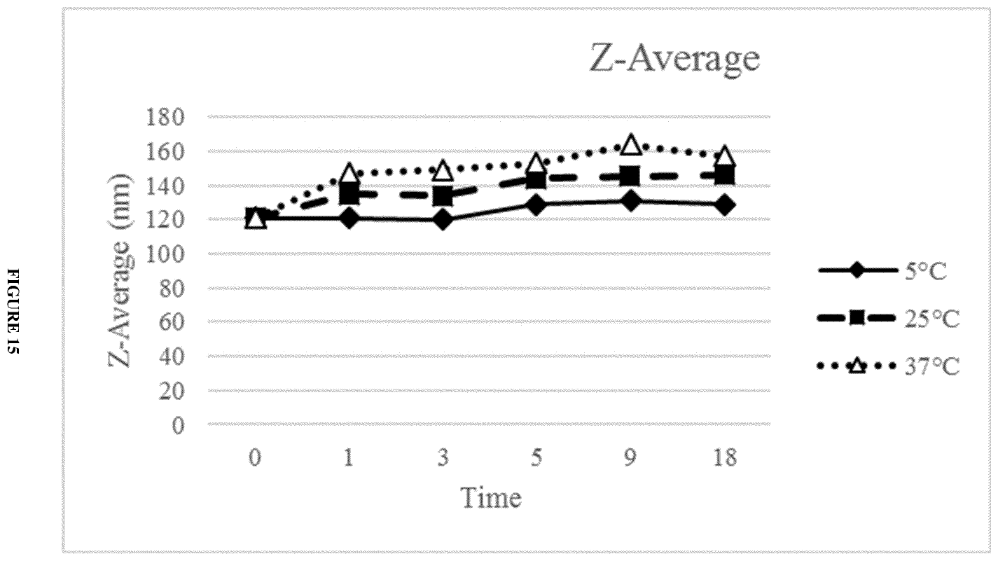

As shown on FIG. 13: the pH of the LNPs Lip. (IV)/DSPC is stable over time at different storage temperatures.

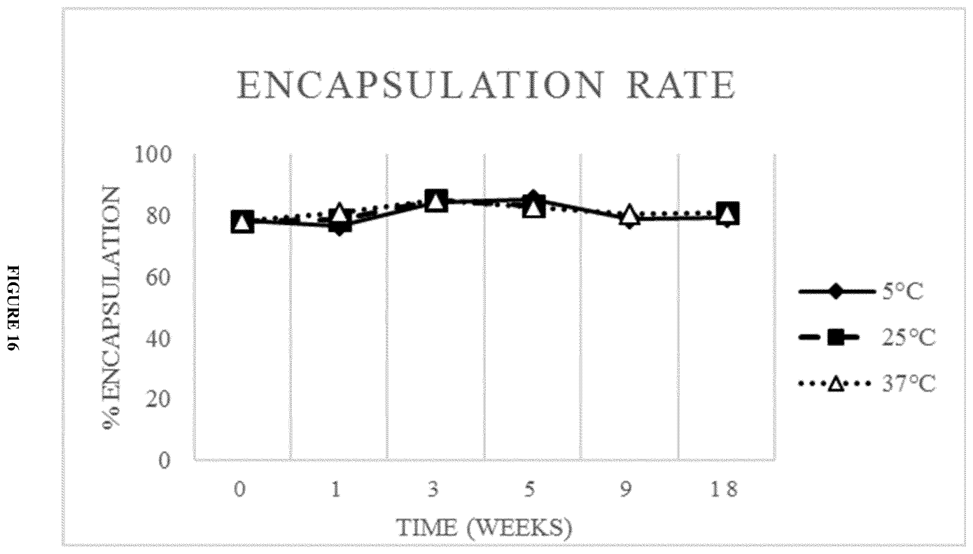

FIG. 14 shows that the osmolality of the LNPs Lip. (IV)/DSPC is stable over time at different storage temperatures.

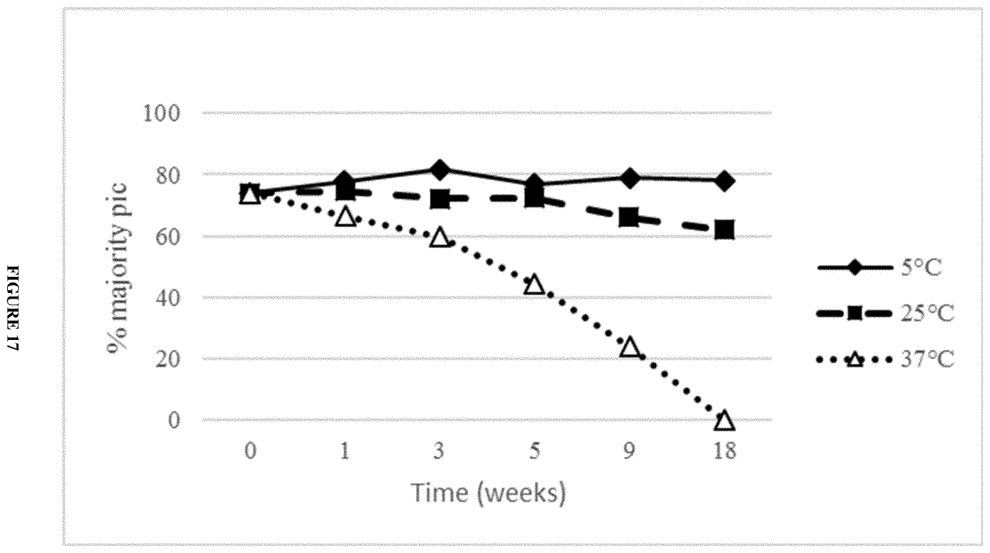

FIG. 15 shows that the particle sizes of the LNPs Lip. (IV)/DSPC are stable over time at different storage temperatures.

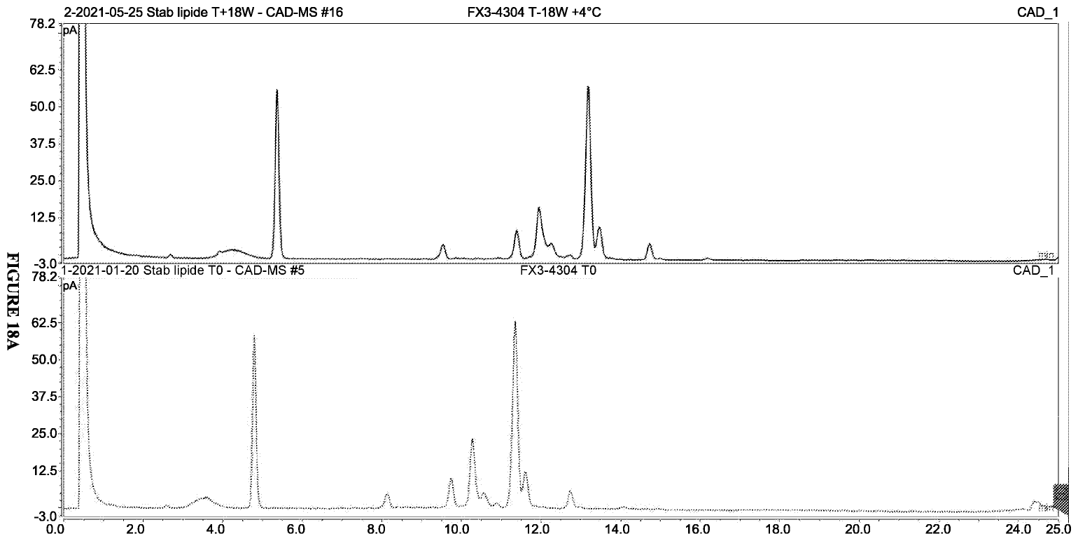

FIG. 16 shows that the mRNA encapsulation rate of the LNPs Lip. (IV)/DSPC is stable over time at different storage temperatures.

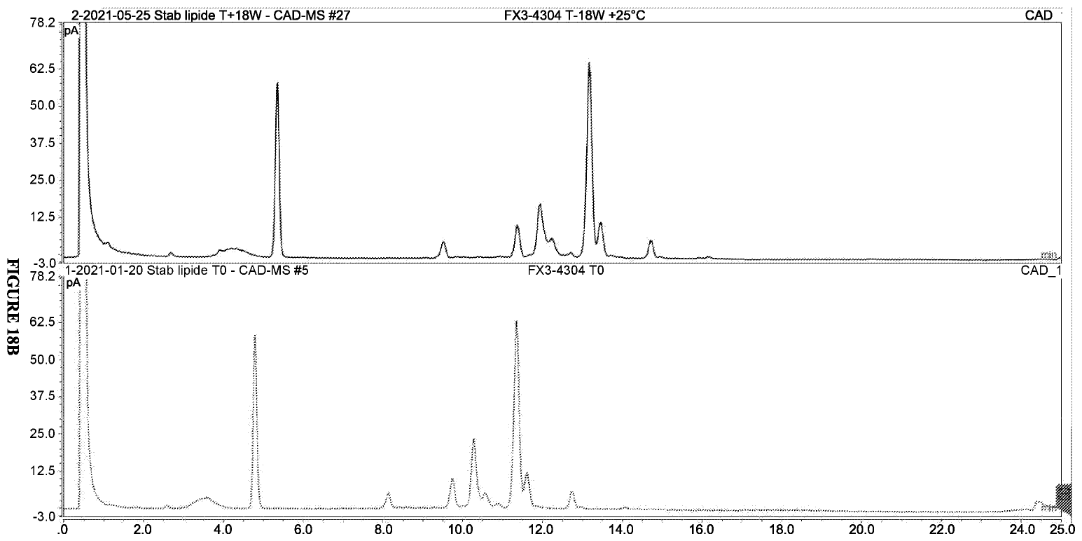

FIG. 17 shows that the mRNA integrity in the LNPs Lip. (IV)/DSPC is stable over time at different storage temperatures.

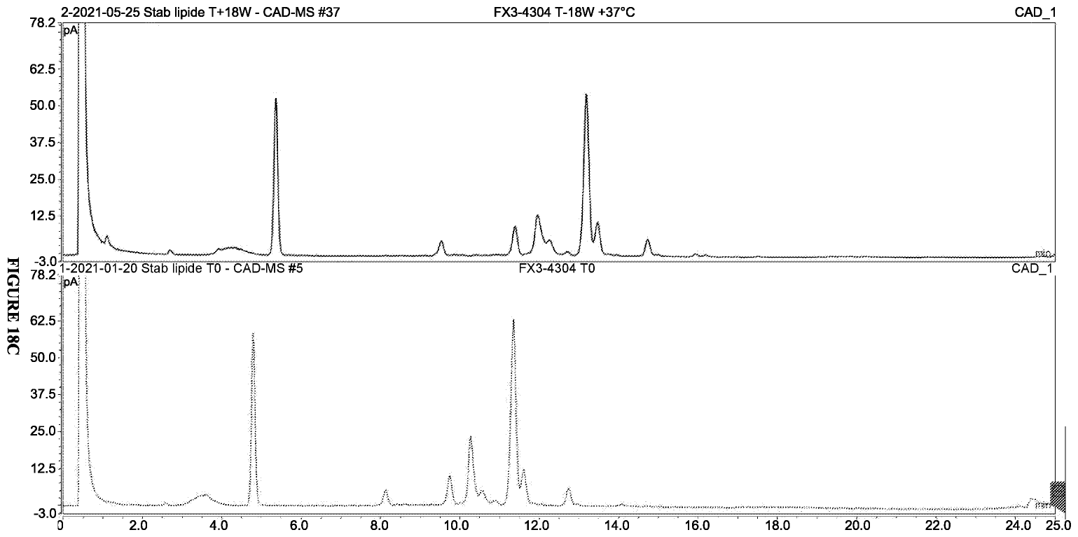

FIG. 18 shows that the lipid chromatogram for LNPs Lip. (IV)/DSPC is stable over time at different storage temperatures. On FIG. 18A, the upper panel shows that the LNPs Lip.(IV)/DSPC after 18 weeks at 4° C. The lower panel shows the same LNPs at T0. On FIG. 18B, the upper panel: shows that the LNPs Lip.(IV)/DSPC after 18 weeks at 25° C. The lower panel shows the same LNPs at T0. On FIG. 18C, the upper panel shows that the LNPs Lip.(IV)/DSPC after 18 weeks at 37° C. The lower panel shows the same LNPs at T0.

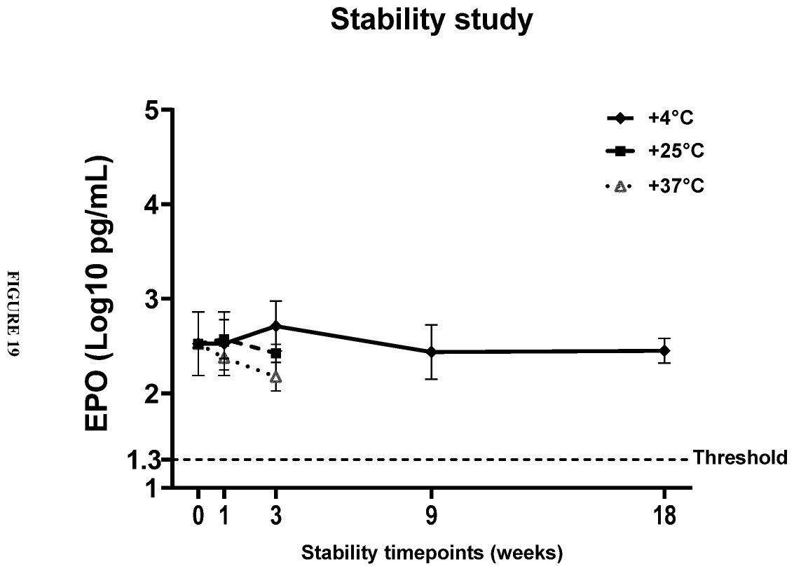

On FIG. 19, it is shown that the hEPO expression from LNPs Lip. (IV)/DSPC is stable over time at different storage temperatures.

Conclusion

LNPs Lip. (IV)/DSPC containing hEPO mRNA displayed a remarkable stability when stored as a liquid formulation in PBS at 4° C. Stability could be observed by following different physico-chemical characteristics as well as hEPO expression upon IM administration to mice (used as a potency assay). The formulation was stable for at least 18 weeks at 4° C. and for at least one week at 25° C.

### Example 36: Immunogenicity of LNPs Comprising Influenza HA mRNA (1MpU-Modified from Amptec) in Non-Human Primates

Materials and Methods:

LNPs prepared from Lip. (IV) [or respectively L319 in the control group], DSPC, Chol and DMG-PEG2000 at a molar ratio of 50:10:3.5:1.5, and 1MpU-modified HA mRNA at a N/P charge ratio=6, were prepared as described in Example 27 and used for the immunization of cynomolgus macaques.

Groups of 5 female cynomolgus macaques were immunized twice four weeks apart (D0, D28) with 50 μg of mRNA in LNPs injected IM into the biceps under a volume of 500 μl.

Blood samples were collected on D-33 (pre-immunization) and at different time intervals following immunization for antibody response analysis by HI assay (as described in Example 6).

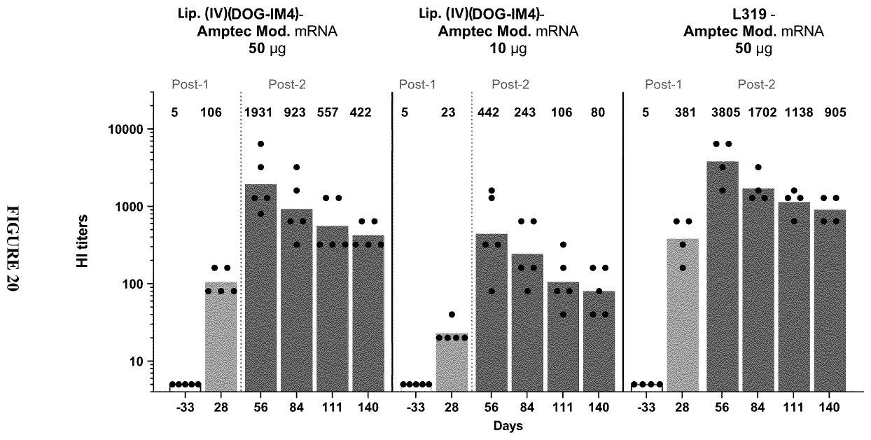

The results are shown on FIG. 20.

Conclusion

LNPs Lip. (IV)/DSPC containing 1MpU-modified HA mRNA induce strong HI responses in macaques after two IM administrations.

### Example 37: Immunogenicity of LNPs Comprising Influenza HA mRNA in Mice

The aim of the study was to evaluate the immunogenicity induced with different LNPs made with different lipidic compounds as disclosed herein and containing non-replicative mRNA encoding full-length hemagglutinin (HA) of influenza virus.

BALBc/ByJ mice (8 weeks old at D0; 8 per group) were immunized as described in Example 29 with 5 μg of natural, non-replicative mRNA encoding full-length hemagglutinin (HA) of influenza virus strain A/Netherlands/602/2009 (H1N1) formulated in 5 different lipid nanoparticles, LNPs L319, LNPs Lip. (IV) [DOG-IM4], LNPs Lip. (IX), LNPs Lip. (XII) and LNPs Lip. (XVI).

LNPs were prepared as described in Example 27 and were always composed of Ionizable lipid/DSPC/Chol/DMG-PEG2000 at a 50:10:38.5:1.5 molar ratio. Ratio N/P was always equal to 6.

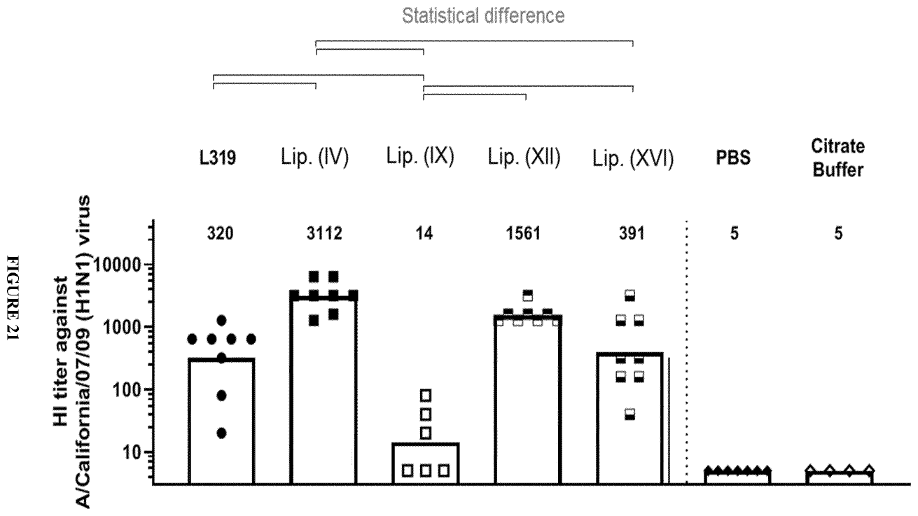

HI titers were measured 3 weeks following the second immunization as described in Example 29 and reported on FIG. 21.

1. A lipidic compound comprising at least one terminal radical of formula (I):
*—NH—CX—(NH)ₙ-A (I)
wherein:
*- represents a single bond linking said radical of formula (I), directly or not, to one C₁₀ to C₅₅ lipophilic or hydrophobic tail-group;
n is 0 or 1;
X is an oxygen or a sulfur atom, and
A represents an optionally substituted 5- or 6-membered unsaturated heterocyclic radical or 5- or 6-membered heteroaromatic ring radical, both containing at least one nitrogen atom;
or one of the pharmaceutically acceptable salts of said radical of formula (I); and said compound is in all the possible racemic, enantiomeric and diastereoisomeric isomer forms.

2. The compound of claim 1 is cationic.

3-4. (canceled)

5. The compound of claim 1 is one compound of formula (II):
R1-Z—NH—CX—(NH)ₙ-A (II)
wherein:
X, n and A are as defined in claim 1;
R1 is one C₁₀ to C₅₅ lipophilic or hydrophobic tail-group;
Z is a spacer arm having from 2 to 24, from 2 to 18, or from 4 to 12 carbon atoms in a branched or unbranched linear saturated or unsaturated hydrocarbon chain, said chain that is interrupted by one or several atoms of oxygen and/or moieties selected among —S—S—; —(O═C)—; —(C═O)—O—; —O—(O═C)—; —S—; —NH—, —NH—(O═C)—; —(O═C)—NH— and —NH—(C═O)—O and preferably by —(C═O)—O—; —O—(O═C)— and —NH—(C═O)—O— and optionally ended by an oxygen atom or a moiety selected among —NH—(O═C)—O—(O═C)—; —(C═O)—O—; and —(O═C)— to be linked to the hydrophobic tail-group;
p is 0 or 1;
or one of the pharmaceutically acceptable salts of said compound of formula (II); and any of its racemic, enantiomeric and diastereoisomeric isomer forms.

6-8. (canceled)

9. The compound of anyone of claim 1, wherein the C₁₀ to C₅₅ lipophilic or hydrophobic tail-group is an optionally substituted, branched or unbranched linear, saturated or unsaturated, C₁₀ to C₅₅ hydrocarbon radical, and which hydrocarbon skeleton that is optionally interrupted by one or several atoms of oxygen or nitrogen and/or one or several —O—CO— or —CO—O— and which one nitrogen atom if present in the skeleton can be linked, directly or not, to said radical having the formula (I) of claim 1.

10. The compound of anyone of claim 1, wherein the C₁₀ to C₅₅ lipophilic or hydrophobic tail-group is selected from the group consisting of: 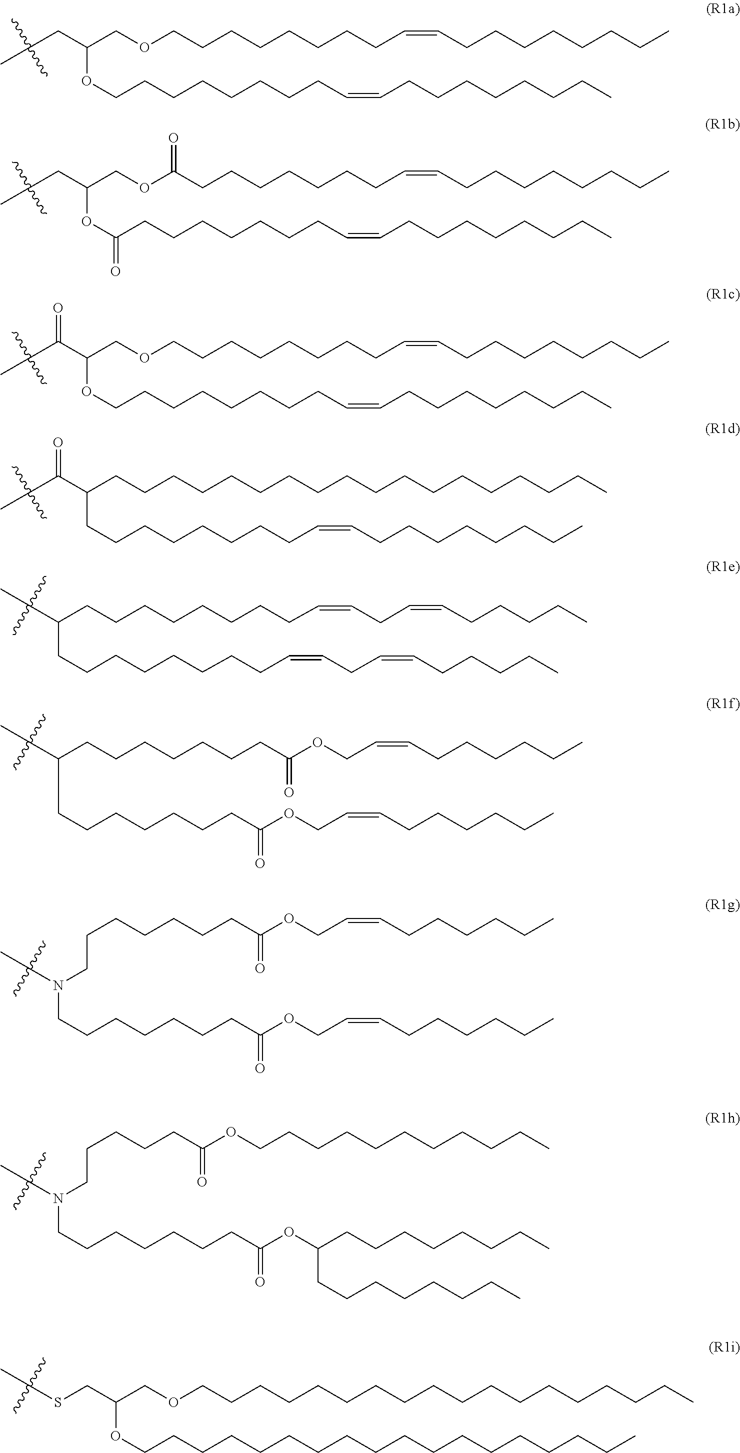 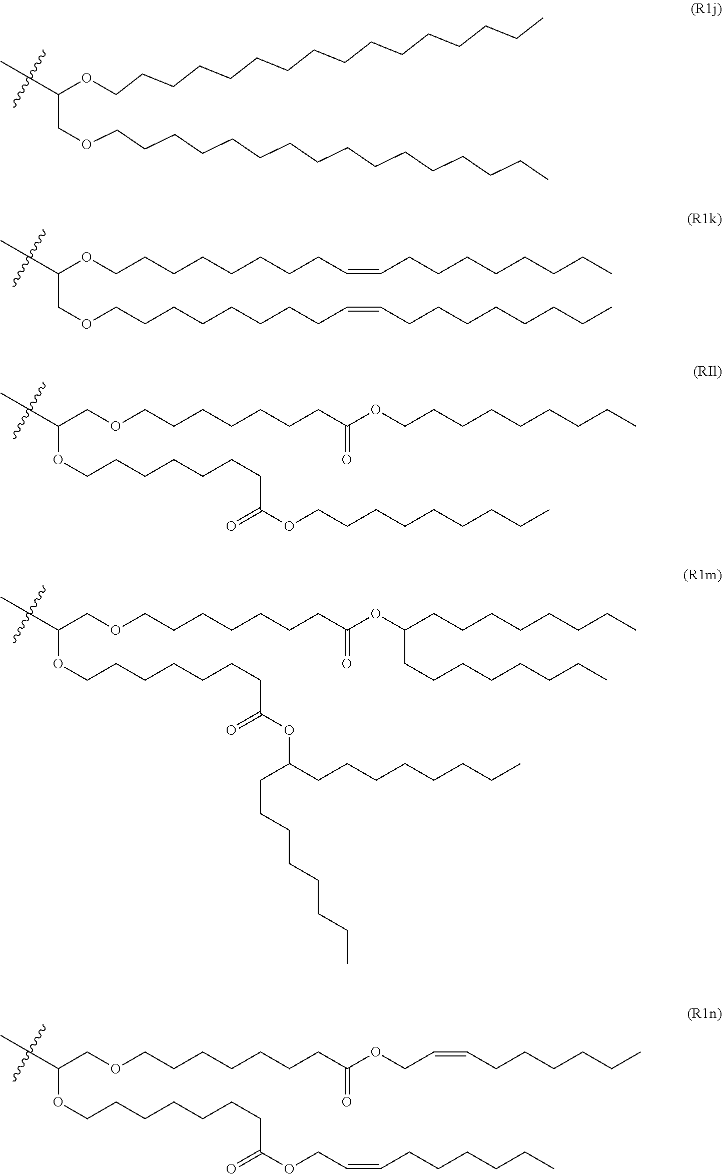 [SMILES: *C(COCCCCCCCCCCCCCCCC)OCCCCCCCCCCCCCCCC ; *C(COCCCCCCCC/C=C\CCCCCCCC)OCCCCCCCC/C=C\CCCCCCCC ; *C(COCCCCCCCC(=O)OCCCCCCCCC)OCCCCCCCC(=O)OCCCCCCCCC ; *C(COCCCCCCCC(=O)OC(CCCCCCCC)CCCCCCCC)OCCCCCCCC(=O)OC(CCCCCCCC)CCCCCCCC ; *C(COCCCCCCCC(=O)OC/C=C\CCCCCC)OCCCCCCCC(=O)OC/C=C\CCCCCC] 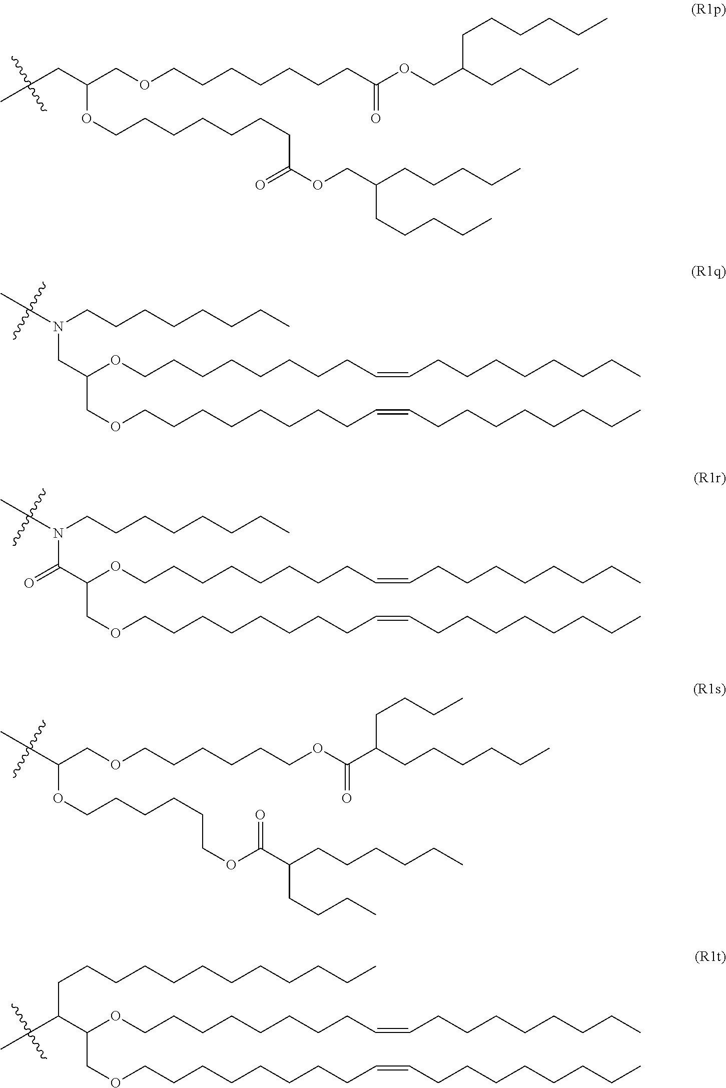 [SMILES: *CC(COCCCCCCCC(=O)OCC(CCCC)CCCCCC)OCCCCCCCC(=O)OCC(CCCCC)CCCCC ; *N(CCCCCCCC)CC(COCCCCCCCC/C=C\CCCCCCCC)OCCCCCCCC/C=C\CCCCCCCC ; *N(CCCCCCCC)C(=O)C(COCCCCCCCC/C=C\CCCCCCCC)OCCCCCCCC/C=C\CCCCCCCC ; *C(COCCCCCCOC(=O)C(CCCC)CCCCCC)OCCCCCCOC(=O)C(CCCC)CCCCCC ; *C(CCCCCCCCCCCC)C(COCCCCCCCC/C=C\CCCCCCCC)OCCCCCCCC/C=C\CCCCCCCC] 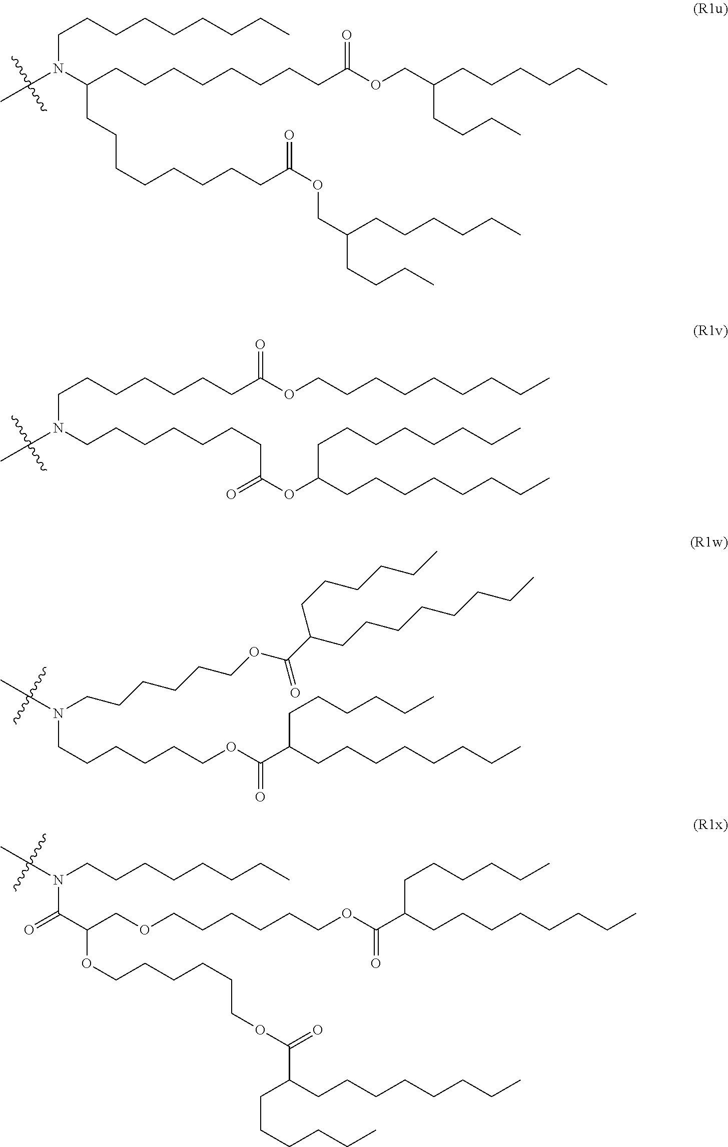 [SMILES: *N(CCCCCCCCC)C(CCCCCCCCC(=O)OCC(CCCC)CCCCCC)CCCCCCCCC(=O)OCC(CCCC)CCCCCC ; *N(CCCCCCCC(=O)OCCCCCCCCC)CCCCCCCC(=O)OC(CCCCCCCC)CCCCCCCC ; *N(CCCCCCOC(=O)C(CCCCCC)CCCCCCCC)CCCCCCOC(=O)C(CCCCCC)CCCCCCCC ; *N(CCCCCCCC)C(=O)C(COCCCCCCOC(=O)C(CCCCCC)CCCCCCCC)OCCCCCCOC(=O)C(CCCCCC)CCCCCCCC] 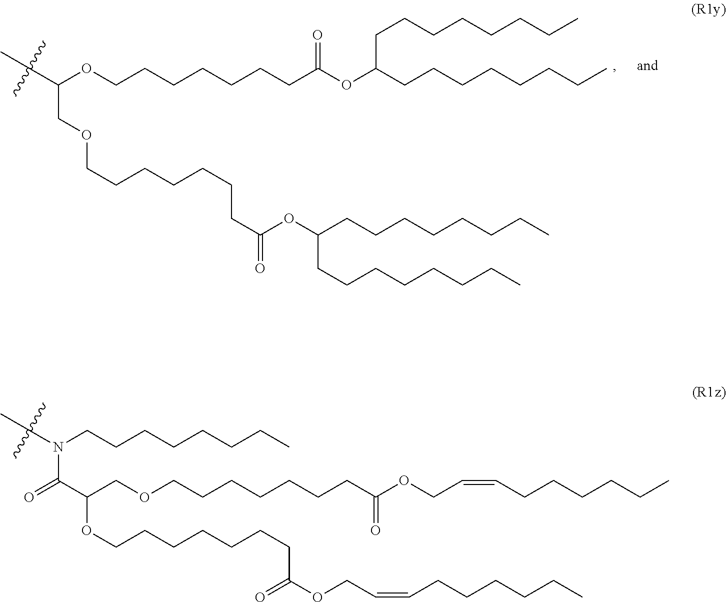 [SMILES: *C(COCCCCCCCC(=O)OC(CCCCCCCC)CCCCCCCC)OCCCCCCCC(=O)OC(CCCCCCCC)CCCCCCCC ; *N(CCCCCCCC)C(=O)C(COCCCCCCCC(=O)OC/C=C\CCCCCC)OCCCCCCCC(=O)OC/C=C\CCCCCC]
and preferably is (R1a) or (R1b).

11-13. (canceled)

14. The compound of claim 5, wherein the spacer arm Z is selected from the group consisting of:

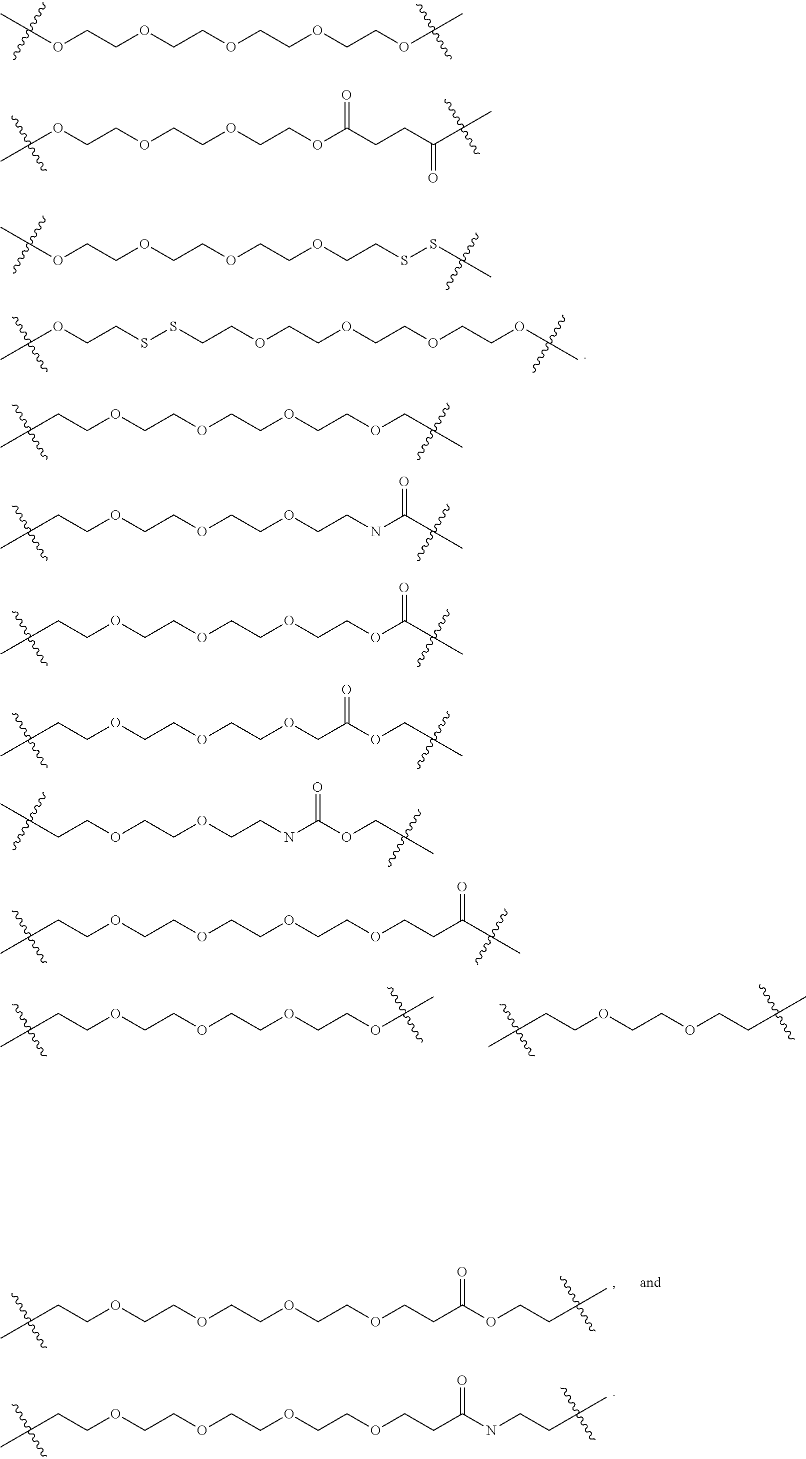

15. The compound of claim 5, wherein the spacer arm Z comprises from 1 to 24, from 2 to 15, or from 3 to 12 ethylene oxide units and further incorporates at least one NH—(C═O)—O—.

16. The compound of claim 1 is selected from a group consisting of: 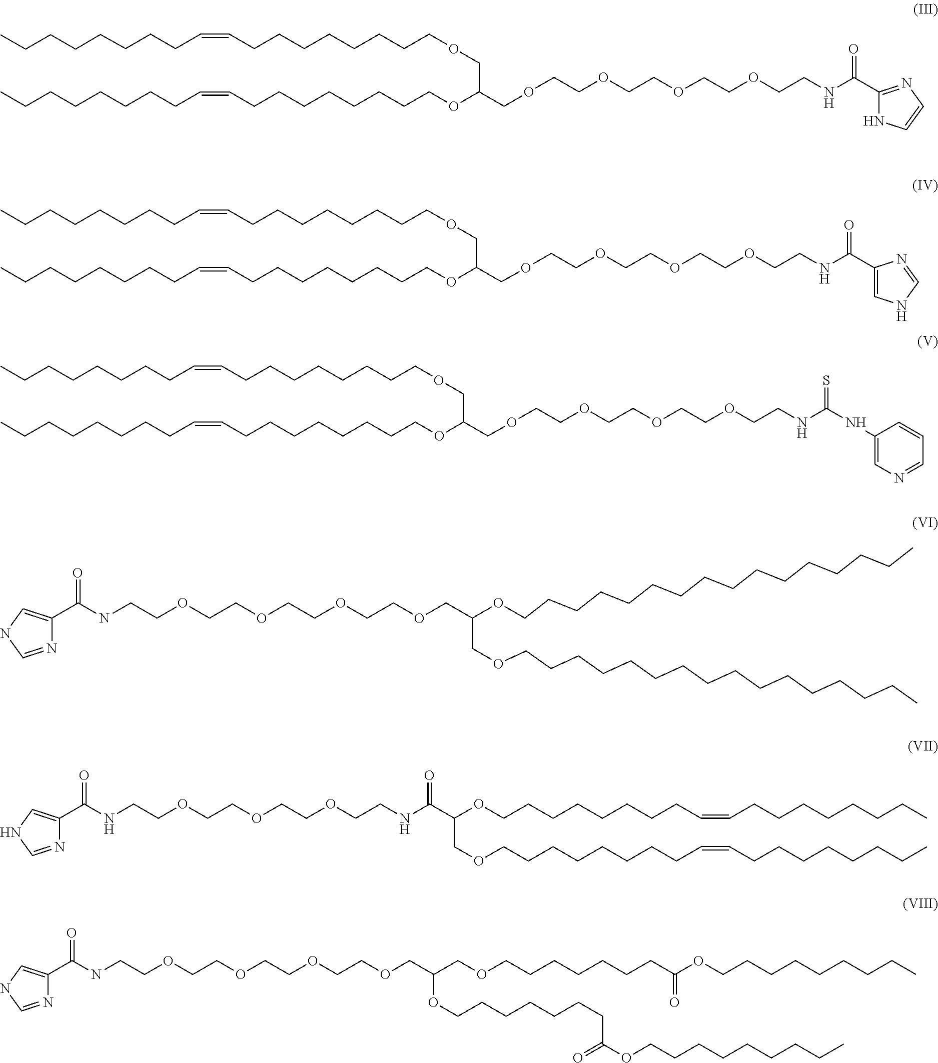 [SMILES: CCCCCCCC/C=C\CCCCCCCCOCC(COCCOCCOCCOCCNC(=O)c1ncc[nH]1)OCCCCCCCC/C=C\CCCCCCCC ; CCCCCCCC/C=C\CCCCCCCCOCC(COCCOCCOCCOCCNC(=O)c1c[nH]cn1)OCCCCCCCC/C=C\CCCCCCCC ; CCCCCCCC/C=C\CCCCCCCCOCC(COCCOCCOCCOCCNC(=S)Nc1cccnc1)OCCCCCCCC/C=C\CCCCCCCC ; CCCCCCCCCCCCCCCCOCC(COCCOCCOCCOCCNC(=O)c1c[nH]cn1)OCCCCCCCCCCCCCCCC ; CCCCCCCC/C=C\CCCCCCCCOCC(OCCCCCCCC/C=C\CCCCCCCC)C(=O)NCCOCCOCCOCCNC(=O)c1c[nH]cn1 ; CCCCCCCCCOC(=O)CCCCCCCOCC(COCCOCCOCCOCCNC(=O)c1c[nH]cn1)OCCCCCCCC(=O)OCCCCCCCCC] 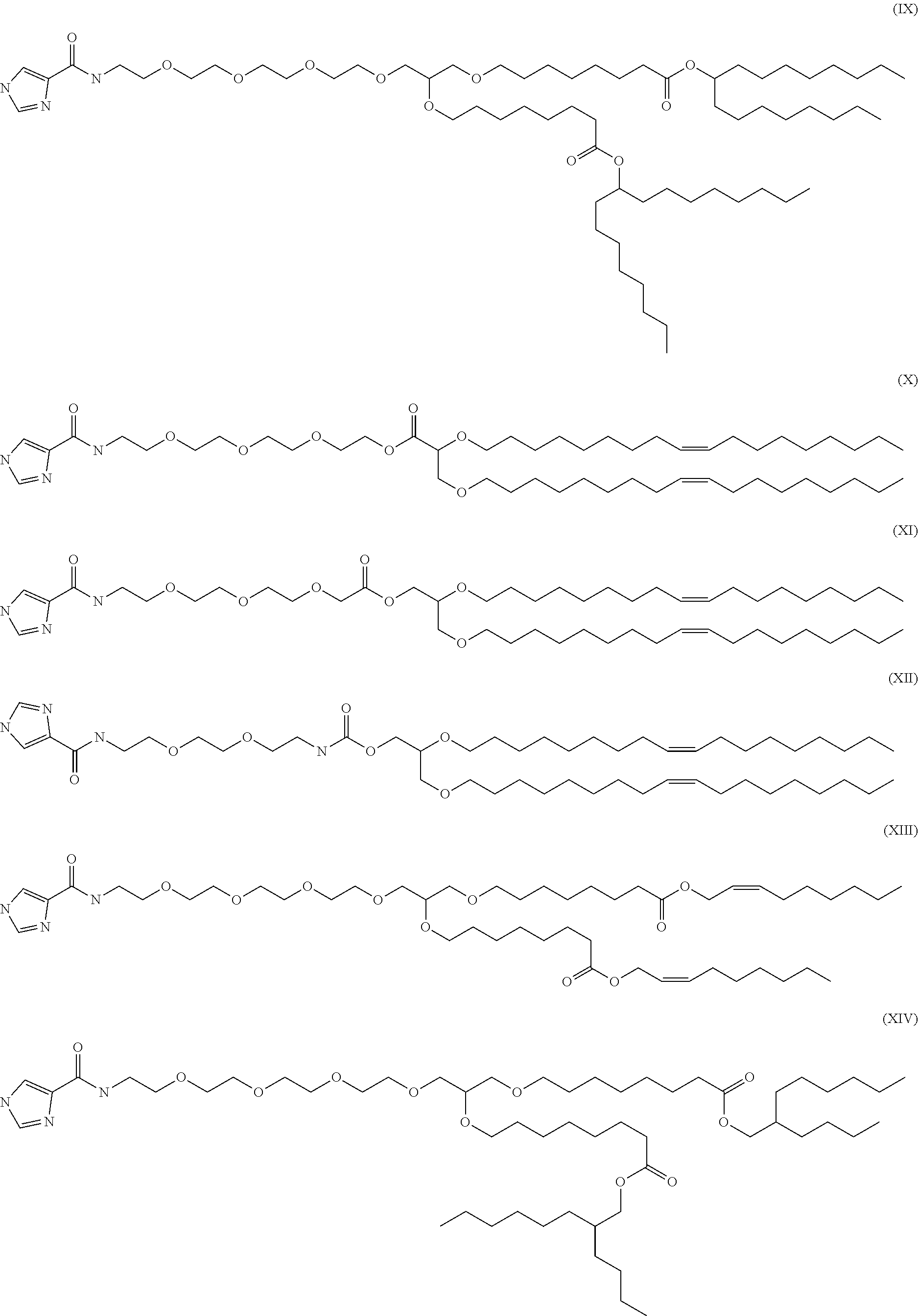 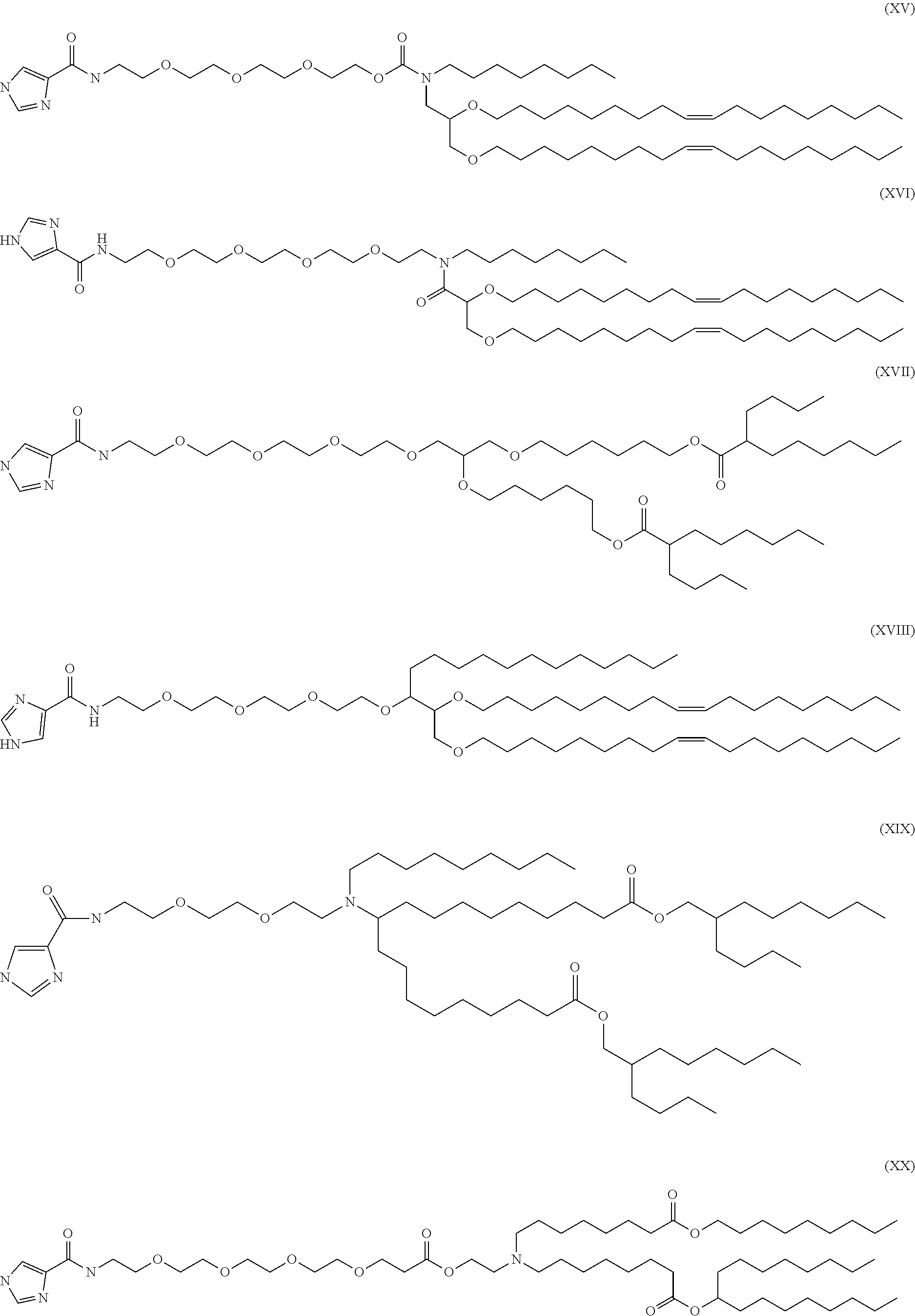 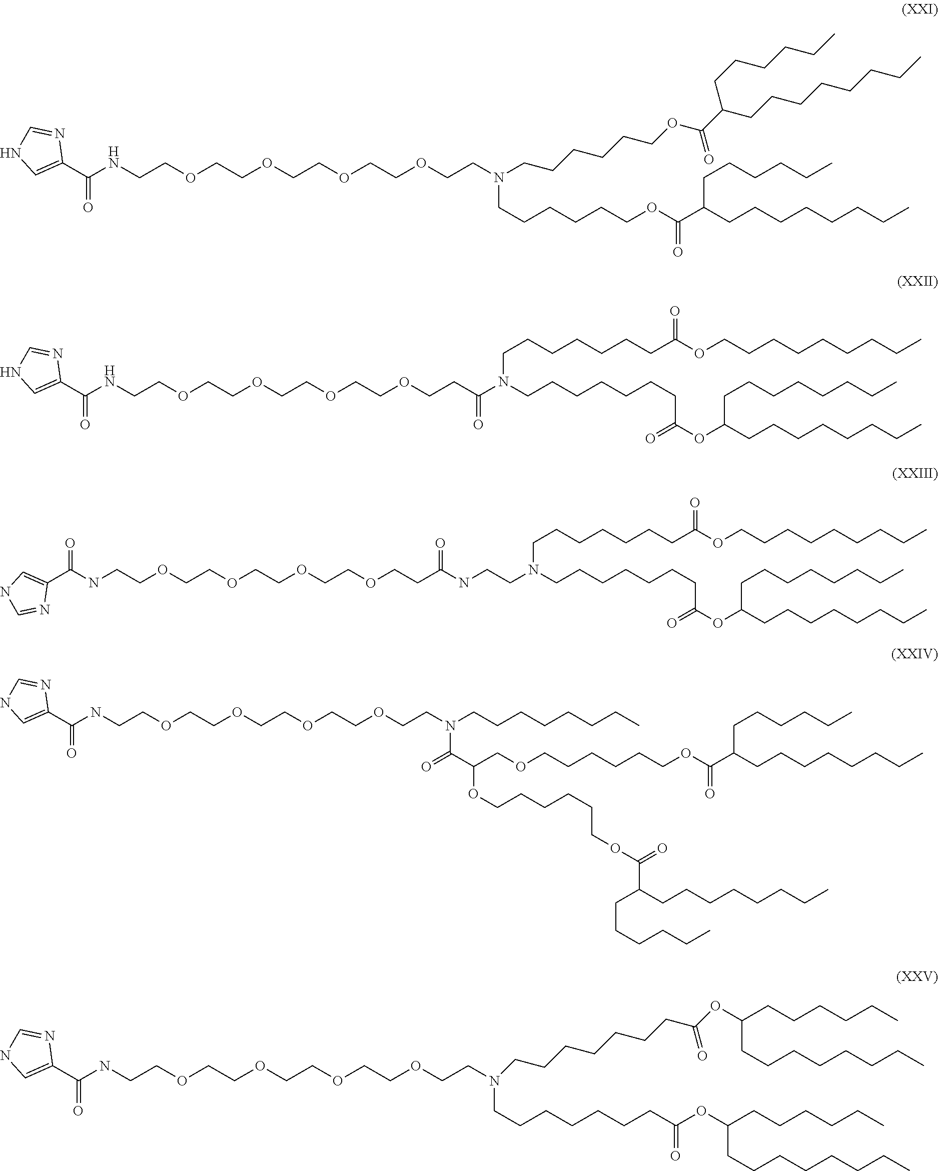 [SMILES: CCCCCCCCC(CCCCCC)C(=O)OCCCCCCN(CCCCCCOC(=O)C(CCCCCC)CCCCCCCC)CCOCCOCCOCCOCCNC(=O)c1c[nH]cn1 ; CCCCCCCCCOC(=O)CCCCCCCN(CCCCCCCC(=O)OC(CCCCCCCC)CCCCCCCC)C(=O)CCOCCOCCOCCOCCNC(=O)c1c[nH]cn1 ; CCCCCCCCCOC(=O)CCCCCCCN(CCCCCCCC(=O)OC(CCCCCCCC)CCCCCCCC)CCNC(=O)CCOCCOCCOCCOCCNC(=O)c1c[nH]cn1 ; CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCNC(=O)c1c[nH]cn1 ; CCCCCCCCC(CCCCCC)OC(=O)CCCCCCCN(CCCCCCCC(=O)OC(CCCCCC)CCCCCCCC)CCOCCOCCOCCOCCNC(=O)c1c[nH]cn1] 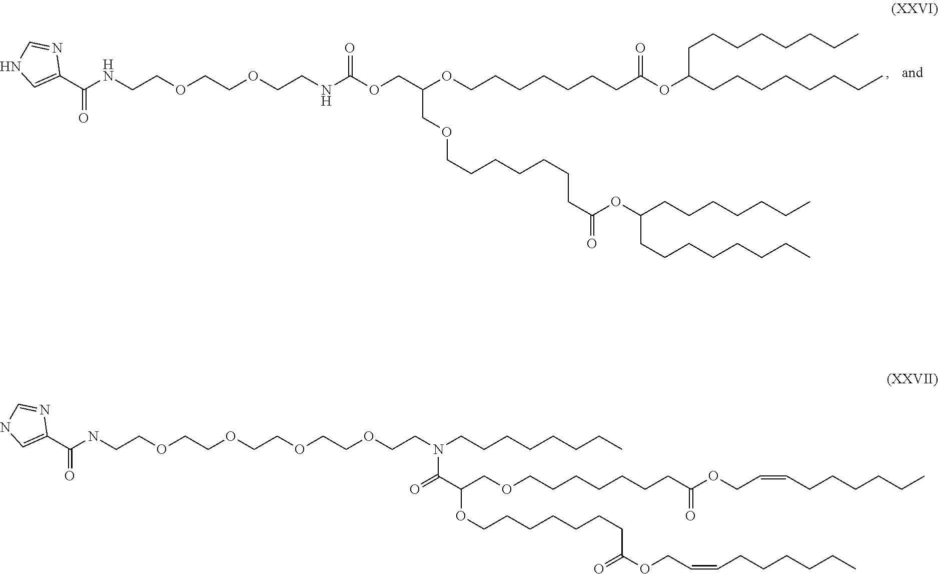 [SMILES: CCCCCCCCC(CCCCCCC)OC(=O)CCCCCCCOCC(COC(=O)NCCOCCOCCNC(=O)c1c[nH]cn1)OCCCCCCCC(=O)OC(CCCCCCCC)CCCCCCCC ; CCCCCC/C=C\COC(=O)CCCCCCCOCC(OCCCCCCCC(=O)OC/C=C\CCCCCC)C(=O)N(CCCCCCCC)CCOCCOCCOCCOCCNC(=O)c1c[nH]cn1]
and their pharmaceutically acceptable salts, and their racemic, enantiomeric and diastereoisomeric isomer forms.

17. The compound of claim 16 is the compound of formula (IV): 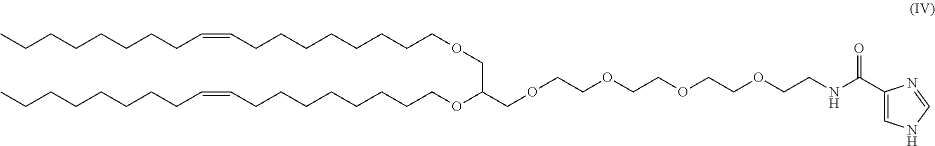 [SMILES: CCCCCCCC/C=C\CCCCCCCCOCC(COCCOCCOCCOCCNC(=O)c1c[nH]cn1)OCCCCCCCC/C=C\CCCCCCCC]
and theirs salts or racemic, enantiomeric and diastereoisomeric isomer forms.

18. A composition comprising at least one lipidic compound of claim 1 and at least one lipid selected from the group consisting of neutral lipids, steroid alcohols or esters thereof, and PEGylated lipids.

19. The composition of claim 18, wherein the neutral lipid is selected from the group consisting of phosphatidylcholines, such as DSPC, DPPC, DMPC, POPC, DOPC; phosphatidylethanolamines, such as DOPE, DPPE, DMPE, DSPE, DLPE; DPPS; DOPG; sphingomyelins; and ceramides.

20-21. (canceled)

22. The composition a of claim 18, comprising at least one neutral lipid, at least one steroid alcohol or ester thereof, and at least one PEGylated lipid, and wherein said lipidic compound, said neutral lipid, said steroid alcohol or ester thereof and said PEGylated lipid are present in a molar amount of about 30% to about 70% of lipidic compound, of about 0% to about 50% of neutral lipid, of 20% to about 50% of steroid alcohol or ester thereof, and of about 1% to about 15% of PEGylated, relative to the total amount of lipid and lipidic compound.

23. The composition of claim 18, further comprising at least one nucleic acid.

24. The composition of claim 23, wherein the at least one nucleic acid encodes for an antigen.

25. A lipid nanoparticle comprising at least one lipidic compound of claim 1 and at least one nucleic acid.

26. A lipid nanoparticle of claim 25, further comprising at least one lipid selected from the group consisting of neutral lipids, steroid alcohols or esters thereof, and PEGylated lipids.

27. A pharmaceutical composition comprising (i) at least one nucleic acid and at least one lipidic compound of claim 1, or (ii) at least one nucleic acid and at least one composition comprising at least one lipidic compound of claim 1 and at least one lipid selected from the group consisting of neutral lipids, steroid alcohols or esters thereof, and PEGylated lipids, or (iii) at least one lipid nanoparticle comprising at least one lipidic compound of claim 1 and at least one nucleic acid.

28. An immunogenic composition comprising (i) at least one nucleic acid encoding for an antigen and at least one lipidic compound of claim 1, or (ii) at least one nucleic acid encoding for an antigen and at least one composition comprising at least one lipidic compound of claim 1 and at least one lipid selected from the group consisting of neutral lipids, steroid alcohols or esters thereof, and PEGylated lipids, or (iii) at least one lipid nanoparticle comprising at least one lipidic compound of claim 1 and at least one nucleic acid, wherein the nucleic acid encodes for at least one antigen.

29. A composition comprising (i) at least one nucleic acid and at least one lipidic compound of claim 1, or (ii) at least one nucleic acid encoding for an antigen and at least one composition according comprising at least one lipidic compound of claim 1 and at least one lipid selected from the group consisting of neutral lipids, steroid alcohols or esters thereof, and PEGylated lipids, or (iii) at least one lipid nanoparticle comprising at least one lipidic compound of claim 1 and at least one nucleic acid, for use as a medicament.

30. A composition comprising (i) at least one nucleic acid and at least one lipidic compound of claim 1, or (ii) at least one nucleic acid encoding for an antigen and at least one composition comprising at least one lipidic compound of claim 1 and at least one lipid selected from the group consisting of neutral lipids, steroid alcohols or esters thereof, and PEGylated lipids, or (iii) at least one lipid nanoparticle comprising at least one lipidic compound of claim 1 and at least one nucleic acid, for use in a therapeutic method for preventing and/or treating a disease selected in a group consisting of infectious diseases, allergies, autoimmune diseases, rare blood disorders, rare metabolic diseases, rare neurologic diseases, and tumour or cancer diseases.
